SEQUENCE LISTING (23 sequences)

SEQ ID NO: 1 - DNA, 3464 bp, Homo sapiens
  source 1..3464: mol_type = other DNA; note = misc_feature - cDNA of 191P4D12
  CDS 264..1796: protein_id = 2; translation = MPLSLGAEMWGPEAWLLLLLLLASFTGRCPAGELETSDVVTVVL GQDAKLPCFYRGDSGEQVGQVAWARVDAGEGAQELALLHSKYGLHVSPAYEGRVEQPP PPRNPLDGSVLLRNAVQADEGEYECRVSTFPAGSFQARLRLRVLVPPLPSLNPGPALE EGQGLTLAASCTAEGSPAPSVTWDTEVKGTTSSRSFKHSRSAAVTSEFHLVPSRSMNG QPLTCVVSHPGLLQDQRITHILHVSFLAEASVRGLEDQNLWHIGREGAMLKCLSEGQP PPSYNWTRLDGPLPSGVRVDGDTLGFPPLTTEHSGIYVCHVSNEFSSRDSQVTVDVLD PQEDSGKQVDLVSASVVVVGVIAALLFCLLVVVVVLMSRYHRRKAQQMTQKYEEELTL TRENSIRRLHSHHTDPRSQPEESVGLRAEGHPDSLKDNSSCSVMSEEPEGRSYSTLTT VREIETQTELLSPGSGRAEEEEDQDEGIKQAMNHFVQENGTLRAKPTGNGIYINGRGH LV
ggccgtcgttgttggccacagcgtgggaagcagctctgggggagctcggagctcccgatcacggcttcttgggggtagctacggctgggtgtgtagaacggggccggggctggggctgggtcccctagtggagacccaagtgcgagaggcaagaactctgcagcttcctgccttctgggtcagttccttattcaagtctgcagccggctcccagggagatctcggtggaacttcagaaacgctgggcagtctgcctttcaaccatgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctgacccaggcctgcctcccttccctaggcctggctccttctgttgacatgggagattttagctcatcttgggggcctccttaaacacccccatttcttgcggaagatgctccccatcccactgactgcttgacctttacctccaacccttctgttcatcgggagggctccaccaattgagtctctcccaccatgcatgcaggtcactgtgtgtgtgcatgtgtgcctgtgtgagtgttgactgactgtgtgtgtgtggaggggtgactgtccgtggaggggtgactgtgtccgtggtgtgtattatgctgtcatatcagagtcaagtgaactgtggtgtatgtgccacgggatttgagtggttgcgtgggcaacactgtcagggtttggcgtgtgtgtcatgtggctgtgtgtgacctctgcctgaaaaagcaggtattttctcagaccccagagcagtattaatgatgcagaggttggaggagagaggtggagactgtggctcagacccaggtgtgcgggcatagctggagctggaatctgcctccggtgtgagggaacctgtctcctaccacttcggagccatgggggcaagtgtgaagcagccagtccctgggtcagccagaggcttgaactgttacagaagccctctgccctctggtggcctctgggcctgctgcatgtacatattttctgtaaatatacatgcgccgggagcttcttgcaggaatactgctccgaatcacttttaatttttttcttttttttttcttgccctttccattagttgtattttttatttatttttatttttatttttttttagagatggagtctcactatgttgctcaggctggccttgaactcctgggctcaagcaatcctcctgcctcagcctccctagtagctgggactttaagtgtacaccactgtgcctgctttgaatcctttacgaagagaaaaaaaaaattaaagaaagcctttagatttatccaatgtttactactgggattgcttaaagtgaggcccctccaacaccagggggttaattcctgtgattgtgaaaggggctacttccaaggcatcttcatgcaggcagccccttgggagggcacctgagagctggtagagtctgaaattagggatgtgagcctcgtggttactgagtaaggtaaaattgcatccaccattgtttgtgataccttagggaattgcttggacctggtgacaagggctcctgttcaatagtggtgttggggagagagagagcagtgattatagaccgagagagtaggagttgaggtgaggtgaaggaggtgctgggggtgagaatgtcgcctttccccctgggttttggatcactaattcaaggctcttctggatgtttctctgggttggggctggagttcaatgaggtttatttttagctggcccacccagatacactcagccagaatacctagatttagtacccaaactcttcttagtctgaaatctgctggatttctggcctaagggagaggctcccatccttcgttccccagccagcctaggacttcgaatgtggagcctgaagatctaagatcctaacatgtacattttatgtaaatatgtgcatatttgtacataaaatgatattctgtttttaaataaacagacaaaacttgaaaaa

SEQ ID NO: 2 - protein, 510 aa, Homo sapiens
  source 1..510: note = misc_feature - 191P4D12
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 3 - DNA, 1432 bp, Homo sapiens
  source 1..1432: mol_type = other DNA; note = misc_feature - cDNA of Ha22-2(2,4)6.1 heavy chain
  CDS 32..1432: protein_id = 4; translation = MELGLCWVFLVAILEGVQCEVQLVESGGGLVQPGGSLRLSCAAS GFTFSSYNMNWVRQAPGKGLEWVSYISSSSSTIYYADSVKGRFTISRDNAKNSLSLQM NSLRDEDTAVYYCARAYYYGMDVWGQGTTVTVSSASTKGPSVFPLAPSSKSTSGGTAA LGCLVKDYFPEPVTVSWNSGALTSGVHTFPAVLQSSGLYSLSSVVTVPSSSLGTQTYI CNVNHKPSNTKVDKRVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLN GKEYKCKVSNKALPAPIEKTISKAKGQPREPQVYTLPPSREEMTKNQVSLTCLVKGFY PSDIAVEWESNGQPENNYKTTPPVLDSDGSFFLYSKLTVDKSRWQQGNVFSCSVMHEA LHNHYTQKSLSLSPGK
ggtgatcagcactgaacacagaggactcaccatggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagctataacatgaactgggtccgccaggctccagggaaggggctggagtgggtttcatacattagtagtagtagtagtaccatatactacgcagactctgtgaagggccgattcaccatctccagagacaatgccaagaactcactgtctctgcaaatgaacagcctgagagacgaggacacggctgtgtattactgtgcgagagcatactactacggtatggacgtctggggccaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 4 - protein, 466 aa, Homo sapiens
  source 1..466: note = misc_feature - Ha22-2(2,4)6.1 heavy chain
MELGLCWVFLVAILEGVQCEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 735 bp, Homo sapiens
  source 1..735: mol_type = other DNA; note = misc_feature - cDNA of Ha22-2(2,4)6.1 light chain
  CDS 25..735: protein_id = 6; translation = MDMRVPAQLLGLLLLWFPGSRCDIQMTQSPSSVSASVGDRVTIT CRASQGISGWLAWYQQKPGKAPKFLIYAASTLQSGVPSRFSGSGSGTDFTLTISSLQP EDFATYYCQQANSFPPTFGGGTKVEIKRTVAAPSVFIFPPSDEQLKSGTASVVCLLNN FYPREAKVQWKVDNALQSGNSQESVTEQDSKDSTYSLSSTLTLSKADYEKHKVYACEV THQGLSSPVTKSFNRGEC
agtcagacccagtcaggacacagcatggacatgagggtccccgctcagctcctggggctcctgctgctctggttcccaggttccagatgcgacatccagatgacccagtctccatcttccgtgtctgcatctgttggagacagagtcaccatcacttgtcgggcgagtcagggtattagcggctggttagcctggtatcagcagaaaccagggaaagcccctaagttcctgatctatgctgcatccactttgcaaagtggggtcccatcaaggttcagcggcagtggatctgggacagatttcactctcaccatcagcagcctgcagcctgaagattttgcaacttactattgtcaacaggctaacagtttccctcccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 6 - protein, 236 aa, Homo sapiens
  source 1..236: note = misc_feature - Ha22-2(2,4)6.1 light chain
MDMRVPAQLLGLLLLWFPGSRCDIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 466 aa, Homo sapiens; identical to SEQ ID NO: 4
  source 1..466: note = misc_feature - Ha22-2(2,4)6.1 heavy chain

SEQ ID NO: 8 - protein, 236 aa, Homo sapiens; identical to SEQ ID NO: 6
  source 1..236: note = misc_feature - Ha22-2(2,4)6.1 light chain

SEQ ID NO: 9 - protein, 5 aa, Homo sapiens
  source 1..5: note = CDR1 of heavy chain
SYNMN

SEQ ID NO: 10 - protein, 17 aa, Homo sapiens
  source 1..17: note = CDR2 of heavy chain
YISSSSSTIYYADSVKG

SEQ ID NO: 11 - protein, 8 aa, Homo sapiens
  source 1..8: note = CDR3 of heavy chain
AYYYGMDV

SEQ ID NO: 12 - protein, 11 aa, Homo sapiens
  source 1..11: note = CDR1 of light chain
RASQGISGWLA

SEQ ID NO: 13 - protein, 7 aa, Homo sapiens
  source 1..7: note = CDR2 of light chain
AASTLQS

SEQ ID NO: 14 - protein, 9 aa, Homo sapiens
  source 1..9: note = CDR3 of light chain
QQANSFPPT

SEQ ID NO: 15 - protein, 4 aa, synthetic construct
  source 1..4: note = linker
GFLG

SEQ ID NO: 16 - protein, 8 aa, synthetic construct
  source 1..8: note = VH CDR1 according to IMGT
GFTFSSYN

SEQ ID NO: 17 - protein, 8 aa, synthetic construct
  source 1..8: note = VH CDR2 according to IMGT
ISSSSSTI

SEQ ID NO: 18 - protein, 10 aa, synthetic construct
  source 1..10: note = VH CDR3 according to IMGT
ARAYYYGMDV

SEQ ID NO: 19 - protein, 6 aa, synthetic construct
  source 1..6: note = VL CDR1 according to IMGT
QGISGW

SEQ ID NO: 20 - skipped

SEQ ID NO: 21 - protein, 9 aa, synthetic construct
  source 1..9: note = VL CDR3 according to IMGT
QQANSFPPT

SEQ ID NO: 22 - protein, 117 aa, synthetic construct
  source 1..117: note = heavy chain variable region (VH), the 20th amino acid (glutamic acid) to the 136th amino acid (serine) of SEQ ID NO:7
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 108 aa, synthetic construct
  source 1..108: note = light chain variable region (VL), the 23rd amino acid (aspartic acid) to the 130th amino acid (arginine) of SEQ ID NO:8
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKR

(19) United States
(12) Patent Application Publication
(10) Pub. No.: US 2025/0064956 A1
(43) Pub. Date: Feb. 27, 2025

(54) METHODS FOR TREATING MUSCLE INVASIVE UROTHELIAL CANCER OR MUSCLE INVASIVE BLADDER CANCER WITH ANTIBODY DRUG CONJUGATES (ADC) THAT BIND TO 191P4D12 PROTEINS

(71) Applicants: AGENSYS, INC., Northbrook, IL (US); SEAGEN INC., Bothell, WA (US)

(72) Inventors: Anne-Sophie CARRET, Kirkland, WA (US); Oyewale O. ABIDOYE, Atherton, CA (US)

(73) Assignees: AGENSYS, INC., Northbrook, IL (US); SEAGEN INC., Bothell, WA (US)

(21) Appl. No.: 18/724,594

(22) PCT Filed: Jan. 4, 2023
(86) PCT No.: PCT/US2023/060062

§ 371 Date: Jun. 26, 2024

Related U.S. Application Data
(60) Provisional application No. 63/304,136, filed on Jan. 28, 2022.
(60) Provisional application No. 63/296,831, filed on Jan. 5, 2022.

(51) Int. Cl. A61K 47/68; A61K 47/22; A61K 47/26; A61P 35/00

(52) U.S. Cl. CPC A61K 47/68031; A61K 47/22; A61K 47/26; A61K 47/6849; A61K 47/6889; A61P 35/00

(57) ABSTRACT

Provided herein are methods for treating urothelial or bladder cancers with antibody drug conjugates (ADC) that bind to 191P4D12 protein (Nectin-4).

### CROSS-REFERENCE TO RELATED APPLICATIONS

This application claims the benefit of U.S. Application No. 63/296,831, filed on Jan. 5, 2022, and U.S. Application No. 63/304,136, filed on Jan. 28, 2022, the disclosure of each of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14369-279-228_SequenceListing.xml”, was created on Dec. 15, 2022, and is 34,925 bytes in size.

### 1. FIELD

Provided herein are methods for treating urothelial or bladder cancers, such as muscle invasive urothelial cancer (MIUC) or muscle invasive bladder cancer (MIBC), with antibody drug conjugates (ADC) that bind to 191P4D12 protein (Nectin-4).

### 2. BACKGROUND

191P4D12 (which is also known as Nectin-4) is a 66 kDa type I transmembrane protein that belongs to the nectin family of adhesion molecules. It is composed of an extracellular domain (ECD) containing 3 immunoglobulin (Ig)-like subdomains, a transmembrane helix, and an intracellular region (Takai et al., Annu Rev Cell Dev Biol (2008); 24: 309-42). Nectins are thought to mediate Ca²⁺-independent cell-cell adhesion via both homophilic and heterophilic trans-interactions at adherens junctions where they can recruit cadherins and modulate cytoskeletal rearrangements (Rikitake et al., Cell Mol Life Sci (2008); 65(2): 253-63.). Sequence identity of Nectin-4 to other Nectin family members is low and ranges between 25%-30% in the ECD (Reymond et al., Biol Chem (2001); 276(46): 43205-15).

The 3 Ig-like subdomains in the ECD of Nectin-4 are designated V, C1 and C2. The C1 domain is responsible for cis-interaction (homodimerization), while V domains of most Nectin molecules contribute to trans-interaction and cell-cell adhesion (Mandai et al., Curr Top Dev Biol (2015); 112: 197-231; Takai et al., Nat Rev Mol Cell Biol (2008); 9(8): 603-15.).

Nectin-4 was originally identified by bioinformatics and cloned from human trachea (Reymond et al., J Biol Chem (2001) 276(46): 43205-15.). Nectin-4 was identified as markedly upregulated in urothelial cancer using suppression subtractive hybridization on a pool of urothelial cancer specimens. Characterization of expression in multiple tumor specimens, both at the ribonucleic acid (RNA) level and by immunohistochemistry (IHC), also demonstrated high levels of Nectin-4 in breast, pancreatic, lung, and other cancers (Challita-Eid et al., Cancer Res (2016); 76(10): 3003-13.).

Nectin-4 has been found to be expressed in multiple cancers, particularly urothelial, breast, lung, pancreatic, and ovarian cancers. Higher levels of expression are associated with disease progression and/or poor prognosis (Fabre-Lafay et al., BMC Cancer (2007); 7: 73).

### Urothelial Cancer

Urothelial cancer is the most common form of cancer arising within the genitourinary tract, which is a major cause of morbidity and mortality. Approximately 151,000 new cases of bladder cancer, the most common primary location for urothelial cancer, are diagnosed annually in Europe (Ervik 2016). In the US the National Cancer Institute estimates more than 79,000 new cases of bladder cancer were diagnosed in 2017, and more than 16,800 people died from the disease (National Cancer Institute 2018).

### Bladder Cancer

Of all new cases of cancer in the United States, bladder cancer represents approximately 5 percent in men (fifth most common neoplasm) and 3 percent in women (eighth most common neoplasm). The incidence is increasing slowly, concurrent with an increasing older population. American Cancer Society (cancer.org) estimates that there are 81,400 new cases annually, including 62,100 in men and 19,300 in women, which accounts for 4.5% of all cancer cases. The age-adjusted incidence in the United States is 20 per 100,000 for men and women. There are an estimated 17,980 deaths from bladder cancer in annually (13,050 in men and 4,930 in women), which accounts for 3% of cancer related deaths. Bladder cancer incidence and mortality strongly increase with age and will be an increasing problem as the population becomes more elderly. Globally, approximately 580,000 people will be diagnosed with bladder cancer in 2020, and bladder cancer will be attributed to approximately 210,000 deaths worldwide.

Most bladder cancers recur in the bladder. Bladder cancer is managed with a combination of transurethral resection of the bladder (TUR) and intravesical chemotherapy or immunotherapy. The multifocal and recurrent nature of bladder cancer points out the limitations of TUR. Most muscle-invasive cancers are not cured by TUR alone. Radical cystectomy and urinary diversion is the most effective means to eliminate the cancer but carry an undeniable impact on urinary and sexual function. There continues to be a significant need for treatment modalities that are beneficial for bladder cancer patients.

There is a significant need for additional therapeutic methods for urothelial and bladder cancers. These include the use of antibodies and antibody drug conjugates as treatment modalities.

### Muscle Invasive Bladder Cancer (MIBC) and Muscle Invasive Urothelial Cancer (MIUC)

Muscle invasive urothelial cancer (MIUC) includes cancers originating in all parts of the urinary tract (renal pelvis or upper tract, bladder, and lower tract (urethra)). Muscle invasive bladder cancer (MIBC) refers to cancers of the bladder, excluding upper and lower tracts. Up to 25% of all patients diagnosed with urothelial cancers present with muscle-invasive disease, for whom the risk for progression or metastasis is substantial. Muscle invasive bladder cancer (MIBC) has a five-year mortality rate of approximately 69% (American Cancer Society (ACS), Survival rates for bladder cancer, 2019, www.cancer.org/cancer/bladder-cancer/detection-diagnosis-staging/survival-rates.html. Accessed: Feb. 23, 2018). Prognosis and recurrence vary by stage of disease, as well as other prognostic features, including lymph node involvement, lymphovascular invasion, tumor stage, presence of variant histology, and molecular subtyping (Lotan et al., J. Clin. Oncol., 2005, 23(27):6533-9; Karakiewicz et al., J Urol., 2006, 176(4 Pt 1):1354-61; Choi 2014).

The treatment of MIBC is complex and requires a multi disciplinary collaboration among surgeons, radiation therapists, and medical oncologists (Aragon-Ching et al., Am Soc Clin Oncol Educ Book, 2018, 38:307-18). Cisplatin based neoadjuvant chemotherapy (NAC), followed by radical cystectomy (RC) and lymph node dissection, has been the SOC in MIBC for almost 2 decades, as established by evidence from several randomized trials and a large meta-analysis that was initially published by the Advanced Bladder Cancer Meta-Analysis Collaboration in 2003 and updated in 2005 (Advanced Bladder Cancer Meta-analysis, Eur Urol., 2005, 48(2):202-5). The final version of the meta-analysis included 11 randomized trials and 3005 patients, and compared platinum-based NAC (all trials but one involved cisplatin) plus definitive local treatment (cystectomy or radiation) with the same local treatment alone. The results showed a significant benefit for patients who received cisplatin-based combination NAC, with a 14% reduction of risk of death (HR, 0,86; 95% confidence interval [CI], 0.77-0.95; P=0.003), which translated into a 5% improvement in OS and a 9% improvement in disease free survival (DFS) at 5 years. For patients who received cisplatin-based NAC, the 5 year OS was 50% compared with 45% for patients who received definitive local therapy alone. The survival benefit has not been shown for patients who received carboplatin-based combinations. More recently, additional NAC regimens, including accelerated or dose dense MVAC and the combination of gemcitabine and cisplatin (GC), have been studied with the aim of improving patient tolerance and shortening treatment duration with comparable pathologic response rates. Around 30% to 40% of patients achieved pCR rate and 50% had pathologic downstaging with accelerated or dose dense MVAC (Choueiri et al., J Clin Oncol, 2014, 32(18):1889-94; Plimack et al., J Clin Oncol., 2014, 32(18):1895-901).

Although NAC prior to RC is effective, residual high-risk disease (≥pT2) is still present in more than 50% of patients and is associated with a poor prognosis. In addition, many patients are unfit for surgery or are cisplatin-ineligible, and considerations for bladder-preservation strategies not only are increasingly recognized as optimal treatment alternatives, but also should feature in the range of management options presented to patients at the time of diagnosis (Aragon-Ching et al., Am Soc Clin Oncol Educ Book, 2018, 38:307-18). These studies are nevertheless limited to biomarker selected patients who are expected to respond well to neoadjuvant treatment.

Apart from chemotherapy, immunotherapy has been successfully used in locally advanced and metastatic bladder cancer, and is moving into the MIBC space as monotherapy or in combination with chemotherapy agents. Two recent investigator initiated studies were designed to obtain biomarker results and to assess the efficacy of single-agent programmed cell death 1/programmed cell death-ligand 1 (PD 1/PD L1) inhibitors in patients with MIBC in the neoadjuvant setting. Both have published preliminary data. In the PURE-01 study, patients with T2 T3bN0M0 disease, regardless of cisplatin eligibility, received 3 cycles of 200 mg of pembrolizumab before RC (Necchi et al., J Clin Oncol., 2018, 36(34):3353-60). Pembrolizumab was safely administered and resulted in a 42% pCR rate among 50 treated patients with a 54.3% pCR rate in patients with PD L1 positive disease and a pathologic downstaging rate of 65.7%. Immune-mediated adverse events (imAEs) were reported but did not delay the planned surgery. Post-surgical complications were similar to those reported in the literature and were related to either open or robotic surgery. Intra-operative performance measures, such as the number of removed lymph nodes, surgical margins, and operation time, were not compromised by neoadjuvant pembrolizumab. In the ABACUS study, 69 cisplatin-ineligible patients with T2 T4N0M0 MIBC were treated with 2 cycles of 1200 mg neoadjuvant atezolizumab prior to RC, with no significant safety findings (Powles et al., 2018, J Clin Oncol., 36(Suppl 15):Abstract 4506).

pCR was observed in 29% of the patients and in 40% of patients with PD-L1-positive disease compared to only 16% in the PD-L1-negative cohort. A total of 39% of patients were down staged to non-muscle invasive disease. Both studies lack the long-term follow-up data needed to assess PFS and OS in the postoperative setting for patients who receive neoadjuvant immunotherapy.

There is currently no standard neoadjuvant or perioperative treatment options for patients with MIBC who are cisplatin-ineligible. Therefore, newer treatment approaches, including new drug combination strategies, are needed for this patient population.

### 3. SUMMARY

Provided herein are methods for the treatment of various cancers in human subjects, such as urothelial and bladder cancer, including subjects with muscle invasive bladder cancer (MIBC) and muscle invasive urothelial cancer (MIUC) who are cisplatin-ineligible, using an antibody drug conjugate (ADC) that binds 191P4D12.

Embodiment 1. A method of treating muscle invasive urothelial cancer (MIUC) or muscle invasive bladder cancer (MIBC) in a human subject, comprising (a) administering to the subject an effective amount of an antibody drug conjugate (ADC); wherein the ADC comprises an antibody or antigen binding fragment thereof that binds to 191P4D12 (Nectin-4) conjugated to one or more units of monomethyl auristatin E (MMAE); wherein the subject is ineligible to receive cisplatin treatment (cisplatin ineligible); and wherein the pathological complete response rate (pCRR) is at least 30%.

Embodiment 2. A method of treating muscle invasive urothelial cancer (MIUC) or muscle invasive bladder cancer (MIBC) in a human subject, comprising administering to the subject an effective amount of an antibody drug conjugate (ADC); wherein the ADC comprises an antibody or antigen binding fragment thereof that binds to 191P4D12 (Nectin-4) conjugated to one or more units of monomethyl auristatin E (MMAE); wherein the subject is ineligible to receive cisplatin treatment (cisplatin ineligible); and wherein the pathological downstaging rate (pDSR) is at least 50%.

Embodiment 3. The method of embodiment 1 or embodiment 2, wherein the method comprises administering to the subject 3 cycles of the ADC.

Embodiment 4. The method of any one of embodiments 1 to 3, wherein the treatment of the cancer further comprises radical cystectomy and pelvic lymph node dissection (RC+PLND).

Embodiment 5. The method of embodiment 4, wherein the human subject receives the RC+PLND about four (4) to about twelve (12) weeks after the ADC is administered to the human subject.

Embodiment 6. The method of embodiment 1 or 2, further comprising (b) performing radical cystectomy and pelvic lymph node dissection (RC+PLND) on the subject.

Embodiment 7. The method of embodiment 6, wherein (b) is performed about four (4) to about twelve (12) weeks after (a).

Embodiment 8. A method of treating muscle invasive urothelial cancer (MIUC) or muscle invasive bladder cancer (MIBC) in a human subject, comprising: administering to the subject multiple cycles of an effective amount of an antibody drug conjugate (ADC), wherein
(a) the ADC comprises an antibody or antigen binding fragment thereof that binds to 191P4D12 (Nectin-4) conjugated to one or more units of monomethyl auristatin E (MMAE);
(b) the subject is ineligible to receive cisplatin treatment (cisplatin ineligible); and
(c) the number of cycles of ADC treatment administered to the cisplatin ineligible subject is equal to or less than the number of cycles of standard-of-care (SOC) therapy used to treat cisplatin eligible subjects with MIUC or MIBC.

Embodiment 9. The method of embodiment 8, wherein the SOC therapy for a cisplatin eligible subject comprises cisplatin.

Embodiment 10. The method of embodiment 8, wherein the SOC therapy for the cisplatin eligible subject comprises (i) methotrexate, vinblastine, doxorubicin, and cisplatin (MVAC), or (ii) gemcitabine plus cisplatin.

Embodiment 11. The method of embodiment 8, wherein the SOC therapy for the cisplatin eligible subject comprises administering a programmed cell death 1 (PD-1) or a programmed cell death-ligand 1 (PD-L1) inhibitor.

Embodiment 12. The method of any one of embodiments 8 to 11, wherein the number of cycles of ADC treatment administered to the cisplatin ineligible subject is 3 or 4.

Embodiment 13. The method of any one of embodiments 8 to 11, wherein the number of cycles of ADC treatment administered to the cisplatin ineligible subject is 3.

Embodiment 14. A method of administering neoadjuvant or perioperative therapy to treat muscle invasive urothelial cancer (MIUC) or muscle invasive bladder cancer (MIBC) in a human subject, comprising: administering to the subject multiple cycles of an effective amount of an antibody drug conjugate (ADC), wherein
(a) the ADC comprises an antibody or antigen binding fragment thereof that binds to 191P4D12 (Nectin-4) conjugated to one or more units of monomethyl auristatin E (MMAE);
(b) the subject is ineligible to receive cisplatin treatment (cisplatin ineligible); and
(c) the subject remains eligible for radical cystectomy and pelvic lymph node dissection (RC+PLND) surgery following the multiple cycles of treatment with the ADC.

Embodiment 15. The method of embodiment 14, wherein the ADC is administered as a neoadjuvant therapy prior to the surgery.

Embodiment 16. The method of embodiment 14, wherein the ADC is administered prior to and following the surgery.

Embodiment 17. A method of treating muscle invasive urothelial cancer (MIUC) or muscle invasive bladder cancer (MIBC) in a human subject, comprising: administering to the subject multiple cycles of an effective amount of an antibody drug conjugate (ADC), wherein
(a) the ADC comprises an antibody or antigen binding fragment thereof that binds to 191P4D12 (Nectin-4) conjugated to one or more units of monomethyl auristatin E (MMAE);
(b) the subject is ineligible to receive cisplatin treatment (cisplatin ineligible); and
(c) the efficacy of the treatment of the subject with the effective amount of the ADC is similar to the efficacy of treatment observed with cisplatin eligible patients treated with standard-of-care (SOC) therapy used to treat cisplatin eligible subjects with MIUC or MIBC.

Embodiment 18. The method of embodiment 17, wherein the efficacy of the treatment is at least as efficacious as the efficacy of the treatment observed with SOC for cisplatin eligible subjects.

Embodiment 19. The method of embodiment 17 or 18, wherein the measure of efficacy of treatment is one or more of: pathological complete response rate (pCRR), pathological downstaging rates (pDSR), disease free survival (DFS), event-free survival (EFS), overall survival (OS), progression free survival (PFS), and duration of response (DoR).

Embodiment 20. The method of embodiment 19, wherein the pCRR for the cisplatin ineligible subject is at least 30%.

Embodiment 21. The method of embodiment 19, wherein pDSR for the cisplatin ineligible subject is at least 50%.

Embodiment 22. The method of any one of embodiments 18 to 21, wherein the SOC therapy for a cisplatin eligible subject comprises cisplatin.

Embodiment 23. The method of embodiment 22, wherein the SOC therapy for the cisplatin eligible subject comprises (i) methotrexate, vinblastine, doxorubicin, and cisplatin (MVAC), or (ii) gemcitabine plus cisplatin.

Embodiment 24. The method of embodiment 18, wherein the SOC therapy for the cisplatin eligible subject comprises a programmed cell death 1 (PD-1) or a programmed cell death-ligand 1 (PD-L1) inhibitor.

Embodiment 25. The method of embodiment 24, wherein the PD-1 inhibitor is nivolumab or pembrolizumab.

Embodiment 26. The method of embodiment 24, wherein the PD-L1 inhibitor is selected from the group consisting of atezolizumab, avelumab, and durvalumab.

Embodiment 27. The method of any one of embodiments 1 to 26, wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

Embodiment 28. The method of any one of embodiments 1 to 27, wherein the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO: 11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14, or
wherein the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21.

Embodiment 29. The method of any one of embodiments 1 to 27, wherein the antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
wherein the antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

Embodiment 30. The method of any one of embodiments 1 to 29, wherein the antibody or antigen binding fragment thereof comprises a heavy chain variable region comprising the amino acid sequence of SEQ ID NO:22 and a light chain variable region comprising the amino acid sequence of SEQ ID NO:23.

Embodiment 31. The method of any one of embodiments 1 to 30, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

Embodiment 32. The method of any one of embodiments 1 to 30, wherein the antigen binding fragment is an Fab, F(ab′)2, Fv, or scFv.

Embodiment 33. The method of any one of embodiments 1 to 31, wherein the antibody is a fully human antibody.

Embodiment 34. The method of any one of embodiments 1 to 31 and 33, wherein the antibody is an IgG1 and the light chain is a kappa light chain.

Embodiment 35. The method of any one of embodiments 1 to 34, wherein the antibody or antigen binding fragment thereof is recombinantly produced.

Embodiment 36. The method of any one of embodiments 1 to 35, wherein the antibody or antigen binding fragment is conjugated to each unit of MMAE via a linker.

Embodiment 37. The method of embodiment 36, wherein the linker is an enzyme-cleavable linker, and wherein the linker forms a bond with a sulfur atom of the antibody or antigen binding fragment thereof.

Embodiment 38. The method of embodiment 36 or 37, wherein the linker has a formula of: -Aa-Ww-Yy-; wherein -A- is a stretcher unit, a is 0 or 1; -W- is an amino acid unit, w is an integer ranging from 0 to 12; and -Y- is a spacer unit, y is 0, 1, or 2.

Embodiment 39. The method of embodiment 38, wherein the stretcher unit has the structure of Formula (1) below; the amino acid unit is valine-citrulline; and the spacer unit is a PAB group comprising the structure of Formula (2) below:

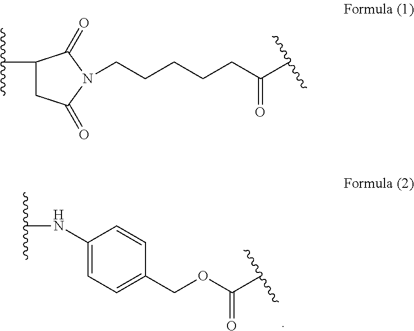 [SMILES: *C(=O)CCCCCN1C(=O)CC(*)C1=O ; *Nc1ccc(COC(*)=O)cc1]

Embodiment 40. The method of embodiment 38 or 39, wherein the stretcher unit forms a bond with a sulfur atom of the antibody or antigen binding fragment thereof, and wherein the spacer unit is linked to MMAE via a carbamate group.

Embodiment 41. The method of any one of embodiments 1 to 40, wherein the ADC comprises from 1 to 20 units of MMAE per antibody or antigen binding fragment thereof.

Embodiment 42. The method of any one of embodiments 1 to 41, wherein the ADC comprises from 1 to 10 units of MMAE per antibody or antigen binding fragment thereof.

Embodiment 43. The method of any one of embodiments 1 to 42, wherein the ADC comprises from 2 to 8 units of MMAE per antibody or antigen binding fragment thereof.

Embodiment 44. The method of any one of embodiments 1 to 43, wherein the ADC comprises from 3 to 5 units of MMAE per antibody or antigen binding fragment thereof.

Embodiment 45. The method of any one of embodiments 1 to 42, wherein the ADC has the following structure:

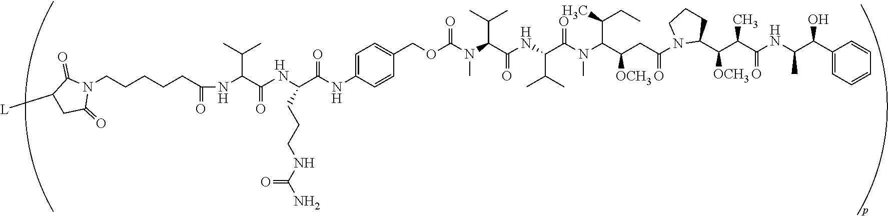

wherein L- represents the antibody or antigen binding fragment thereof and p is from 1 to 10.

Embodiment 46. The method of embodiment 45, wherein p is from 2 to 8.

Embodiment 47. The method of embodiment 45 or 46, wherein p is from 3 to 5.

Embodiment 48. The method of any one of embodiments 45 to 47, wherein p is from 3 to 4.

Embodiment 49. The method of any one of embodiments 45 to 48, wherein p is about 4.

Embodiment 50. The method of any one of embodiments 45 to 48, wherein the average p value of the effective amount of the antibody drug conjugates is about 3.8.

Embodiment 51. The method of any one of embodiments 1 to 50, wherein the ADC is formulated in a pharmaceutical composition comprising L-histidine, polysorbate-20 (TWEEN-20), and trehalose dehydrate.

Embodiment 52. The method of any one of embodiments 1 to 51, wherein the ADC is formulated in a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.

Embodiment 53. The method of any one of embodiments 1 to 51, wherein the ADC is formulated in a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

Embodiment 54. The method of any one of embodiments 1 to 53, wherein the ADC has the following structure:

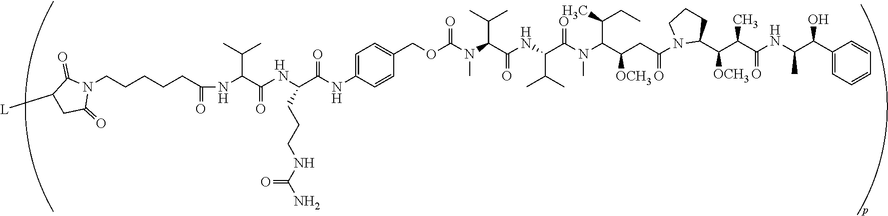

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV infusion on Days 1 and 8 of every three-week cycle.

Embodiment 55. The method of any one of embodiments 1 to 54, wherein the subject has cT2-T4aN0M0 stage MIBC.

Embodiment 56. The method of any one of embodiments 1 to 55, wherein the subject is considered cis-platin ineligible if one or more of the following criteria are satisfied: (a) GFR<60 mL/min but ≥30 mL/min, (wherein the GFR is measured by the Cockcroft-Gault formula, Modification of Diet in Renal Disease equations (MDRD), or 24-hour urine collection); (b) ECOG performance status of 2; (c) NCI CTCAE Version 4.03 Grade ≥2 hearing loss; and (d) NYHA Class III heart failure.

Embodiment 57. The method of any one of embodiments 1 to 56, wherein the subject has not received prior systemic treatment, chemoradiation, and/or radiation therapy for MIBC.

Embodiment 58. The method of any one of embodiments 1 to 57, wherein the subject has not received an immune checkpoint inhibitor (CPI).

Embodiment 59. The method of embodiment 58, wherein the CPI is a programmed cell death 1 (PD-1) inhibitor or a programmed cell death-ligand 1 (PD-L1) inhibitor.

Embodiment 60. The method of any one of embodiments 1 to 59, wherein the subject has not received a CD137 agonist, a CTLA-4 inhibitor, or an OX-40 agonist.

Embodiment 61. The method of any one of embodiments 1 to 60, wherein the effective amount of the ADC is about 1 to about 10 mg/kg of the subject's body weight, about 1 to about 5 mg/kg of the subject's body weight, about 1 to about 2.5 mg/kg of the subject's body weight, or about 1 to about 1.25 mg/kg of the subject's body weight.

Embodiment 62. The method of any one of embodiments 1 to 60, wherein the effective amount of the ADC is about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

Embodiment 63. The method of any one of embodiments 1 to 60, wherein the effective amount of the ADC is about 1 mg/kg of the subject's body weight.

Embodiment 64. The method of any one of embodiments 1 to 60, wherein the effective amount of ADC is about 1.25 mg/kg of the subject's body weight.

Embodiment 65. The method of any one of embodiments 1 to 60, wherein in (a) the effective amount of the ADC is administered to the subject on days 1 and 8 of every 3 week cycle for a total of 3 cycles.

Embodiment 66. The method of embodiment 65, wherein the effective amount of the ADC is about 1.25 mg/kg of the subject's body weight.

Embodiment 67. The method of embodiment 4 or 5, wherein in (a) the effective amount of the ADC is administered to the subject on days 1 and 8 of every 3 week cycle for a total of 3 cycles.

Embodiment 68. The method of any one of embodiments 4, 5, or 67, wherein the method further comprises (b) about 8 weeks after the subject receives the RC+PLND, administering to the subject on days 1 and 8 of every 3 week cycle the effective amount of the ADC for a total of 6 cycles.

Embodiment 69. The method of embodiment 67 or 68, wherein the effective amount of the ADC is about 1.25 mg/kg of the subject's body weight.

Embodiment 70. The method of embodiment 6 or 7, wherein in (a) the effective amount of the ADC is administered to the subject on days 1 and 8 of every 3 week cycle for a total of 3 cycles.

Embodiment 71. The method of any one of embodiments 5, 7, or 70, further comprising (c) about 8 weeks after (b), administering to the subject on days 1 and 8 of every 3 week cycle the effective amount of the ADC for a total of 3 cycles.

Embodiment 72. The method of embodiment 70 or 71, wherein the effective amount of the ADC is about 1.25 mg/kg of the subject's body weight.

Embodiment 73. The method of any one of embodiments 8 to 64, wherein the effective amount of the ADC is administered to the human subject on days 1 and 8 of a 3 week cycle for a total of 3 cycles.

Embodiment 74. The method of any one of embodiments 1 to 73, wherein wherein the ADC is administered by intravenous (IV) injection or infusion.

Embodiment 75. The method of any one of embodiments 1 to 74, wherein the cancer is muscle invasive urothelial cancer (MIUC).

Embodiment 76. The method of any one of embodiments 1 to 74, wherein the cancer is muscle invasive bladder cancer (MIBC).

Embodiment 77. The method of any one of embodiments 1 to 76, wherein the ADC is enfortumab vedotin (EV).

Embodiment 78. The method of any one of embodiments 1 to 77, wherein the overall survival of the subject is extended by at least 2, at least 4, at least 6, at least 8, at least 10, or at least 12 months.

Embodiment 79. The method of any one of embodiments 1 to 78, wherein the ADC is administered as a monotherapy.

### 4. BRIEF DESCRIPTION OF THE DRAWINGS

FIGS. 1A-1E depict the nucleotide and amino acid sequences of nectin-4 protein (FIG. 1A), the nucleotide and amino acid sequences of the heavy chain (FIG. 1B) and light chain (FIG. 1C) of Ha22-2(2.4)6.1, and the amino acid sequences of the heavy chain (FIG. 1D) and light chain of Ha22-2(2.4)6.1 (FIG. 1E).

FIG. 2 depicts the overall study design of the clinical study described in Section 6.1. TURBT, trans urethral resection of bladder tumour.

FIG. 3 depicts the European Organization for the Research and Treatment (EORTC) Core Quality of Life (QLQ-C-30) assessment (EORTC-QLQ-C-30, current version, Version 3), as described in Section 6.1.

FIG. 4 depicts the EuroQol-5 Dimensions (EQ-5D-5L) described in Section 6.1.

FIG. 5 depicts the MIUC disease stages (shown in FIG. 5 are Tis, Ta, T1, T2a, T2b, T3a, T3b, T4a, and T4b) relative to the anatomy of the bladder and pelvic region described in Section 6.2 (See, e.g., www.cancer.org/cancer/bladder-cancer/detection-diagnosis-staging/staging.html).

FIG. 6 depicts event-free survival (EFS) observed in the clinical study described in Section 6.2. MIBC EV Mono, Muscle invasive bladder cancer Enfortumab Vedotin monotherapy-treated subjects.

FIG. 7 depicts the overall study design of the clinical study described in Section 6.3. TURBT, trans urethral resection of bladder tumour.

### 5. DETAILED DESCRIPTION

Before the present disclosure is further described, it is to be understood that the disclosure is not limited to the particular embodiments set forth herein, and it is also to be understood that the terminology used herein is for describing particular embodiments only, and is not intended to be limiting.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell, for example, a cancer cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “antigen binding fragment,” “antigen binding domain,” “antigen binding region,” and similar terms refer to that portion of an antibody, which comprises the amino acid residues that interact with an antigen and confer on the binding agent its specificity and affinity for the antigen (e.g., the CDRs). “Antigen-binding fragment” as used herein include “antibody fragment,” which comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single variable domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well-known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody or antigen binding fragment thereof described herein terms such as “bind to,” “that specifically bind to,” and analogous terms are also used interchangeably herein and refer to binding molecules of antigen binding domains that specifically bind to an antigen, such as a polypeptide. An antibody or antigen binding fragment that binds to or specifically binds to an antigen may be cross-reactive with related antigens. In certain embodiments, an antibody or antigen binding fragment that binds to or specifically binds to an antigen does not cross-react with other antigens. An antibody or antigen binding fragment that binds to or specifically binds to an antigen can be identified, for example, by immunoassays, Octet®, Biacore®, or other techniques known to those of skill in the art. In some embodiments, an antibody or antigen binding fragment binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of an antibody or antigen binding fragment to a “non-target” protein is less than about 10% of the binding of the binding molecule or antigen binding domain to its particular target antigen, for example, as determined by fluorescence activated cell sorting (FACS) analysis or RIA. With regard terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding fragment that binds to an antigen includes one that is capable of binding the antigen with sufficient affinity such that the binding molecule is useful, for example, as a diagnostic agent in targeting the antigen. In certain embodiments, an antibody or antigen binding fragment that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding fragment binds to an epitope of an antigen that is conserved among the antigen from different species (e.g., between human and cyno species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using biolayer interferometry (BLI) or surface plasmon resonance (SPR) assays by Octet®, using, for example, a Octet®QK384 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®QK384, the Biacore®TM-2000, or the Biacore®TM-3000 system.

In certain embodiments, the antibodies or antigen binding fragments can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

In certain embodiments, the antibodies or antigen binding fragments can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate comprising the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

In certain embodiments, the antibodies or antigen binding fragments can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies comprising variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising nonhuman antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1: 755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Bruggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

In certain embodiments, the antibodies or antigen binding fragments can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies or antigen binding fragments can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well-known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each the α and γ chains and four CH domains for and F isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail in the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a R sheet configuration, connected by three hypervariable regions, which form loops connecting, and in some cases form part of, the R sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while p and F contain approximately 550 amino acids. When combined with a light chain, these distinct types of heavy chains give rise to five well-known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL j-sheet framework. Accordingly, CDRs are variable region sequences interspersed within the framework region sequences.

CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted below in Table 1

TABLE 1

| | Kabat | AbM | Chothia | Contact | IMGT |
|---|---|---|---|---|---|
| CDR-LI | L24--L34 | L24--L34 | L24--L34 | L30--L36 | L27--L38 |
| CDR-L2 | L50--L56 | L50--L56 | L50--L56 | L46--L55 | L56--L65 |
| CDR-L3 | L89--L97 | L89--L97 | L89--L97 | L89--L96 | L105-L117 |
| CDR-H1 | H31--H35B | H26--H35B | H26-- | H30--H35B | H27--H38 |
| | (Kabat | | H32 . . . 34 | | |
| | Numbering | | | | |
| CDR-H1 | H31--H35 | H26--H35 | H26--H32 | H30--H35 | |
| | (Chothia | | | | |
| | Numbering) | | | | |
| CDR-H2 | H50--H65 | H50--H58 | H52--H56 | H47--H58 | H56--H65 |
| CDR-H3 | H95--H102 | H95--H102 | H95--H102 | H93--H101 | H105-H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule comprising a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations comprising a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

The abbreviation “MMAE” refers to monomethyl auristatin E.

Unless otherwise indicated by context, a hyphen (-) designates the point of attachment to the pendant molecule.

The term “Chemotherapeutic Agent” refers to all chemical compounds that are effective in inhibiting tumor growth. Non-limiting examples of chemotherapeutic agents include alkylating agents; for example, nitrogen mustards, ethyleneimine compounds and alkyl sulphonates; antimetabolites, for example, folic acid, purine or pyrimidine antagonists; mitotic inhibitors, for example, anti-tubulin agents such as vinca alkaloids, auristatins and derivatives of podophyllotoxin; cytotoxic antibiotics; compounds that damage or interfere with DNA expression or replication, for example, DNA minor groove binders; and growth factor receptor antagonists. In addition, chemotherapeutic agents include cytotoxic agents (as defined herein), antibodies, biological molecules and small molecules.

As used herein, the term “conservative substitution” refers to substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)). Such exemplary substitutions are preferably made in accordance with those set forth in Table 2 and Table 3. For example, such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table 3 herein; pages 13-15 “Biochemistry” 2nd ED. Lubert Stryer ed (Stanford University); Henikoff et al., PNAS 1992 Vol 89 10915-10919; Lei et al., J Biol Chem 1995 May 19; 270(20):11882-11886). Other substitutions are also permissible and may be determined empirically or in accord with known conservative substitutions.

TABLE 2
Amino Acid Abbreviations

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE 3
Amino Acid Substitution or Similarity Matrix
Adapted from the GCG Software 9.0 BLOSUM62 amino acid
substitution matrix (block substitution matrix). The higher the value,
the more likely a substitution is found in related, natural proteins.

| A | C | D | E | F | G | H | I | K | L | M | N | P | Q | R | S | T | V | W | Y | . |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

The term “homology” or “homologous” is intended to mean a sequence similarity between two polynucleotides or between two polypeptides. Similarity can be determined by comparing a position in each sequence, which can be aligned for purposes of comparison. If a given position of two polypeptide sequences is not identical, the similarity or conservativeness of that position can be determined by assessing the similarity of the amino acid of the position, for example, according to Table 3. A degree of similarity between sequences is a function of the number of matching or homologous positions shared by the sequences. The alignment of two sequences to determine their percent sequence similarity can be done using software programs known in the art, such as, for example, those described in Ausubel et al., Current Protocols in Molecular Biology, John Wiley and Sons, Baltimore, MD (1999). Preferably, default parameters are used for the alignment, examples of which are set forth below. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

The term “homologs” of to a given amino acid sequence or a nucleic acid sequence is intended to indicate that the corresponding sequences of the “homologs” having substantial identity or homology to the given amino acid sequence or nucleic acid sequence.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Antibodies may also be conjugated to an anti-cancer pro-drug activating enzyme capable of converting the pro-drug to its active form.

The term “effective amount” or “therapeutically effective amount” as used herein refers to the amount of binding molecule (e.g., an antibody) or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., a cancer).

The term “cancer” or “cancer cell” is used herein to denote a tissue or cell found in a neoplasm which possesses characteristics which differentiate it from normal tissue or tissue cells. Among such characteristics include but are not limited to: degree of anaplasia, irregularity in shape, indistinctness of cell outline, nuclear size, changes in structure of nucleus or cytoplasm, other phenotypic changes, presence of cellular proteins indicative of a cancerous or pre-cancerous state, increased number of mitoses, and ability to metastasize. Words pertaining to “cancer” include carcinoma, sarcoma, tumor, epithelioma, leukemia, lymphoma, polyp, and scirrus, transformation, neoplasm, and the like.

As used herein, a “locally advanced” cancer refers to a cancer that has spread from where it started to nearby tissue or lymph nodes.

As used herein, a “metastatic” cancer refers to a cancer that has spread from where it started to different part of the body.

As used herein, by “nearly as efficacious” it is meant that the treatment is at least 60%, at least 65%, at least 70%, at least 75%, at least 80%, at least 85%, at least 90%, at least 95%, at least 96%, at least 97%, at least 98%, or at least 99% as efficactious as the SOC therapy,

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

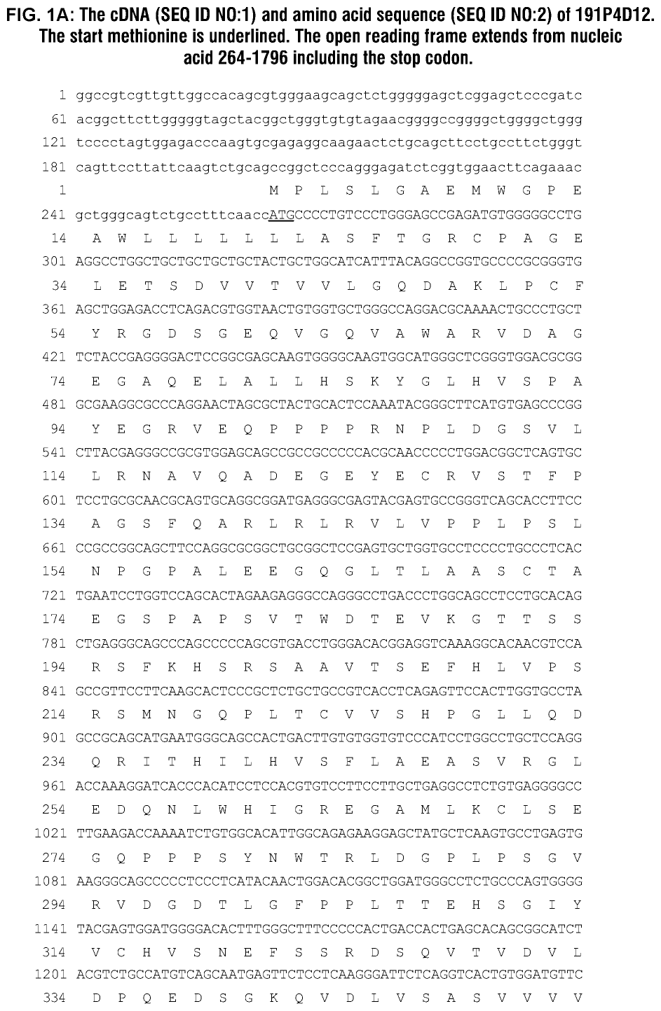

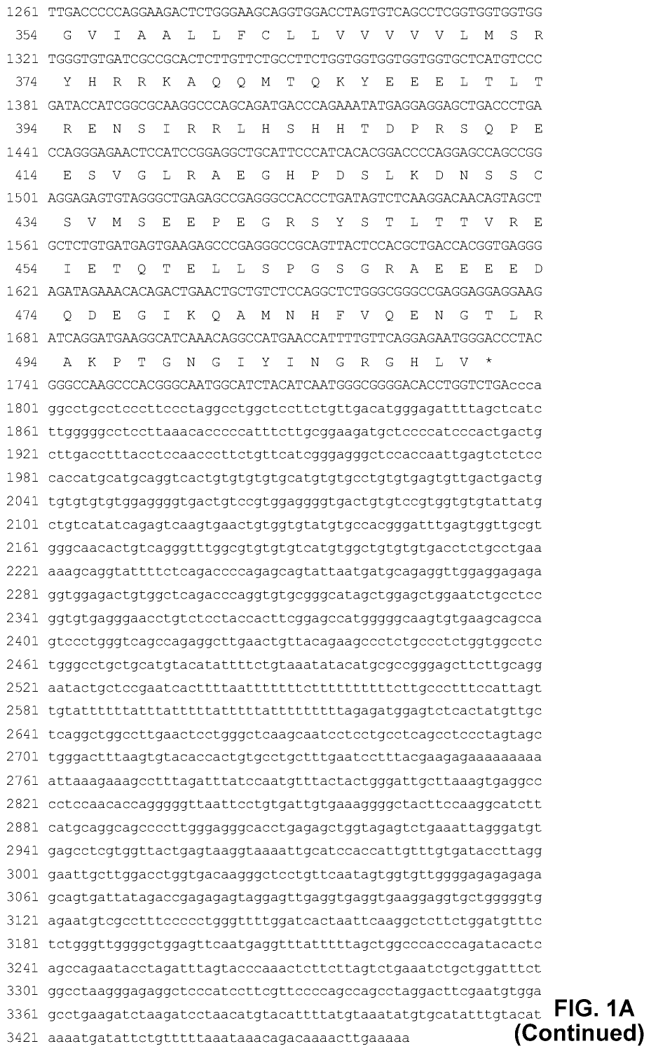

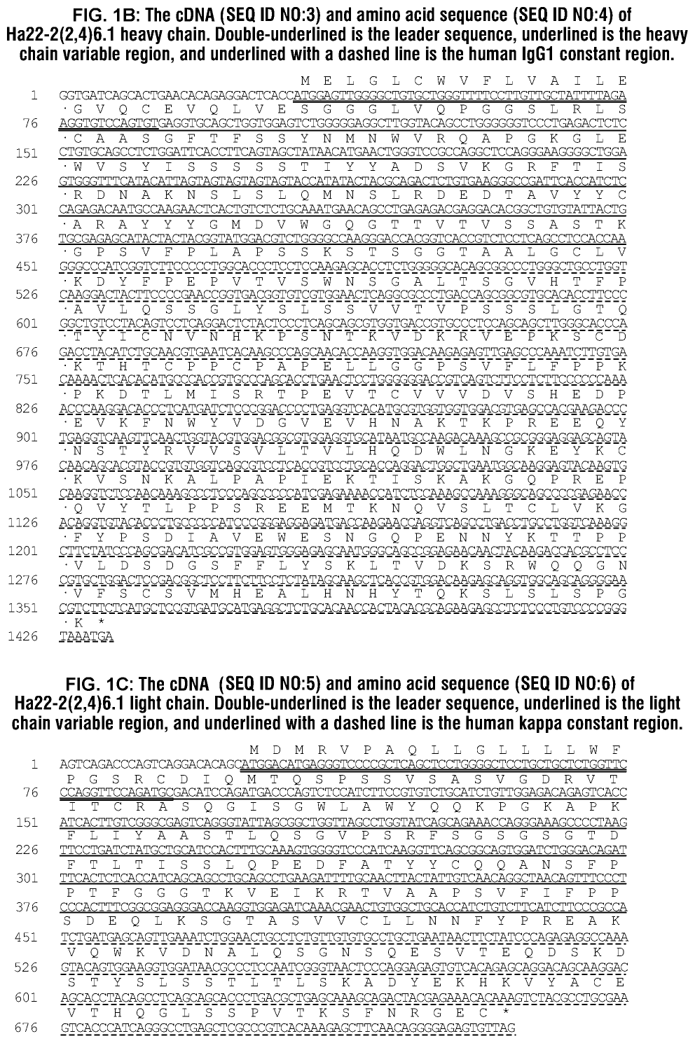

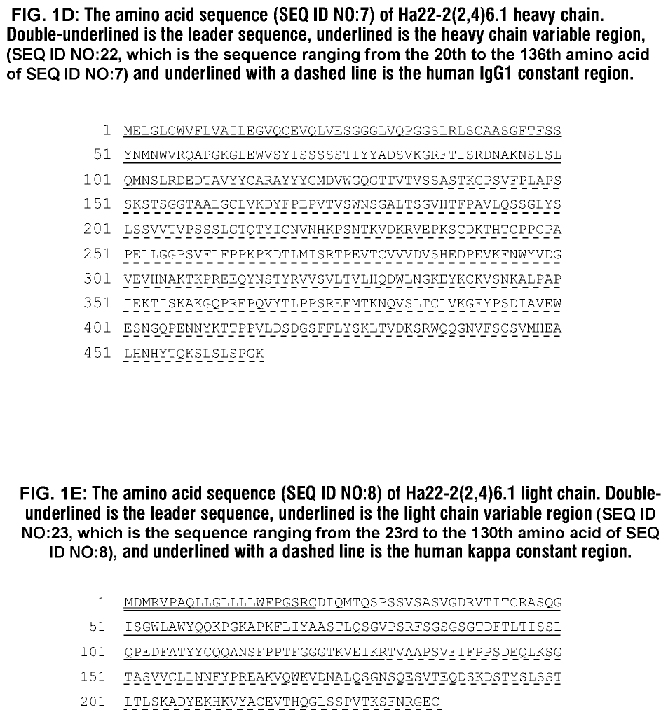

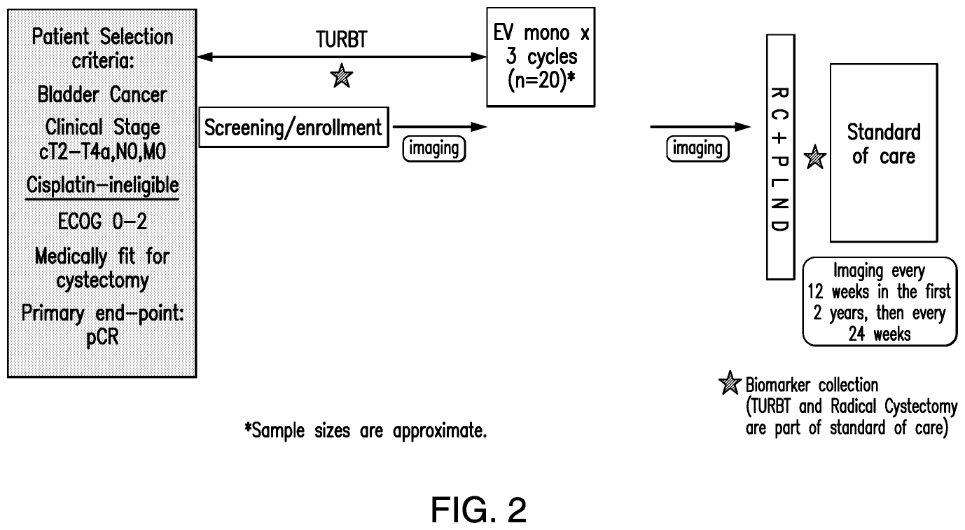

The term “variant” refers to a molecule that exhibits a variation from a described type or norm, such as a protein that has one or more different amino acid residues in the corresponding position(s) of a specifically described protein (e.g. the 191P4D12 protein shown in FIG. 1A.) An analog is an example of a variant protein. Splice isoforms and single nucleotides polymorphisms (SNPs) are further examples of variants.

The “191P4D12 proteins” and/or “191P4D12 related proteins” of the disclosure include those specifically identified herein (see, FIG. 1A), as well as allelic variants, conservative substitution variants, analogs and homologs that can be isolated/generated and characterized without undue experimentation following the methods outlined herein or readily available in the art. Fusion proteins that combine parts of different 191P4D12 proteins or fragments thereof, as well as fusion proteins of a 191P4D12 protein and a heterologous polypeptide are also included. Such 191P4D12 proteins are collectively referred to as the 191P4D12-related proteins, the proteins of the disclosure, or 191P4D12. The term “191P4D12-related protein” refers to a polypeptide fragment or a 191P4D12 protein sequence of 4, 5, 6, 7, 8, 9, 10, 11, 12, 13, 14, 15, 16, 17, 18, 19, 20, 21, 22, 23, 24, 25, or more than 25 amino acids; or, at least 30, 35, 40, 45, 50, 55, 60, 65, 70, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 330, 335, 339 or more amino acids. The term “191P4D12” is used interchangeably with nectin-4.

### 5.2 Methods of Treating Cancer

Urothelial cancer and bladder cancer, including muscle invasive urothelial cancer (MIUC) or muscle invasive bladder cancer (MIBC), in patients who are ineligible for cisplatin are particularly difficult diseases to treat. Typically, cis-ineligible MIUC and MIBC patients are somewhat frail, and suffer from multiple comorbidities beyond their urothelial cancer/bladder cancer. As noted in the Background section above, there currently are no standard neoadjuvant treatment options for cisplatin ineligible patients with MIBC or MIUC. Instead, these patients typically are directly treated with surgery. This disclosure provides efficacious and safe neoadjuvant and perioperative methods to treat patients with urothelial cancer and/or bladder cancer, and in particular, muscle invasive urothelial cancer (MIUC) or muscle invasive bladder cancer (MIBC), who are ineligible for cisplatin in this setting.

For instance, the results described herein demonstrate that in some embodiments the efficacy of treatment of a subject with an effective amount of an ADC as described herein is similar to the efficacy of treatment observed with cisplatin-eligible patients (i.e., healthier patients) treated with standard-of-care (SOC) therapy used to treat cisplatin-eligible subjects with MIUC or MIBC. The results described herein further demonstrate that in certain embodiments even following multiple cycles of treatment with the ADC described herein, a subject remains eligible for radical cystectomy and pelvic lymph node dissection (RC+PLND) surgery, i.e., that the method is sufficiently tolerated such that the subject remains healthy enough to receive surgery following neoadjuvant treatement. Finally, the results described herein further demonstrate that in certain embodiments the number of cycles of treatment with the ADC described herein administered to a cisplatin-ineligible subject can be equal to or less than the number of cycles of SOC therapy used to treat cisplatin-eligible subjects (i.e., healthier subjects) with MIUC or MIBC. Prior to the results described herein, there was considerable uncertainty whether the methods which are provided herein would be efficacious and tolerated given that this patient population has historically proven so difficult to treat and given that there is currently no standard treatment for cis-ineligble MIBC and MIBC patients. As such, the level of efficacy and safety obtained using the methods described herein was particularly notable and surprising.

### 5.2.1 Methods of Treating Cancer in General and for Selected Patients

Provided herein are methods for the treatment of various cancers in subjects, including subjects with urothelial cancer using an antibody drug conjugate (ADC) that binds 191P4D12 (Nectin-4).

In one aspect, provided herein are methods for the treatment of cancer in a human subject using an ADC that binds 191P4D12 (Nectin-4). In some embodiments, the cancer is muscle invasive urothelial cancer (MIUC) or muscle invasive bladder cancer (MIBC). In some embodiments, the method comprises administering to the subject an effective amount of an antibody drug conjugate (ADC). In some embodiments, the ADC comprises an antibody or antigen binding fragment thereof that binds to 191P4D12 (Nectin-4). In some embodiments, the ADC comprises an antibody or antigen binding fragment thereof that binds to 191P4D12 (Nectin-4) conjugated to one or more units of a cytotoxic or cytostatic agent. In some embodiments, the ADC comprises an antibody or antigen binding fragment thereof that binds to 191P4D12 (Nectin-4) conjugated to one or more units of an auristatin agent. In some embodiments, the ADC comprises an antibody or antigen binding fragment thereof that binds to 191P4D12 (Nectin-4) conjugated to one or more units of monomethyl auristatin E (MMAE). Additional examples of suitable ADCs that can be used in the methods disclosed herein are provided in section 5.3.2. In some embodiments, the subject is ineligible to receive cisplatin treatment (cisplatin ineligible). In some embodiments, the pathological downstaging rate (pDSR) is at least 50%. In some embodiments, the pathological downstaging rate (pDSR) is at least 50% In some embodiments, the effective amount of the ADC is administered to the subject on days 1 and 8 of every 3 week cycle for a total of 3 cycles. In some embodiments, the effective amount of the ADC is about 1.25 mg/kg of the subject's body weight. In some embodiments, the effective amount of the ADC is about 1.0 mg/kg of the subject's body weight.

In some embodiments, after the administering step, the subject receives a radical cystectomy and pelvic lymph node dissection (RC+PLND) as part of a treatment of the cancer (i.e., the treatment of the cancer further comprises radical cystectomy and pelvic lymph node dissection (RC+PLND)). In some embodiments, the method comprises administering to the subject 2 or 3 cycles of an effective amount of an antibody drug conjugate (ADC). In some embodiments, the method comprises administering to the subject 2 cycles of an effective amount of an antibody drug conjugate (ADC). In some embodiments, the method comprises administering to the subject 3 cycles of an effective amount of an antibody drug conjugate (ADC). In some embodiments, the subject has urothelial cancer or bladder cancer. In some embodiments, the subject has muscle invasive urothelial cancer (MIUC) or muscle invasive bladder cancer (MIBC). In some embodiments, the subject is ineligible to receive cisplatin treatment (cisplatin ineligible).

In one aspect, the method comprises administering to the subject 3 cycles of an effective amount of an antibody drug conjugate (ADC); wherein after the administering step, the subject receives a radical cystectomy and pelvic lymph node dissection (RC+PLND) as part of a treatment of the cancer; wherein the ADC comprises an antibody or antigen binding fragment thereof that binds to 191P4D12 (Nectin-4) conjugated to one or more units of monomethyl auristatin E (MMAE); wherein the subject has muscle invasive urothelial cancer (MIUC) or muscle invasive bladder cancer (MIBC); wherein the subject is ineligible to receive cisplatin treatment (cisplatin ineligible).

In one aspect, the method comprises (i) administering to the subject on days 1 and 8 of every 3 week cycle an effective amount of an ADC, wherein the ADC comprises an antibody or antigen binding fragment thereof that binds to 191P4D12 (Nectin-4) conjugated to one or more units of monomethyl auristatin E (MMAE); (ii) performing radical cystectomy and pelvic lymph node dissection (RC+PLND) on the subject; and (iii) about 8 weeks after step (ii), administering to the subject on days 1 and 8 of every 3 week cycle an effective amount of an ADC for a total of 6 cycles; wherein the subject is ineligible to receive cisplatin treatment (cisplatin ineligible). In some embodiments, the effective amount of the ADC is 1.25 mg/kg. In some embodiments, in step (iii), the effective amount of an ADC is administered for a total of 5 cycles. In some embodiments, in step (iii), the effective amount of an ADC is administered for a total of 4 cycles. In some embodiments, in step (iii), the effective amount of an ADC is administered for a total of 3 cycles. In some embodiments, in step (iii), the effective amount of an ADC is administered for a total of 2 cycles. In some embodiments, in step (iii), the effective amount of an ADC is administered for a total of 1 cycle.

In one aspect, the method comprises (i) administering to the subject on days 1 and 8 of every 3 week cycle an effective amount of an ADC, wherein the ADC comprises an antibody or antigen binding fragment thereof that binds to 191P4D12 (Nectin-4) conjugated to one or more units of monomethyl auristatin E (MMAE), wherein after the administration step, the subject receives a RC+PLND as part of a treatment of the cancer; and (ii) after the subject receives the RC+PLND, administering to the subject an effective amount of the ADC on days 1 and 8 of every 3 week cycle for a total of 6 cycles about 8 weeks after the subject receives the RC+PLND; wherein the subject is ineligible to receive cisplatin treatment (cisplatin ineligible). In some embodiments, the effective amount of the ADC is 1.25 mg/kg. In some embodiments, in step (ii), the effective amount of an ADC is administered for a total of 5 cycles. In some embodiments, in step (ii), the effective amount of an ADC is administered for a total of 4 cycles. In some embodiments, in step (ii), the effective amount of an ADC is administered for a total of 3 cycles. In some embodiments, in step (ii), the effective amount of an ADC is administered for a total of 2 cycles. In some embodiments, in step (ii), the effective amount of an ADC is administered for a total of 1 cycle.

In some embodiments of the methods provided herein, the human subject receives the RC+PLND about four (4) to about twelve (12) weeks after the ADC is administered to the human subject. In some embodiments, the human subject receives the RC+PLND about five (5) to about twelve (12) weeks after the ADC is administered to the human subject. In some embodiments, the human subject receives the RC+PLND about six (6) to about twelve (12) weeks after the ADC is administered to the human subject. In some embodiments, the human subject receives the RC+PLND about seven (7) to about twelve (12) weeks after the ADC is administered to the human subject. In some embodiments, the human subject receives the RC+PLND about eight (8) to about twelve (12) weeks after the ADC is administered to the human subject. In some embodiments, the human subject receives the RC+PLND about nine (9) to about twelve (12) weeks after the ADC is administered to the human subject. In some embodiments, the human subject receives the RC+PLND about ten (10) to about twelve (12) weeks after the ADC is administered to the human subject. In some embodiments, the human subject receives the RC+PLND about eleven (11) to about twelve (12) weeks after the ADC is administered to the human subject. In some embodiments, the human subject receives the RC+PLND about four (4) to about eleven (11) weeks after the ADC is administered to the human subject. In some embodiments, the human subject receives the RC+PLND about four (4) to about ten (10) weeks after the ADC is administered to the human subject. In some embodiments, the human subject receives the RC+PLND about four (4) to about nine (9) weeks after the ADC is administered to the human subject. In some embodiments, the human subject receives the RC+PLND about four (4) to about eight (8) weeks after the ADC is administered to the human subject. In some embodiments, the human subject receives the RC+PLND about four (4) to about seven (7) weeks after the ADC is administered to the human subject. In some embodiments, the human subject receives the RC+PLND about four (4) to about six (6) weeks after the ADC is administered to the human subject. In some embodiments, the human subject receives the RC+PLND about four (4) to about five (5) weeks after the ADC is administered to the human subject. In some embodiments, the human subject receives the RC+PLND about six (6) to about eight (8) weeks after the ADC is administered to the human subject. In some embodiments, the human subject receives the RC+PLND about seven (7) to about nine (9) weeks after the ADC is administered to the human subject. In some embodiments, the human subject receives the RC+PLND about eight (8) to about ten (10) weeks after the ADC is administered to the human subject. In some embodiments, the human subject receives the RC+PLND about nine (9) to about eleven (11) weeks after the ADC is administered to the human subject.

In some embodiments of the methods provided herein, the human subject receives the RC+PLND about four (4) weeks after the ADC is administered to the human subject. In some embodiments, the human subject receives the RC+PLND about five (5) weeks after the ADC is administered to the human subject. In some embodiments, the human subject receives the RC+PLND about six (6) weeks after the ADC is administered to the human subject. In some embodiments, the human subject receives the RC+PLND about seven (7) weeks after the ADC is administered to the human subject. In some embodiments, the human subject receives the RC+PLND about eight (8) weeks after the ADC is administered to the human subject. In some embodiments, the human subject receives the RC+PLND about nine (9) weeks after the ADC is administered to the human subject. In some embodiments, the human subject receives the RC+PLND about ten (10) weeks after the ADC is administered to the human subject. In some embodiments, the human subject receives the RC+PLND about eleven (11) weeks after the ADC is administered to the human subject. In some embodiments, the human subject receives the RC+PLND about twelve (12) weeks after the ADC is administered to the human subject.

In some embodiments, the method further comprises step (b) performing radical cystectomy and pelvic lymph node dissection (RC+PLND) on the subject. In some embodiments, step (b) is performed about four (4) to about twelve (12) weeks after step (a). In some embodiments, step (b) is performed about five (5) to about twelve (12) weeks after step (a). In some embodiments, step (b) is performed about six (6) to about twelve (12) weeks after step (a). In some embodiments, step (b) is performed about seven (7) to about twelve (12) weeks after step (a). In some embodiments, step (b) is performed about eight (8) to about twelve (12) weeks after step (a). In some embodiments, step (b) is performed about nine (9) to about twelve (12) weeks after step (a). In some embodiments, step (b) is performed about ten (10) to about twelve (12) weeks after step (a). In some embodiments, step (b) is performed about eleven (11) to about twelve (12) weeks after step (a). In some embodiments, step (b) is performed about four (4) to about eleven (11) weeks after step (a). In some embodiments, step (b) is performed about four (4) to about ten (10) weeks after step (a). In some embodiments, step (b) is performed about four (4) to about nine (9) weeks after step (a). In some embodiments, step (b) is performed about four (4) to about eight (8) weeks after step (a). In some embodiments, step (b) is performed about four (4) to about seven (7) weeks after step (a). In some embodiments, step (b) is performed about four (4) to about six (6) weeks after step (a). In some embodiments, step (b) is performed about four (4) to about five (5) weeks after step (a). In some embodiments, step (b) is performed about six (6) to about eight (8) weeks after step (a). In some embodiments, step (b) is performed about seven (7) to about nine (9) weeks after step (a). In some embodiments, step (b) is performed about eight (8) to about ten (10) weeks after step (a). In some embodiments, step (b) is performed about nine (9) to about eleven (11) weeks after step (a).

In some embodiments of the methods provided herein, step (b) is performed about four (4) weeks after step (a). In some embodiments, step (b) is performed about five (5) weeks after step (a). In some embodiments, step (b) is performed about six (6) weeks after step (a). In some embodiments, step (b) is performed about seven (7) weeks after step (a). In some embodiments, step (b) is performed about eight (8) weeks after step (a). In some embodiments, step (b) is performed about nine (9) weeks after step (a). In some embodiments, step (b) is performed about ten (10) weeks after step (a). In some embodiments, step (b) is performed about eleven (11) weeks after step (a). In some embodiments, step (b) is performed about twelve (12) weeks after step (a).

In some embodiments of the methods provided herein, the pathological complete response rate (pCRR) for the subject is at least 30%. In some embodiments, the pathological complete response rate (pCRR) for the subject is at least 35%. In some embodiments, the pathological complete response rate (pCRR) for the subject is at least 40%. In some embodiments, the pathological complete response rate (pCRR) for the subject is at least 45%. In some embodiments, the pathological complete response rate (pCRR) for the subject is at least 50%. In some embodiments, the pathological complete response rate (pCRR) for the subject is at least 55%. In some embodiments, the pathological complete response rate (pCRR) for the subject is at least 60%. In some embodiments, the pathological complete response rate (pCRR) for the subject is at least 65%. In some embodiments, the pathological complete response rate (pCRR) for the subject is at least 70%. In some embodiments, the pathological complete response rate (pCRR) for the subject is at least 75%.

In some embodiments of the methods provided herein, a population of the subjects is treated by the methods, and the pathological complete response rate (pCRR) in the treated population is at least 30%. In some embodiments, a population of the subjects is treated by the methods, and the pathological complete response rate (pCRR) in the treated population is at least 35%. In some embodiments, a population of the subjects is treated by the methods, and the pathological complete response rate (pCRR) in the treated population is at least 40%. In some embodiments, a population of the subjects is treated by the methods, and the pathological complete response rate (pCRR) in the treated population is at least 45%. In some embodiments, a population of the subjects is treated by the methods, and the pathological complete response rate (pCRR) in the treated population is at least 50%. In some embodiments, a population of the subjects is treated by the methods, and the pathological complete response rate (pCRR) in the treated population is at least 55%. In some embodiments, a population of the subjects is treated by the methods, and the pathological complete response rate (pCRR) in the treated population is at least 60%. In some embodiments, a population of the subjects is treated by the methods, and the pathological complete response rate (pCRR) in the treated population is at least 65%. In some embodiments, a population of the subjects is treated by the methods, and the pathological complete response rate (pCRR) in the treated population is at least 70%. In some embodiments, a population of the subjects is treated by the methods, and the pathological complete response rate (pCRR) in the treated population is at least 75%.

In some embodiments of the methods provided herein, the pathological complete response rate (pCRR) for a population of subjects treated with the ADC is at least 30%. In some embodiments, the pathological complete response rate (pCRR) for a population of subjects treated with the ADC is at least 35%. In some embodiments, the pathological complete response rate (pCRR) for a population of subjects treated with the ADC is at least 40%. In some embodiments, the pathological complete response rate (pCRR) for a population of subjects treated with the ADC is at least 45%. In some embodiments, the pathological complete response rate (pCRR) for a population of subjects treated with the ADC is at least 50%. In some embodiments, the pathological complete response rate (pCRR) for a population of subjects treated with the ADC is at least 55%. In some embodiments, the pathological complete response rate (pCRR) for a population of subjects treated with the ADC is at least 60%. In some embodiments, the pathological complete response rate (pCRR) for a population of subjects treated with the ADC is at least 65%. In some embodiments, the pathological complete response rate (pCRR) for a population of subjects treated with the ADC is at least 70%. In some embodiments, the pathological complete response rate (pCRR) for a population of subjects treated with the ADC is at least 75%.

In some embodiments of the methods provided herein, the pathological downstaging rate (pDSR) for the subject is at least 50%. In some embodiments, the pathological downstaging rate (pDSR) for the subject is at least 55%. In some embodiments, the pathological downstaging rate (pDSR) for the subject is at least 60%. In some embodiments, the pathological downstaging rate (pDSR) for the subject is at least 65%. In some embodiments, the pathological downstaging rate (pDSR) for the subject is at least 70%. In some embodiments, the pathological downstaging rate (pDSR) for the subject is at least 75%. In some embodiments, the pathological downstaging rate (pDSR) for the subject is at least 80%. In some embodiments, the pathological downstaging rate (pDSR) for the subject is at least 85%. In some embodiments, the pathological downstaging rate (pDSR) for the subject is at least 90%.

In some embodiments of the methods provided herein, a population of the subjects is treated by the methods, and the pathological downstaging rate (pDSR) in the treated population is at least 50%. In some embodiments, a population of the subjects is treated by the methods, and the pathological downstaging rate (pDSR) in the treated population is at least 55%. In some embodiments, a population of the subjects is treated by the methods, and the pathological downstaging rate (pDSR) in the treated population is at least 60%. In some embodiments, a population of the subjects is treated by the methods, and the pathological downstaging rate (pDSR) in the treated population is at least 65%. In some embodiments, a population of the subjects is treated by the methods, and the pathological downstaging rate (pDSR) in the treated population is at least 70%. In some embodiments, a population of the subjects is treated by the methods, and the pathological downstaging rate (pDSR) in the treated population is at least 75%. In some embodiments, a population of the subjects is treated by the methods, and the pathological downstaging rate (pDSR) in the treated population is at least 80%. In some embodiments, a population of the subjects is treated by the methods, and the pathological downstaging rate (pDSR) in the treated population is at least 85%. In some embodiments, a population of the subjects is treated by the methods, and the pathological downstaging rate (pDSR) in the treated population is at least 90%.

In some embodiments of the methods provided herein, the pathological downstaging rate (pDSR) for a population of subjects treated with the ADC is at least 50%. In some embodiments, the pathological downstaging rate (pDSR) for a population of subjects treated with the ADC is at least 55%. In some embodiments, the pathological downstaging rate (pDSR) for a population of subjects treated with the ADC is at least 60%. In some embodiments, the pathological downstaging rate (pDSR) for a population of subjects treated with the ADC is at least 65%. In some embodiments, the pathological downstaging rate (pDSR) for a population of subjects treated with the ADC is at least 70%. In some embodiments, the pathological downstaging rate (pDSR) for a population of subjects treated with the ADC is at least 75%.

In one aspect, provided herein are methods of treating urothelial cancer or bladder cancer in a human subject, comprising: administering to the subject multiple cycles of an effective amount of an antibody drug conjugate (ADC), wherein (a) the ADC comprises an antibody or antigen binding fragment thereof that binds to 191P4D12 (Nectin-4) conjugated to one or more units of monomethyl auristatin E (MMAE); (b) the subject is ineligible to receive cisplatin treatment (cisplatin ineligible); and (c) the number of cycles of ADC treatment administered to the cisplatin ineligible subject is equal to or less than the number of cycles of standard-of-care (SOC) therapy used to treat cisplatin eligible subjects with MIUC or MIBC. In some embodiments, the effective amount of the ADC is administered to the subject on days 1 and 8 of every 3 week cycle for a total of 3 cycles. In some embodiments, the effective amount of the ADC is about 1.25 mg/kg of the subject's body weight. In some embodiments, the effective amount of the ADC is about 1.0 mg/kg of the subject's body weight.

In one aspect, provided herein are methods of treating muscle invasive urothelial cancer (MIUC) or muscle invasive bladder cancer (MIBC) in a human subject, comprising: administering to the subject multiple cycles of an effective amount of an antibody drug conjugate (ADC), wherein (a) the ADC comprises an antibody or antigen binding fragment thereof that binds to 191P4D12 (Nectin-4) conjugated to one or more units of monomethyl auristatin E (MMAE); (b) the subject is ineligible to receive cisplatin treatment (cisplatin ineligible); and (c) the number of cycles of ADC treatment administered to the cisplatin ineligible subject is equal to or less than the number of cycles of standard-of-care (SOC) therapy used to treat cisplatin eligible subjects with MIUC or MIBC.

In some embodiments of the methods provided herein, the SOC therapy for a cisplatin eligible subject comprises cisplatin. In some embodiments, the SOC therapy for the cisplatin eligible subject comprises (i) methotrexate, vinblastine, doxorubicin, and cisplatin (MVAC), or (ii) gemcitabine plus cisplatin. In some embodiments, the SOC therapy for the cisplatin eligible subject comprises methotrexate, vinblastine, doxorubicin, and cisplatin (MVAC). In some embodiments, the SOC therapy for the cisplatin eligible subject comprises gemcitabine plus cisplatin. In some embodiments, the SOC therapy for the cisplatin eligible subject comprises administering a programmed cell death 1 (PD-1) inhibitor or a programmed cell death-ligand 1 (PD-L1) inhibitor. In some embodiments, the SOC therapy for a cisplatin eligible subject comprises: i. cisplatin; ii. methotrexate, vinblastine, doxorubicin, and cisplatin (MVAC); iii. gemcitabine plus cisplatin; iv. administering a programmed cell death 1 (PD-1) inhibitor; or v. administering a programmed cell death-ligand 1 (PD-L1) inhibitor.

In some embodiments of the methods provided herein, the number of cycles of ADC treatment administered to the cisplatin ineligible subject is 1, 2, 3, or 4. In some embodiments, the number of cycles of ADC treatment administered to the cisplatin ineligible subject is 3 or 4. In some embodiments, the number of cycles of ADC treatment administered to the cisplatin ineligible subject is 4. In some embodiments, the number of cycles of ADC treatment administered to the cisplatin ineligible subject is 3. In some embodiments, the number of cycles of ADC treatment administered to the cisplatin ineligible subject is 2. In some embodiments, the number of cycles of ADC treatment administered to the cisplatin ineligible subject is 1.

In some embodiments of the methods provided herein, the method further comprises performing radical cystectomy and pelvic lymph node dissection (RC+PLND) surgery on the subject. In some embodiments, the RC+PLND is performed about four (4) to about twelve (12) weeks after the ADC is administered to the human subject. In some embodiments, the RC+PLND is performed about five (5) to about twelve (12) weeks after the ADC is administered to the human subject. In some embodiments, the RC+PLND is performed about six (6) to about twelve (12) weeks after the ADC is administered to the human subject. In some embodiments, the RC+PLND is performed about seven (7) to about twelve (12) weeks after the ADC is administered to the human subject. In some embodiments, the RC+PLND is performed about eight (8) to about twelve (12) weeks after the ADC is administered to the human subject. In some embodiments, the RC+PLND is performed about nine (9) to about twelve (12) weeks after the ADC is administered to the human subject. In some embodiments, the RC+PLND is performed about ten (10) to about twelve (12) weeks after the ADC is administered to the human subject. In some embodiments, the RC+PLND is performed about eleven (11) to about twelve (12) weeks after the ADC is administered to the human subject. In some embodiments, the RC+PLND is performed about four (4) to about eleven (11) weeks after the ADC is administered to the human subject. In some embodiments, the RC+PLND is performed about four (4) to about ten (10) weeks after the ADC is administered to the human subject. In some embodiments, the RC+PLND is performed about four (4) to about nine (9) weeks after the ADC is administered to the human subject. In some embodiments, the RC+PLND is performed about four (4) to about eight (8) weeks after the ADC is administered to the human subject. In some embodiments, the RC+PLND is performed about four (4) to about seven (7) weeks after the ADC is administered to the human subject. In some embodiments, the RC+PLND is performed about four (4) to about six (6) weeks after the ADC is administered to the human subject. In some embodiments, the RC+PLND is performed about four (4) to about five (5) weeks after the ADC is administered to the human subject. In some embodiments, the RC+PLND is performed about six (6) to about eight (8) weeks after the ADC is administered to the human subject. In some embodiments, the RC+PLND is performed about seven (7) to about nine (9) weeks after the ADC is administered to the human subject. In some embodiments, the RC+PLND is performed about eight (8) to about ten (10) weeks after the ADC is administered to the human subject. In some embodiments, the RC+PLND is performed about nine (9) to about eleven (11) weeks after the ADC is administered to the human subject.

In some embodiments, the RC+PLND is performed about four (4) weeks after the ADC is administered to the human subject. In some embodiments, the RC+PLND is performed about five (5) weeks after the ADC is administered to the human subject. In some embodiments, the RC+PLND is performed about six (6) weeks after the ADC is administered to the human subject. In some embodiments, the RC+PLND is performed about seven (7) weeks after the ADC is administered to the human subject. In some embodiments, the RC+PLND is performed about eight (8) weeks after the ADC is administered to the human subject. In some embodiments, the RC+PLND is performed about nine (9) weeks after the ADC is administered to the human subject. In some embodiments, the RC+PLND is performed about ten (10) weeks after the ADC is administered to the human subject. In some embodiments, the RC+PLND is performed about eleven (11) weeks after the ADC is administered to the human subject. In some embodiments, the RC+PLND is performed about twelve (12) weeks after the ADC is administered to the human subject.

In one aspect, provided herein are methods of administering neoadjuvant or perioperative therapy to treat muscle invasive urothelial cancer (MIUC) or muscle invasive bladder cancer (MIBC) in a human subject, comprising: administering to the subject multiple cycles of an effective amount of an antibody drug conjugate (ADC), wherein (a) the ADC comprises an antibody or antigen binding fragment thereof that binds to 191P4D12 (Nectin-4) conjugated to one or more units of monomethyl auristatin E (MMAE); (b) the subject is ineligible to receive cisplatin treatment (cisplatin ineligible); and (c) the subject remains eligible for radical cystectomy and pelvic lymph node dissection (RC+PLND) surgery following the multiple cycles of treatment with the ADC. In some embodiments, the effective amount of the ADC is administered to the subject on days 1 and 8 of every 3 week cycle for a total of 3 cycles. In some embodiments, the effective amount of the ADC is about 1.25 mg/kg of the subject's body weight. In some embodiments, the effective amount of the ADC is about 1.0 mg/kg of the subject's body weight.

In some embodiments, the method further comprises performing radical cystectomy and pelvic lymph node dissection (RC+PLND) surgery on the subject. In some embodiments, the ADC is administered as a neoadjuvant therapy prior to the surgery. In some embodiments, the ADC is administered prior to and following the surgery. In some embodiments, the ADC is administered as a neoadjuvant therapy (i) prior to the surgery or (ii) prior to and following the surgery. In some embodiments, the RC+PLND is performed about four (4) to about twelve (12) weeks after the ADC is administered to the human subject. In some embodiments, the RC+PLND is performed about five (5) to about twelve (12) weeks after the ADC is administered to the human subject. In some embodiments, the RC+PLND is performed about six (6) to about twelve (12) weeks after the ADC is administered to the human subject. In some embodiments, the RC+PLND is performed about seven (7) to about twelve (12) weeks after the ADC is administered to the human subject. In some embodiments, the RC+PLND is performed about eight (8) to about twelve (12) weeks after the ADC is administered to the human subject. In some embodiments, the RC+PLND is performed about nine (9) to about twelve (12) weeks after the ADC is administered to the human subject. In some embodiments, the RC+PLND is performed about ten (10) to about twelve (12) weeks after the ADC is administered to the human subject. In some embodiments, the RC+PLND is performed about eleven (11) to about twelve (12) weeks after the ADC is administered to the human subject. In some embodiments, the RC+PLND is performed about four (4) to about eleven (11) weeks after the ADC is administered to the human subject. In some embodiments, the RC+PLND is performed about four (4) to about ten (10) weeks after the ADC is administered to the human subject. In some embodiments, the RC+PLND is performed about four (4) to about nine (9) weeks after the ADC is administered to the human subject. In some embodiments, the RC+PLND is performed about four (4) to about eight (8) weeks after the ADC is administered to the human subject. In some embodiments, the RC+PLND is performed about four (4) to about seven (7) weeks after the ADC is administered to the human subject. In some embodiments, the RC+PLND is performed about four (4) to about six (6) weeks after the ADC is administered to the human subject. In some embodiments, the RC+PLND is performed about four (4) to about five (5) weeks after the ADC is administered to the human subject. In some embodiments, the RC+PLND is performed about six (6) to about eight (8) weeks after the ADC is administered to the human subject. In some embodiments, the RC+PLND is performed about seven (7) to about nine (9) weeks after the ADC is administered to the human subject. In some embodiments, the RC+PLND is performed about eight (8) to about ten (10) weeks after the ADC is administered to the human subject. In some embodiments, the RC+PLND is performed about nine (9) to about eleven (11) weeks after the ADC is administered to the human subject.

In some embodiments, the RC+PLND is performed about four (4) weeks after the ADC is administered to the human subject. In some embodiments, the RC+PLND is performed about five (5) weeks after the ADC is administered to the human subject. In some embodiments, the RC+PLND is performed about six (6) weeks after the ADC is administered to the human subject. In some embodiments, the RC+PLND is performed about seven (7) weeks after the ADC is administered to the human subject. In some embodiments, the RC+PLND is performed about eight (8) weeks after the ADC is administered to the human subject. In some embodiments, the RC+PLND is performed about nine (9) weeks after the ADC is administered to the human subject. In some embodiments, the RC+PLND is performed about ten (10) weeks after the ADC is administered to the human subject. In some embodiments, the RC+PLND is performed about eleven (11) weeks after the ADC is administered to the human subject. In some embodiments, the RC+PLND is performed about twelve (12) weeks after the ADC is administered to the human subject.

In some embodiments, the ADC is administered about one (1) week following the surgery. In some embodiments, the ADC is administered about two (2) weeks following the surgery. In some embodiments, the ADC is administered about three (3) weeks following the surgery. In some embodiments, the ADC is administered about four (4) weeks following the surgery. In some embodiments, the ADC is administered about five (5) weeks following the surgery. In some embodiments, the ADC is administered about six (6) weeks following the surgery. In some embodiments, the ADC is administered about two (2) months following the surgery. In some embodiments, the ADC is administered about three (3) months following the surgery. In some embodiments, the ADC is administered about four (4) months following the surgery. In some embodiments, the ADC is administered about five (5) months following the surgery. In some embodiments, the ADC is administered about six (6) months following the surgery. In some embodiments, the ADC is administered about seven (7) months following the surgery. In some embodiments, the ADC is administered about eight (8) months following the surgery. In some embodiments, the ADC is administered about nine (9) months following the surgery. In some embodiments, the ADC is administered about ten (10) months following the surgery. In some embodiments, the ADC is administered about eleven (11) months following the surgery. In some embodiments, the ADC is administered about twelve (12) months following the surgery.

Standard-of-care (SOC) therapies in the treatment of muscle invasive bladder cancer (MIBC) include cisplatin-based neoadjuvant chemotherapy (NAC) (plus definitive local treatment (cystectomy or radiation)) (See, e.g., Advanced Bladder Cancer Meta-analysis, Eur Urol., 2005, 48(2):202-5), accelerated or dose dense methotrexate, vinblastine, doxorubicin, and cisplatin (MVAC) (See, e.g., Choueiri et al., J Clin Oncol, 2014, 32(18):1889-94; Plimack et al., J Clin Oncol., 2014, 32(18):1895-901), gemcitabine plus cisplatin, programmed cell death 1 (PD-1) inhibitors (See, e.g., Necchi et al., J Clin Oncol., 2018, 36(34):3353-60), and programmed cell death-ligand 1 (PD-L1) inhibitors (See, e.g., Powles et al., 2018, J Clin Oncol., 36(Suppl 15):Abstract 4506). Exemplary measures of efficacy of treatment include pathological complete response rate (pCRR), pathological downstaging rates (pDSR), disease free survival (DFS), event-free survival (EFS), overall survival (OS), progression free survival (PFS), and/or duration of response (DoR). For example, a 5-year OS of 50% was observed for patients treated with cisplatin-based NAC, compared to a 45% OS for patients who received definitive local therapy alone (See, e.g., Advanced Bladder Cancer Meta-analysis, Eur Urol., 2005, 48(2):202-5). For patients treated with accelerated or dose dense MVAC, around 30% to 40% of patients achieved pCR rate and 50% had pathologic downstaging (Choueiri 2014; Plimack 2014). For patients treated with pembrolizumab (which is a PD-1 inhibitor), a 42% pCR rate among 50 treated patients with a 54.3% pCR rate in patients with PD L1 positive disease and a pathologic downstaging rate of 65.7% has been observed (Necchi 2018). For patients treated with atezolizumab (which is a PD-L1 inhibitor), pCR was observed in 29% of patients and in 40% of patients with PD-L1-positive disease compared to only 16% in the PD-L1-negative cohort, and a total of 39% of patients were down staged to non-muscle invasive disease (Powles 2018).

In one aspect, provided herein are methods of treating muscle invasive urothelial cancer (MIUC) or muscle invasive bladder cancer (MIBC) in a human subject, comprising: administering to the subject multiple cycles of an effective amount of an antibody drug conjugate (ADC), wherein (a) the ADC comprises an antibody or antigen binding fragment thereof that binds to 191P4D12 (Nectin-4) conjugated to one or more units of monomethyl auristatin E (MMAE); (b) the subject is ineligible to receive cisplatin treatment (cisplatin ineligible); and (c) the efficacy of the treatment of the subject with the effective amount of the ADC is nearly as efficacious as treatment observed with cisplatin eligible patients treated with standard-of-care (SOC) therapy used to treat cisplatin eligible subjects with MIUC or MIBC.

In one aspect, provided herein are methods of treating muscle invasive urothelial cancer (MIUC) or muscle invasive bladder cancer (MIBC) in a human subject, comprising: administering to the subject multiple cycles of an effective amount of an antibody drug conjugate (ADC), wherein (a) the ADC comprises an antibody or antigen binding fragment thereof that binds to 191P4D12 (Nectin-4) conjugated to one or more units of monomethyl auristatin E (MMAE); (b) the subject is ineligible to receive cisplatin treatment (cisplatin ineligible); and (c) the efficacy of the treatment of the subject with the effective amount of the ADC is the same as the efficacy of treatment observed with cisplatin eligible patients treated with standard-of-care (SOC) therapy used to treat cisplatin eligible subjects with MIUC or MIBC.

In one aspect, provided herein are methods of treating muscle invasive urothelial cancer (MIUC) or muscle invasive bladder cancer (MIBC) in a human subject, comprising: administering to the subject multiple cycles of an effective amount of an antibody drug conjugate (ADC), wherein (a) the ADC comprises an antibody or antigen binding fragment thereof that binds to 191P4D12 (Nectin-4) conjugated to one or more units of monomethyl auristatin E (MMAE); (b) the subject is ineligible to receive cisplatin treatment (cisplatin ineligible); and (c) the efficacy of the treatment of the subject with the effective amount of the ADC is similar to the efficacy of treatment observed with cisplatin eligible patients treated with standard-of-care (SOC) therapy used to treat cisplatin eligible subjects with MIUC or MIBC. In some embodiments, the effective amount of the ADC is administered to the subject on days 1 and 8 of every 3 week cycle for a total of 3 cycles. In some embodiments, the effective amount of the ADC is about 1.25 mg/kg of the subject's body weight. In some embodiments, the effective amount of the ADC is about 1.0 mg/kg of the subject's body weight.

In some embodiments, the efficacy of the treatment is at least as efficacious as the efficacy of the treatment observed with SOC for cisplatin eligible subjects. In some embodiments, the measure of efficacy of treatment is one or more of: pathological complete response rate (pCRR), pathological downstaging rate (pDSR), disease free survival (DFS), event-free survival (EFS), overall survival (OS), progression free survival (PFS), and duration of response (DoR). In some embodiments, the measure of efficacy of treatment is pathological complete response rate (pCRR). In some embodiments, the measure of efficacy of treatment is pathological downstaging rate (pDSR). In some embodiments, the measure of efficacy of treatment is disease free survival (DFS). In some embodiments, the measure of efficacy of treatment is event-free survival (EFS). In some embodiments, the measure of efficacy of treatment is overall survival (OS). In some embodiments, the measure of efficacy of treatment is progression free survival (PFS). In some embodiments, the measure of efficacy of treatment is duration of response (DoR)

In some embodiments of the methods provided herein, the pathological complete response rate (pCRR) for the subject is at least 30%. In some embodiments, the pathological complete response rate (pCRR) for the subject is at least 35%. In some embodiments, the pathological complete response rate (pCRR) for the subject is at least 40%. In some embodiments, the pathological complete response rate (pCRR) for the subject is at least 45%. In some embodiments, the pathological complete response rate (pCRR) for the subject is at least 50%. In some embodiments, the pathological complete response rate (pCRR) for the subject is at least 55%. In some embodiments, the pathological complete response rate (pCRR) for the subject is at least 60%. In some embodiments, the pathological complete response rate (pCRR) for the subject is at least 65%. In some embodiments, the pathological complete response rate (pCRR) for the subject is at least 70%. In some embodiments, the pathological complete response rate (pCRR) for the subject is at least 75%.

In some embodiments of the methods provided herein, the pathological complete response rate (pCRR) for a population of subjects treated with the ADC is at least 30%. In some embodiments, the pathological complete response rate (pCRR) for a population of subjects treated with the ADC is at least 35%. In some embodiments, the pathological complete response rate (pCRR) for a population of subjects treated with the ADC is at least 40%. In some embodiments, the pathological complete response rate (pCRR) for a population of subjects treated with the ADC is at least 45%. In some embodiments, the pathological complete response rate (pCRR) for a population of subjects treated with the ADC is at least 50%. In some embodiments, the pathological complete response rate (pCRR) for a population of subjects treated with the ADC is at least 55%. In some embodiments, the pathological complete response rate (pCRR) for a population of subjects treated with the ADC is at least 60%. In some embodiments, the pathological complete response rate (pCRR) for a population of subjects treated with the ADC is at least 65%. In some embodiments, the pathological complete response rate (pCRR) for a population of subjects treated with the ADC is at least 70%. In some embodiments, the pathological complete response rate (pCRR) for a population of subjects treated with the ADC is at least 75%.

In some embodiments, the pathological downstaging rate (pDSR) for the subject is at least 50%. In some embodiments, the pathological downstaging rate (pDSR) for the subject is at least 55%. In some embodiments, the pathological downstaging rate (pDSR) for the subject is at least 60%. In some embodiments, the pathological downstaging rate (pDSR) for the subject is at least 65%. In some embodiments, the pathological downstaging rate (pDSR) for the subject is at least 70%. In some embodiments, the pathological downstaging rate (pDSR) for the subject is at least 75%. In some embodiments, the pathological downstaging rate (pDSR) for the subject is at least 80%. In some embodiments, the pathological downstaging rate (pDSR) for the subject is at least 85%. In some embodiments, the pathological downstaging rate (pDSR) for the subject is at least 90%.

In some embodiments of the methods provided herein, the pathological downstaging rate (pDSR) for a population of subjects treated with the ADC is at least 50%. In some embodiments, the pathological downstaging rate (pDSR) for a population of subjects treated with the ADC is at least 55%. In some embodiments, the pathological downstaging rate (pDSR) for a population of subjects treated with the ADC is at least 60%. In some embodiments, the pathological downstaging rate (pDSR) for a population of subjects treated with the ADC is at least 65%. In some embodiments, the pathological downstaging rate (pDSR) for a population of subjects treated with the ADC is at least 70%. In some embodiments, the pathological downstaging rate (pDSR) for a population of subjects treated with the ADC is at least 75%.

In some embodiments of the methods provided herein, the SOC therapy for a cisplatin eligible subject comprises cisplatin. In some embodiments, the SOC therapy for the cisplatin eligible subject comprises (i) methotrexate, vinblastine, doxorubicin, and cisplatin (MVAC), or (ii) gemcitabine plus cisplatin. In some embodiments, the SOC therapy for the cisplatin eligible subject comprises methotrexate, vinblastine, doxorubicin, and cisplatin (MVAC). In some embodiments, the SOC therapy for the cisplatin eligible subject comprises gemcitabine plus cisplatin. In some embodiments, the SOC therapy for the cisplatin eligible subject comprises a programmed cell death 1 (PD-1) inhibitor or a programmed cell death-ligand 1 (PD-L1) inhibitor. In some embodiments, the SOC therapy for a cisplatin eligible subject comprises: i. cisplatin; ii. methotrexate, vinblastine, doxorubicin, and cisplatin (MVAC); iii. gemcitabine plus cisplatin; iv. a programmed cell death 1 (PD-1) inhibitor; or v. a programmed cell death-ligand 1 (PD-L1) inhibitor. In some embodiments, the SOC therapy for the cisplatin eligible subject comprises a programmed cell death 1 (PD-1) inhibitor. In some embodiments, the SOC therapy for the cisplatin eligible subject comprises a programmed cell death-ligand 1 (PD-L1) inhibitor. In some embodiments, the PD-1 inhibitor is nivolumab or pembrolizumab. In some embodiments, the PD-1 inhibitor is nivolumab. In some embodiments, the PD-1 inhibitor is pembrolizumab. In some embodiments, the PD-L1 inhibitor is selected from the group consisting of atezolizumab, avelumab, and durvalumab. In some embodiments, the PD-L1 inhibitor is atezolizumab, avelumab, or durvalumab. In some embodiments, the PD-L1 inhibitor is atezolizumab. In some embodiments, the PD-L1 inhibitor is avelumab. In some embodiments, the PD-L1 inhibitor is durvalumab. In some embodiments, (i) the PD-1 inhibitor is nivolumab or pembrolizumab; or (ii) the PD-L1 inhibitor is selected from the group consisting of atezolizumab, avelumab, and durvalumab. In some embodiments of the methods provided herein, the subject has cT2-T4aN0M0 stage MIBC stage MIBC.

In some of the embodiments of the methods provided herein, the effective amount of ADC is about 1.25 mg/kg.

In some of the embodiments of the methods provided herein, the cancer is urothelial or bladder cancer. In some embodiments, the cancer is urothelial cancer. In some embodiments, the cancer is bladder cancer. In some embodiments, the cancer is muscle invasive urothelial cancer (MIUC). In some embodiments, the cancer is muscle invasive bladder cancer (MIBC).

### 5.2.1.1 Cisplatin Ineligible Patients

Various conditions can be used to determine the cisplatin ineligibility for the human subjects for the methods provided herein, including but not limited to the methods of the preceding paragraphs.

In one embodiment, the conditions for determining the cisplatin ineligibility comprise or consist of GFR<60 mL/min but ≥30 mL/min, wherein the GFR is measured by the Cockcroft-Gault formula, Modification of Diet in Renal Disease equations (MDRD), or 24-hour urine collection. In some embodiments, the conditions for determining the cisplatin ineligibility comprise or consist of ECOG performance status score of 2. In some embodiments, the conditions for determining the cisplatin ineligibility comprise or consist of NCI CTCAE Version 4.03 Grade ≥2 hearing loss. In some embodiments, the conditions for determining the cisplatin ineligibility comprise or consist of NYHA Class III heart failure. In some embodiments, the subject is considered cis-platin ineligible if one or more of the following criteria are satisfied: (a) GFR<60 mL/min but ≥30 mL/min, (wherein the GFR is measured by the Cockcroft-Gault formula, Modification of Diet in Renal Disease equations (MDRD), or 24-hour urine collection); (b) ECOG performance status of 2; (c) NCI CTCAE Version 4.03 Grade ≥2 hearing loss; and (d) NYHA Class III heart failure. In some embodiments, the conditions for determining the cisplatin ineligibility comprise or consist of GFR<60 mL/min but ≥30 mL/min, wherein the GFR is measured by the Cockcroft-Gault formula, Modification of Diet in Renal Disease equations (MDRD), or 24-hour urine collection, and ECOG performance status score of 2. In some embodiments, the conditions for determining the cisplatin ineligibility comprise or consist of GFR<60 mL/min but ≥30 mL/min, wherein the GFR is measured by the Cockcroft-Gault formula, Modification of Diet in Renal Disease equations (MDRD), or 24-hour urine collection, and NCI CTCAE Version 4.03 Grade ≥2 hearing loss. In some embodiments, the conditions for determining the cisplatin ineligibility comprise or consist of GFR<60 mL/min but ≥30 mL/min, wherein the GFR is measured by the Cockcroft-Gault formula, Modification of Diet in Renal Disease equations (MDRD), or 24-hour urine collection, and NYHA Class III heart failure. In some embodiments, the conditions for determining the cisplatin ineligibility comprise or consist of ECOG performance status score of 2 and NCI CTCAE Version 4.03 Grade ≥2 hearing loss. In some embodiments, the conditions for determining the cisplatin ineligibility comprise or consist of ECOG performance status score of 2 and NYHA Class III heart failure. In some embodiments, the conditions for determining the cisplatin ineligibility comprise or consist of NCI CTCAE Version 4.03 Grade ≥2 hearing loss and NYHA Class III heart failure. In some embodiments, the human subjects for whom the methods provided herein can have any three of GFR<60 mL/min but ≥30 mL/min, wherein the GFR is measured by the Cockcroft-Gault formula, Modification of Diet in Renal Disease equations (MDRD), or 24-hour urine collection, ECOG performance status score of 2, NCI CTCAE Version 4.03 Grade ≥2 hearing loss, and NYHA Class III heart failure. In some embodiments, the subject is considered cis-platin ineligible if one or more of the following criteria are satisfied: (a) GFR<60 mL/min but ≥30 mL/min, wherein the GFR is measured by the Cockcroft-Gault formula, Modification of Diet in Renal Disease equations (MDRD), or 24-hour urine collection, (b) ECOG performance status score of 2, (c) NCI CTCAE Version 4.03 Grade ≥2 hearing loss, and (d) NYHA Class III heart failure. In some embodiments, the human subjects for whom the methods provided herein can have one or more of GFR<60 mL/min but ≥30 mL/min, wherein the GFR is measured by the Cockcroft-Gault formula, Modification of Diet in Renal Disease equations (MDRD), or 24-hour urine collection, ECOG performance status score of 2, NCI CTCAE Version 4.03 Grade 2 hearing loss, and NYHA Class III heart failure.

In other embodiments, the conditions for determining the cisplatin ineligibility comprise or consist of ECOG performance status score of 2. In some embodiments, the conditions for determining the cisplatin ineligibility comprise or consist of impaired renal function. In certain embodiments, the conditions for determining the cisplatin ineligibility comprise or consist of no less than Grade 2 hearing loss. In one embodiment, the conditions for determining the cisplatin ineligibility comprise or consist of ECOG performance status score of 2 and impaired renal function. In some embodiments, the conditions for determining the cisplatin ineligibility comprise or consist of ECOG performance status score of 2 and no less than Grade 2 hearing loss. In further embodiments, the conditions for determining the cisplatin ineligibility comprise or consist of impaired renal function and no less than Grade 2 hearing loss. In yet other embodiments, the conditions for determining the cisplatin ineligibility comprise or consist of ECOG performance status score of 2, impaired renal function, and no less than Grade 2 hearing loss. In some embodiments, the conditions for determining the cisplatin ineligibility comprise or consist of any one of ECOG performance status score of 2, impaired renal function, and no less than Grade 2 hearing loss. In some embodiments, the conditions for determining the cisplatin ineligibility comprise or consist of any two of ECOG performance status score of 2, impaired renal function, and no less than Grade 2 hearing loss, in any combination or permutation. In some embodiments, the conditions for determining the cisplatin ineligibility comprise or consist of all three of ECOG performance status score of 2, impaired renal function, and no less than Grade 2 hearing loss.

Impaired renal function can be determined as various means known and available in the art. Various embodiments are provided herein to determine the impaired renal function for the human subjects for the methods provided herein, including but not limited to the methods of the preceding paragraph. In one embodiment, the impaired renal function is determined by creatinine clearance (CrCl) less than 60 mL/min. In some embodiments, the impaired renal function is determined by CrCl less than 60 but no less than 30 mL/min. In certain embodiments, the impaired renal function is determined by CrCl less than 30 but no less than 15 mL/min. In some embodiments of the methods provided in this paragraph, the CrCl is measured by 24 hour urine collection. In other embodiments of the methods provided in this paragraph, the CrCl is estimated by the Cockcroft-Gault criteria.

As such, some specific conditions based on creatinine clearance can be used to determine the cisplatin ineligibility for the human subjects for the methods provided herein, including but not limited to the methods of the preceding paragraphs. In some embodiments, the conditions for determining the cisplatin ineligibility comprise or consist of CrCl less than 60 mL/min. In one embodiment, the conditions for determining the cisplatin ineligibility comprise or consist of ECOG performance status score of 2 and CrCl less than 60 mL/min. In further embodiments, the conditions for determining the cisplatin ineligibility comprise or consist of CrCl less than 60 mL/min and no less than Grade 2 hearing loss. In yet other embodiments, the conditions for determining the cisplatin ineligibility comprise or consist of ECOG performance status score of 2, CrCl less than 60 mL/min, and no less than Grade 2 hearing loss. In some embodiments, the conditions for determining the cisplatin ineligibility comprise or consist of any one of ECOG performance status score of 2, CrCl less than 60 mL/min, and no less than Grade 2 hearing loss. In some embodiments, the conditions for determining the cisplatin ineligibility comprise or consist of any two of ECOG performance status score of 2, CrCl less than 60 mL/min, and no less than Grade 2 hearing loss, in any combination or permutation. In some embodiments, the conditions for determining the cisplatin ineligibility comprise or consist of all three of ECOG performance status score of 2, CrCl less than 60 mL/min, and no less than Grade 2 hearing loss. In some embodiments of the methods provided in this paragraph, the CrCl is measured by 24 hour urine collection. In other embodiments of the methods provided in this paragraph, the CrCl is estimated by the Cockcroft-Gault criteria.

Alternatively, other specific conditions based on creatinine clearance can be used to determine the cisplatin ineligibility for the human subjects for the methods provided herein, including but not limited to the methods of the preceding paragraphs. In some embodiments, the conditions for determining the cisplatin ineligibility comprise or consist of CrCl less than 60 but no less than 30 mL/min. In one embodiment, the conditions for determining the cisplatin ineligibility comprise or consist of ECOG performance status score of 2 and CrCl less than 60 but no less than 30 mL/min. In further embodiments, the conditions for determining the cisplatin ineligibility comprise or consist of CrCl less than 60 but no less than 30 mL/min and no less than Grade 2 hearing loss. In yet other embodiments, the conditions for determining the cisplatin ineligibility comprise or consist of ECOG performance status score of 2, CrCl less than 60 but no less than 30 mL/min, and no less than Grade 2 hearing loss. In some embodiments, the conditions for determining the cisplatin ineligibility comprise or consist of any one of ECOG performance status score of 2, CrCl less than 60 but no less than 30 mL/min, and no less than Grade 2 hearing loss. In some embodiments, the conditions for determining the cisplatin ineligibility comprise or consist of any two of ECOG performance status score of 2, CrCl less than 60 but no less than 30 mL/min, and no less than Grade 2 hearing loss, in any combination or permutation. In some embodiments, the conditions for determining the cisplatin ineligibility comprise or consist of all three of ECOG performance status score of 2, CrCl less than 60 but no less than 30 mL/min, and no less than Grade 2 hearing loss. In some embodiments of the methods provided in this paragraph, the CrCl is measured by 24 hour urine collection. In other embodiments of the methods provided in this paragraph, the CrCl is estimated by the Cockcroft-Gault criteria.

Similarly, further specific conditions based on creatinine clearance can be used to determine the cisplatin ineligibility for the human subjects for the methods provided herein, including but not limited to the methods of the preceding paragraphs. In some embodiments, the conditions for determining the cisplatin ineligibility comprise or consist of CrCl less than 30 but no less than 15 mL/min. In one embodiment, the conditions for determining the cisplatin ineligibility comprise or consist of ECOG performance status score of 2 and CrCl less than 30 but no less than 15 mL/min. In further embodiments, the conditions for determining the cisplatin ineligibility comprise or consist of CrCl less than 30 but no less than 15 mL/min and no less than Grade 2 hearing loss. In yet other embodiments, the conditions for determining the cisplatin ineligibility comprise or consist of ECOG performance status score of 2, CrCl less than 30 but no less than 15 mL/min, and no less than Grade 2 hearing loss. In some embodiments, the conditions for determining the cisplatin ineligibility comprise or consist of any one of ECOG performance status score of 2, CrCl less than 30 but no less than 15 mL/min, and no less than Grade 2 hearing loss. In some embodiments, the conditions for determining the cisplatin ineligibility comprise or consist of any two of ECOG performance status score of 2, CrCl less than 30 but no less than 15 mL/min, and no less than Grade 2 hearing loss, in any combination or permutation. In some embodiments, the conditions for determining the cisplatin ineligibility comprise or consist of all three of ECOG performance status score of 2, CrCl less than 30 but no less than 15 mL/min, and no less than Grade 2 hearing loss. In some embodiments of the methods provided in this paragraph, the CrCl is measured by 24 hour urine collection. In other embodiments of the methods provided in this paragraph, the CrCl is estimated by the Cockcroft-Gault criteria.

### 5.2.1.2 Additional Patient Demographics

Additionally, the human subjects for whom the methods provided herein can be used are human subjects having various other conditions. In one embodiment, the human subjects for whom the methods provided herein may have histologically confirmed MIBC with predominant >50% urothelial histology (previously known as transitional cell carcinoma). In other embodiments, the human subject has cT2-T4aN0M0 stage MIBC. In other embodiments, the human subjects for whom the methods provided herein may have clinical stage cT2-T4aN0M0 determined by TURBT within 90 days prior to the first dose of treatment and by CT with IV contrast of the chest and a CT urogram of the abdomen and pelvis. In other embodiments, the human subjects for whom the methods provided herein may have mixed cell types, wherein urothelial cancer is predominant (>50%). In other embodiments, the human subjects for whom the methods provided herein may have an ECOG performance status of 0, 1, or 2. In other embodiments, the human subjects for whom the methods provided herein may have an anticipated life expectancy of ≥3 months. In other embodiments, the human subjects for whom the methods provided herein may be deemed eligible for RC+PLND by his/her urologist and/or oncologist. In some embodiments, the human subjects for whom the methods provided herein may have any two of: histologically confirmed MIBC with predominant >50% urothelial histology (previously known as transitional cell carcinoma); clinical stage cT2-T4aN0M0 determined by TURBT within 90 days prior to the first dose of treatment and by CT with IV contrast of the chest and a CT urogram of the abdomen and pelvis; mixed cell types, wherein urothelial cancer is predominant (>50%); an ECOG performance status of 0, 1, or 2; an anticipated life expectancy of ≥3 months; and deemed eligible for RC+PLND by his/her urologist and/or oncologist. In some embodiments, the human subjects for whom the methods provided herein may have any three of: histologically confirmed MIBC with predominant >50% urothelial histology (previously known as transitional cell carcinoma); clinical stage cT2-T4aN0M0 determined by TURBT within 90 days prior to the first dose of treatment and by CT with IV contrast of the chest and a CT urogram of the abdomen and pelvis; mixed cell types, wherein urothelial cancer is predominant (>50%); an ECOG performance status of 0, 1, or 2; an anticipated life expectancy of ≥3 months; and deemed eligible for RC+PLND by his/her urologist and/or oncologist. In some embodiments, the human subjects for whom the methods provided herein may have any four of: histologically confirmed MIBC with predominant >50% urothelial histology (previously known as transitional cell carcinoma); clinical stage cT2-T4aN0M0 determined by TURBT within 90 days prior to the first dose of treatment and by CT with IV contrast of the chest and a CT urogram of the abdomen and pelvis; mixed cell types, wherein urothelial cancer is predominant (>50%); an ECOG performance status of 0, 1, or 2; an anticipated life expectancy of ≥3 months; and deemed eligible for RC+PLND by his/her urologist and/or oncologist. In some embodiments, the human subjects for whom the methods provided herein may have any five of: histologically confirmed MIBC with predominant >50% urothelial histology (previously known as transitional cell carcinoma); clinical stage cT2-T4aN0M0 determined by TURBT within 90 days prior to the first dose of treatment and by CT with IV contrast of the chest and a CT urogram of the abdomen and pelvis; mixed cell types, wherein urothelial cancer is predominant (>50%); an ECOG performance status of 0, 1, or 2; an anticipated life expectancy of ≥3 months; and deemed eligible for RC+PLND by his/her urologist and/or oncologist. In some embodiments, the human subjects for whom the methods provided herein may have all five of: histologically confirmed MIBC with predominant >50% urothelial histology (previously known as transitional cell carcinoma); clinical stage cT2-T4aN0M0 determined by TURBT within 90 days prior to the first dose of treatment and by CT with IV contrast of the chest and a CT urogram of the abdomen and pelvis; mixed cell types, wherein urothelial cancer is predominant (>50%); an ECOG performance status of 0, 1, or 2; an anticipated life expectancy of ≥3 months; and deemed eligible for RC+PLND by his/her urologist and/or oncologist. In some embodiments, the human subjects for whom the methods provided herein may have one or more of: histologically confirmed MIBC with predominant >50% urothelial histology (previously known as transitional cell carcinoma); clinical stage cT2-T4aN0M0 determined by TURBT within 90 days prior to the first dose of treatment and by CT with IV contrast of the chest and a CT urogram of the abdomen and pelvis; mixed cell types, wherein urothelial cancer is predominant (>50%); an ECOG performance status of 0, 1, or 2; an anticipated life expectancy of ≥3 months; and deemed eligible for RC+PLND by his/her urologist and/or oncologist.

In some embodiments, the human subjects for whom the methods provided herein may have received prior intravesical Bacillus Calmette-Guérin (BCG) or intravesical chemotherapy for NMIBC.

In further embodiments of the methods provided herein, including the methods of the preceding paragraphs, the human subjects for whom the methods provided herein can be used are human subjects having various other conditions. In one embodiment, the human subjects for whom the methods provided herein can be used also has the condition of absolute neutrophil count (ANC) no less than 1500/μL. In other embodiments, the human subjects for whom the methods provided herein can be used also have the condition of platelet count no less than 100,000/μL. In other embodiments, the human subjects for whom the methods provided herein can be used also have the condition of hemoglobin no less than 9.0 g/dL or 5.6 mmol/L. In other embodiments, the human subjects for whom the methods provided herein can be used also have the condition of CrCl no more than 1.5 times of upper limit of normal (ULN). In other embodiments, the human subjects for whom the methods provided herein can be used also have the condition of creatinine no more than 1.5 times of upper limit of normal (ULN). In other embodiments, the human subjects for whom the methods provided herein can be used also have the condition of CrCl no less than 30 mL/min. In other embodiments, the human subjects for whom the methods provided herein can be used also have the condition of (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease. In other embodiments, the human subjects for whom the methods provided herein can be used also have the condition of AST (SGOT) and ALT (SGPT) no more than 3 times of ULN. In other embodiments, the human subjects for whom the methods provided herein can be used also have the condition of INR or PT no more than 1.5 times of upper limit of normal (ULN). In other embodiments, the human subjects for whom the methods provided herein can be used also have the condition of aPTT or PTT no more than 1.5 times of upper limit of normal (ULN). In some embodiments, the human subjects for whom the methods provided herein can be used also have any two of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN). In some embodiments, the human subjects for whom the methods provided herein can be used also have any three of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN). In some embodiments, the human subjects for whom the methods provided herein can be used also have any four of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN). In some embodiments, the human subjects for whom the methods provided herein can be used also have any five of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN). In some embodiments, the human subjects for whom the methods provided herein can be used also have any six of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN). In some embodiments, the human subjects for whom the methods provided herein can be used also have any seven of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN). In some embodiments, the human subjects for whom the methods provided herein can be used also have any eight of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN). In some embodiments, the human subjects for whom the methods provided herein can be used also have all nine of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN). In some embodiments, the human subjects for whom the methods provided herein can be used also have one or more of: absolute neutrophil count (ANC) no less than 1500/μL; platelet count no less than 100,000/μL; hemoglobin no less than 9.0 g/dL or 5.6 mmol/L; CrCl no more than 1.5 times of upper limit of normal (ULN); creatinine no more than 1.5 times of upper limit of normal (ULN); CrCl no less than 30 mL/min; (i) serum total bilirubin no more than 1.5 times of upper limit of normal (ULN), (ii) direct bilirubin no more than ULN for patients with total bilirubin levels greater than 1.5 times of ULN, or (iii) serum total bilirubin no more than 3 times of ULN for patients with Gilbert's disease; AST (SGOT) and ALT (SGPT) no more than 3 times of ULN; INR or PT no more than 1.5 times of upper limit of normal (ULN); and aPTT or PTT no more than 1.5 times of upper limit of normal (ULN).

In other embodiments of the methods provided herein, including the methods of the preceding paragraphs, the human subjects for whom the methods provided herein can be used are human subjects free from certain conditions. In one embodiment, the human subjects for whom the methods provided herein have not received prior systemic treatment, chemoradiation, or radiation therapy for MIBC. In one embodiment, the human subjects for whom the methods provided herein have not received any prior treatment with an immune checkpoint inhibitor (CPI) (e.g., a PD-1 inhibitor, PD-L1 inhibitor, or PD-L2 inhibitor, such as atezolizumab, pembrolizumab, nivolumab, durvalumab, or avelumab). In one embodiment, the human subjects for whom the methods provided herein has not received any prior treatment with an agent directed to another stimulatory or co inhibitory T-cell receptor (e.g., a CD137 agonist, a CTLA-4 inhibitor, or an OX-40 agonist). In one embodiment, the human subjects for whom the methods provided herein does not have evidence of nodal disease on imaging. In one embodiment, the human subjects for whom the methods provided herein does not have evidence of metastatic disease on imaging. In one embodiment, the human subjects for whom the methods provided herein have not undergone partial cystectomy of the bladder to remove any NMIBC or MIBC. In one embodiment, the human subjects for whom the methods provided herein do not have ongoing sensory or motor neuropathy Grade 2 or higher. In one embodiment, the human subjects for whom the methods provided herein do not have a condition requiring high doses of steroids (>10 mg/day of prednisone or equivalent) or other immunosuppressive medications. In one embodiment, the human subjects for whom the methods provided herein have not had prior treatment with enfortumab vedotin or other MMAE-based ADCs for urothelial cancer. In one embodiment, the human subjects for whom the methods provided herein have not had a history of another invasive malignancy within 3 years before the first dose of enfortumab vedotin (EV), or any evidence of residual disease from a previously diagnosed malignancy. In one embodiment, the human subjects for whom the methods provided herein. In one embodiment, the human subjects for whom the methods provided herein are not currently receiving systemic antimicrobial treatment for active infection (i.e., viral, bacterial, or fungal) at the time of first dose of enfortumab vedotin. In one embodiment, the human subjects for whom the methods provided herein are not positive for a hepatitis B surface antigen and/or an antihepatitis B core antibody. In one embodiment, the human subjects for whom the methods provided herein do not have an active hepatitis C infection or known HIV infection. In one embodiment, the human subjects for whom the methods provided herein do not have active tuberculosis. In one embodiment, the human subjects for whom the methods provided herein do not have a documented history of a cerebral vascular event (stroke or transient ischemic attack), unstable angina, myocardial infarction, or cardiac symptoms (including congestive heart failure) consistent with NYHA Class IV within 6 months prior to the first dose of enfortumab vedotin. In one embodiment, the human subjects for whom the methods provided herein do not have active keratitis or corneal ulcerations. In one embodiment, the human subjects for whom the methods provided herein do not have an active autoimmune disease that has required systemic treatment in past 2 years (i.e., use of disease modifying agents, corticosteroids, or immunosuppressive drugs). In one embodiment, the human subjects for whom the methods provided herein do not have a history of idiopathic pulmonary fibrosis; organizing pneumonia, drug-induced pneumonitis, idiopathic pneumonitis, or evidence of active pneumonitis on screening chest CT scan. In one embodiment, the human subjects for whom the methods provided herein have not had a prior allogeneic stem cell or solid organ transplant. In one embodiment, the human subjects for whom the methods provided herein have not been administered a live, attenuated vaccine within 30 days prior to first dose of study drug. In one embodiment, the human subjects for whom the methods provided herein do not have uncontrolled diabetes, wherein uncontrolled diabetes is defined as HbA1c≥8% or HbA1c 7% to <8% with associated diabetes symptoms (polyuria or polydipsia) that are not otherwise explained.

In all the methods provided herein and specifically those described in the preceding paragraphs: the ADCs that can be used are described in Sections 3, 5.2, 5.3, 5.4, 5.5, and 6, selection of patients for treatment is described herein and exemplified in this Section (Section 5.2) and Sections 3 and 6, dosing regimens and pharmaceutical composition for administering the therapeutic agent are described in this Section (Section 5.2), Sections 5.4, 5.7 and 6 below, the biomarkers that can be used for identifying the therapeutic agents, selecting the patients, determining the outcome of these methods, and/or serving as criteria in any way for these methods are described herein and exemplified in this Section (Section 5.2, including 5.2.1 and 5.2.2) and Section 6, the biomarkers can be determined as described in Section 5.8 or as known in the art, therapeutic outcomes for the methods provided herein are described in this Section (Section 5.2 including Section 5.2.1.4) and Sections 3 and 6, additional therapeutic outcomes for the methods provided herein can be improvement of the biomarkers described herein, for example, those described and exemplified in this Section (Section 5.2 including 5.2.2) and Sections 3 and 6, and combination therapies including the ADCs and other therapeutic agents are described in this Section (Section 5.2) and in Section 5.5. Therefore, a person skilled in the art would understand that the methods provided herein include all permutations and combinations of the patients, therapeutic agents, dosing regiments, biomarkers, and therapeutic outcomes as described above and below.

In certain embodiments, the methods provided herein are used for treating subjects having urothelial cancers that express 191P4D12 RNA, express 191P4D12 protein, or express both 191P4D12 RNA and 191P4D12 protein. In one embodiment, the methods provided herein are used for treating subjects who have urothelial cancers that express 191P4D12 RNA, or express 191P4D12 protein.

In certain embodiments, the methods provided herein are used for treating subjects having urothelial cancers that express 191P4D12 RNA, express 191P4D12 protein, or express both 191P4D12 RNA and 191P4D12 protein. In one embodiment, the methods provided herein are used for treating subjects who have urothelial cancers that express 191P4D12 RNA, or express 191P4D12 protein.

In some embodiments, the 191P4D12 RNA expression in the cancers is determined by polynucleotide hybridization, sequencing (assessing the relative abundance of the sequences), and/or PCR (including RT-PCR). In some embodiments, the 191P4D12 protein expression in the cancers is determined by IHC, analysis in fluorescence-activated cell sorting (FACS), and/or western blotting. In some embodiments, the 191P4D12 protein expression in the cancers is determined by more than one method. In some embodiments, the 191P4D12 protein expression in the cancers is determined by two methods of IHC.

In some embodiments, the subjects that can be treated in the methods provided herein have certain phenotypic or genotypic characteristics. In some embodiments, the subjects have any permutation and combination of the phenotypic or genotypic characteristics described herein.

In some embodiments, the phenotypic or genotypic characteristics are determined histologically, cytologically, or both histologically and cytologically. In some embodiments of methods provided herein, the histological and/or the cytological determination of the phenotypic and/or genotypic characteristics are performed as described in American Society of Clinical Oncology/College of American Pathologists (ASCO/CAP) guidelines based on the most recently analyzed tissue, which is incorporated herein in their entirety by reference. In some embodiments, the phenotypic or genotypic characteristics are determined by sequencing including the next generation sequencing (e.g. NGS from Illumina, Inc), DNA hybridization, and/or RNA hybridization.

5.2.1.3 Optional Exclusion of Patients with Prior Checkpoint Inhibitor (CPI) Treatment

In various aspects or embodiments of the methods provided herein, including the methods provided in this Section (Section 5.2) such as the methods provided in this and the preceding paragraphs, the methods may refer to subjects who have not received any prior treatment with an immune checkpoint inhibitor (e.g., the human subjects for whom the methods provided herein have not received any prior treatment with an immune checkpoint inhibitor (CPI) (e.g., a PD-1 inhibitor, PD-L1 inhibitor, or PD-L2 inhibitor, such as atezolizumab, pembrolizumab, nivolumab, durvalumab, or avelumab)).

As used herein, the term “immune checkpoint inhibitor” or “checkpoint inhibitor” refers to molecules that totally or partially reduce, inhibit, interfere with or modulate one or more checkpoint proteins. Numerous checkpoint proteins are known, such as CTLA-4 and its ligands CD80 and CD86; and PD-1 with its ligands PD-L1 and PD-L2 (Pardoll, Nature Reviews Cancer, 2012, 12, 252-264). Other exemplary checkpoint proteins include LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. These proteins appear responsible for co-stimulatory or inhibitory interactions of T-cell responses. Immune checkpoint proteins appear to regulate and maintain self-tolerance and the duration and amplitude of physiological immune responses. Immune checkpoint inhibitors include antibodies or are derived from antibodies.

In certain embodiments, the checkpoint inhibitor can be inhibitors or activators against a checkpoint protein that is upregulated in cancer. In some specific embodiments, the checkpoint inhibitor can be inhibitor or activator against a checkpoint protein including LAG-3, B7, TIM3 (HAVCR2), OX40 (CD134), GITR, CD137, CD40, VTCN1, IDO1, CD276, PVRIG, TIGIT, CD25 (IL2RA), IFNAR2, IFNAR1, CSF1R, VSIR (VISTA), or HLA. In some embodiments, the checkpoint inhibitor can be an inhibitors or activators selected from the group consisting of a PD-1 inhibitor, a PD-L1 inhibitor, a PD-L2 inhibitor, a CTLA-4 inhibitor, a LAG-3 inhibitor, a B7 inhibitor, a TIM3 (HAVCR2) inhibitor, an OX40 (CD134) inhibitor, a GITR agonist, a CD137 agonist, or a CD40 agonist, a VTCN1 inhibitor, an IDO1 inhibitor, a CD276 inhibitor, a PVRIG inhibitor, a TIGIT inhibitor, a CD25 (IL2RA) inhibitor, an IFNAR2 inhibitor, an IFNAR1 inhibitor, a CSF1R inhibitor, a VSIR (VISTA) inhibitor, or a therapeutic agent targeting HLA. Such inhibitors, activators, or therapeutic agents are further provided below.

In some embodiments, the checkpoint inhibitor is a CTLA-4 inhibitor. In one embodiment, the CTLA-4 inhibitor is an anti-CTLA-4 antibody. Examples of anti-CTLA-4 antibodies include, but are not limited to, those described in U.S. Pat. Nos. 5,811,097; 5,811,097; 5,855,887; 6,051,227; 6,207,157; 6,682,736; 6,984,720; and 7,605,238, all of which are incorporated herein in their entireties. In one embodiment, the anti-CTLA-4 antibody is tremelimumab (also known as ticilimumab or CP-675,206). In another embodiment, the anti-CTLA-4 antibody is ipilimumab (also known as MDX-010 or MDX-101). Ipilimumab is a fully human monoclonal IgG antibody that binds to CTLA-4. Ipilimumab is marketed under the trade name Yervoy™.

In certain embodiments, the checkpoint inhibitor is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In some embodiments, the checkpoint inhibitor is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106) or pembrolizumab (also known as MK-3475, SCH 900475, or lambrolizumab). In one embodiment, the anti-PD-1 antibody is nivolumab. Nivolumab is a human IgG4 anti-PD-1 monoclonal antibody, and is marketed under the trade name Opdivo™. In another embodiment, the anti-PD-1 antibody is pembrolizumab. Pembrolizumab is a humanized monoclonal IgG4 antibody and is marketed under the trade name Keytruda™. In yet another embodiment, the anti-PD-1 antibody is CT-011, a humanized antibody. CT-011 administered alone has failed to show response in treating acute myeloid leukemia (AML) at relapse. In yet another embodiment, the anti-PD-1 antibody is AMP-224, a fusion protein. In another embodiment, the PD-1 antibody is BGB-A317. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab. In another embodiment, the PD-1 antibody is camrelizumab. In a further embodiment, the PD-1 antibody is sintilimab. In some embodiments, the PD-1 antibody is tislelizumab. In certain embodiments, the PD-1 antibody is TSR-042. In yet another embodiment, the PD-1 antibody is PDR001. In yet another embodiment, the PD-1 antibody is toripalimab.

In certain embodiments, the checkpoint inhibitor is a PD-L1 inhibitor. In one embodiment, the PD-L1 inhibitor is an anti-PD-L1 antibody. In one embodiment, the anti-PD-L1 antibody is MEDI4736 (durvalumab). In another embodiment, the anti-PD-L1 antibody is BMS-936559 (also known as MDX-1105-01). In yet another embodiment, the PD-L1 inhibitor is atezolizumab (also known as MPDL3280A, and Tecentriq®). In a further embodiment, the PD-L1 inhibitor is avelumab.

In one embodiment, the checkpoint inhibitor is a PD-L2 inhibitor. In one embodiment, the PD-L2 inhibitor is an anti-PD-L2 antibody. In one embodiment, the anti-PD-L2 antibody is rHIgM12B7A.

In one embodiment, the checkpoint inhibitor is a lymphocyte activation gene-3 (LAG-3) inhibitor. In one embodiment, the LAG-3 inhibitor is IMP321, a soluble Ig fusion protein (Brignone et al., J. Immunol., 2007, 179, 4202-4211). In another embodiment, the LAG-3 inhibitor is BMS-986016.

In one embodiment, the checkpoint inhibitors is a B7 inhibitor. In one embodiment, the B7 inhibitor is a B7-H3 inhibitor or a B7-H4 inhibitor. In one embodiment, the B7-H3 inhibitor is MGA271, an anti-B7-H3 antibody (Loo et al., Clin. Cancer Res., 2012, 3834).

In one embodiment, the checkpoint inhibitors is a TIM3 (T-cell immunoglobulin domain and mucin domain 3) inhibitor (Fourcade et al., J. Exp. Med., 2010, 207, 2175-86; Sakuishi et al., J. Exp. Med., 2010, 207, 2187-94).

In one embodiment, the checkpoint inhibitor is an OX40 (CD134) agonist. In one embodiment, the checkpoint inhibitor is an anti-OX40 antibody. In one embodiment, the anti-OX40 antibody is anti-OX-40. In another embodiment, the anti-OX40 antibody is MEDI6469.

In one embodiment, the checkpoint inhibitor is a GITR agonist. In one embodiment, the checkpoint inhibitor is an anti-GITR antibody. In one embodiment, the anti-GITR antibody is TRX518.

In one embodiment, the checkpoint inhibitor is a CD137 agonist. In one embodiment, the checkpoint inhibitor is an anti-CD137 antibody. In one embodiment, the anti-CD137 antibody is urelumab. In another embodiment, the anti-CD137 antibody is PF-05082566.

In one embodiment, the checkpoint inhibitor is a CD40 agonist. In one embodiment, the checkpoint inhibitor is an anti-CD40 antibody. In one embodiment, the anti-CD40 antibody is CF-870,893.

In one embodiment, the checkpoint inhibitor is recombinant human interleukin-15 (rhIL-15).

In one embodiment, the checkpoint inhibitor is a VTCN inhibitor. In one embodiment, the VTCN inhibitor is FPA150.

In one embodiment, the checkpoint inhibitor is an IDO inhibitor. In one embodiment, the IDO inhibitor is INCB024360. In another embodiment, the IDO inhibitor is indoximod. In one embodiment, the IDO inhibitor is epacadostat. In another embodiment, the IDO inhibitor is BMS986205. In yet another embodiment, the IDO inhibitor is Navoximod. In one embodiment, the IDO inhibitor is PF-06840003. In another embodiment, the IDO inhibitor is KHK2455. In yet another embodiment, the IDO inhibitor is RG70099. In one embodiment, the IDO inhibitor is IOM-E. In another embodiment, the IDO inhibitor is or IOM-D.

In some embodiments, the checkpoint inhibitor is a TIGIT inhibitor. In certain embodiments, the TIGIT inhibitor is an anti-TIGIT antibody. In one embodiment, the TIGIT inhibitor is MTIG7192A. In another embodiment, the TIGIT inhibitor is BMS-986207. In yet another embodiment, the TIGIT inhibitor is OMP-313M32. In one embodiment, the TIGIT inhibitor is MK-7684. In another embodiment, the TIGIT inhibitor is AB154. In yet another embodiment, the TIGIT inhibitor is CGEN-15137. In one embodiment, the TIGIT inhibitor is SEA-TIGIT. In another embodiment, the TIGIT inhibitor is ASP8374. In yet another embodiment, the TIGIT inhibitor is AJUD008.

In some embodiments, the checkpoint inhibitor is a VSIR inhibitor. In certain embodiments, the VSIR inhibitor is an anti-VSIR antibody. In one embodiment, the VSIR inhibitor is MTIG7192A. In another embodiment, the VSIR inhibitor is CA-170. In yet another embodiment, the VSIR inhibitor is JNJ 61610588. In one embodiment, the VSIR inhibitor is HMBD-002.

In some embodiments, the checkpoint inhibitor is a TIM3 inhibitor. In certain embodiments, the TIM3 inhibitor is an anti-TIM3 antibody. In one embodiment, the TIM3 inhibitor is AJUD009.

In some embodiments, the checkpoint inhibitor is a CD25 (IL2RA) inhibitor. In certain embodiments, the CD25 (IL2RA) inhibitor is an anti-CD25 (IL2RA) antibody. In one embodiment, the CD25 (IL2RA) inhibitor is daclizumab. In another embodiment, the CD25 (IL2RA) inhibitor is basiliximab.

In some embodiments, the checkpoint inhibitor is an IFNAR1 inhibitor. In certain embodiments, the IFNAR1 inhibitor is an anti-IFNAR1 antibody. In one embodiment, the IFNAR1 inhibitor is anifrolumab. In another embodiment, the IFNAR1 inhibitor is sifalimumab.

In some embodiments, the checkpoint inhibitor is a CSF1R inhibitor. In certain embodiments, the CSF1R inhibitor is an anti-CSF1R antibody. In one embodiment, the CSF1R inhibitor is pexidartinib. In another embodiment, the CSF1R inhibitor is emactuzumab. In yet another embodiment, the CSF1R inhibitor is cabiralizumab. In one embodiment, the CSF1R inhibitor is ARRY-382. In another embodiment, the CSF1R inhibitor is BLZ945. In yet another embodiment, the CSF1R inhibitor is AJUD010. In one embodiment, the CSF1R inhibitor is AMG820. In another embodiment, the CSF1R inhibitor is IMC-CS4. In yet another embodiment, the CSF1R inhibitor is JNJ-40346527. In one embodiment, the CSF1R inhibitor is PLX5622. In another embodiment, the CSF1R inhibitor is FPA008.

In some embodiments, the checkpoint inhibitor is a therapeutic agent targeting HLA. In certain embodiments, the therapeutic agent targeting HLA is an anti-HLA antibody. In one embodiment, the therapeutic agent targeting HLA is GSK01. In another embodiment, the therapeutic agent targeting HLA is IMC-C103C. In yet another embodiment, the therapeutic agent targeting HLA is IMC-F106C. In one embodiment, the therapeutic agent targeting HLA is IMC-G107C. In another embodiment, the therapeutic agent targeting HLA is ABBV-184.

In certain embodiments, the immune checkpoint inhibitors described herein include two or more of the checkpoint inhibitors described herein (including checkpoint inhibitors of the same or different class).

In some embodiments, the subjects that can be treated in the methods provided herein is a mammal. In some embodiments, the subjects that can be treated in the methods provided herein is a human.

### 5.2.1.4 Therapeutic Outcome of the Methods Provided Herein

Despite the poor prognosis for cisplatin ineligible human subjects who as described above are frail, suffer from multiple comorbidities beyond their urothelial cancer/bladder cancer (e.g., muscle invasive urothelial cancer (MIUC) and muscle invasive bladder cancer (MIBC)) and are not able to tolerate additional treatment beyond immunotherapy, the methods provided herein, including in methods described in this Section (Section 5.2) and Sections 3 and 6, can provide beneficial therapeutic outcomes for these cisplatin ineligible human subjects. In one embodiment, the human subject has a complete response following the treatment by a method provided herein. In another embodiment, the human subject has a partial response following the treatment by a method provided herein.

In some embodiments, the response (complete or partial response) is determined by evaluating the tumor or cancer site (lesions). The criteria for determining complete response (CR), partial response (PR), progressive disease (PD), and stable disease (SD) are described in Table 15.

In some embodiments of the methods provided herein, the pathological complete response rate (pCRR) for the subject is at least 30%. In some embodiments, the pathological complete response rate (pCRR) for the subject is at least 35%. In some embodiments, the pathological complete response rate (pCRR) for the subject is at least 40%. In some embodiments, the pathological complete response rate (pCRR) for the subject is at least 45% In some embodiments, the pathological complete response rate (pCRR) for the subject is at least 50%. In some embodiments, the pathological complete response rate (pCRR) for the subject is at least 55%. In some embodiments, the pathological complete response rate (pCRR) for the subject is at least 60%. In some embodiments, the pathological complete response rate (pCRR) for the subject is at least 65%. In some embodiments, the pathological complete response rate (pCRR) for the subject is at least 70%. In some embodiments, the pathological complete response rate (pCRR) for the subject is at least 75%.

In some embodiments of the methods provided herein, the pathological complete response rate (pCRR) is at least 30%. In some embodiments, the pathological complete response rate (pCRR) is at least 35%. In some embodiments, the pathological complete response rate (pCRR) is at least 40%. In some embodiments, the pathological complete response rate (pCRR) is at least 45%. In some embodiments, the pathological complete response rate (pCRR) is at least 50%. In some embodiments, the pathological complete response rate (pCRR) is at least 55%. In some embodiments, the pathological complete response rate (pCRR) is at least 60%. In some embodiments, the pathological complete response rate (pCRR) is at least 65%. In some embodiments, the pathological complete response rate (pCRR) is at least 70%. In some embodiments, the pathological complete response rate (pCRR) is at least 75%.

In some embodiments of the methods provided herein, the pathological complete response rate (pCRR) for a population of subjects treated with the ADC is at least 30%. In some embodiments, the pathological complete response rate (pCRR) for a population of subjects treated with the ADC is at least 35%. In some embodiments, the pathological complete response rate (pCRR) for a population of subjects treated with the ADC is at least 40%. In some embodiments, the pathological complete response rate (pCRR) for a population of subjects treated with the ADC is at least 45%. In some embodiments, the pathological complete response rate (pCRR) for a population of subjects treated with the ADC is at least 50%. In some embodiments, the pathological complete response rate (pCRR) for a population of subjects treated with the ADC is at least 55%. In some embodiments, the pathological complete response rate (pCRR) for a population of subjects treated with the ADC is at least 60%. In some embodiments, the pathological complete response rate (pCRR) for a population of subjects treated with the ADC is at least 65%. In some embodiments, the pathological complete response rate (pCRR) for a population of subjects treated with the ADC is at least 70%. In some embodiments, the pathological complete response rate (pCRR) for a population of subjects treated with the ADC is at least 75%.

In some embodiments of the methods provided herein, the human subject has a disease-free survival of at least or about 2 months following the treatment. In another embodiment, the human subject has a disease-free survival of at least or about 3 months following the treatment. In another embodiment, the human subject has a disease-free survival of at least or about 4 months following the treatment. In another embodiment, the human subject has a disease-free survival of at least or about 5 months following the treatment. In another embodiment, the human subject has a disease-free survival of at least or about 6 months following the treatment. In a further embodiment, the human subject has a disease-free survival of at least or about 7 months following the treatment. In yet another embodiment, the human subject has a disease-free survival of at least or about 8 months following the treatment. In one embodiment, the human subject has a disease-free survival of at least or about 9 months following the treatment. In another embodiment, the human subject has a disease-free survival of at least or about 10 months following the treatment. In a further embodiment, the human subject has a disease-free survival of at least or about 11 months following the treatment. In yet another embodiment, the human subject has a disease-free survival of at least or about 12 months following the treatment. In one embodiment, the human subject has a disease-free survival of at least or about 13 months following the treatment. In another embodiment, the human subject has a disease-free survival of at least or about 14 months following the treatment. In yet another embodiment, the human subject has a disease-free survival of at least or about 15 months following the treatment. In some embodiments, the overall survival of the subject is extended by at least 2, at least 4, at least 6, at least 8, at least 10, or at least 12 months.

In one embodiment, the human subject has a disease-free survival ranging from 2 to 15 months following the treatment. In another embodiment, the human subject has a disease-free survival ranging from 2 to 14 months following the treatment. In a further embodiment, the human subject has a disease-free survival ranging from 2 to 13 months following the treatment. In one embodiment, the human subject has a disease-free survival ranging from 2 to 12 months following the treatment. In another embodiment, the human subject has a disease-free survival ranging from 3 to 12 months following the treatment. In a further embodiment, the human subject has a disease-free survival ranging from 3 to 11 months following the treatment. In yet another embodiment, the human subject has a disease-free survival ranging from 4 to 11 months following the treatment. In one embodiment, the human subject has a disease-free survival ranging from 4 to 10 months following the treatment. In another embodiment, the human subject has a disease-free survival ranging from 5 to 10 months following the treatment. In a further embodiment, the human subject has a disease-free survival ranging from 5 to 9 months following the treatment. In yet another embodiment, the human subject has a disease-free survival ranging from 5 to 8 months following the treatment. In one embodiment, the human subject has a disease-free survival ranging from 5 to 7 months following the treatment. In one embodiment, the human subject has a disease-free survival ranging from 5 to 6 months following the treatment. In one embodiment, the human subject has a disease-free survival ranging from 6 to 7 months following the treatment. In another embodiment, the human subject has a disease-free survival ranging from 6 to 8 months following the treatment.

Additionally, in some embodiments, the disease-free survival is evaluated for a population of human subjects treated by a method provided herein by evaluating the median or mean disease-free survival in the treated population. In one embodiment, a population of the human subjects is treated by a method provided herein, wherein the median or mean disease-free survival in the treated population is at least or about 2 months. In a further embodiment, a population of the human subjects is treated by a method provided herein, wherein the median or mean disease-free survival in the treated population is at least or about 3 months. In a further embodiment, a population of the human subjects is treated by a method provided herein, wherein the median or mean disease-free survival in the treated population is at least or about 4 months. In a further embodiment, a population of the human subjects is treated by a method provided herein, wherein the median or mean disease-free survival in the treated population is at least or about 5 months. In a further embodiment, a population of the human subjects is treated by a method provided herein, wherein the median or mean disease-free survival in the treated population is at least or about 6 months. In a further embodiment, a population of the human subjects is treated by a method provided herein, wherein the median or mean disease-free survival in the treated population is at least or about 7 months. In yet another embodiment, a population of the human subjects is treated by a method provided herein, wherein the median or mean disease-free survival in the treated population is at least or about 8 months. In one embodiment, a population of the human subjects is treated by a method provided herein, wherein the median or mean disease-free survival in the treated population is at least or about 9 months. In another embodiment, a population of the human subjects is treated by a method provided herein, wherein the median or mean disease-free survival in the treated population is at least or about 10 months. In a further embodiment, a population of the human subjects is treated by a method provided herein, wherein the median or mean disease-free survival in the treated population is at least or about 11 months. In yet another embodiment, a population of the human subjects is treated by a method provided herein, wherein the median or mean disease-free survival in the treated population is at least or about 12 months. In one embodiment, a population of the human subjects is treated by a method provided herein, wherein the median or mean disease-free survival in the treated population is at least or about 13 months. In another embodiment, a population of the human subjects is treated by a method provided herein, wherein the median or mean disease-free survival in the treated population is at least or about 14 months. In yet another embodiment, a population of the human subjects is treated by a method provided herein, wherein the median or mean disease-free survival in the treated population is at least or about 15 months.

In one embodiment, a population of the human subjects is treated by a method provided herein, wherein the disease-free survival in the treated population ranges from 2 to 15 months. In another embodiment, a population of the human subjects is treated by a method provided herein, wherein the disease-free survival in the treated population ranges from 2 to 14 months. In a further embodiment, a population of the human subjects is treated by a method provided herein, wherein the disease-free survival in the treated population ranges from 2 to 13 months. In yet another embodiment, a population of the human subjects is treated by a method provided herein, wherein the disease-free survival in the treated population ranges from 2 to 12.32 months. In one embodiment, a population of the human subjects is treated by a method provided herein, wherein the disease-free survival in the treated population ranges from 2 to 12 months. In another embodiment, a population of the human subjects is treated by a method provided herein, wherein the disease-free survival in the treated population ranges from 3 to 12 months. In a further embodiment, a population of the human subjects is treated by a method provided herein, wherein the disease-free survival in the treated population ranges from 3 to 11 months. In yet another embodiment, a population of the human subjects is treated by a method provided herein, wherein the disease-free survival in the treated population ranges from 4 to 11 months. In one embodiment, a population of the human subjects is treated by a method provided herein, wherein the disease-free survival in the treated population ranges from 4 to 10 months. In another embodiment, a population of the human subjects is treated by a method provided herein, wherein the disease-free survival in the treated population ranges from 5 to 10 months. In a further embodiment, a population of the human subjects is treated by a method provided herein, wherein the disease-free survival in the treated population ranges from 5 to 9 months. In one embodiment, a population of the human subjects is treated by a method provided herein, wherein the disease-free survival in the treated population ranges from 5 to 8 months. In a further embodiment, a population of the human subjects is treated by a method provided herein, wherein the disease-free survival in the treated population ranges from 5 to 7 months. In a further embodiment, a population of the human subjects is treated by a method provided herein, wherein the disease-free survival in the treated population ranges from 5 to 6 months. In one embodiment, a population of the human subjects is treated by a method provided herein, wherein the disease-free survival in the treated population ranges from 6 to 7 months. In yet another embodiment, a population of the human subjects is treated by a method provided herein, wherein the disease-free survival in the treated population ranges from 6 to 8 months.

In some embodiments of the methods provided herein, the human subject has an event-free survival of at least or about 2 months following the treatment. In another embodiment, the human subject has an event-free survival of at least or about 2.6 months following the treatment. In another embodiment, the human subject has an event-free survival of at least or about 3 months following the treatment. In another embodiment, the human subject has an event-free survival of at least or about 4 months following the treatment. In another embodiment, the human subject has an event-free survival of at least or about 5 months following the treatment. In another embodiment, the human subject has an event-free survival of at least or about 6 months following the treatment. In another embodiment, the human subject has an event-free survival of at least or about 6.54 months following the treatment. In a further embodiment, the human subject has an event-free survival of at least or about 7 months following the treatment. In yet another embodiment, the human subject has an event-free survival of at least or about 8 months following the treatment. In one embodiment, the human subject has an event-free survival of at least or about 9 months following the treatment. In another embodiment, the human subject has an event-free survival of at least or about 10 months following the treatment. In a further embodiment, the human subject has an event-free survival of at least or about 11 months following the treatment. In yet another embodiment, the human subject has an event-free survival of at least or about 12 months following the treatment. In yet another embodiment, the human subject has an event-free survival of at least or about 12.32 months following the treatment. In one embodiment, the human subject has an event-free survival of at least or about 13 months following the treatment. In another embodiment, the human subject has an event-free survival of at least or about 14 months following the treatment. In yet another embodiment, the human subject has an event-free survival of at least or about 15 months following the treatment.

In one embodiment, the human subject has an event-free survival ranging from 2 to 15 months following the treatment. In another embodiment, the human subject has an event-free survival ranging from 2 to 14 months following the treatment. In a further embodiment, the human subject has an event-free survival ranging from 2 to 13 months following the treatment. In yet another embodiment, the human subject has an event-free survival ranging from 2 to 12.32 months following the treatment. In one embodiment, the human subject has an event-free survival ranging from 2 to 12 months following the treatment. In another embodiment, the human subject has an event-free survival ranging from 3 to 12 months following the treatment. In a further embodiment, the human subject has an event-free survival ranging from 3 to 11 months following the treatment. In yet another embodiment, the human subject has an event-free survival ranging from 4 to 11 months following the treatment. In one embodiment, the human subject has an event-free survival ranging from 4 to 10 months following the treatment. In another embodiment, the human subject has an event-free survival ranging from 5 to 10 months following the treatment. In a further embodiment, the human subject has an event-free survival ranging from 5 to 9 months following the treatment. In yet another embodiment, the human subject has an event-free survival ranging from 5 to 8 months following the treatment. In one embodiment, the human subject has an event-free survival ranging from 5 to 7 months following the treatment. In one embodiment, the human subject has an event-free survival ranging from 5 to 6 months following the treatment. In one embodiment, the human subject has an event-free survival ranging from 6 to 7 months following the treatment. In another embodiment, the human subject has an event-free survival ranging from 6 to 8 months following the treatment.

Additionally, in some embodiments, the event-free survival is evaluated for a population of human subjects treated by a method provided herein by evaluating the median or mean event-free survival in the treated population. In one embodiment, a population of the human subjects is treated by a method provided herein, wherein the median or mean event-free survival in the treated population is at least or about 2 months. In another embodiment, a population of the human subjects is treated by a method provided herein, wherein the median or mean event-free survival in the treated population is at least or about 2.6 months. In a further embodiment, a population of the human subjects is treated by a method provided herein, wherein the median or mean event-free survival in the treated population is at least or about 3 months. In a further embodiment, a population of the human subjects is treated by a method provided herein, wherein the median or mean event-free survival in the treated population is at least or about 4 months. In a further embodiment, a population of the human subjects is treated by a method provided herein, wherein the median or mean event-free survival in the treated population is at least or about 5 months. In a further embodiment, a population of the human subjects is treated by a method provided herein, wherein the median or mean event-free survival in the treated population is at least or about 6 months. In a further embodiment, a population of the human subjects is treated by a method provided herein, wherein the median or mean event-free survival in the treated population is at least or about 7 months. In yet another embodiment, a population of the human subjects is treated by a method provided herein, wherein the median or mean event-free survival in the treated population is at least or about 8 months. In one embodiment, a population of the human subjects is treated by a method provided herein, wherein the median or mean event-free survival in the treated population is at least or about 9 months. In another embodiment, a population of the human subjects is treated by a method provided herein, wherein the median or mean event-free survival in the treated population is at least or about 10 months. In a further embodiment, a population of the human subjects is treated by a method provided herein, wherein the median or mean event-free survival in the treated population is at least or about 11 months. In yet another embodiment, a population of the human subjects is treated by a method provided herein, wherein the median or mean event-free survival in the treated population is at least or about 12 months. In yet another embodiment, a population of the human subjects is treated by a method provided herein, wherein the median or mean event-free survival in the treated population is at least or about 12.32 months. In one embodiment, a population of the human subjects is treated by a method provided herein, wherein the median or mean event-free survival in the treated population is at least or about 13 months. In another embodiment, a population of the human subjects is treated by a method provided herein, wherein the median or mean event-free survival in the treated population is at least or about 14 months. In yet another embodiment, a population of the human subjects is treated by a method provided herein, wherein the median or mean event-free survival in the treated population is at least or about 15 months.

In one embodiment, a population of the human subjects is treated by a method provided herein, wherein the event-free survival in the treated population ranges from 2 to 15 months. In another embodiment, a population of the human subjects is treated by a method provided herein, wherein the event-free survival in the treated population ranges from 2 to 14 months. In a further embodiment, a population of the human subjects is treated by a method provided herein, wherein the event-free survival in the treated population ranges from 2 to 13 months. In yet another embodiment, a population of the human subjects is treated by a method provided herein, wherein the event-free survival in the treated population ranges from 2 to 12.32 months. In one embodiment, a population of the human subjects is treated by a method provided herein, wherein the event-free survival in the treated population ranges from 2 to 12 months. In another embodiment, a population of the human subjects is treated by a method provided herein, wherein the event-free survival in the treated population ranges from 3 to 12 months. In a further embodiment, a population of the human subjects is treated by a method provided herein, wherein the event-free survival in the treated population ranges from 3 to 11 months. In yet another embodiment, a population of the human subjects is treated by a method provided herein, wherein the event-free survival in the treated population ranges from 4 to 11 months. In one embodiment, a population of the human subjects is treated by a method provided herein, wherein the event-free survival in the treated population ranges from 4 to 10 months. In another embodiment, a population of the human subjects is treated by a method provided herein, wherein the event-free survival in the treated population ranges from 5 to 10 months. In a further embodiment, a population of the human subjects is treated by a method provided herein, wherein the event-free survival in the treated population ranges from 5 to 9 months. In one embodiment, a population of the human subjects is treated by a method provided herein, wherein the event-free survival in the treated population ranges from 5 to 8 months. In a further embodiment, a population of the human subjects is treated by a method provided herein, wherein the event-free survival in the treated population ranges from 5 to 7 months. In a further embodiment, a population of the human subjects is treated by a method provided herein, wherein the event-free survival in the treated population ranges from 5 to 6 months. In one embodiment, a population of the human subjects is treated by a method provided herein, wherein the event-free survival in the treated population ranges from 6 to 7 months. In yet another embodiment, a population of the human subjects is treated by a method provided herein, wherein the event-free survival in the treated population ranges from 6 to 8 months.

In some embodiments of the methods provided herein, the human subject has an overall survival of at least or about 5 months following the treatment. In another embodiment, the human subject has an overall survival of at least or about 6 months following the treatment. In a further embodiment, the human subject has an overall survival of at least or about 7 months following the treatment. In yet another embodiment, the human subject has an overall survival of at least or about 8 months following the treatment. In one embodiment, the human subject has an overall survival of at least or about 9 months following the treatment. In another embodiment, the human subject has an overall survival of at least or about 10 months following the treatment. In a further embodiment, the human subject has an overall survival of at least or about 11 months following the treatment. In yet another embodiment, the human subject has an overall survival of at least or about 12 months following the treatment. In one embodiment, the human subject has an overall survival of at least or about 13 months following the treatment. In another embodiment, the human subject has an overall survival of at least or about 14 months following the treatment. In yet another embodiment, the human subject has an overall survival of at least or about 15 months following the treatment. In one embodiment, the human subject has an overall survival of at least or about 16 months following the treatment. In another embodiment, the human subject has an overall survival of at least or about 17 months following the treatment. In a further embodiment, the human subject has an overall survival of at least or about 18 months following the treatment. In yet another embodiment, the human subject has an overall survival of at least or about 19 months following the treatment. In one embodiment, the human subject has an overall survival of at least or about 20 months following the treatment. In another embodiment, the human subject has an overall survival of at least or about 21 months following the treatment. In a further embodiment, the human subject has an overall survival of at least or about 22 months following the treatment. In a further embodiment, the human subject has an overall survival of at least or about 23 months following the treatment. In yet another embodiment, the human subject has an overall survival of at least or about 24 months following the treatment. In one embodiment, the human subject has an overall survival of at least or about 25 months following the treatment. In another embodiment, the human subject has an overall survival of at least or about 26 months following the treatment. In a further embodiment, the human subject has an overall survival of at least or about 27 months following the treatment. In one embodiment, the human subject has an overall survival of at least or about 28 months following the treatment. In another embodiment, the human subject has an overall survival of at least or about 29 months following the treatment. In a further embodiment, the human subject has an overall survival of at least or about 30 months following the treatment.

In one embodiment, the human subject has an overall survival ranging from 10 to 19 months following the treatment. In another embodiment, the human subject has an overall survival ranging from 10 to 18 months following the treatment. In a further embodiment, the human subject has an overall survival ranging from 10 to 17 months following the treatment. In yet another embodiment, the human subject has an overall survival ranging from 10 to 16 months following the treatment. In one embodiment, the human subject has an overall survival ranging from 10 to 15 months following the treatment. In another embodiment, the human subject has an overall survival ranging from 10 to 14 months following the treatment. In a further embodiment, the human subject has an overall survival ranging from 10 to 13 months following the treatment. In yet another embodiment, the human subject has an overall survival ranging from 10 to 12 months following the treatment. In one embodiment, the human subject has an overall survival ranging from 10 to 11 months following the treatment. In another embodiment, the human subject has an overall survival ranging from 11 to 19 months following the treatment. In a further embodiment, the human subject has an overall survival ranging from 12 to 19 months following the treatment. In yet another embodiment, the human subject has an overall survival ranging from 13 to 19 months following the treatment. In one embodiment, the human subject has an overall survival ranging from 14 to 18 months following the treatment. In one embodiment, the human subject has an overall survival ranging from 14 to 19 months following the treatment. In one embodiment, the human subject has an overall survival ranging from 15 to 18 months following the treatment. In another embodiment, the human subject has an overall survival ranging from 15 to 19 months following the treatment. In a further embodiment, the human subject has an overall survival ranging from 16 to 19 months following the treatment. In yet another embodiment, the human subject has an overall survival ranging from 17 to 19 months following the treatment. In one embodiment, the human subject has an overall survival ranging from 18 to 19 months following the treatment. In another embodiment, the human subject has an overall survival ranging from 11 to 18 months following the treatment. In a further embodiment, the human subject has an overall survival ranging from 12 to 17 months following the treatment. In a further embodiment, the human subject has an overall survival ranging from 13 to 16 months following the treatment. In yet another embodiment, the human subject has an overall survival ranging from 14 to 15 months following the treatment. In one embodiment, the human subject has an overall survival ranging from 10 to 20 months following the treatment. In another embodiment, the human subject has an overall survival ranging from 11 to 20 months following the treatment. In one embodiment, the human subject has an overall survival ranging from 11 to 24 months following the treatment. In one embodiment, the human subject has an overall survival ranging from 11 to 25 months following the treatment. In one embodiment, the human subject has an overall survival ranging from 12 to 24 months following the treatment. In one embodiment, the human subject has an overall survival ranging from 12 to 25 months following the treatment. In a further embodiment, the human subject has an overall survival ranging from 12 to 20 months following the treatment. In one embodiment, the human subject has an overall survival ranging from 13 to 20 months following the treatment. In another embodiment, the human subject has an overall survival ranging from 14 to 20 months following the treatment. In a further embodiment, the human subject has an overall survival ranging from 15 to 20 months following the treatment. In yet another embodiment, the human subject has an overall survival ranging from 16 to 20 months following the treatment. In one embodiment, the human subject has an overall survival ranging from 17 to 20 months following the treatment. In another embodiment, the human subject has an overall survival ranging from 18 to 20 months following the treatment. In a further embodiment, the human subject has an overall survival ranging from 19 to 20 months following the treatment. In one embodiment, the human subject has an overall survival ranging from 9 to 20 months following the treatment. In another embodiment, the human subject has an overall survival ranging from 9 to 19 months following the treatment. In a further embodiment, the human subject has an overall survival ranging from 9 to 18 months following the treatment. In one embodiment, the human subject has an overall survival ranging from 9 to 17 months following the treatment. In another embodiment, the human subject has an overall survival ranging from 9 to 16 months following the treatment. In a further embodiment, the human subject has an overall survival ranging from 9 to 15 months following the treatment. In one embodiment, the human subject has an overall survival ranging from 9 to 14 months following the treatment. In another embodiment, the human subject has an overall survival ranging from 9 to 13 months following the treatment. In a further embodiment, the human subject has an overall survival ranging from 9 to 12 months following the treatment. In one embodiment, the human subject has an overall survival ranging from 9 to 11 months following the treatment. In another embodiment, the human subject has an overall survival ranging from 9 to 10 months following the treatment.

Additionally, in some embodiments, the overall survival is evaluated for a population of human subjects treated by a method provided herein by evaluating the median or mean overall survival in the treated population. In one embodiment, a population of the human subjects is treated by a method provided herein, wherein the median or mean overall survival in the treated population is at least or about 5 months. In another embodiment, a population of the human subjects is treated by a method provided herein, wherein the median or mean overall survival in the treated population is at least or about 6 months. In a further embodiment, a population of the human subjects is treated by a method provided herein, wherein the median or mean overall survival in the treated population is at least or about 7 months. In yet another embodiment, a population of the human subjects is treated by a method provided herein, wherein the median or mean overall survival in the treated population is at least or about 8 months. In one embodiment, a population of the human subjects is treated by a method provided herein, wherein the median or mean overall survival in the treated population is at least or about 9 months. In another embodiment, a population of the human subjects is treated by a method provided herein, wherein the median or mean overall survival in the treated population is at least or about 10 months. In a further embodiment, a population of the human subjects is treated by a method provided herein, wherein the median or mean overall survival in the treated population is at least or about 11 months. In yet another embodiment, a population of the human subjects is treated by a method provided herein, wherein the median or mean overall survival in the treated population is at least or about 12 months. In one embodiment, a population of the human subjects is treated by a method provided herein, wherein the median or mean overall survival in the treated population is at least or about 13 months. In another embodiment, a population of the human subjects is treated by a method provided herein, wherein the median or mean overall survival in the treated population is at least or about 14 months. In yet another embodiment, a population of the human subjects is treated by a method provided herein, wherein the median or mean overall survival in the treated population is at least or about 15 months. In one embodiment, a population of the human subjects is treated by a method provided herein, wherein the median or mean overall survival in the treated population is at least or about 16 months. In another embodiment, a population of the human subjects is treated by a method provided herein, wherein the median or mean overall survival in the treated population is at least or about 17 months. In a further embodiment, a population of the human subjects is treated by a method provided herein, wherein the median or mean overall survival in the treated population is at least or about 18 months. In yet another embodiment, a population of the human subjects is treated by a method provided herein, wherein the median or mean overall survival in the treated population is at least or about 19 months. In one embodiment, a population of the human subjects is treated by a method provided herein, wherein the median or mean overall survival in the treated population is at least or about 20 months. In another embodiment, a population of the human subjects is treated by a method provided herein, wherein the median or mean overall survival in the treated population is at least or about 21 months. In a further embodiment, a population of the human subjects is treated by a method provided herein, wherein the median or mean overall survival in the treated population is at least or about 22 months. In yet another embodiment, a population of the human subjects is treated by a method provided herein, wherein the median or mean overall survival in the treated population is at least or about 23 months. In one embodiment, a population of the human subjects is treated by a method provided herein, wherein the median or mean overall survival in the treated population is at least or about 24 months. In another embodiment, a population of the human subjects is treated by a method provided herein, wherein the median or mean overall survival in the treated population is at least or about 25 months. In a further embodiment, a population of the human subjects is treated by a method provided herein, wherein the median or mean overall survival in the treated population is at least or about 26 months. In yet another embodiment, a population of the human subjects is treated by a method provided herein, wherein the median or mean overall survival in the treated population is at least or about 27 months. In one embodiment, a population of the human subjects is treated by a method provided herein, wherein the median or mean overall survival in the treated population is at least or about 28 months. In another embodiment, a population of the human subjects is treated by a method provided herein, wherein the median or mean overall survival in the treated population is at least or about 29 months. In a further embodiment, a population of the human subjects is treated by a method provided herein, wherein the median or mean overall survival in the treated population is at least or about 30 months.

In one embodiment, a population of the human subjects is treated by a method provided herein, wherein the overall survival in the treated population ranges from 10 to 19 months. In another embodiment, a population of the human subjects is treated by a method provided herein, wherein the overall survival in the treated population ranges from 10 to 18 months. In a further embodiment, a population of the human subjects is treated by a method provided herein, wherein the overall survival in the treated population ranges from 10 to 17 months. In yet another embodiment, a population of the human subjects is treated by a method provided herein, wherein the overall survival in the treated population ranges from 10 to 16 months. In one embodiment, a population of the human subjects is treated by a method provided herein, wherein the overall survival in the treated population ranges from 10 to 15 months. In another embodiment, a population of the human subjects is treated by a method provided herein, wherein the overall survival in the treated population ranges from 10 to 14 months. In a further embodiment, a population of the human subjects is treated by a method provided herein, wherein the overall survival in the treated population ranges from 10 to 13 months. In yet another embodiment, a population of the human subjects is treated by a method provided herein, wherein the overall survival in the treated population ranges from 10 to 12 months. In one embodiment, a population of the human subjects is treated by a method provided herein, wherein the overall survival in the treated population ranges from 10 to 11 months. In another embodiment, a population of the human subjects is treated by a method provided herein, wherein the overall survival in the treated population ranges from 10 to 19 months. In a further embodiment, a population of the human subjects is treated by a method provided herein, wherein the overall survival in the treated population ranges from 11 to 19 months. In one embodiment, a population of the human subjects is treated by a method provided herein, wherein the overall survival in the treated population ranges from 11 to 24 months. In a further embodiment, a population of the human subjects is treated by a method provided herein, wherein the overall survival in the treated population ranges from 11 to 25 months. In a further embodiment, a population of the human subjects is treated by a method provided herein, wherein the overall survival in the treated population ranges from 12 to 24 months. In one embodiment, a population of the human subjects is treated by a method provided herein, wherein the overall survival in the treated population ranges from 12 to 25 months. In yet another embodiment, a population of the human subjects is treated by a method provided herein, wherein the overall survival in the treated population ranges from 12 to 19 months. In one embodiment, a population of the human subjects is treated by a method provided herein, wherein the overall survival in the treated population ranges from 13 to 19 months. In another embodiment, a population of the human subjects is treated by a method provided herein, wherein the overall survival in the treated population ranges from 14 to 19 months. In a further embodiment, a population of the human subjects is treated by a method provided herein, wherein the overall survival in the treated population ranges from 15 to 19 months. In yet another embodiment, a population of the human subjects is treated by a method provided herein, wherein the overall survival in the treated population ranges from 16 to 19 months. In one embodiment, a population of the human subjects is treated by a method provided herein, wherein the overall survival in the treated population ranges from 17 to 19 months. In another embodiment, a population of the human subjects is treated by a method provided herein, wherein the overall survival in the treated population ranges from 18 to 19 months. In a further embodiment, a population of the human subjects is treated by a method provided herein, wherein the overall survival in the treated population ranges from 11 to 18 months. In yet another embodiment, a population of the human subjects is treated by a method provided herein, wherein the overall survival in the treated population ranges from 12 to 17 months. In one embodiment, a population of the human subjects is treated by a method provided herein, wherein the overall survival in the treated population ranges from 13 to 16 months. In another embodiment, a population of the human subjects is treated by a method provided herein, wherein the overall survival in the treated population ranges from 14 to 15 months. In a further embodiment, a population of the human subjects is treated by a method provided herein, wherein the overall survival in the treated population ranges from 10 to 20 months. In yet another embodiment, a population of the human subjects is treated by a method provided herein, wherein the overall survival in the treated population ranges from 11 to 20 months. In one embodiment, a population of the human subjects is treated by a method provided herein, wherein the overall survival in the treated population ranges from 12 to 20 months. In another embodiment, a population of the human subjects is treated by a method provided herein, wherein the overall survival in the treated population ranges from 13 to 20 months. In a further embodiment, a population of the human subjects is treated by a method provided herein, wherein the overall survival in the treated population ranges from 14 to 20 months. In yet another embodiment, a population of the human subjects is treated by a method provided herein, wherein the overall survival in the treated population ranges from 15 to 20 months. In one embodiment, a population of the human subjects is treated by a method provided herein, wherein the overall survival in the treated population ranges from 16 to 20 months. In another embodiment, a population of the human subjects is treated by a method provided herein, wherein the overall survival in the treated population ranges from 17 to 20 months. In a further embodiment, a population of the human subjects is treated by a method provided herein, wherein the overall survival in the treated population ranges from 18 to 20 months. In yet another embodiment, a population of the human subjects is treated by a method provided herein, wherein the overall survival in the treated population ranges from 19 to 20 months. In one embodiment, a population of the human subjects is treated by a method provided herein, wherein the overall survival in the treated population ranges from 9 to 20 months. In another embodiment, a population of the human subjects is treated by a method provided herein, wherein the overall survival in the treated population ranges from 9 to 19 months. In a further embodiment, a population of the human subjects is treated by a method provided herein, wherein the overall survival in the treated population ranges from 9 to 18 months. In yet another embodiment, a population of the human subjects is treated by a method provided herein, wherein the overall survival in the treated population ranges from 9 to 17 months. In one embodiment, a population of the human subjects is treated by a method provided herein, wherein the overall survival in the treated population ranges from 9 to 16 months. In another embodiment, a population of the human subjects is treated by a method provided herein, wherein the overall survival in the treated population ranges from 9 to 15 months. In a further embodiment, a population of the human subjects is treated by a method provided herein, wherein the overall survival in the treated population ranges from 9 to 14 months. In yet another embodiment, a population of the human subjects is treated by a method provided herein, wherein the overall survival in the treated population ranges from 9 to 13 months. In one embodiment, a population of the human subjects is treated by a method provided herein, wherein the overall survival in the treated population ranges from 9 to 12 months. In another embodiment, a population of the human subjects is treated by a method provided herein, wherein the overall survival in the treated population ranges from 9 to 11 months. In a further embodiment, a population of the human subjects is treated by a method provided herein, wherein the overall survival in the treated population ranges from 9 to 10 months.

In some embodiments of the methods provided herein, the pathological downstaging rate (pDSR) for the subject is at least 50%. In some embodiments, the pathological downstaging rate (pDSR) for the subject is at least 55%. In some embodiments, the pathological downstaging rate (pDSR) for the subject is at least 60%. In some embodiments, the pathological downstaging rate (pDSR) for the subject is at least 65%. In some embodiments, the pathological downstaging rate (pDSR) for the subject is at least 70%. In some embodiments, the pathological downstaging rate (pDSR) for the subject is at least 75%. In some embodiments, the pathological downstaging rate (pDSR) for the subject is at least 80%. In some embodiments, the pathological downstaging rate (pDSR) for the subject is at least 85%. In some embodiments, the pathological downstaging rate (pDSR) for the subject is at least 90%.

In some embodiments of the methods provided herein, the pathological downstaging rate (pDSR) is at least 50%. In some embodiments, the pathological downstaging rate (pDSR) is at least 55%. In some embodiments, the pathological downstaging rate (pDSR) is at least 60%. In some embodiments, the pathological downstaging rate (pDSR) is at least 65%. In some embodiments, the pathological downstaging rate (pDSR) is at least 70%. In some embodiments, the pathological downstaging rate (pDSR) is at least 75%. In some embodiments, the pathological downstaging rate (pDSR) is at least 80%. In some embodiments, the pathological downstaging rate (pDSR) is at least 85%. In some embodiments, the pathological downstaging rate (pDSR) is at least 90%.

In some embodiments of the methods provided herein, the pathological downstaging rate (pDSR) for a population of subjects treated with the ADC is at least 50%. In some embodiments, the pathological downstaging rate (pDSR) for a population of subjects treated with the ADC is at least 55%. In some embodiments, the pathological downstaging rate (pDSR) for a population of subjects treated with the ADC is at least 60%. In some embodiments, the pathological downstaging rate (pDSR) for a population of subjects treated with the ADC is at least 65%. In some embodiments, the pathological downstaging rate (pDSR) for a population of subjects treated with the ADC is at least 70%. In some embodiments, the pathological downstaging rate (pDSR) for a population of subjects treated with the ADC is at least 75%.

In some embodiments, the human subjects and patients are used interchangeably. Therefore, a person skilled in the art would understand that the human subjects can be interchangeable with patients in any of the methods provided herein.

### 5.2.2 Methods of Treating Cancer in Patient Populations Based on Additional Selection Criteria

Provided herein are methods for the treatment of various cancers in subjects, wherein the cancers have any of the suitable markers and/or characteristics as provided in Section 6. Also provided herein are methods for the treatment of various cancers in subjects, wherein the subjects have any of the suitable characteristics as provided in Section 6.

In one aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the subject has any of the suitable characteristics as provided in Section 6.

In some aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23; and wherein the cancer has any of the suitable markers and/or characteristics as provided in Section 6.

In another aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), and wherein the subject has any of the suitable characteristics as provided in Section 6. In a further aspect, provided herein is a method of preventing or treating cancer in a subject, comprising administering to the subject an effective amount of an antibody drug conjugate, wherein the antibody drug conjugate comprises an antibody or antigen binding fragment thereof that binds to 191P4D12 conjugated to one or more units of monomethyl auristatin E (MMAE), and wherein the cancer has any of the suitable markers and/or characteristics as provided in Section 6.

In some embodiments of the methods provided herein including in Section 5.2 including Sections 5.2.1 and 5.2.2 and Sections 3 and 6, the subject is a human subject.

In all the methods provided herein and specifically those described in the Sections 5.2.1 and 5.2.2: the therapeutic agents including ADCs that can be used are described in Sections 3, 5.2, 5.3, 5.4, 5.5, and 6, selection of patients for treatment is described herein and exemplified in Section 5.2 including Sections 5.2.1 and 5.2.2 and Sections 3 and 6, dosing regimens and pharmaceutical composition for administering the therapeutic agent are described in Section 5.4, 5.7, and Section 6 below, the biomarkers that can be used for identifying the therapeutic agents, selecting the patients, determining the outcome of these methods, and/or serving as criteria in any way for these methods are described herein and exemplified in Section 5.2 including Sections 5.2.1 and 5.2.2 and Sections 3 and 6, the biomarkers can be determined as described in Section 5.8 or as known in the art, therapeutic outcomes for the methods provided herein are described in this Section (Section 5.2 including Section 5.2.1.4) and Sections 3 and 6, additional therapeutic outcomes for the methods provided herein can be improvement of the biomarkers described herein, for example, those described and exemplified in in Section 5.2 including Sections 5.2.1 and 5.2.2 and Sections 3 and 6, and combination therapies including the ADCs and other therapeutic agents are described in this Section (Section 5.2) and in Section 5.5. Therefore, a person skilled in the art would understand that the methods provided herein include all permutations and combinations of the patients, therapeutic agents, dosing regiments, biomarkers, and therapeutic outcomes as described above and below.

### 5.3 Antibody Drug Conjugates for the Methods

In various embodiments of the methods provided herein, including the methods provided in Section 5.2, the ADC used in the methods comprises or is an anti-191P4D12 ADC described herein and/or in U.S. Pat. No. 8,637,642, which is herein incorporated in its entirety by reference. In some embodiments, the anti-191P4D12 antibody drug conjugate provided for the methods herein comprises an antibody or antigen binding fragment thereof that binds to 191P4D12 as provided herein, including in Sections 3, 5.3.1, and 6, conjugated to one or more units of cytotoxic agents (drug units, or D) as provided herein, including in Sections 3 and 6 and this Section (Section 5.3) with further disclosures in Sections 5.3.2 and 5.3.4. In certain embodiments, the cytotoxic agents (drug units, or D) can be covalently linked directly or via a linker unit (LU) as provided herein, including in Sections 3 and 6 and this Section (Section 5.3) with further disclosures in Section 5.3.3.

In some embodiments, the antibody drug conjugate compound has the following formula:

L-(LU-D)ₚ (I)
or a pharmaceutically acceptable salt or solvate thereof; wherein:
L is the antibody unit, e.g., the anti-nectin-4 antibody or an antigen binding fragment thereof for example as provided in Sections 3, 5.3.1, and 6, and
(LU-D) is a linker unit-drug unit moiety, wherein:
LU- is a linker unit for example as provided in Sections 3 and 6 and this Section (Section 5.3) with further disclosures in Section 5.3.3, and
D is a drug unit having cytostatic or cytotoxic activity against a target cell for example as provided Sections 3 and 6 and this Section (Section 5.3) with further disclosures in Sections 5.3.2 and 5.3.4; and
p is an integer from 1 to 20 with further examples provided in Sections 3 and 6 and this Section (Section 5.3).

In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20.

In some embodiments, the antibody drug conjugate compound has the following formula:

L-(Aₐ-W_{w}-Y_{y}-D)ₚ (II)
or a pharmaceutically acceptable salt or solvate thereof, wherein:
L is the Antibody unit, e.g., the anti-nectin-4 antibody or an antigen binding fragment thereof for example as provided in Sections 3, 5.3.1, and 6; and
-Aₐ-W_{w}-Y_{y}- is a linker unit (LU), wherein:
-A- is a stretcher unit,
a is 0 or 1,
each -W- is independently an amino acid unit,
w is an integer ranging from 0 to 12,
-Y- is a self-immolative spacer unit,
y is 0, 1 or 2,
each for example as provided in Sections 3 and 6 and this Section (Section 5.3) with further disclosures in Section 5.3.3;
D is a drug units having cytostatic or cytotoxic activity against the target cell for example as provided Sections 3 and 6 and this Section (Section 5.3) with further disclosures in Sections 5.3.2 and 5.3.4; and
p is an integer from 1 to 20 with further examples provided in Sections 3 and 6 and this Section (Section 5.3).

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments, p is about 1. In some embodiments, p is about 2. In some embodiments, p is about 3. In some embodiments, p is about 4. In some embodiments, p is about 3.8. In some embodiments, p is about 5. In some embodiments, p is about 6. In some embodiments, p is about 7. In some embodiments, p is about 8. In some embodiments, p is about 9. In some embodiments, p is about 10. In some embodiments, p is about 11. In some embodiments, p is about 12. In some embodiments, p is about 13. In some embodiments, p is about 14. In some embodiments, p is about 15. In some embodiments, p is about 16. In some embodiments, p is about 17. In some embodiments, p is about 18. In some embodiments, p is about 19. In some embodiments, p is about 20. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

In some specific embodiments of the methods provided herein, including the methods provided in Section 5.2, the cytotoxic agent as part of any of the ADCs provided herein for the methods comprises, consists of, or is MMAE.

For compositions comprising a plurality antibodies or antigen binding fragments thereof, the drug loading is represented by p, the average number of drug molecules per antibody unit. Drug loading can range from 1 to 20 drugs (D) per antibody. The average number of drugs per antibody in preparation of conjugation reactions can be characterized by conventional means such as mass spectroscopy, ELISA assay, and HPLC. The quantitative distribution of antibody drug conjugates in terms of p can also be determined. In some instances, separation, purification, and characterization of homogeneous antibody drug conjugates where p is a certain value from antibody drug conjugates with other drug loadings can be achieved by means such as reverse phase HPLC or electrophoresis. In certain exemplary embodiments, p is from 2 to 8.

Additional embodiments of the ADC for the methods provided herein have been described in U.S. Pat. No. 8,637,642 and International Application No. PCT/US2019/056214 (Publication No. WO2020/117373), both of which are hereby incorporated in their entireties by reference.

In some embodiments of the methods provided herein, including in Sections 3, 5.2, and 6 and this Section (Section 5.3), the ADC is enfortumab vedotin. In certain embodiments of the methods provided herein, including in Sections 3, 5.2, and 6 and this Section (Section 5.3), the ADC is a biosimilar of enfortumab vedotin.

In some embodiments of the methods provided herein, the ADC is administered as a monotherapy.

### 5.3.1 Anti-191P4D12 Antibodies or Antigen Binding Fragments

In one embodiment, the antibody or antigen binding fragment thereof that binds to nectin-4-related proteins is an antibody or antigen binding fragment that specifically binds to nectin-4 protein comprising amino acid sequence of SEQ ID NO:2 (see FIG. 1A). The corresponding cDNA encoding the 191P4D12 protein has a sequence of SEQ ID NO:1 (see FIG. 1A).

The antibody that specifically binds to nectin-4 protein comprising amino acid sequence of SEQ ID NO:2 includes antibodies that can bind to other nectin-4-related proteins. For example, antibodies that bind nectin-4 protein comprising amino acid sequence of SEQ ID NO:2 can bind nectin-4-related proteins such as nectin-4 variants and the homologs or analogs thereof.

In some embodiments, the anti-nectin-4 antibody provided herein is a monoclonal antibody.

In some embodiments, the antigen binding fragment is an Fab, F(ab□)2, Fv, or scFv. In some embodiments, the antibody is a fully human antibody. In some embodiments, the antibody is an IgG1 and the light chain is a kappa light chain. In some embodiments, the antibody or antigen binding fragment thereof is recombinantly produced.

In some embodiments, the antibody comprises a heavy chain comprising an amino acid sequence of SEQ ID NO:4 (cDNA sequence of SEQ ID NO:3), and/or a light chain comprising an amino acid sequence of SEQ ID NO:6 (cDNA sequence of SEQ ID NO:5), as shown in FIGS. 1B and 1C.

In some embodiments, the anti-nectin-4 antibody or antigen binding fragment thereof comprises a heavy chain variable region comprising complementarity determining regions (CDRs) comprising the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs comprising the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 comprising the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 comprising the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). In some embodiments, the anti-nectin-4 antibody or antigen binding fragment thereof comprises a heavy chain variable region comprising complementarity determining regions (CDRs) consisting of the amino acid sequences of the CDRs of the heavy chain variable region set forth in SEQ ID NO:22 (which is the amino acid sequence ranging from the 20th amino acid (glutamic acid) to the 136th amino acid (serine) of SEQ ID NO:7) and a light chain variable region comprising CDRs consisting of the amino acid sequences of the CDRs of the light chain variable region set forth in SEQ ID NO:23 (which is the amino acid sequence ranging from the 23rd amino acid (aspartic acid) to the 130th amino acid (arginine) of SEQ ID NO:8). In certain embodiments, the anti-nectin-4 antibody or antigen binding fragment thereof comprises a heavy chain variable region comprising complementarity determining region 1 (CDR-H1), CDR-H2, and CDR-H3 consisting of the amino acid sequences of the corresponding CDR-H1, CDR-H2, and CDR-H3 in the heavy chain variable region sequence set forth in SEQ ID NO:22 (which is the amino acid sequence ranging from the 20th amino acid (glutamic acid) to the 136th amino acid (serine) of SEQ ID NO:7) and a light chain variable region comprising CDR-L1, CDR-L2, and CDR-L3 consisting of the amino acid sequences of the corresponding CDR-L1, CDR-L2, and CDR-L3 in the light chain variable region sequence set forth in SEQ ID NO:23 (which is the amino acid sequence ranging from the 23rd amino acid (aspartic acid) to the 130th amino acid (arginine) of SEQ ID NO:8). SEQ ID NO:22, SEQ ID NO:23, SEQ ID NO:7 and SEQ ID NO:8 are as shown in FIGS. 1D and 1E and listed below:

SEQ ID NO: 22
EVQLVESGGGLVQPGGSLRLSCAASGFTFSSYNMNWVRQAPGKGLEWVS
YISSSSSTIYYADSVKGRFTISRDNAKNSLSLQMNSLRDEDTAVYYCAR
AYYYGMDVWGQGTTVTVSS
SEQ ID NO: 23
DIQMTQSPSSVSASVGDRVTITCRASQGISGWLAWYQQKPGKAPKFLIY
AASTLQSGVPSRFSGSGSGTDFTLTISSLQPEDFATYYCQQANSFPPTF
GGGTKVEIKR
SEQ ID NO: 7
MELGLCWVFLVAILEGVQCEVQLVESGGGLVQPGGSLRLSCAASGFTFS
SYNMNWVRQAPGKGLEWVSYISSSSSTIYYADSVKGRFTISRDNAKNSL
SLQMNSLRDEDTAVYYCARAYYYGMDVWGQGTTVTVSSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNV
FSCSVMHEALHNHYTQKSLSLSPGK
SEQ ID NO: 8
MDMRVPAQLLGLLLLWFPGSRCDIQMTQSPSSVSASVGDRVTITCRASQ
GISGWLAWYQQKPGKAPKFLIYAASTLQSGVPSRFSGSGSGTDFTLTIS
SLQPEDFATYYCQQANSFPPTFGGGTKVEIKRTVAAPSVFIFPPSDEQL
KSGTASVVCLLNNFYPREAKVQWKVDNALQSGNSQESVTEQDSKDSTYS
LSSTLTLSKADYEKHKVYACEVTHQGLSSPVTKSFNRGEC

CDR sequences can be determined according to well-known numbering systems. As described above, CDR regions are well-known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System© (Lafranc et al., 2003, Dev. Comp. Immunol. 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309: 657-70. Correspondence between the numbering system, including, for example, the Kabat numbering and the IMGT unique numbering system, is well-known to one skilled in the art (see, e.g., Kabat, supra; Chothia and Lesk, supra; Martin, supra; Lefranc et al., supra). The residues from each of these hypervariable regions or CDRs are noted in Table 1 above.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Kabat numbering and a light chain variable region comprising CDRs comprising the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to AbM numbering and a light chain variable region comprising CDRs comprising the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Chothia numbering and a light chain variable region comprising CDRs comprising the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to Contact numbering and a light chain variable region comprising CDRs comprising the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) comprising the amino acid sequences of the CDRs of the heavy chain variable region set forth in SEQ ID NO:22 according to IMGT numbering and a light chain variable region comprising CDRs comprising the amino acid sequences of the CDRs of the light chain variable region set forth in SEQ ID NO:23 according to IMGT numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Kabat numbering and a light chain variable region comprising CDRs consisting of the amino acid sequences of the CDRs of the light chain variable region set forth in SEQ ID NO:23 according to Kabat numbering.

In some embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to AbM numbering and a light chain variable region comprising CDRs consisting of the amino acid sequences of the CDRs of the light chain variable region set forth in SEQ ID NO:23 according to AbM numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Chothia numbering and a light chain variable region comprising CDRs consisting of the amino acid sequences of the CDRs of the light chain variable region set forth in SEQ ID NO:23 according to Chothia numbering.

In other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to Contact numbering and a light chain variable region comprising CDRs consisting of the amino acid sequences of the CDRs of the light chain variable region set forth in SEQ ID NO:23 according to Contact numbering.

In yet other embodiments, the anti-nectin-4 antibody or antigen binding fragment thereof comprises a heavy chain variable region comprising CDRs (CDR-H1, CDR-H2, CDR-H3, CDR-L1, CDR-L2, and CDR-L3) consisting of the amino acid sequences of the CDRs of the heavy chain variable region set forth in SEQ ID NO:22 according to IMGT numbering and a light chain variable region comprising CDRs consisting of the amino acid sequences of the CDRs of the light chain variable region set forth in SEQ ID NO:23 according to IMGT numbering.

As described above, the CDR sequences according to different numbering systems can be readily determined, e.g., using online tools such as the one provided by Antigen receptor Numbering And Receptor ClassificatIon (ANARCI). For example, the heavy chain CDR sequences within SEQ ID NO:22, and the light chain CDR sequences within SEQ ID NO:23 according to Kabat numbering as determined by ANARCI are listed in Table 4 below.

TABLE 4

| | VH of SEQ ID NO: 22 | VL of SEQ ID NO: 23 |
|---|---|---|
| CDR1 | SYNMN (SEQ ID NO: 9) | RASQGISGWLA (SEQ ID NO: 12) |
| CDR2 | YISSSSSTIYYADSVKG | AASTLQS (SEQ ID NO: 13) |
| | (SEQ ID NO: 10) | |
| CDR3 | AYYYGMDV (SEQ ID NO: 11) | QQANSFPPT (SEQ ID NO: 14) |

For another example, the heavy chain CDR sequences within SEQ ID NO:22, and the light chain CDR sequences within SEQ ID NO:23 according to IMGT numbering as determined by ANARCI are listed in Table 5 below.

TABLE 5

| | VH of SEQ ID NO: 22 | VL of SEQ ID NO: 23 |
|---|---|---|
| CDR1 | GFTFSSYN (SEQ ID NO: 16) | QGISGW (SEQ ID NO: 19) |
| CDR2 | ISSSSSTI (SEQ ID NO: 17) | AAS (SEQ ID NO: 20) |
| CDR3 | ARAYYYGMDV (SEQ ID NO: 18) | QQANSFPPT (SEQ ID NO: 21) |

In some embodiments, the antibody or antigen binding fragment thereof comprises CDR-H1 comprising an amino acid sequence of SEQ ID NO:9, CDR-H2 comprising an amino acid sequence of SEQ ID NO:10, CDR-H3 comprising an amino acid sequence of SEQ ID NO:11, CDR-L1 comprising an amino acid sequence of SEQ ID NO:NO:12, CDR-L2 comprising an amino acid sequence of SEQ ID NO:NO:13, and CDR-L3 comprising an amino acid sequence of SEQ ID NO:NO:14.

In some embodiments, the antibody or antigen binding fragment thereof comprises CDR-H1 comprising an amino acid sequence of SEQ ID NO:16, CDR-H2 comprising an amino acid sequence of SEQ ID NO:17, CDR-H3 comprising an amino acid sequence of SEQ ID NO:18, CDR-L1 comprising an amino acid sequence of SEQ ID NO:NO:19, CDR-L2 comprising an amino acid sequence of SEQ ID NO:NO:20, and CDR-L3 comprising an amino acid sequence of SEQ ID NO:NO:21.

In some embodiments, the antibody or antigen binding fragment thereof comprises CDR-H1 consisting of an amino acid sequence of SEQ ID NO:9, CDR-H2 consisting of an amino acid sequence of SEQ ID NO:10, CDR-H3 consisting of an amino acid sequence of SEQ ID NO:11, CDR-L1 consisting of an amino acid sequence of SEQ ID NO:NO:12, CDR-L2 consisting of an amino acid sequence of SEQ ID NO:NO:13, and CDR-L3 consisting of an amino acid sequence of SEQ ID NO:NO:14.

In some embodiments, the antibody or antigen binding fragment thereof comprises CDR-H1 consisting of an amino acid sequence of SEQ ID NO:16, CDR-H2 consisting of an amino acid sequence of SEQ ID NO:17, CDR-H3 consisting of an amino acid sequence of SEQ ID NO:18, CDR-L1 consisting of an amino acid sequence of SEQ ID NO:NO:19, CDR-L2 consisting of an amino acid sequence of SEQ ID NO:NO:20, and CDR-L3 consisting of an amino acid sequence of SEQ ID NO:NO:21.

In some embodiments, the antibody or antigen binding fragment thereof comprises a heavy chain variable region comprising the amino acid sequence of SEQ ID NO:22 and a light chain variable region comprising the amino acid sequence of SEQ ID NO:23.

In some embodiments, the antibody or antigen binding fragment thereof comprises a heavy chain variable region consisting of the amino acid sequence of SEQ ID NO:22 and a light chain variable region consisting of the amino acid sequence of SEQ ID NO:23.

In some embodiments, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

In some embodiments, the antibody comprises a heavy chain consisting of the amino acid sequence ranging from the 20th amino acid (glutamic acid) to the 466th amino acid (lysine) of SEQ ID NO:7 and a light chain consisting of the amino acid sequence ranging from the 23rd amino acid (aspartic acid) to the 236th amino acid (cysteine) of SEQ ID NO:8.

In some embodiments, amino acid sequence modification(s) of antibodies described herein are contemplated. For example, it may be desirable to optimize the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes can alter post-translational processes of the antibody, such as changing the number or position of glycosylation sites or altering the membrane anchoring characteristics.

In some embodiments, the antibodies provided herein are chemically modified, for example, by the covalent attachment of any type of molecule to the antibody. The antibody derivatives can include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formulation, metabolic synthesis of tunicamycin, etc. Additionally, the antibody can contain one or more non-classical amino acids.

Variations can be a substitution, deletion, or insertion of one or more codons encoding the single domain antibody or polypeptide that results in a change in the amino acid sequence as compared with the original antibody or polypeptide. Amino acid substitutions can be the result of replacing one amino acid with another amino acid comprising similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions can optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed can be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue comprising a side chain with a similar charge. As described above, families of amino acid residues comprising side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined conservative (e.g., within an amino acid group with similar properties and/or side chains) substitutions can be made, so as to maintain or not significantly change the properties.

Amino acids can be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

For example, any cysteine residue not involved in maintaining the proper conformation of the antibody also can be substituted, for example, with another amino acid, such as alanine or serine, to improve the oxidative stability of the molecule and to prevent aberrant crosslinking.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-anti-MSLN antibody variant DNA.

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

In certain embodiments, the antibody or antigen binding fragment provided herein comprises a heavy chain having certain homology or identity to the heavy chain as set forth in SEQ ID NO:7 and a light chain having certain homology or identity to the light chain as set forth in SEQ ID NO:8. Such embodiments of heavy/light chains with homology or identity are further provided as follows. In some embodiments, the antibody or antigen binding fragment provided herein comprises a heavy chain having more than 70% homology or identity to the heavy chain as set forth in SEQ ID NO:7. In some embodiments, the antibody or antigen binding fragment provided herein comprises a heavy chain having more than 75% homology or identity to the heavy chain as set forth in SEQ ID NO:7. In some embodiments, the antibody or antigen binding fragment provided herein comprises a heavy chain having more than 80% homology or identity to the heavy chain as set forth in SEQ ID NO:7. In some embodiments, the antibody or antigen binding fragment provided herein comprises a heavy chain having more than 85% homology or identity to the heavy chain as set forth in SEQ ID NO:7. In some embodiments, the antibody or antigen binding fragment provided herein comprises a heavy chain having more than 90% homology or identity to the heavy chain as set forth in SEQ ID NO:7. In some embodiments, the antibody or antigen binding fragment provided herein comprises a heavy chain having more than 95% homology or identity to the heavy chain as set forth in SEQ ID NO:7. In certain embodiments, the antibody or antigen binding fragment provided herein comprises a heavy chain having any of the provided homology or identity to the heavy chain as set forth in SEQ ID NO:7, wherein the CDRs (CDR-H1, CDR-H2, and CDR-H3) are identical to the CDRs in the heavy chain as set forth in SEQ ID NO:7. In some embodiments, the antibody or antigen binding fragment provided herein comprises a light chain having more than 70% homology or identity to the light chain as set forth in SEQ ID NO:8. In some embodiments, the antibody or antigen binding fragment provided herein comprises a light chain having more than 75% homology or identity to the light chain as set forth in SEQ ID NO:8. In some embodiments, the antibody or antigen binding fragment provided herein comprises a light chain having more than 80% homology or identity to the light chain as set forth in SEQ ID NO:8. In some embodiments, the antibody or antigen binding fragment provided herein comprises a light chain having more than 85% homology or identity to the light chain as set forth in SEQ ID NO:8. In some embodiments, the antibody or antigen binding fragment provided herein comprises a light chain having more than 90% homology or identity to the light chain as set forth in SEQ ID NO:8. In some embodiments, the antibody or antigen binding fragment provided herein comprises a light chain having more than 95% homology or identity to the light chain as set forth in SEQ ID NO:8. In certain embodiments, the antibody or antigen binding fragment provided herein comprises a light chain having any of the provided homology or identity to the light chain as set forth in SEQ ID NO:8, wherein the CDRs (CDR-L1, CDR-L2, and CDR-L3) are identical to the CDRs in the light chain as set forth in SEQ ID NO:8. In certain embodiments, the antibody or antigen binding fragment provided herein comprises any homologous light chain and any homologous heavy chain as provided in this paragraph in any combination or permutation.

In certain embodiments, the antibody or antigen binding fragment provided herein comprises a heavy chain variable region having certain homology or identity to the heavy chain variable region as set forth in SEQ ID NO:22 and a light chain variable region having certain homology or identity to the light chain variable region as set forth in SEQ ID NO:23. Such embodiments of heavy chain variable regions and light chain variable regions with homology or identity are further provided as follows. In some embodiments, the antibody or antigen binding fragment provided herein comprises a heavy chain variable region having more than 70% homology or identity to the heavy chain variable region as set forth in SEQ ID NO:22. In some embodiments, the antibody or antigen binding fragment provided herein comprises a heavy chain variable region having more than 75% homology or identity to the heavy chain variable region as set forth in SEQ ID NO:22. In some embodiments, the antibody or antigen binding fragment provided herein comprises a heavy chain variable region having more than 80% homology or identity to the heavy chain variable region as set forth in SEQ ID NO:22. In some embodiments, the antibody or antigen binding fragment provided herein comprises a heavy chain variable region having more than 85% homology or identity to the heavy chain variable region as set forth in SEQ ID NO:22. In some embodiments, the antibody or antigen binding fragment provided herein comprises a heavy chain variable region having more than 90% homology or identity to the heavy chain variable region as set forth in SEQ ID NO:22. In some embodiments, the antibody or antigen binding fragment provided herein comprises a heavy chain variable region having more than 95% homology or identity to the heavy chain variable region as set forth in SEQ ID NO:22. In certain embodiments, the antibody or antigen binding fragment provided herein comprises a heavy chain variable region having any of the provided homology or identity to the heavy chain variable region as set forth in SEQ ID NO:22, wherein the CDRs (CDR-H1, CDR-H2, and CDR-H3) are identical to the CDRs in the heavy chain variable region as set forth in SEQ ID NO:22. In some embodiments, the antibody or antigen binding fragment provided herein comprises a light chain variable region having more than 70% homology or identity to the light chain variable region as set forth in SEQ ID NO:23. In some embodiments, the antibody or antigen binding fragment provided herein comprises a light chain variable region having more than 75% homology or identity to the light chain variable region as set forth in SEQ ID NO:23. In some embodiments, the antibody or antigen binding fragment provided herein comprises a light chain variable region having more than 80% homology or identity to the light chain variable region as set forth in SEQ ID NO:23. In some embodiments, the antibody or antigen binding fragment provided herein comprises a light chain variable region having more than 85% homology or identity to the light chain variable region as set forth in SEQ ID NO:23. In some embodiments, the antibody or antigen binding fragment provided herein comprises a light chain variable region having more than 90% homology or identity to the light chain variable region as set forth in SEQ ID NO:23. In some embodiments, the antibody or antigen binding fragment provided herein comprises a light chain variable region having more than 95% homology or identity to the light chain variable region as set forth in SEQ ID NO:23. In certain embodiments, the antibody or antigen binding fragment provided herein comprises a light chain variable region having any of the provided homology or identity to the light chain variable region as set forth in SEQ ID NO:23, wherein the CDRs (CDR-L1, CDR-L2, and CDR-L3) are identical to the CDRs in the light chain variable region as set forth in SEQ ID NO:23. In certain embodiments, the antibody or antigen binding fragment provided herein comprises any homologous light chain variable region and any homologous heavy chain variable region as provided in this paragraph in any combination or permutation.

In some embodiments, the anti-nectin-4 antibody provided herein comprises heavy and light chain CDR regions of an antibody designated Ha22-2(2,4)6.1 produced by a hybridoma deposited under the American Type Culture Collection (ATCC) Accession NO: PTA-11267, or heavy and light chain CDR regions comprising amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the anti-nectin-4 antibody provided herein comprises heavy and light chain CDR regions (CDR-H1, CDR-H2, CDR-H3, CDR-L1, CDR-L2, and CDR-L3) of an antibody designated Ha22-2(2,4)6.1 produced by a hybridoma deposited under the American Type Culture Collection (ATCC) Accession NO: PTA-11267, or heavy and light chain CDR regions consisting of amino acid sequences that are homologous to the amino acid sequences of the heavy and light chain CDR regions of Ha22-2(2,4)6.1, and wherein the antibodies retain the desired functional properties of the anti-nectin-4 antibody designated Ha22-2(2,4)6.1 produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) comprising the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) comprising the amino acid sequences of the light chain variable region CDRs set forth in the antibody produced by a hybridoma deposited under the American Type Culture Collection (ATCC) Accession NO: PTA-11267.

In some embodiments, the antibody or antigen binding fragment thereof provided herein comprises a humanized heavy chain variable region and a humanized light chain variable region, wherein:
(a) the heavy chain variable region comprises CDRs (CDR-H1, CDR-H2, and CDR-H3) consisting of the amino acid sequences of the heavy chain variable region CDRs set forth in the antibody produced by a hybridoma deposited under the American Type Culture Collection (ATCC) Accession NO: PTA-11267;
(b) the light chain variable region comprises CDRs (CDR-L1, CDR-L2, and CDR-L3) consisting of the amino acid sequences of the light chain variable region CDRs set forth in the antibody produced by a hybridoma deposited under the American Type Culture Collection (ATCC) Accession NO: PTA-11267.

In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions comprising amino acid sequences that are homologous to the amino acid sequences of the heavy and light chain variable regions of Ha22-2(2,4)6.1, and wherein the antibodies retain the desired functional properties of the anti-nectin-4 antibody provided herein. In some embodiments, the anti-nectin-4 antibody provided herein comprises heavy and light chain variable regions of an antibody designated Ha22-2(2,4)6.1 produced by a hybridoma deposited under the American Type Culture Collection (ATCC) Accession NO: PTA-11267, or heavy and light variable regions consisting of amino acid sequences that are homologous to the amino acid sequences of the heavy and light chain variable regions of Ha22-2(2,4)6.1, and wherein the antibodies retain the desired functional properties of the anti-nectin-4 antibody provided herein. As the constant region of the antibody of the disclosure, any subclass of constant region can be chosen. In one embodiment, human IgG1 constant region as the heavy chain constant region and human Ig kappa constant region as the light chain constant region can be used.

In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains comprising amino acid sequences that are homologous to the amino acid sequences of the heavy and light chains of Ha22-2(2,4)6.1, and wherein the antibodies retain the desired functional properties of the anti-nectin-4 antibody provided herein. In some embodiments, the anti-nectin-4 antibody provided herein comprises heavy and light chains of an antibody designated Ha22-2(2,4)6.1 produced by a hybridoma deposited under the American Type Culture Collection (ATCC) Accession NO: PTA-11267, or heavy and light chains consisting of amino acid sequences that are homologous to the amino acid sequences of the heavy and light chains of Ha22-2(2,4)6.1, and wherein the antibodies retain the desired functional properties of the anti-nectin-4 antibody provided herein.

In some embodiments, the antibody or antigen binding fragment thereof provided herein comprises a heavy chain variable region and a light chain variable region, wherein:
(a) the heavy chain variable region comprises an amino acid sequence that is at least 80% homologous or identical to the heavy chain variable region amino acid sequence of the antibody produced by a hybridoma deposited under the American Type Culture Collection (ATCC) Accession NO: PTA-11267; and
(b) the light chain variable region comprises an amino acid sequence that is at least 80% homologous or identical to the light chain variable region amino acid sequence of the antibody produced by a hybridoma deposited under the American Type Culture Collection (ATCC) Accession NO: PTA-11267.

In certain embodiments, the antibody or antigen binding fragment provided herein comprises a heavy chain variable region having certain homology or identity to the heavy chain variable region amino acid sequence of the antibody produced by a hybridoma deposited under the American Type Culture Collection (ATCC) Accession NO: PTA-11267 and a light chain variable region having certain homology or identity to the light chain variable region amino acid sequence of the antibody produced by a hybridoma deposited under the American Type Culture Collection (ATCC) Accession NO: PTA-11267. Such embodiments of heavy chain variable regions and light chain variable regions with homology or identity are further provided as follows. In some embodiments, the heavy chain variable region comprises an amino acid sequence that is at least 85% homologous or identical to the heavy chain variable region amino acid sequence of the antibody produced by a hybridoma deposited under the American Type Culture Collection (ATCC) Accession NO: PTA-11267. In other embodiments, the heavy chain variable region comprises an amino acid sequence that is at least 90% homologous or identical to the heavy chain variable region amino acid sequence of the antibody produced by a hybridoma deposited under the American Type Culture Collection (ATCC) Accession NO: PTA-11267. In yet other embodiments, the heavy chain variable region comprises an amino acid sequence that is at least 95% homologous or identical to the heavy chain variable region amino acid sequence of the antibody produced by a hybridoma deposited under the American Type Culture Collection (ATCC) Accession NO: PTA-11267. In other embodiments, the heavy chain variable region can be 85%, 86%, 87%, 88%, 89%, 90%, 91%, 92%, 93%, 94%, 95%, 96%, 97%, 98% or 99% homologous or identical to the heavy chain variable region amino acid sequence of the antibody produced by a hybridoma deposited under the American Type Culture Collection (ATCC) Accession NO: PTA-11267. In some embodiments, the light chain variable region comprises an amino acid sequence that is at least 85% homologous or identical to the light chain variable region amino acid sequence of the antibody produced by a hybridoma deposited under the American Type Culture Collection (ATCC) Accession NO: PTA-11267. In other embodiments, the light chain variable region comprises an amino acid sequence that is at least 90% homologous or identical to the light chain variable region amino acid sequence of the antibody produced by a hybridoma deposited under the American Type Culture Collection (ATCC) Accession NO: PTA-11267. In yet other embodiments, the light chain variable region comprises an amino acid sequence that is at least 95% homologous or identical to the light chain variable region amino acid sequence of the antibody produced by a hybridoma deposited under the American Type Culture Collection (ATCC) Accession NO: PTA-11267. In other embodiments, the light chain variable region can be 85%, 86%, 87%, 88%, 89%, 90%, 91%, 92%, 93%, 94%, 95%, 96%, 97%, 98% or 99% homologous or identical to the light chain variable region amino acid sequence of the antibody produced by a hybridoma deposited under the American Type Culture Collection (ATCC) Accession NO: PTA-11267. In certain embodiments, the antibody or antigen binding fragment provided herein comprises any homologous light chain variable region and any homologous heavy chain variable region as provided in this paragraph in any combination or permutation.

In other embodiments, the antibody or antigen binding fragment thereof provided herein comprises a heavy chain and a light chain, wherein:
(a) the heavy chain comprises an amino acid sequence that is at least 80% homologous or identical to the heavy chain amino acid sequence of the antibody produced by a hybridoma deposited under the American Type Culture Collection (ATCC) Accession NO: PTA-11267; and
(b) the light chain comprises an amino acid sequence that is at least 80% homologous or identical to the light chain amino acid sequence of the antibody produced by a hybridoma deposited under the American Type Culture Collection (ATCC) Accession NO: PTA-11267.

In certain embodiments, the antibody or antigen binding fragment provided herein comprises a heavy chain having certain homology or identity to the heavy chain amino acid sequence of the antibody produced by a hybridoma deposited under the American Type Culture Collection (ATCC) Accession NO: PTA-11267 and a light chain having certain homology or identity to the light chain amino acid sequence of the antibody produced by a hybridoma deposited under the American Type Culture Collection (ATCC) Accession NO: PTA-11267. Such embodiments of heavy chains and light chains with homology or identity are further provided as follows. In some embodiments, the heavy chain comprises an amino acid sequence that is at least 85% homologous or identical to the heavy chain amino acid sequence of the antibody produced by a hybridoma deposited under the American Type Culture Collection (ATCC) Accession NO: PTA-11267. In other embodiments, the heavy chain comprises an amino acid sequence that is at least 90% homologous or identical to the heavy chain amino acid sequence of the antibody produced by a hybridoma deposited under the American Type Culture Collection (ATCC) Accession NO: PTA-11267. In yet other embodiments, the heavy chain comprises an amino acid sequence that is at least 95% homologous or identical to the heavy chain amino acid sequence of the antibody produced by a hybridoma deposited under the American Type Culture Collection (ATCC) Accession NO: PTA-11267. In other embodiments, the heavy chain can be 85%, 86%, 87%, 88%, 89%, 90%, 91%, 92%, 93%, 94%, 95%, 96%, 97%, 98% or 99% homologous or identical to the heavy chain amino acid sequence of the antibody produced by a hybridoma deposited under the American Type Culture Collection (ATCC) Accession NO: PTA-11267. In some embodiments, the light chain comprises an amino acid sequence that is at least 85% homologous or identical to the light chain amino acid sequence of the antibody produced by a hybridoma deposited under the American Type Culture Collection (ATCC) Accession NO: PTA-11267. In other embodiments, the light chain comprises an amino acid sequence that is at least 90% homologous or identical to the light chain amino acid sequence of the antibody produced by a hybridoma deposited under the American Type Culture Collection (ATCC) Accession NO: PTA-11267. In yet other embodiments, the light chain comprises an amino acid sequence that is at least 95% homologous or identical to the light chain amino acid sequence of the antibody produced by a hybridoma deposited under the American Type Culture Collection (ATCC) Accession NO: PTA-11267. In other embodiments, the light chain can be 85%, 86%, 87%, 88%, 89%, 90%, 91%, 92%, 93%, 94%, 95%, 96%, 97%, 98% or 99% homologous or identical to the light chain amino acid sequence of the antibody produced by a hybridoma deposited under the American Type Culture Collection (ATCC) Accession NO: PTA-11267. In certain embodiments, the antibody or antigen binding fragment provided herein comprises any homologous light chain and any homologous heavy chain as provided in this paragraph in any combination or permutation.

In some embodiments, the antibody or antigen binding fragment thereof provided herein binds to a specific epitope in 191P4D12. In some embodiments, the antibody or antigen binding fragment thereof provided herein binds to VC1 domain of 191P4D12. In some embodiments, the antibody or antigen binding fragment thereof provided herein binds to VC1 domain but not to C1C2 domain of 191P4D12. In some embodiments, the antibody or antigen binding fragment thereof provided herein binds to the 1st to 147th amino acid residues of 191P4D12. In some embodiments, the antibody or antigen binding fragment thereof provided herein binds to an epitope located in the 1st to 147th amino acid residues of 191P4D12. In some embodiments, the antibody or antigen binding fragment thereof provided herein binds to the 1st to 10th amino acid residues of 191P4D12. In some embodiments, the antibody or antigen binding fragment thereof provided herein binds to the 11th to 20th amino acid residues of 191P4D12. In some embodiments, the antibody or antigen binding fragment thereof provided herein binds to the 21st to 30th amino acid residues of 191P4D12. In some embodiments, the antibody or antigen binding fragment thereof provided herein binds to the 31st to 40th amino acid residues of 191P4D12. In some embodiments, the antibody or antigen binding fragment thereof provided herein binds to the 41st to 50th amino acid residues of 191P4D12. In some embodiments, the antibody or antigen binding fragment thereof provided herein binds to the 51st to 60th amino acid residues of 191P4D12. In some embodiments, the antibody or antigen binding fragment thereof provided herein binds to the 61st to 70th amino acid residues of 191P4D12. In some embodiments, the antibody or antigen binding fragment thereof provided herein binds to the 71st to 80th amino acid residues of 191P4D12. In some embodiments, the antibody or antigen binding fragment thereof provided herein binds to the 81st to 90th amino acid residues of 191P4D12. In some embodiments, the antibody or antigen binding fragment thereof provided herein binds to the 91st to 100th amino acid residues of 191P4D12. In some embodiments, the antibody or antigen binding fragment thereof provided herein binds to the 101st to 110th amino acid residues of 191P4D12. In some embodiments, the antibody or antigen binding fragment thereof provided herein binds to the 111th to 120th amino acid residues of 191P4D12. In some embodiments, the antibody or antigen binding fragment thereof provided herein binds to the 121st to 130th amino acid residues of 191P4D12. In some embodiments, the antibody or antigen binding fragment thereof provided herein binds to the 131st to 140th amino acid residues of 191P4D12. In some embodiments, the antibody or antigen binding fragment thereof provided herein binds to the 141st to 147th amino acid residues of 191P4D12. The binding epitopes of certain embodiments the antibodies or antigen binding fragments thereof provided herein have been determined and described in WO 2012/047724, which is incorporated herein in its entirety by reference.

In some embodiments, the antibody or antigen binding fragment thereof provided herein binds to epitopes in 191P4D12 that are common between the 191P4D12 variants observed in human. In some embodiments, the antibody or antigen binding fragment thereof provided herein binds to epitopes in 191P4D12 that are common between the 191P4D12 polymorphism observed in human. In some embodiments, the antibody or antigen binding fragment thereof provided herein binds to epitopes in 191P4D12 that are common between the 191P4D12 polymorphism observed in human cancers. In some embodiments, the antibody or antigen binding fragment thereof provided herein binds to epitopes in 191P4D12 that would bind, internalize, disrupt or modulate the biological function of 191P4D12 or 191P4D12 variants. In some embodiments, the antibody or antigen binding fragment thereof provided herein binds to epitopes in 191P4D12 that would disrupt the interaction between 191P4D12 with ligands, substrates, and binding partners.

Engineered antibodies provided herein include those in which modifications have been made to framework residues within VH and/or VL (e.g. to improve the properties of the antibody). Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis (e.g., “backmutated” from leucine to methionine). Such “backmutated” antibodies are also intended to be encompassed by the disclosure.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In addition or alternative to modifications made within the framework or CDR regions, antibodies of the disclosure can be engineered to include modifications within the Fc region, typically to alter one or more functional properties of the antibody, such as serum half-life, complement fixation, Fc receptor binding, and/or antigen-dependent cellular cytotoxicity. Furthermore, an anti-191P4D12 antibody provided herein can be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the anti-191P4D12 antibody.

In another embodiment, the Fc hinge region of an antibody is mutated to decrease the biological half-life of the anti-191P4D12 antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the anti-191P4D12 antibody is modified to increase its biological half-life. Various approaches are possible. For example, mutations can be introduced as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid specific residues can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

Reactivity of the anti-191P4D12 antibodies with a 191P4D12-related protein can be established by a number of well-known means, including Western blot, immunoprecipitation, ELISA, and FACS analyses using, as appropriate, 191P4D12-related proteins, 191P4D12-expressing cells or extracts thereof. A 191P4D12 antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 191P4D12 epitopes are generated using methods generally known in the art. Homodimeric antibodies can also be generated by cross-linking techniques known in the art (e.g., Wolff et al., Cancer Res. 53: 2560-2565).

In yet another specific embodiment, the anti-191P4D12 antibody provided herein is an antibody comprising heavy and light chain of an antibody designated Ha22-2(2,4)6.1. The heavy chain of Ha22-2(2,4)6.1 consists of the amino acid sequence ranging from 20^{th} E residue to the 466^{th} K residue of SEQ ID NO:7 and the light chain of Ha22-2(2,4)6.1 consists of amino acid sequence ranging from 23^{rd} D residue to the 236^{th} C residue of SEQ ID NO:8 sequence.

The hybridoma producing the antibody designated Ha22-2(2,4)6.1 was sent (via Federal Express) to the American Type Culture Collection (ATCC), P.O. Box 1549, Manassas, VA 20108 on 18 Aug. 2010 and assigned Accession number PTA-11267.

Additional embodiments of anti-nectin-4 antibody have been described in U.S. Pat. No. 8,637,642 and International Application No. PCT/US2019/056214 (Publication No. WO2020/117373), both of which are hereby incorporated in their entireties by reference.

### 5.3.2 Cytotoxic Agents (Drug Units)

As the ADC used in the methods provided herein comprises an antibody or antigen binding fragment thereof conjugated to a cytotoxic agent, the disclosure further provides various embodiments for the cytotoxic agent as part of the ADC for use in the methods. In various embodiments of the methods provided herein, including the methods provided in Section 5.2, the cytotoxic agent as part of any of the ADCs provided herein for the methods comprises, consists of, or is a tubulin disrupting agent. In one embodiment, the cytotoxic agent is a tubulin disrupting agent. In some embodiments, the tubulin disrupting agent is selected from the group consisting of a dolastatin, an auristatin, a hemiasterlin, a vinca alkaloid, a maytansinoid, an eribulin, a colchicine, a plocabulin, a phomopsin, an epothilone, a cryptophycin, and a taxane. In one specific embodiment, the tubulin disrupting agent is an auristatin. In a further specific embodiment, the auristatin is monomethyl auristatin E (MMAE), monomethyl auristatin F (MMAF), AFP, or auristain T. In yet another specific embodiment, the auristatin is monomethyl auristatin E (MMAE).

In various embodiments of the methods provided herein, including the methods provided in Section 5.2, the cytotoxic agent as part of any of the ADCs provided herein for the methods comprises, consists of, or is any agent selected from the cytotoxic agents described in U.S. Pat. No. 8,637,642 and International Application No. PCT/US2019/056214 (Publication No. WO2020/117373), both of which are hereby incorporated in their entireties by reference

In some embodiments, the auristatin is MMAE (wherein the wavy line indicates the covalent attachment to a linker of an antibody drug conjugate).

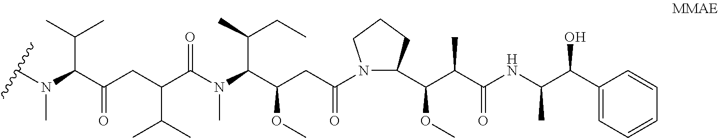 [SMILES: *N(C)[C@H](C(=O)CC(C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, an exemplary embodiment comprising MMAE and a linker component (described further herein) has the following structure (wherein L presents the antibody (e.g. anti-nectin-4 antibody or antigen binding fragment thereof) and p ranges from 1 to 12):

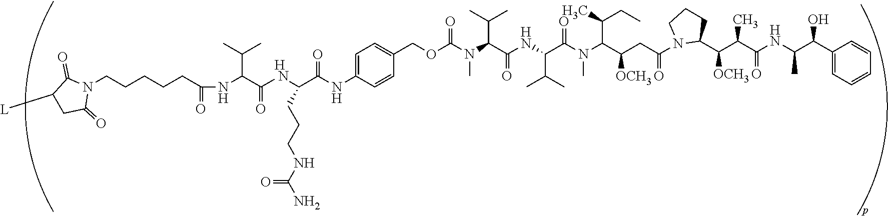

In some embodiments of the formula described in the preceding paragraph, p ranges from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments of the formula described in the preceding paragraph, p ranges from 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4 or 2 to 3. In some embodiments of the formula described in the preceding paragraph, p ranges from 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, or 3 to 4. In some embodiments of the formula described in the preceding paragraph, p is about 1. In some embodiments of the formula described in the preceding paragraph, p is about 2. In some embodiments of the formula described in the preceding paragraph, p is about 3. In some embodiments of the formula described in the preceding paragraph, p is about 4. In some embodiments of the formula described in the preceding paragraph, p is about 3.8. In some embodiments of the formula described in the preceding paragraph, p is about 5. In some embodiments of the formula described in the preceding paragraph, p is about 6. In some embodiments of the formula described in the preceding paragraph, p is about 7. In some embodiments of the formula described in the preceding paragraph, p is about 8. In some embodiments of the formula described in the preceding paragraph, p is about 9. In some embodiments of the formula described in the preceding paragraph, p is about 10. In some embodiments of the formula described in the preceding paragraph, p is about 11. In some embodiments of the formula described in the preceding paragraph, p is about 12. In some embodiments of the formula described in the preceding paragraph, p is about 13. In some embodiments of the formula described in the preceding paragraph, p is about 14. In some embodiments of the formula described in the preceding paragraph, p is about 15. In some embodiments of the formula described in the preceding paragraph, p is about 16. In some embodiments of the formula described in the preceding paragraph, p is about 17. In some embodiments of the formula described in the preceding paragraph, p is about 18. In some embodiments of the formula described in the preceding paragraph, p is about 19. In some embodiments of the formula described in the preceding paragraph, p is about 20.

Typically, peptide-based drug units can be prepared by forming a peptide bond between two or more amino acids and/or peptide fragments. Such peptide bonds can be prepared, for example, according to the liquid phase synthesis method (see E. Schroder and K. Lubke, “The Peptides”, volume 1, pp 76-136, 1965, Academic Press) that is well-known in the field of peptide chemistry. The auristatin/dolastatin drug units can be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat Biotechnol 21(7):778-784.

Additional embodiments of cytotoxic agent have been described in U.S. Pat. No. 8,637,642 and International Application No. PCT/US2019/056214 (Publication No. WO2020/117373), both of which are hereby incorporated in their entireties by reference.

### 5.3.3 Linkers

Typically, the antibody drug conjugates comprise a linker unit between the drug unit (e.g., MMAE) and the antibody unit (e.g., the anti-191P4D12 antibody or antigen binding fragment thereof). In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO:15)). In some embodiments, the peptidyl linker is at least two amino acids long or at least three amino acids long. In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT.

A “linker unit” (LU) is a bifunctional compound that can be used to link a drug unit and an antibody unit to form an antibody drug conjugate. In some embodiments, the linker unit has the formula:

-Aₐ-W_{w}-Y_{y}-
wherein: -A- is a stretcher unit,
a is 0 or 1,
each -W- is independently an amino acid unit,
w is an integer ranging from 0 to 12,
-Y- is a self-immolative spacer unit, and
y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0. The linker and each of the stretcher unit, the amino acid unit, and the spacer unit have been described in U.S. Pat. No. 8,637,642 and International Application No. PCT/US2019/056214 (Publication No. WO2020/117373), both of which are hereby incorporated in their entireties by reference.

Embodiments of the antibody-drug conjugates can include:

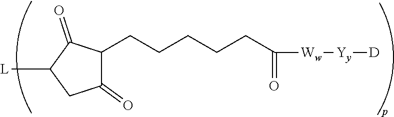

wherein w and y are each 0, 1 or 2, and,

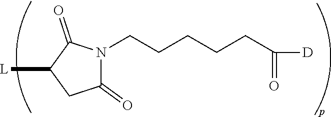

wherein w and y are each 0,

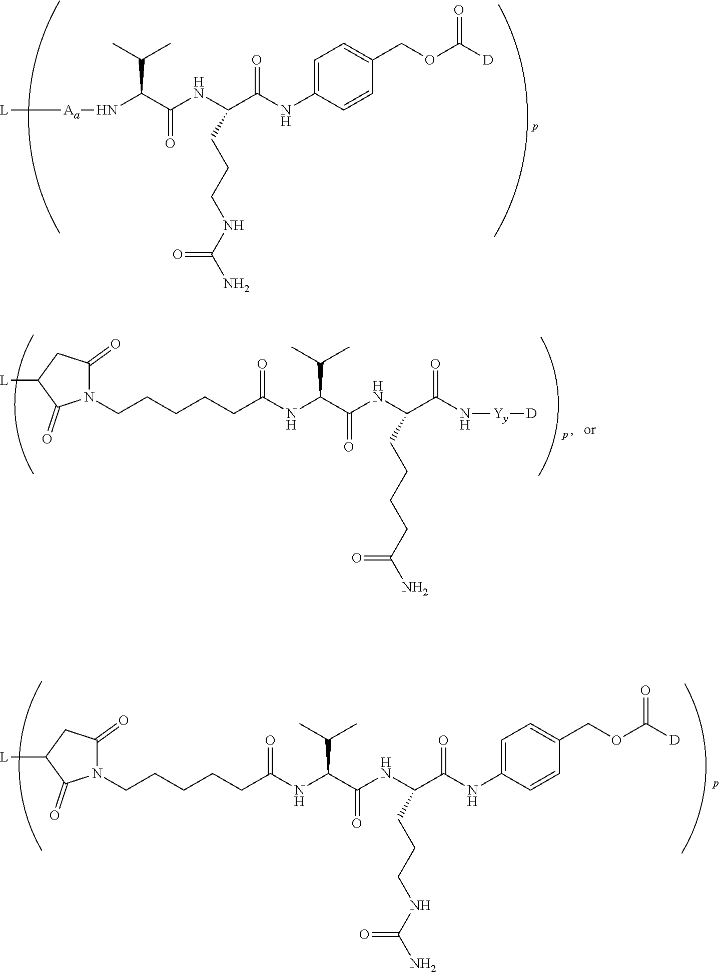

### 5.3.4 Drug Loading

Drug loading is represented by p and is the average number of drug units per antibody in a molecule. Drug loading can range from 1 to 20 drug units (D) per antibody. The ADCs provided herein include collections of antibodies or antigen binding fragments conjugated with a range of drug units, e.g., from 1 to 20. The average number of drug units per antibody in preparations of ADC from conjugation reactions can be characterized by conventional means such as mass spectroscopy and, ELISA assay. The quantitative distribution of ADC in terms of p can also be determined. In some instances, separation, purification, and characterization of homogeneous ADC where p is a certain value from ADC with other drug loadings can be achieved by means such as electrophoresis.

In certain embodiments, the drug loading for an ADC provided herein ranges from 1 to 20. In certain embodiments, the drug loading for an ADC provided herein ranges from 1 to 18. In certain embodiments, the drug loading for an ADC provided herein ranges from 1 to 15. In certain embodiments, the drug loading for an ADC provided herein ranges from 1 to 12. In certain embodiments, the drug loading for an ADC provided herein ranges from 1 to 10. In certain embodiments, the drug loading for an ADC provided herein ranges from 1 to 9. In certain embodiments, the drug loading for an ADC provided herein ranges from 1 to 8. In certain embodiments, the drug loading for an ADC provided herein ranges from 1 to 7. In certain embodiments, the drug loading for an ADC provided herein ranges from 1 to 6. In certain embodiments, the drug loading for an ADC provided herein ranges from 1 to 5. In certain embodiments, the drug loading for an ADC provided herein ranges from 1 to 4. In certain embodiments, the drug loading for an ADC provided herein ranges from 1 to 3. In certain embodiments, the drug loading for an ADC provided herein ranges from 2 to 12. In certain embodiments, the drug loading for an ADC provided herein ranges from 2 to 10. In certain embodiments, the drug loading for an ADC provided herein ranges from 2 to 9. In certain embodiments, the drug loading for an ADC provided herein ranges from 2 to 8. In certain embodiments, the drug loading for an ADC provided herein ranges from 2 to 7. In certain embodiments, the drug loading for an ADC provided herein ranges from 2 to 6. In certain embodiments, the drug loading for an ADC provided herein ranges from 2 to 5. In certain embodiments, the drug loading for an ADC provided herein ranges from 2 to 4. In certain embodiments, the drug loading for an ADC provided herein ranges from 3 to 12. In certain embodiments, the drug loading for an ADC provided herein ranges from 3 to 10. In certain embodiments, the drug loading for an ADC provided herein ranges from 3 to 9. In certain embodiments, the drug loading for an ADC provided herein ranges from 3 to 8. In certain embodiments, the drug loading for an ADC provided herein ranges from 3 to 7. In certain embodiments, the drug loading for an ADC provided herein ranges from 3 to 6. In certain embodiments, the drug loading for an ADC provided herein ranges from 3 to 5. In certain embodiments, the drug loading for an ADC provided herein ranges from 3 to 4.

In certain embodiments, the drug loading for an ADC provided herein ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7.

In certain embodiments, the drug loading for an ADC provided herein is about 1, about 2, about 3, about 4, about 5, about 6, about 7, about 8, about 9, about 10, about 11, about 12, or more. In some embodiments, the drug loading for an ADC provided herein is about 3.1, about 3.2, about 3.3, about 3.4, about 3.5, about 3.6, about 3.7, about 3.8, or about 3.9.

In some embodiments, the drug loading for an ADC provided herein ranges from 2 to 20, 2 to 19, 2 to 18, 2 to 17, 2 to 16, 2 to 15, 2 to 14, or 2 to 13. In some embodiments, the drug loading for an ADC provided herein ranges from 3 to 20, 3 to 19, 3 to 18, 3 to 17, 3 to 16, 3 to 15, 3 to 14, or 3 to 13. In some embodiments, the drug loading for an ADC provided herein is about 1. In some embodiments, the drug loading for an ADC provided herein is about 2. In some embodiments, the drug loading for an ADC provided herein is about 3. In some embodiments, the drug loading for an ADC provided herein is about 4. In some embodiments, the drug loading for an ADC provided herein is about 3.8. In some embodiments, the drug loading for an ADC provided herein is about 5. In some embodiments, the drug loading for an ADC provided herein is about 6. In some embodiments, the drug loading for an ADC provided herein is about 7. In some embodiments, the drug loading for an ADC provided herein is about 8. In some embodiments, the drug loading for an ADC provided herein is about 9. In some embodiments, the drug loading for an ADC provided herein is about 10. In some embodiments, the drug loading for an ADC provided herein is about 11. In some embodiments, the drug loading for an ADC provided herein is about 12. In some embodiments, the drug loading for an ADC provided herein is about 13. In some embodiments, the drug loading for an ADC provided herein is about 14. In some embodiments, the drug loading for an ADC provided herein is about 15. In some embodiments, the drug loading for an ADC provided herein is about 16. In some embodiments, the drug loading for an ADC provided herein is about 17. In some embodiments, the drug loading for an ADC provided herein is about 18. In some embodiments, the drug loading for an ADC provided herein is about 19. In some embodiments, the drug loading for an ADC provided herein is about 20.

In certain embodiments, fewer than the theoretical maximum of drug units are conjugated to an antibody during a conjugation reaction. An antibody can contain, for example, lysine residues that do not react with the drug-linker intermediate or linker reagent. Generally, antibodies do not contain many free and reactive cysteine thiol groups which can be linked to a drug unit; indeed most cysteine thiol residues in antibodies exist as disulfide bridges. In certain embodiments, an antibody can be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine. In some embodiments, the linker unit or a drug unit is conjugated via a lysine residue on the antibody unit. In some embodiments, the linker unit or a drug unit is conjugated via a cysteine residue on the antibody unit.

In some embodiments, the amino acid that attaches to a linker unit or a drug unit is in the heavy chain of an antibody or antigen binding fragment thereof. In some embodiments, the amino acid that attaches to a linker unit or a drug unit is in the light chain of an antibody or antigen binding fragment thereof. In some embodiments, the amino acid that attaches to a linker unit or a drug unit is in the hinge region of an antibody or antigen binding fragment thereof. In some embodiments, the amino acid that attaches to a linker unit or a drug unit is in the Fc region of an antibody or antigen binding fragment thereof. In other embodiments, the amino acid that attaches to a linker unit or a drug unit is in the constant region (e.g., CH1, CH2, or CH3 of a heavy chain, or CH1 of a light chain) of an antibody or antigen binding fragment thereof. In yet other embodiments, the amino acid that attaches to a linker unit or a drug unit is in the VH framework regions of an antibody or antigen binding fragment thereof. In yet other embodiments, the amino acid that attaches to a linker unit or a drug unit is in the VL framework regions of an antibody or antigen binding fragment thereof.

The loading (drug/antibody ratio) of an ADC can be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug unit reagent, then the resulting product is a mixture of ADC compounds with a distribution of one or more drug unit attached to an antibody unit. The average number of drugs per antibody can be calculated from the mixture by a dual ELISA antibody assay, which is specific for antibody and specific for the drug. Individual ADC molecules can be identified in the mixture by mass spectroscopy and separated by HPLC, e.g. hydrophobic interaction chromatography (see, e.g., Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value can be isolated from the conjugation mixture by electrophoresis or chromatography.

Methods for preparing, screening, and characterizing the antibody drug conjugates are known to a person of ordinary skill in the art, for example, as described in U.S. Pat. No. 8,637,642, which is herein incorporated in its entirety by reference.

In some embodiments, the antibody drug conjugate for the methods provided herein is AGS-22M6E, which is prepared according to the methods described in U.S. Pat. No. 8,637,642 and has the following formula:

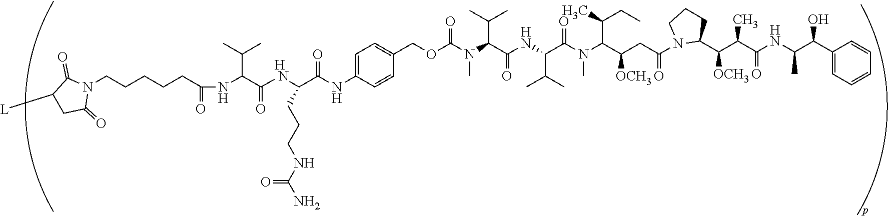

wherein L is Ha22-2(2,4)6.1 and p is from 1 to 20.

In some embodiments, p ranges from 1 to 20, 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is about 1. In other embodiments, p is about 2. In other embodiments, p is about 3. In other embodiments, p is about 4. In other embodiments, p is about 5. In other embodiments, p is about 6. In other embodiments, p is about 7. In other embodiments, p is about 8. In other embodiments, p is about 9. In other embodiments, p is about 10. In some embodiments, p is about 3.1. In some embodiments, p is about 3.2. In some embodiments, p is about 3.3. In some embodiments, p is about 3.4. In some embodiments, p is about 3.5. In other embodiments, p is about 3.6. In some embodiments, p is about 3.7. In some embodiments, p is about 3.8. In some embodiments, p is about 3.9. In some embodiments, p is about 4.0. In some embodiments, p is about 4.1. In some embodiments, p is about 4.2. In some embodiments, p is about 4.3. In some embodiments, p is about 4.4. In some embodiments, p is about 4.5. In other embodiments, p is about 4.6. In some embodiments, p is about 4.7. In some embodiments, p is about 4.8. In some embodiments, p is about 4.9. In some embodiments, p is about 5.0.

In some embodiments, the ADC used in the methods provided herein is enfortumab vedotin. Enfortumab vedotin is an ADC comprised of a fully human immunoglobulin G1 kappa (IgG1_{K}) antibody conjugated to the microtubule-disrupting agent (MMAE) via a protease-cleavable linker (Challita-Eid P M et al, Cancer Res. 2016; 76(10):3003-13]. Enfortumab vedotin induces antitumor activity by binding to 191P4D12 protein on the cell surface leading to internalization of the ADC-191P4D12 complex, which then traffics to the lysosomal compartment where MMAE is released via proteolytic cleavage of the linker. Intracellular release of MMAE subsequently disrupts tubulin polymerization resulting in G2/M phase cell cycle arrest and apoptotic cell death (Francisco J A et al, Blood. 2003 Aug. 15; 102(4):1458-65).

As described above and in in U.S. Pat. No. 8,637,642, AGS-22M6E is an ADC derived from a murine hybridoma cell line. Enfortumab vedotin is a Chinese hamster ovary (CHO) cell line-derived equivalent of AGS-22M6E ADC and is an exemplary product used for human treatment. Enfortumab vedotin has the same amino acid sequence, linker and cytotoxic drug as AGS-22M6E. The comparability between enfortumab vedotin and AGS-22M6E was confirmed through extensive analytical and biological characterization studies, such as binding affinity to 191P4D12, in vitro cytotoxicity, and in vivo antitumor activity.

In one embodiment, the ADC provided herein is enfortumab vedotin, also known as EV, PADCEV, AGS-22M6E, AGS-22C3E, ASG-22C3E. The enfortumab vedotin includes an anti-191P4D12 antibody, wherein the antibody or antigen binding fragment thereof comprises a heavy chain comprising amino acid residue 20 to amino acid residue 466 of SEQ ID NO:7 and a light chain comprising amino acid residue 23 to amino acid residue 236 of SEQ ID NO:8.

Enfortumab vedotin is a Nectin-4 directed antibody-drug conjugate (ADC) comprised of a fully human anti-nectin-4 IgG1 kappa monoclonal antibody (AGS-22C3) conjugated to the small molecule microtubule disrupting agent, monomethyl auristatin E (MMAE) via a protease-cleavable maleimidocaproyl valine-citrulline (vc) linker (SGD-1006). Conjugation takes place on cysteine residues that comprise the interchain disulfide bonds of the antibody to yield a product with a drug-to-antibody ratio of approximately 3.8:1. The molecular weight is approximately 152 kDa.

Enfortumab vedotin has the following structural formula:

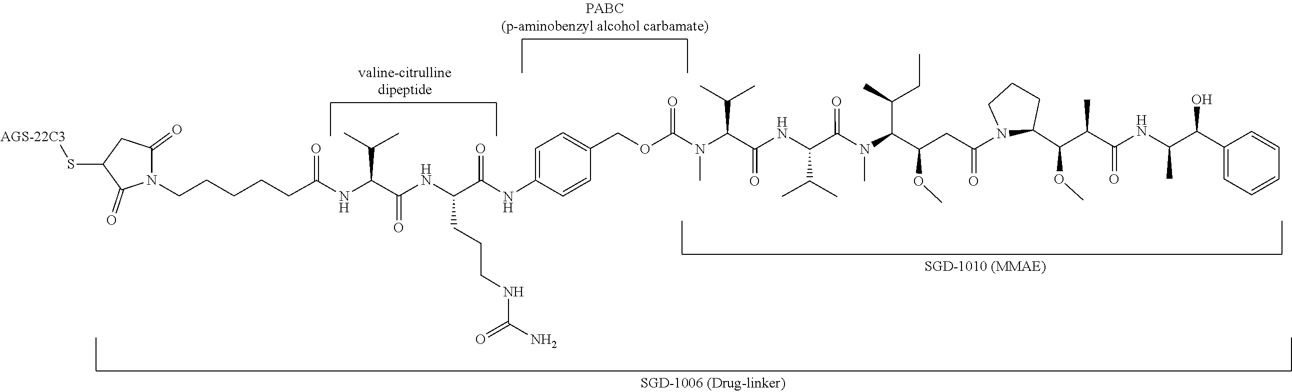

Approximately 4 molecules of MMAE are attached to each antibody molecule. Enfortumab vedotin is produced by chemical conjugation of the antibody and small molecule components. The antibody is produced by mammalian (Chinese hamster ovary) cells and the small molecule components are produced by chemical synthesis.

Enfortumab vedotin injection is provided as a sterile, preservative-free, white to off-white lyophilized powder in single-dose vials for intravenous use. Enfortumab vedotin is supplied as a 20 mg per vial and a 30 mg per vial and requires reconstitution with Sterile Water for Injection, USP, (2.3 mL and 3.3 mL, respectively) resulting in a clear to slightly opalescent, colorless to slightly yellow solution with a final concentration of 10 mg/mL. After reconstitution, each vial allows the withdrawal of 2 mL (20 mg) and 3 mL (30 mg). Each mL of reconstituted solution contains 10 mg of enfortumab vedotin, histidine (1.4 mg), histidine hydrochloride monohydrate (2.31 mg), polysorbate 20 (0.2 mg) and trehalose dihydrate (55 mg) with a pH of 6.0.

### 5.4 Pharmaceutical Compositions

In certain embodiments of the methods provided herein, the ADC used in the methods is provided in “pharmaceutical compositions.” Such pharmaceutical compositions include an antibody drug conjugate provided herein, and one or more pharmaceutically acceptable or physiologically acceptable excipients. In certain embodiments, the antibody drug conjugate are provided in combination with, or separate from, one or more additional agents. Also provided is a composition comprising such one or more additional agents and one or more pharmaceutically acceptable or physiologically acceptable excipients. In particular embodiments, the antibody drug conjugate and an additional agent(s) are present in a therapeutically acceptable amount. The pharmaceutical compositions can be used in accordance with the methods and uses provided herein. Thus, for example, the pharmaceutical compositions can be administered ex vivo or in vivo to a subject in order to practice treatment methods and uses provided herein. Pharmaceutical compositions provided herein can be formulated to be compatible with the intended method or route of administration; exemplary routes of administration are set forth herein.

In some embodiments, provided are pharmaceutical compositions of antibody drug conjugates that modulate a cancer or tumor.

In certain embodiments of the methods provided herein, the pharmaceutical compositions comprising the ADCs can further comprise other therapeutically active agents or compounds disclosed herein or known to the skilled artisan which can be used in the treatment or prevention of various diseases and disorders as set forth herein (e.g., a cancer). As set forth above, the additional therapeutically active agents or compounds can be present in a separate pharmaceutical composition(s).

Pharmaceutical compositions typically comprise a therapeutically effective amount of at least one of the antibody drug conjugates provided herein and one or more pharmaceutically acceptable formulation agents. In certain embodiments, the pharmaceutical composition further comprises one or more additional agents described herein.

In one embodiment, a pharmaceutical composition comprises an antibody drug conjugate provided herein. In some embodiments, a pharmaceutical composition comprises a therapeutically effective amount of an antibody drug conjugate provided herein. In certain embodiments, the pharmaceutical composition comprises a pharmaceutically acceptable excipient.

In some embodiments, the antibody drug conjugate in the pharmaceutical composition provided herein is selected from the antibody drug conjugates described in Section 5.3 above.

In certain embodiments, the pharmaceutical composition comprises the antibody drug conjugate at a concentration of from 0.1-100 mg/mL. In some embodiments, the pharmaceutical composition comprises the antibody drug conjugate at a concentration of from 1 to 20 mg/mL. In other embodiments, the pharmaceutical composition comprises the antibody drug conjugate at a concentration of from 5 to 15 mg/mL. In other embodiments, the pharmaceutical composition comprises the antibody drug conjugate at a concentration of from 8 to 12 mg/mL. In other embodiments, the pharmaceutical composition comprises the antibody drug conjugate at a concentration of from 9 to 11 mg/mL. In some embodiments, the pharmaceutical composition comprises the antibody drug conjugate at a concentration of about 9.5 mg/mL. In some embodiments, the pharmaceutical composition comprises the antibody drug conjugate at a concentration of about 9.6 mg/mL. In some embodiments, the pharmaceutical composition comprises the antibody drug conjugate at a concentration of about 9.7 mg/mL. In some embodiments, the pharmaceutical composition comprises the antibody drug conjugate at a concentration of about 9.8 mg/mL. In some embodiments, the pharmaceutical composition comprises the antibody drug conjugate at a concentration of about 9.9 mg/mL. In yet other embodiments, the pharmaceutical composition comprises the antibody drug conjugate at a concentration of about 10 mg/mL. In yet other embodiments, the pharmaceutical composition comprises the antibody drug conjugate at a concentration of about 10.1 mg/mL. In some embodiments, the pharmaceutical composition comprises the antibody drug conjugate at a concentration of about 10.2 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.3 mg/mL. In some embodiments, the pharmaceutical composition comprises the antibody drug conjugate at a concentration of about 10.4 mg/mL. In some embodiments, the pharmaceutical composition comprises the antibody drug conjugate at a concentration of about 10.5 mg/mL.

In some embodiments, the pharmaceutical composition provided herein comprises L-histidine, TWEEN-20, and at least one of trehalose dihydrate or sucrose. In some embodiments, the pharmaceutical composition provided herein further comprises hydrochloric acid (HCl) or succinic acid.

In some embodiments, the concentration of L-histidine useful in the pharmaceutical compositions provided herein is in the range of between 5 and 50 mM. In some embodiments, the concentration of L-histidine in the pharmaceutical compositions provided herein is in the range of between 10 and 40 mM. In some embodiments, the concentration of L-histidine in the pharmaceutical compositions provided herein is in the range of between 15 and 35 mM.

In some embodiments, the concentration of L-histidine in the pharmaceutical compositions provided herein is in the range of between 15 and 30 mM. In some embodiments, the concentration of L-histidine in the pharmaceutical compositions provided herein is in the range of between 15 and 25 mM. In some embodiments, the concentration of L-histidine in the pharmaceutical compositions provided herein is in the range of between 15 and 35 mM. In some embodiments, the concentration of L-histidine in the pharmaceutical compositions provided herein is about 16 mM. In some embodiments, the concentration of L-histidine in the pharmaceutical compositions provided herein is about 17 mM. In some embodiments, the concentration of L-histidine in the pharmaceutical compositions provided herein is about 18 mM. In some embodiments, the concentration of L-histidine in the pharmaceutical compositions provided herein is about 19 mM. In some embodiments, the concentration of L-histidine in the pharmaceutical compositions provided herein is about 20 mM. In some embodiments, the concentration of L-histidine in the pharmaceutical compositions provided herein is about 21 mM. In some embodiments, the concentration of L-histidine in the pharmaceutical compositions provided herein is about 22 mM. In some embodiments, the concentration of L-histidine in the pharmaceutical compositions provided herein is about 23 mM. In some embodiments, the concentration of L-histidine in the pharmaceutical compositions provided herein is about 24 mM. In some embodiments, the concentration of L-histidine in the pharmaceutical compositions provided herein is about 25 mM.

In some embodiments, the concentration of TWEEN-20 useful in the pharmaceutical compositions provided herein is in the range of from 0.001 to 0.1% (v/v). In another embodiment, the concentration of TWEEN-20 is in the range of from 0.0025 to 0.075% (v/v). In one embodiment, the concentration of TWEEN-20 is in the range of from 0.005 to 0.05% (v/v). In another embodiment, the concentration of TWEEN-20 is in the range of from 0.0075 to 0.025% (v/v). In another embodiment, the concentration of TWEEN-20 is in the range of from 0.0075 to 0.05% (v/v). In another embodiment, the concentration of TWEEN-20 is in the range of from 0.01 to 0.03% (v/v). In one particular embodiment, the concentration of TWEEN-20 is about 0.01% (v/v). In one particular embodiment, the concentration of TWEEN-20 is about 0.015% (v/v). In one particular embodiment, the concentration of TWEEN-20 is about 0.016% (v/v). In one particular embodiment, the concentration of TWEEN-20 is about 0.017% (v/v). In one particular embodiment, the concentration of TWEEN-20 is about 0.018% (v/v). In one particular embodiment, the concentration of TWEEN-20 is about 0.019% (v/v). In one particular embodiment, the concentration of TWEEN-20 is about 0.02% (v/v). In one particular embodiment, the concentration of TWEEN-20 is about 0.021% (v/v). In one particular embodiment, the concentration of TWEEN-20 is about 0.022% (v/v). In one particular embodiment, the concentration of TWEEN-20 is about 0.023% (v/v). In one particular embodiment, the concentration of TWEEN-20 is about 0.024% (v/v). In one particular embodiment, the concentration of TWEEN-20 is about 0.025% (v/v).

In one embodiment, the concentration of trehalose dihydrate useful in the pharmaceutical compositions provided herein is in the range of between 1% and 20% (w/v). In another embodiment, the concentration of trehalose dihydrate is in the range of 2% and 15% (w/v). In one embodiment, the concentration of trehalose dihydrate is in the range of 3% and 10% (w/v). In another embodiment, the concentration of trehalose dihydrate is in the range of 4% and 9% (w/v). In another embodiment, the concentration of trehalose dihydrate is in the range of 4% and 8% (w/v). In another embodiment, the concentration of trehalose dihydrate is in the range of 4% and 7% (w/v). In another embodiment, the concentration of trehalose dihydrate is in the range of 4% and 6% (w/v). In another embodiment, the concentration of trehalose dihydrate is in the range of 4.5% and 6% (w/v). In another embodiment, the concentration of trehalose dihydrate is about 4.6% (w/v). In another embodiment, the concentration of trehalose dihydrate is about 4.7% (w/v). In another embodiment, the concentration of trehalose dihydrate is about 4.8% (w/v). In another embodiment, the concentration of trehalose dihydrate is about 4.9% (w/v). In another embodiment, the concentration of trehalose dihydrate is about 5.0% (w/v). In another embodiment, the concentration of trehalose dihydrate is about 5.1% (w/v). In another embodiment, the concentration of trehalose dihydrate is about 5.2% (w/v). In another embodiment, the concentration of trehalose dihydrate is about 5.3% (w/v). In another embodiment, the concentration of trehalose dihydrate is about 5.4% (w/v). In another embodiment, the concentration of trehalose dihydrate is about 5.5% (w/v). In another embodiment, the concentration of trehalose dihydrate is about 5.6% (w/v). In another embodiment, the concentration of trehalose dihydrate is about 5.7% (w/v). In another embodiment, the concentration of trehalose dihydrate is about 5.8% (w/v). In another embodiment, the concentration of trehalose dihydrate is about 5.9% (w/v). In another embodiment, the concentration of trehalose dihydrate is about 6.0% (w/v). In another embodiment, the concentration of trehalose dihydrate is about 6.1% (w/v). In another embodiment, the concentration of trehalose dihydrate is about 6.2% (w/v). In another embodiment, the concentration of trehalose dihydrate is about 6.3% (w/v). In another embodiment, the concentration of trehalose dihydrate is about 6.4% (w/v). In another embodiment, the concentration of trehalose dihydrate is about 6.5% (w/v).

In certain embodiments, the molarity of the trehalose dihydrate is from 50 to 300 mM. In other embodiments, the molarity of the trehalose dihydrate is from 75 to 250 mM. In some embodiments, the molarity of the trehalose dihydrate is from 100 to 200 mM. In other embodiments, the molarity of the trehalose dihydrate is from 130 to 150 mM. In some embodiments, the molarity of the trehalose dihydrate is from 135 to 150 mM. In certain embodiments, the molarity of the trehalose dihydrate is about 135 mM. In certain embodiments, the molarity of the trehalose dihydrate is about 136 mM. In certain embodiments, the molarity of the trehalose dihydrate is about 137 mM. In certain embodiments, the molarity of the trehalose dihydrate is about 138 mM. In certain embodiments, the molarity of the trehalose dihydrate is about 139 mM. In certain embodiments, the molarity of the trehalose dihydrate is about 140 mM. In certain embodiments, the molarity of the trehalose dihydrate is about 141 mM. In certain embodiments, the molarity of the trehalose dihydrate is about 142 mM. In certain embodiments, the molarity of the trehalose dihydrate is about 143 mM. In certain embodiments, the molarity of the trehalose dihydrate is about 144 mM. In certain embodiments, the molarity of the trehalose dihydrate is about 145 mM. In certain embodiments, the molarity of the trehalose dihydrate is about 146 mM. In certain embodiments, the molarity of the trehalose dihydrate is about 150 mM. In certain embodiments, the molarity of the trehalose dihydrate is about 151 mM. In certain embodiments, the molarity of the trehalose dihydrate is about 151 mM. In certain embodiments, the molarity of the trehalose dihydrate is about 152 mM. In certain embodiments, the molarity of the trehalose dihydrate is about 153 mM. In certain embodiments, the molarity of the trehalose dihydrate is about 154 mM. In certain embodiments, the molarity of the trehalose dihydrate is about 155 mM.

In one embodiment, the concentration of sucrose useful in the pharmaceutical compositions provided herein is in the range of between 1% and 20% (w/v). In another embodiment, the concentration of sucrose is in the range of 2% and 15% (w/v). In one embodiment, the concentration of sucrose is in the range of 3% and 10% (w/v). In another embodiment, the concentration of sucrose is in the range of 4% and 9% (w/v). In another embodiment, the concentration of sucrose is in the range of 4% and 8% (w/v). In another embodiment, the concentration of sucrose is in the range of 4% and 7% (w/v). In another embodiment, the concentration of sucrose is in the range of 4% and 6% (w/v). In another embodiment, the concentration of sucrose is in the range of 4.5% and 6% (w/v). In another embodiment, the concentration of sucrose is about 4.6% (w/v). In another embodiment, the concentration of sucrose is about 4.7% (w/v). In another embodiment, the concentration of sucrose is about 4.8% (w/v). In another embodiment, the concentration of sucrose is about 4.9% (w/v). In another embodiment, the concentration of sucrose is about 5.0% (w/v). In another embodiment, the concentration of sucrose is about 5.1% (w/v). In another embodiment, the concentration of sucrose is about 5.2% (w/v). In another embodiment, the concentration of sucrose is about 5.3% (w/v). In another embodiment, the concentration of sucrose is about 5.4% (w/v). In another embodiment, the concentration of sucrose is about 5.5% (w/v). In another embodiment, the concentration of sucrose is about 5.6% (w/v). In another embodiment, the concentration of sucrose is about 5.7% (w/v). In another embodiment, the concentration of sucrose is about 5.8% (w/v). In another embodiment, the concentration of sucrose is about 5.9% (w/v). In another embodiment, the concentration of sucrose is about 6.0% (w/v). In another embodiment, the concentration of sucrose is about 6.1% (w/v). In another embodiment, the concentration of sucrose is about 6.2% (w/v). In another embodiment, the concentration of sucrose is about 6.3% (w/v). In another embodiment, the concentration of sucrose is about 6.4% (w/v). In another embodiment, the concentration of sucrose is about 6.5% (w/v).

In certain embodiments, the molarity of the sucrose is from 50 to 300 mM. In other embodiments, the molarity of the sucrose is from 75 to 250 mM. In some embodiments, the molarity of the sucrose is from 100 to 200 mM. In other embodiments, the molarity of the sucrose is from 130 to 150 mM. In some embodiments, the molarity of the sucrose is from 135 to 150 mM. In certain embodiments, the molarity of the sucrose is about 135 mM. In certain embodiments, the molarity of the sucrose is about 136 mM. In certain embodiments, the molarity of the sucrose is about 137 mM. In certain embodiments, the molarity of the sucrose is about 138 mM. In certain embodiments, the molarity of the sucrose is about 139 mM. In certain embodiments, the molarity of the sucrose is about 140 mM. In certain embodiments, the molarity of the sucrose is about 141 mM. In certain embodiments, the molarity of the sucrose is about 142 mM. In certain embodiments, the molarity of the sucrose is about 143 mM. In certain embodiments, the molarity of the sucrose is about 144 mM. In certain embodiments, the molarity of the sucrose is about 145 mM. In certain embodiments, the molarity of the sucrose is about 146 mM. In certain embodiments, the molarity of the sucrose is about 150 mM. In certain embodiments, the molarity of the sucrose is about 151 mM. In certain embodiments, the molarity of the sucrose is about 151 mM. In certain embodiments, the molarity of the sucrose is about 152 mM. In certain embodiments, the molarity of the sucrose is about 153 mM. In certain embodiments, the molarity of the sucrose is about 154 mM. In certain embodiments, the molarity of the sucrose is about 155 mM.

In some embodiments, the pharmaceutical composition provided herein comprises HCl. In other embodiments, the pharmaceutical composition provided herein comprises succinic acid.

In some embodiments, the pharmaceutical composition provided herein has a pH in a range of 5.5 to 6.5. In other embodiments, the pharmaceutical composition provided herein has a pH in a range of 5.7 to 6.3. In some embodiments, the pharmaceutical composition provided herein has a pH of about 5.7. In some embodiments, the pharmaceutical composition provided herein has a pH of about 5.8. In some embodiments, the pharmaceutical composition provided herein has a pH of about 5.9. In some embodiments, the pharmaceutical composition provided herein has a pH of about 6.0. In some embodiments, the pharmaceutical composition provided herein has a pH of about 6.1. In some embodiments, the pharmaceutical composition provided herein has a pH of about 6.2. In some embodiments, the pharmaceutical composition provided herein has a pH of about 6.3.

In some embodiments, the pH is taken at room temperature. In other embodiments, the pH is taken at 15° C. to 27° C. In yet other embodiments, the pH is taken at 4° C. In yet other embodiments, the pH is taken at 25° C.

In some embodiments, the pH is adjusted by HCl. In some embodiments, the pharmaceutical composition comprises HCl, and the pharmaceutical composition has a pH in a range of 5.5 to 6.5 at room temperature. In some embodiments, the pharmaceutical composition comprises HCl, and the pharmaceutical composition has a pH in a range of 5.7 to 6.3 at room temperature. In some more specific embodiments, the pharmaceutical composition comprises HCl, and the pharmaceutical composition has a pH of about of 5.7 at room temperature. In some more specific embodiments, the pharmaceutical composition comprises HCl, and the pharmaceutical composition has a pH of about of 5.8 at room temperature. In some more specific embodiments, the pharmaceutical composition comprises HCl, and the pharmaceutical composition has a pH of about of 5.9 at room temperature. In some more specific embodiments, the pharmaceutical composition comprises HCl, and the pharmaceutical composition has a pH of about of 6.0 at room temperature. In some more specific embodiments, the pharmaceutical composition comprises HCl, and the pharmaceutical composition has a pH of about of 6.1 at room temperature. In some more specific embodiments, the pharmaceutical composition comprises HCl, and the pharmaceutical composition has a pH of about of 6.2 at room temperature. In some more specific embodiments, the pharmaceutical composition comprises HCl, and the pharmaceutical composition has a pH of about of 6.3 at room temperature.

In some embodiments, the pharmaceutical composition comprises HCl, and the pharmaceutical composition has a pH in a range of 5.5 to 6.5 at 15° C. to 27° C. In some embodiments, the pharmaceutical composition comprises HCl, and the pharmaceutical composition has a pH in a range of 5.7 to 6.3 at 15° C. to 27° C. In some more specific embodiments, the pharmaceutical composition comprises HCl, and the pharmaceutical composition has a pH of about of 5.7 at 15° C. to 27° C. In some more specific embodiments, the pharmaceutical composition comprises HCl, and the pharmaceutical composition has a pH of about of 5.8 at 15° C. to 27° C. In some more specific embodiments, the pharmaceutical composition comprises HCl, and the pharmaceutical composition has a pH of about of 5.9 at 15° C. to 27° C. In some more specific embodiments, the pharmaceutical composition comprises HCl, and the pharmaceutical composition has a pH of about of 6.0 at 15° C. to 27° C. In some more specific embodiments, the pharmaceutical composition comprises HCl, and the pharmaceutical composition has a pH of about of 6.1 at 15° C. to 27° C. In some more specific embodiments, the pharmaceutical composition comprises HCl, and the pharmaceutical composition has a pH of about of 6.2 at 15° C. to 27° C. In some more specific embodiments, the pharmaceutical composition comprises HCl, and the pharmaceutical composition has a pH of about of 6.3 at 15° C. to 27° C.

In some embodiments, the pH is adjusted by succinic acid. In some embodiments, the pharmaceutical composition comprises succinic acid, and the pharmaceutical composition has a pH in a range of 5.5 to 6.5 at room temperature. In some embodiments, the pharmaceutical composition comprises succinic acid, and the pharmaceutical composition has a pH in a range of 5.7 to 6.3 at room temperature. In some more specific embodiments, the pharmaceutical composition comprises succinic acid, and the pharmaceutical composition has a pH of about of 5.7 at room temperature. In some more specific embodiments, the pharmaceutical composition comprises succinic acid, and the pharmaceutical composition has a pH of about of 5.8 at room temperature. In some more specific embodiments, the pharmaceutical composition comprises succinic acid, and the pharmaceutical composition has a pH of about of 5.9 at room temperature. In some more specific embodiments, the pharmaceutical composition comprises succinic acid, and the pharmaceutical composition has a pH of about of 6.0 at room temperature. In some more specific embodiments, the pharmaceutical composition comprises succinic acid, and the pharmaceutical composition has a pH of about of 6.1 at room temperature. In some more specific embodiments, the pharmaceutical composition comprises succinic acid, and the pharmaceutical composition has a pH of about of 6.2 at room temperature. In some more specific embodiments, the pharmaceutical composition comprises succinic acid, and the pharmaceutical composition has a pH of about of 6.3 at room temperature.

In some embodiments, the pharmaceutical composition comprises succinic acid, and the pharmaceutical composition has a pH in a range of 5.5 to 6.5 at 15° C. to 27° C. In some embodiments, the pharmaceutical composition comprises succinic acid, and the pharmaceutical composition has a pH in a range of 5.7 to 6.3 at 15° C. to 27° C. In some more specific embodiments, the pharmaceutical composition comprises succinic acid, and the pharmaceutical composition has a pH of about of 5.7 at 15° C. to 27° C. In some more specific embodiments, the pharmaceutical composition comprises succinic acid, and the pharmaceutical composition has a pH of about of 5.8 at 15° C. to 27° C. In some more specific embodiments, the pharmaceutical composition comprises succinic acid, and the pharmaceutical composition has a pH of about of 5.9 at 15° C. to 27° C. In some more specific embodiments, the pharmaceutical composition comprises succinic acid, and the pharmaceutical composition has a pH of about of 6.0 at 15° C. to 27° C. In some more specific embodiments, the pharmaceutical composition comprises succinic acid, and the pharmaceutical composition has a pH of about of 6.1 at 15° C. to 27° C. In some more specific embodiments, the pharmaceutical composition comprises succinic acid, and the pharmaceutical composition has a pH of about of 6.2 at 15° C. to 27° C. In some more specific embodiments, the pharmaceutical composition comprises succinic acid, and the pharmaceutical composition has a pH of about of 6.3 at 15° C. to 27° C.

In some specific embodiments, the pharmaceutical composition provided herein comprises about 20 mM L-histidine, about 0.02% (w/v) TWEEN-20, and at least one of about 5.5% (w/v) trehalose dihydrate or about 5% (w/v) sucrose. In some embodiments, the pharmaceutical composition provided herein further comprises HCl or succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5.5% (w/v) trehalose dihydrate and HCl. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and HCl. In some embodiments, the pH is about 6.0 at room temperature. In some embodiments, the pH is about 6.0 at 25° C.

In other specific embodiments, the pharmaceutical composition provided herein comprises about 20 mM L-histidine, about 0.02% (w/v) TWEEN-20, about 5.5% (w/v) trehalose dihydrate and succinic acid. In some embodiments, the pH is about 6.0 at room temperature. In some embodiments, the pH is about 6.0 at 25° C.

In some specific embodiments, the pharmaceutical composition provided herein comprises about 20 mM L-histidine, about 0.02% (w/v) TWEEN-20, about 5% (w/v) sucrose and succinic acid. In some embodiments, the pH is about 6.0 at room temperature. In some embodiments, the pH is about 6.0 at 25° C.

In some specific embodiments, the ADC is formulated in (i) a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.; or (ii) a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

In a specific embodiment, provided herein comprises
(a) an antibody drug conjugate comprising the following structure:

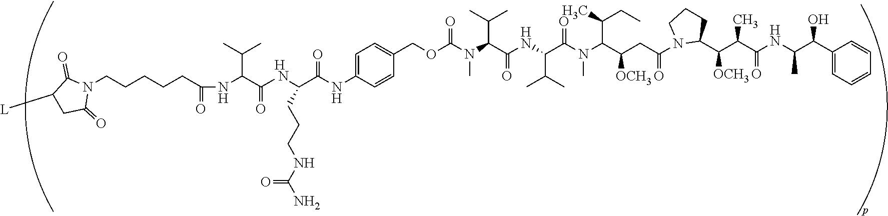

wherein L- represents the antibody or antigen binding fragment (e.g. anti-nectin-4 antibody or antigen binding fragment thereof) thereof and p is from 1 to 10; and
(b) a pharmaceutically acceptable excipient comprising about 20 mM L-histidine, about 0.02% (w/v) TWEEN-20, about 5.5% (w/v) trehalose dihydrate, and HCl, wherein the antibody drug conjugate is at the concentration of about 10 mg/mL, and wherein the pH is about 6.0 at 25° C.

In another specific embodiment, the pharmaceutical composition provided herein comprises:
(a) an antibody drug conjugate comprising the following structure:

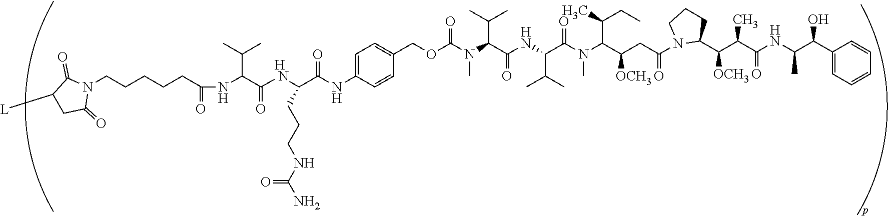

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.5% (w/v) trehalose dihydrate, and succinic acid, wherein the antibody drug conjugate is at the concentration of about 10 mg/mL, and wherein the pH is about 6.0 at 25° C.

In yet another specific embodiment, the pharmaceutical composition provided herein comprises:
(a) an antibody drug conjugate comprising the following structure:

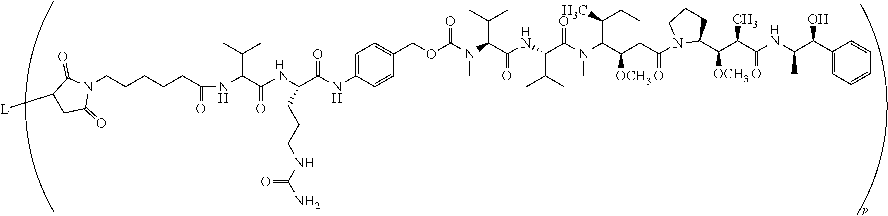

wherein L- represents the antibody or antigen binding fragment thereof (e.g. anti-nectin-4 antibody or antigen binding fragment thereof) and p is from 1 to 10; and
(b) a pharmaceutically acceptable excipient comprising about 20 mM L-histidine, about 0.02% (w/v) TWEEN-20, about 5.0% (w/v) sucrose, and HCl, wherein the antibody drug conjugate is at the concentration of about 10 mg/mL, and wherein the pH is about 6.0 at 25° C.

Although certain numbers (and numerical ranges thereof) are provided, it is understood that, in certain embodiments, numerical values within, e.g., 2%, 5%, 10%, 15% or 20% of said numbers (or numerical ranges) are also contemplated.

A primary solvent in a vehicle can be either aqueous or non-aqueous in nature. In addition, the vehicle can contain other pharmaceutically acceptable excipients for modifying or maintaining the pH, osmolarity, viscosity, sterility or stability of the pharmaceutical composition. In certain embodiments, the pharmaceutically acceptable vehicle is an aqueous buffer. In other embodiments, a vehicle comprises, for example, sodium chloride and/or sodium citrate.

Pharmaceutical compositions provided herein can contain still other pharmaceutically acceptable formulation agents for modifying or maintaining the rate of release of an antibody drug conjugate and/or an additional agent, as described herein. Such formulation agents include those substances known to artisans skilled in preparing sustained-release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712, The Merck Index, 12th Ed. (1996, Merck Publishing Group, Whitehouse, NJ); and Pharmaceutical Principles of Solid Dosage Forms (1993, Technonic Publishing Co., Inc., Lancaster, Pa.). Additional pharmaceutical compositions appropriate for administration are known in the art and are applicable in the methods and compositions provided herein.

In some embodiments, the pharmaceutical composition provided herein is in a liquid form. In other embodiments, the pharmaceutical composition provided herein is lyophilized.

A pharmaceutical composition can be formulated to be compatible with its intended route of administration. Thus, pharmaceutical compositions include excipients suitable for administration by routes including parenteral (e.g., subcutaneous (s.c.), intravenous, intramuscular, or intraperitoneal), intradermal, oral (e.g., ingestion), inhalation, intracavity, intracranial, and transdermal (topical). Other exemplary routes of administration are set forth herein.

Pharmaceutical compositions can be in the form of a sterile injectable aqueous or oleagenous suspension. This suspension can be formulated using suitable dispersing or wetting agents and suspending agents disclosed herein or known to the skilled artisan. The sterile injectable preparation can also be a sterile injectable solution or suspension in a nontoxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil can be employed, including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

In one embodiment, the pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In one embodiment, the pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, e.g., Remington, The Science and Practice of Pharmacy, supra).

In one embodiment, the pharmaceutical compositions intended for parenteral administration can include one or more pharmaceutically acceptable excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

In one embodiment, suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

In one embodiment, suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL©, CyDex, Lenexa, KS).

In one embodiment, the pharmaceutical compositions provided herein can be formulated for single or multiple dosage administration. The single dosage formulations are packaged in an ampoule, a vial, or a syringe. The multiple dosage parenteral formulations can contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

In one embodiment, the pharmaceutical compositions provided herein can be formulated as immediate or modified release dosage forms, including delayed-, sustained, pulsed-, controlled, targeted-, and programmed-release forms.

Dispersible powders and granules suitable for preparation of an aqueous suspension by addition of water provide the active ingredient in admixture with a dispersing or wetting agent, suspending agent and one or more preservatives. Suitable dispersing or wetting agents and suspending agents are exemplified herein.

Pharmaceutical compositions can also include excipients to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed. Prolonged absorption of injectable pharmaceutical compositions can be achieved by including an agent that delays absorption, for example, aluminum monostearate or gelatin. Prevention of the action of microorganisms can be achieved by various antibacterial and antifungal agents, for example, parabens, chlorobutanol, phenol, ascorbic acid, thimerosal, and the like.

The pharmaceutical composition provided herein can be stored at −80° C., 4° C., 25° C. or 37° C.

A lyophilized composition can be made by freeze-drying the liquid pharmaceutical composition provided herein. In a specific embodiment, the pharmaceutical composition provided here is a lyophilized pharmaceutical composition. In some embodiments, the pharmaceutical formulations are lyophilized powders, which can be reconstituted for administration as solutions, emulsions and other mixtures. They can also be reconstituted and formulated as solids or gels.

In some embodiments, preparation of the lyophilized formulation provided herein involves batching of the formulated bulk solution for lyophilization, aseptic filtration, filling in vials, freezing vials in a freeze-dryer chamber, followed by lyophilization, stoppering and capping.

A lyophilizer can be used in preparing the lyophilized formulation. For example, a VirTis Genesis Model EL pilot unit can be employed. The unit incorporates a chamber with three working shelves (to a total usable shelf area of ca 0.4 square meters), an external condenser, and a mechanical vacuum pumping system. Cascaded mechanical refrigeration allows the shelves to be cooled to −70° C. or lower, and the external condenser to −90° C. or lower. Shelf temperature and chamber pressure were controlled automatically to +/−0.5° C. and +/−2 microns (milliTorr), respectively. The unit was equipped with a capacitance manometer vacuum gauge, a Pirani vacuum gauge, a pressure transducer (to measure from 0 to 1 atmosphere), and a relative humidity sensor.

The lyophilized powder can be prepared by dissolving an antibody drug conjugate provided herein, or a pharmaceutically acceptable derivative thereof, in a suitable solvent. In some embodiments, the lyophilized powder is sterile. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the antibody drug conjugate. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable excipient. Such amount can be empirically determined and adjusted according to specific needs.

An exemplary reconstitution procedure is illustrated as follows: (1) fit the 5 mL or 3 mL syringe with a with a 18 or 20 Gauge needle and filled the syringe with water of the grade Water for Injection (WFI); (2) measure appropriate amount of WFI using the syringe graduations, ensuring that the syringe was free of air bubbles; (3) inserted the needle through the rubber stopper; (4) dispense the entire contents of the syringe into the container down the vial wall, removed the syringe and needle and put into the sharp container; (4) swirl the vial continuously to carefully solubilize the entire vial contents until fully reconstituted (e.g., about 20-40 seconds) and minimize excessive agitation of the protein solution that could result in foaming.

In some embodiments, the pharmaceutical composition provided herein is supplied as a dry sterilized lyophilized powder or water free concentrate in a hermetically sealed container and can be reconstituted, e.g., with water or saline to the appropriate concentration for administration to a subject. In certain embodiments, the antibody drug conjugate is supplied as a dry sterile lyophilized powder in a hermetically sealed container at a unit dosage of at least 0.1 mg, at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, at least 75 mg, at least 80 mg, at least 85 mg, at least 90 mg, at least 95 mg, or at least 100 mg. The lyophilized antibody drug conjugate can be stored at between 2 and 8° C. in its original container and the antibody drug conjugate can be administered within 12 hours, such as within 6 hours, within 5 hours, within 3 hours, or within 1 hour after being reconstituted. In an alternative embodiment, the pharmaceutical composition comprising the antibody drug conjugate provided herein is supplied in liquid form in a hermetically sealed container indicating the quantity and concentration of the antibody drug conjugate. In certain embodiments, the liquid form of the antibody drug conjugate is supplied in a hermetically sealed container at least 0.1 mg/ml, at least 0.5 mg/ml, at least 1 mg/ml, at least 5 mg/ml, at least 10 mg/ml, at least 15 mg/ml, at least 25 mg/ml, at least 30 mg/ml, at least 40 mg/ml, at least 50 mg/ml, at least 60 mg/ml, at least 70 mg/ml, at least 80 mg/ml, at least 90 mg/ml, or at least 100 mg/ml.

Additional embodiments for the pharmaceutical compositions have been described in U.S. Pat. No. 8,637,642 and International Application No. PCT/US2019/056214 (Publication No. WO2020/117373), both of which are hereby incorporated in their entireties by reference.

### 5.5 Methods for a Combination Therapy

The method for inhibiting growth of tumor cells using the pharmaceutical composition provided herein in combination with chemotherapy or radiation or both comprises administering the present pharmaceutical composition before, during, or after commencing chemotherapy or radiation therapy, as well as any combination thereof (i.e. before and during, before and after, during and after, or before, during, and after commencing the chemotherapy and/or radiation therapy). Depending on the treatment protocol and the specific patient needs, the method is performed in a manner that will provide the most efficacious treatment and ultimately prolong the life of the patient. Additional embodiments for such combination therapy have been described in U.S. Pat. No. 8,637,642 and International Application No. PCT/US2019/056214 (Publication No. WO2020/117373), both of which are hereby incorporated in their entireties by reference.

### 5.6 Doses for the Immune Checkpoint Inhibitors

In some embodiments, the patients treated using the pharmaceutical compositions described herein have not been treated with a checkpoint inhibitor. The exclusionary amount of the checkpoint inhibitor can be determined by standard clinical techniques.

### 5.7 Dosage of the ADCs for the Methods

In some embodiments, the amount of a prophylactic or therapeutic agent (e.g., an antibody drug conjugate provided herein), or a pharmaceutical composition provided herein that will be effective in the prevention and/or treatment of a cancer can be determined by standard clinical techniques.

In some embodiments, the ADC of the methods for which the various dosages are described in this Section (Section 5.7) is enfortumab vedotin (EV).

Accordingly, a dosage of an antibody drug conjugate in the pharmaceutical composition that results in a serum titer of from about 0.1 μg/ml to about 450 μg/ml, and in some embodiments at least 0.1 μg/ml, at least 0.2 μg/ml, at least 0.4 μg/ml, at least 0.5 μg/ml, at least 0.6 μg/ml, at least 0.8 μg/ml, at least 1 μg/ml, at least 1.5 μg/ml, such as at least 2 g/ml, at least 5 μg/ml, at least 10 μg/ml, at least 15 μg/ml, at least 20 μg/ml, at least 25 g/ml, at least 30 μg/ml, at least 35 μg/ml, at least 40 μg/ml, at least 50 μg/ml, at least 75 g/ml, at least 100 μg/ml, at least 125 μg/ml, at least 150 μg/ml, at least 200 μg/ml, at least 250 μg/ml, at least 300 μg/ml, at least 350 μg/ml, at least 400 μg/ml, or at least 450 μg/ml can be administered to a human for the prevention and/or treatment of a cancer. It is to be understood that the precise dose to be employed in the formulation will also depend on the route of administration, and the seriousness of a cancer in a subject, and should be decided according to the judgment of the practitioner and each patient's circumstances.

Effective doses can be extrapolated from dose-response curves derived from in vitro or animal model test systems.

For the pharmaceutical composition comprising the antibody drug conjugate provided herein, the dosage of the antibody drug conjugate administered to a patient is typically 0.1 mg/kg to 100 mg/kg of the subject's body weight. In some embodiments, the dosage administered to the patient is about 1 mg/kg to about 75 mg/kg of the subject's body weight. In some embodiments, the dosage administered to a patient is between 1 mg/kg and 20 mg/kg of the subject's body weight, such as 1 mg/kg to 5 mg/kg of the subject's body weight. In some embodiments, dosage administered to a patient is about 0.5 mg/kg of the subject's body weight. In some embodiments, dosage administered to a patient is about 0.75 mg/kg of the subject's body weight. In some embodiments, dosage administered to a patient is about 1 mg/kg of the subject's body weight. In some embodiments, dosage administered to a patient is about 1.25 mg/kg of the subject's body weight. In some embodiments, dosage administered to a patient is about 1.5 mg/kg of the subject's body weight. In some embodiments, dosage administered to a patient is about 2 mg/kg of the subject's body weight. In some embodiments, dosage administered to a patient is about 2.5 mg/kg of the subject's body weight. In some embodiments, dosage administered to a patient is about 3 mg/kg of the subject's body weight. In some embodiments, dosage administered to a patient is about 3.5 mg/kg of the subject's body weight. In some embodiments, dosage administered to a patient is about 4 mg/kg of the subject's body weight. In some embodiments, dosage administered to a patient is about 4.5 mg/kg of the subject's body weight. In some embodiments, dosage administered to a patient is about 5 mg/kg of the subject's body weight. In some embodiments, dosage administered to a patient is about 5.5 mg/kg of the subject's body weight. In some embodiments, dosage administered to a patient is about 6 mg/kg of the subject's body weight. In some embodiments, dosage administered to a patient is about 6.5 mg/kg of the subject's body weight. In some embodiments, dosage administered to a patient is about 7 mg/kg of the subject's body weight. In some embodiments, dosage administered to a patient is about 7.5 mg/kg of the subject's body weight. In some embodiments, dosage administered to a patient is about 8 mg/kg of the subject's body weight. In some embodiments, dosage administered to a patient is about 8.5 mg/kg of the subject's body weight.

In some embodiments, the antibody drug conjugate formulated in the pharmaceutical composition provided herein is administered based on the patient's actual body weight at baseline and doses will not change unless the patient's weight changes by ≥10% from baseline of the previous cycle, or the dose adjustment criteria is met. In some embodiments, actual weight will be used except for patients weighing greater than 100 kg, in such cases, the dose will be calculated based on a weight of 100 kg. In some embodiments, the maximum doses are 100 mg for patients receiving the 1.00 mg/kg dose level and 125 mg for patients receiving the 1.25 mg/kg dose level.

In one embodiment, approximately 100 mg/kg or less, approximately 75 mg/kg or less, approximately 50 mg/kg or less, approximately 25 mg/kg or less, approximately 10 mg/kg or less, approximately 5 mg/kg or less, approximately 1.5 mg/kg or less, approximately 1.25 mg/kg or less, approximately 1 mg/kg or less, approximately 0.75 mg/kg or less, approximately 0.5 mg/kg or less, or approximately 0.1 mg/kg or less of an antibody drug conjugate formulated in the present pharmaceutical composition is administered 5 times, 4 times, 3 times, 2 times or 1 time to treat a cancer. In some embodiments, the pharmaceutical composition comprising the antibody drug conjugate provided herein is administered about 1-12 times, wherein the doses can be administered as necessary, e.g., weekly, biweekly, monthly, bimonthly, trimonthly, etc., as determined by a physician. In some embodiments, a lower dose (e.g., 0.1-15 mg/kg) can be administered more frequently (e.g., 3-6 times). In other embodiments, a higher dose (e.g., 25-100 mg/kg) can be administered less frequently (e.g., 1-3 times).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every two-week cycle (e.g., about 14 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every three-week cycle (e.g., about 21 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In some embodiments, a single dose of an antibody drug conjugate formulated in the pharmaceutical composition provided herein is administered to a patient to prevent and/or treat a cancer 1, 2, 3, 4, 5, 6, 7, 8, 9, 10, 11, 12, 13, 14, 15, 16, 17, 18, 19, 20, 21, 22, 23, 24, 25, or 26 times for every four-week cycle (e.g., about 28 day) over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, 6, 7, 8, 9, 10, 11, or 12 times at about monthly (e.g., about 30 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In another embodiment, a single dose of an antibody drug conjugate formulated in the pharmaceutical composition provided herein is administered to patient to prevent and/or treat a cancer 1, 2, 3, 4, 5, or 6 times at about bi-monthly (e.g., about 60 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In yet another embodiment, a single dose of an antibody drug conjugate formulated in the pharmaceutical composition provided herein is administered to patient to prevent and/or treat a cancer 1, 2, 3 or 4 times at about tri-monthly (e.g., about 120 day) intervals over a time period (e.g., a year), wherein the dose is selected from the group consisting of about 0.1 mg/kg, about 0.5 mg/kg, about 0.75 mg/kg, about 1 mg/kg, about 1.25 mg/kg, about 1.5 mg/kg, about 2 mg/kg, about 2.5 mg/kg, about 3 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, or a combination thereof (i.e., each dose monthly dose may or may not be identical).

In certain embodiments, the route of administration for a dose of an antibody drug conjugate formulated in the pharmaceutical composition provided herein to a patient is intranasal, intramuscular, intravenous (IV), or a combination thereof, but other routes described herein are also acceptable. Each dose may or may not be administered by an identical route of administration. In some embodiments, an antibody drug conjugate formulated in the pharmaceutical composition provided herein can be administered via multiple routes of administration simultaneously or subsequently to other doses of one or more additional therapeutic agents.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion every three-week cycle.

In some embodiments, the antibody drug conjugate formulated in the pharmaceutical composition is administered by an intravenous (IV) injection or infusion on Days 1 and 8 of every three-week cycle. In some embodiments, the antibody drug conjugate formulated in the pharmaceutical composition is administered by an intravenous (IV) injection on Days 1 and 8 of every three-week cycle. In some embodiments, the antibody drug conjugate formulated in the pharmaceutical composition is administered by an intravenous (IV) infusion on Days 1 and 8 of every three-week cycle. In some embodiments, the antibody drug conjugate formulated in the pharmaceutical composition is administered by an intravenous (IV) injection or infusion on Days 1 and 8 of every three-week cycle for 3 cycles. In some embodiments, the antibody drug conjugate formulated in the pharmaceutical composition is administered by an intravenous (IV) injection on Days 1 and 8 of every three-week cycle for 3 cycles. In some embodiments, the antibody drug conjugate formulated in the pharmaceutical composition is administered by an intravenous (IV) infusion on Days 1 and 8 of every three-week cycle for 3 cycles.

In some more specific embodiments, the antibody drug conjugate formulated in the pharmaceutical composition provided herein is administered at a dose of about 0.5 mg/kg, about 0.75 mg/kg, about 1 mg/kg, about 1.25 mg/kg, or about 1.5 mg/kg of the subject's body weight by an intravenous (IV) injection or infusion over about 30 minutes twice every three-week cycle. In some embodiments, the antibody drug conjugate formulated in the pharmaceutical composition is administered by an intravenous (IV) injection or infusion over about 30 minutes on Days 1 and 8 of every three-week cycle. In some embodiments, the antibody drug conjugate is administered to patients with urothelial or bladder cancer who have shown disease progression or relapse during or after treatment with another cancer treatment. In some embodiments, the antibody drug conjugate is administered to patients with urothelial or bladder cancer who have shown disease progression or relapse during or after treatment with another cancer treatment. In some embodiments, the antibody drug conjugate is administered to patients with urothelial or bladder cancer who have shown disease progression or relapse during or after treatment with another cancer treatment. In some embodiments, the ADC of the methods for which the various dosages are described in this paragraph is enfortumab vedotin (EV).

In other more specific embodiments, the antibody drug conjugate formulated in the pharmaceutical composition provided herein is administered at a dose of about 0.5 mg/kg, about 0.75 mg/kg, 1 mg/kg, about 1.25 mg/kg, or about 1.5 mg/kg of the subject's body weight by an intravenous (IV) injection or infusion over about 30 minutes three times every four-week cycle. In some embodiments, the antibody drug conjugate formulated in the pharmaceutical composition is administered on Days 1, 8 and 15 of every 28-day (four-week) cycle. In some embodiments, the antibody drug conjugate formulated in the pharmaceutical composition is administered by an intravenous (IV) injection or infusion over about 30 minutes on Days 1, 8 and 15 of every 28-day (four-week) cycle. In some embodiments, the antibody drug conjugate is administered to patients with urothelial or bladder cancer who have shown disease progression or relapse during or after treatment with another cancer treatment. In some embodiments, the ADC of the methods for which the various dosages are described in this paragraph is enfortumab vedotin (EV).

In some embodiments of the various methods provided herein, the ADC is administered at a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments, the ADC is administered at a dose of about 1 to about 10 mg/kg of the subject's body weight. In certain embodiments, the ADC is administered at a dose of about 1 to about 5 mg/kg of the subject's body weight. In other embodiments, the ADC is administered at a dose of about 1 to about 2.5 mg/kg of the subject's body weight. In further embodiments, the ADC is administered at a dose of about 1 to about 1.25 mg/kg of the subject's body weight. In some embodiments, the ADC is administered at a dose of about 0.25 mg/kg of the subject's body weight. In some embodiments, the ADC is administered at a dose of about 0.5 mg/kg of the subject's body weight. In some embodiments, the ADC is administered at a dose of about 0.75 mg/kg of the subject's body weight. In some embodiments, the ADC is administered at a dose of about 1.0 mg/kg of the subject's body weight. In some embodiments, the ADC is administered at a dose of about 1.25 mg/kg of the subject's body weight. In some embodiments, the ADC is administered at a dose of about 1.5 mg/kg of the subject's body weight. In some embodiments, the ADC is administered at a dose of about 1.75 mg/kg of the subject's body weight. In some embodiments, the ADC is administered at a dose of about 2.0 mg/kg of the subject's body weight. In some embodiments, the ADC is administered at a dose of about 2.25 mg/kg of the subject's body weight. In some embodiments, the ADC is administered at a dose of about 2.5 mg/kg of the subject's body weight.

In certain embodiments of the various methods provided herein, the ADC is administered at a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In some embodiments, the ADC is administered at a dose of 1 to 10 mg/kg of the subject's body weight. In certain embodiments, the ADC is administered at a dose of 1 to 5 mg/kg of the subject's body weight. In other embodiments, the ADC is administered at a dose of 1 to 2.5 mg/kg of the subject's body weight. In further embodiments, the ADC is administered at a dose of 1 to 1.25 mg/kg of the subject's body weight. In some embodiments, the ADC is administered at a dose of 0.25 mg/kg of the subject's body weight. In some embodiments, the ADC is administered at a dose of 0.5 mg/kg of the subject's body weight. In some embodiments, the ADC is administered at a dose of 0.75 mg/kg of the subject's body weight. In some embodiments, the ADC is administered at a dose of 1.0 mg/kg of the subject's body weight. In some embodiments, the ADC is administered at a dose of 1.25 mg/kg of the subject's body weight. In some embodiments, the ADC is administered at a dose of 1.5 mg/kg of the subject's body weight. In some embodiments, the ADC is administered at a dose of 1.75 mg/kg of the subject's body weight. In some embodiments, the ADC is administered at a dose of 2.0 mg/kg of the subject's body weight. In some embodiments, the ADC is administered at a dose of 2.25 mg/kg of the subject's body weight. In some embodiments, the ADC is administered at a dose of 2.5 mg/kg of the subject's body weight.

In some embodiments of the various methods provided herein, including those methods requiring a first and a second dose, the first dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the first dose of the ADC is a dose of about 1 to about 10 mg/kg of the subject's body weight. In some embodiments of the various methods provided herein, including those methods requiring a first and a second dose, the first dose of the ADC is a dose of about 1 to about 5 mg/kg of the subject's body weight. In some embodiments of the various methods provided herein, including those methods requiring a first and a second dose, the first dose of the ADC is a dose of about 1 to about 2.5 mg/kg of the subject's body weight. In some embodiments of the various methods provided herein, including those methods requiring a first and a second dose, the first dose of the ADC is a dose of about 1 to about 1.25 mg/kg of the subject's body weight. In some embodiments of the various methods provided herein, including those methods requiring a first and a second dose, the first dose of the ADC is a dose of about 0.25 mg/kg of the subject's body weight. In some embodiments of the various methods provided herein, including those methods requiring a first and a second dose, the first dose of the ADC is a dose of about 0.5 mg/kg of the subject's body weight. In some embodiments, the first dose of the ADC is a dose of about 0.75 mg/kg of the subject's body weight. In some embodiments, the first dose of the ADC is a dose of about 1.0 mg/kg of the subject's body weight. In some embodiments, the first dose of the ADC is a dose of about 1.25 mg/kg of the subject's body weight. In some embodiments, the first dose of the ADC is a dose of about 1.5 mg/kg of the subject's body weight. In some embodiments, the first dose of the ADC is a dose of about 1.75 mg/kg of the subject's body weight. In some embodiments, the first dose of the ADC is a dose of about 2.0 mg/kg of the subject's body weight. In some embodiments, the first dose of the ADC is a dose of about 2.25 mg/kg of the subject's body weight. In some embodiments, the first dose of the ADC is a dose of or about 2.5 mg/kg of the subject's body weight.

In certain embodiments of the various methods provided herein, including those methods requiring a first and a second dose, the first dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the first dose of the ADC is a dose of 1 to 10 mg/kg of the subject's body weight. In some embodiments of the various methods provided herein, including those methods requiring a first and a second dose, the first dose of the ADC is a dose of 1 to 5 mg/kg of the subject's body weight. In some embodiments of the various methods provided herein, including those methods requiring a first and a second dose, the first dose of the ADC is a dose of 1 to 2.5 mg/kg of the subject's body weight. In some embodiments of the various methods provided herein, including those methods requiring a first and a second dose, the first dose of the ADC is a dose of 1 to 1.25 mg/kg of the subject's body weight. In some embodiments of the various methods provided herein, including those methods requiring a first and a second dose, the first dose of the ADC is a dose of 0.25 mg/kg of the subject's body weight. In some embodiments of the various methods provided herein, including those methods requiring a first and a second dose, the first dose of the ADC is a dose of 0.5 mg/kg of the subject's body weight. In some embodiments, the first dose of the ADC is a dose of 0.75 mg/kg of the subject's body weight. In some embodiments, the first dose of the ADC is a dose of 1.0 mg/kg of the subject's body weight. In some embodiments, the first dose of the ADC is a dose of 1.25 mg/kg of the subject's body weight. In some embodiments, the first dose of the ADC is a dose of 1.5 mg/kg of the subject's body weight. In some embodiments, the first dose of the ADC is a dose of 1.75 mg/kg of the subject's body weight. In some embodiments, the first dose of the ADC is a dose of 2.0 mg/kg of the subject's body weight. In some embodiments, the first dose of the ADC is a dose of 2.25 mg/kg of the subject's body weight. In some embodiments, the first dose of the ADC is a dose of 2.5 mg/kg of the subject's body weight.

In some embodiments of the various methods provided herein, including those methods requiring a first and a second dose, the second dose of the ADC is lower than the first dose by about 0.1 mg/kg to about 2 mg/kg of the subject's body weight. In some embodiments of the various methods provided herein, including those methods requiring a first and a second dose, the second dose of the ADC is lower than the first dose by about 0.1 mg/kg of the subject's body weight. In some embodiments of the various methods provided herein, including those methods requiring a first and a second dose, the second dose of the ADC is lower than the first dose by about 0.2 mg/kg of the subject's body weight. In some embodiments of the various methods provided herein, including those methods requiring a first and a second dose, the second dose of the ADC is lower than the first dose by about 0.25 mg/kg of the subject's body weight. In some embodiments of the various methods provided herein, including those methods requiring a first and a second dose, the second dose of the ADC is lower than the first dose by about 0.3 mg/kg of the subject's body weight. In some embodiments of the various methods provided herein, including those methods requiring a first and a second dose, the second dose of the ADC is lower than the first dose by about 0.4 mg/kg of the subject's body weight. In some embodiments of the various methods provided herein, including those methods requiring a first and a second dose, the second dose of the ADC is lower than the first dose by about 0.5 mg/kg of the subject's body weight. In some embodiments of the various methods provided herein, including those methods requiring a first and a second dose, the second dose of the ADC is lower than the first dose by about 0.6 mg/kg of the subject's body weight. In some embodiments of the various methods provided herein, including those methods requiring a first and a second dose, the second dose of the ADC is lower than the first dose by about 0.7 mg/kg of the subject's body weight. In some embodiments of the various methods provided herein, including those methods requiring a first and a second dose, the second dose of the ADC is lower than the first dose by about 0.75 mg/kg of the subject's body weight. In some embodiments of the various methods provided herein, including those methods requiring a first and a second dose, the second dose of the ADC is lower than the first dose by about 0.8 mg/kg of the subject's body weight. In some embodiments of the various methods provided herein, including those methods requiring a first and a second dose, the second dose of the ADC is lower than the first dose by about 0.9 mg/kg of the subject's body weight. In some embodiments of the various methods provided herein, including those methods requiring a first and a second dose, the second dose of the ADC is lower than the first dose by about 1 mg/kg of the subject's body weight. In some embodiments of the various methods provided herein, including those methods requiring a first and a second dose, the second dose of the ADC is lower than the first dose by about 1.1 mg/kg of the subject's body weight. In some embodiments of the various methods provided herein, including those methods requiring a first and a second dose, the second dose of the ADC is lower than the first dose by about 1.2 mg/kg of the subject's body weight. In some embodiments of the various methods provided herein, including those methods requiring a first and a second dose, the second dose of the ADC is lower than the first dose by about 1.25 mg/kg of the subject's body weight. In some embodiments of the various methods provided herein, including those methods requiring a first and a second dose, the second dose of the ADC is lower than the first dose by about 1.3 mg/kg of the subject's body weight. In some embodiments of the various methods provided herein, including those methods requiring a first and a second dose, the second dose of the ADC is lower than the first dose by about 1.4 mg/kg of the subject's body weight. In some embodiments of the various methods provided herein, including those methods requiring a first and a second dose, the second dose of the ADC is lower than the first dose by about 1.5 mg/kg of the subject's body weight. In some embodiments of the various methods provided herein, including those methods requiring a first and a second dose, the second dose of the ADC is lower than the first dose by about 1.6 mg/kg of the subject's body weight. In some embodiments of the various methods provided herein, including those methods requiring a first and a second dose, the second dose of the ADC is lower than the first dose by about 1.7 mg/kg of the subject's body weight. In some embodiments of the various methods provided herein, including those methods requiring a first and a second dose, the second dose of the ADC is lower than the first dose by about 1.75 mg/kg of the subject's body weight. In some embodiments of the various methods provided herein, including those methods requiring a first and a second dose, the second dose of the ADC is lower than the first dose by about 1.8 mg/kg of the subject's body weight. In some embodiments of the various methods provided herein, including those methods requiring a first and a second dose, the second dose of the ADC is lower than the first dose by about 1.9 mg/kg of the subject's body weight. In some embodiments of the various methods provided herein, including those methods requiring a first and a second dose, the second dose of the ADC is lower than the first dose by about 2 mg/kg of the subject's body weight.

In certain embodiments of the various methods provided herein, including those methods requiring a first and a second dose, the second dose of the ADC is lower than the first dose by 0.1 mg/kg to 2 mg/kg of the subject's body weight. In some embodiments of the various methods provided herein, including those methods requiring a first and a second dose, the second dose of the ADC is lower than the first dose by 0.1 mg/kg of the subject's body weight. In some embodiments of the various methods provided herein, including those methods requiring a first and a second dose, the second dose of the ADC is lower than the first dose by 0.2 mg/kg of the subject's body weight. In some embodiments of the various methods provided herein, including those methods requiring a first and a second dose, the second dose of the ADC is lower than the first dose by 0.25 mg/kg of the subject's body weight. In some embodiments of the various methods provided herein, including those methods requiring a first and a second dose, the second dose of the ADC is lower than the first dose by 0.3 mg/kg of the subject's body weight. In some embodiments of the various methods provided herein, including those methods requiring a first and a second dose, the second dose of the ADC is lower than the first dose by 0.4 mg/kg of the subject's body weight. In some embodiments of the various methods provided herein, including those methods requiring a first and a second dose, the second dose of the ADC is lower than the first dose by 0.5 mg/kg of the subject's body weight. In some embodiments of the various methods provided herein, including those methods requiring a first and a second dose, the second dose of the ADC is lower than the first dose by 0.6 mg/kg of the subject's body weight. In some embodiments of the various methods provided herein, including those methods requiring a first and a second dose, the second dose of the ADC is lower than the first dose by 0.7 mg/kg of the subject's body weight. In some embodiments of the various methods provided herein, including those methods requiring a first and a second dose, the second dose of the ADC is lower than the first dose by 0.75 mg/kg of the subject's body weight. In some embodiments of the various methods provided herein, including those methods requiring a first and a second dose, the second dose of the ADC is lower than the first dose by 0.8 mg/kg of the subject's body weight. In some embodiments of the various methods provided herein, including those methods requiring a first and a second dose, the second dose of the ADC is lower than the first dose by 0.9 mg/kg of the subject's body weight. In some embodiments of the various methods provided herein, including those methods requiring a first and a second dose, the second dose of the ADC is lower than the first dose by 1 mg/kg of the subject's body weight. In some embodiments of the various methods provided herein, including those methods requiring a first and a second dose, the second dose of the ADC is lower than the first dose by 1.1 mg/kg of the subject's body weight. In some embodiments of the various methods provided herein, including those methods requiring a first and a second dose, the second dose of the ADC is lower than the first dose by 1.2 mg/kg of the subject's body weight. In some embodiments of the various methods provided herein, including those methods requiring a first and a second dose, the second dose of the ADC is lower than the first dose by 1.25 mg/kg of the subject's body weight. In some embodiments of the various methods provided herein, including those methods requiring a first and a second dose, the second dose of the ADC is lower than the first dose by 1.3 mg/kg of the subject's body weight. In some embodiments of the various methods provided herein, including those methods requiring a first and a second dose, the second dose of the ADC is lower than the first dose by 1.4 mg/kg of the subject's body weight. In some embodiments of the various methods provided herein, including those methods requiring a first and a second dose, the second dose of the ADC is lower than the first dose by 1.5 mg/kg of the subject's body weight. In some embodiments of the various methods provided herein, including those methods requiring a first and a second dose, the second dose of the ADC is lower than the first dose by 1.6 mg/kg of the subject's body weight. In some embodiments of the various methods provided herein, including those methods requiring a first and a second dose, the second dose of the ADC is lower than the first dose by 1.7 mg/kg of the subject's body weight. In some embodiments of the various methods provided herein, including those methods requiring a first and a second dose, the second dose of the ADC is lower than the first dose by 1.75 mg/kg of the subject's body weight. In some embodiments of the various methods provided herein, including those methods requiring a first and a second dose, the second dose of the ADC is lower than the first dose by 1.8 mg/kg of the subject's body weight. In some embodiments of the various methods provided herein, including those methods requiring a first and a second dose, the second dose of the ADC is lower than the first dose by 1.9 mg/kg of the subject's body weight. In some embodiments of the various methods provided herein, including those methods requiring a first and a second dose, the second dose of the ADC is lower than the first dose by 2 mg/kg of the subject's body weight.

In some embodiments of the various methods provided herein, including those methods requiring a first and a second dose, the second dose of the ADC is a dose of about 0.25 to about 10 mg/kg of the subject's body weight, about 0.25 to about 5 mg/kg of the subject's body weight, about 0.25 to about 2.5 mg/kg of the subject's body weight, about 0.25 to about 1.25 mg/kg of the subject's body weight, about 0.5 to about 10 mg/kg of the subject's body weight, about 0.5 to about 5 mg/kg of the subject's body weight, about 0.5 to about 2.5 mg/kg of the subject's body weight, about 0.5 to about 1.25 mg/kg of the subject's body weight, about 0.75 to about 10 mg/kg of the subject's body weight, about 0.75 to about 5 mg/kg of the subject's body weight, about 0.75 to about 2.5 mg/kg of the subject's body weight, or about 0.75 to about 1.25 mg/kg of the subject's body weight. In some embodiments of the various methods provided herein, including those methods requiring a first and a second dose, the second dose of the ADC is a dose of about 1 to about 10 mg/kg of the subject's body weight. In some embodiments of the various methods provided herein, including those methods requiring a first and a second dose, the second dose of the ADC is a dose of about 1 to about 5 mg/kg of the subject's body weight. In some embodiments of the various methods provided herein, including those methods requiring a first and a second dose, the second dose of the ADC is a dose of about 1 to about 2.5 mg/kg of the subject's body weight. In some embodiments of the various methods provided herein, including those methods requiring a first and a second dose, the second dose of the ADC is a dose of about 1 to about 1.25 mg/kg of the subject's body weight. In some embodiments of the various methods provided herein, including those methods requiring a first and a second dose, the second dose of the ADC is a dose of about 0.25 mg/kg of the subject's body weight. In some embodiments of the various methods provided herein, including those methods requiring a first and a second dose, the second dose of the ADC is a dose of about 0.5 mg/kg of the subject's body weight. In some embodiments of the various methods provided herein, including those methods requiring a first and a second dose, the second dose of the ADC is a dose of about 0.75 mg/kg of the subject's body weight. In some embodiments of the various methods provided herein, including those methods requiring a first and a second dose, the second dose of the ADC is a dose of about 1.0 mg/kg of the subject's body weight. In some embodiments of the various methods provided herein, including those methods requiring a first and a second dose, the second dose of the ADC is a dose of about 1.25 mg/kg of the subject's body weight. In some embodiments of the various methods provided herein, including those methods requiring a first and a second dose, the second dose of the ADC is a dose of about 1.5 mg/kg of the subject's body weight. In some embodiments of the various methods provided herein, including those methods requiring a first and a second dose, the second dose of the ADC is a dose of about 1.75 mg/kg of the subject's body weight. In some embodiments of the various methods provided herein, including those methods requiring a first and a second dose, the second dose of the ADC is a dose of about 2.0 mg/kg of the subject's body weight. In some embodiments of the various methods provided herein, including those methods requiring a first and a second dose, the second dose of the ADC is a dose of about 2.25 mg/kg of the subject's body weight. In some embodiments of the various methods provided herein, including those methods requiring a first and a second dose, the second dose of the ADC is a dose of about 2.5 mg/kg of the subject's body weight.

In certain embodiments of the various methods provided herein, including those methods requiring a first and a second dose, the second dose of the ADC is a dose of 0.25 to 10 mg/kg of the subject's body weight, 0.25 to 5 mg/kg of the subject's body weight, 0.25 to 2.5 mg/kg of the subject's body weight, 0.25 to 1.25 mg/kg of the subject's body weight, 0.5 to 10 mg/kg of the subject's body weight, 0.5 to 5 mg/kg of the subject's body weight, 0.5 to 2.5 mg/kg of the subject's body weight, 0.5 to 1.25 mg/kg of the subject's body weight, 0.75 to 10 mg/kg of the subject's body weight, 0.75 to 5 mg/kg of the subject's body weight, 0.75 to 2.5 mg/kg of the subject's body weight, or 0.75 to 1.25 mg/kg of the subject's body weight. In certain embodiments of the various methods provided herein, including those methods requiring a first and a second dose, the second dose of the ADC is a dose of 1 to 10 mg/kg of the subject's body weight. In some embodiments of the various methods provided herein, including those methods requiring a first and a second dose, the second dose of the ADC is a dose of 1 to 5 mg/kg of the subject's body weight. In some embodiments of the various methods provided herein, including those methods requiring a first and a second dose, the second dose of the ADC is a dose of 1 to 2.5 mg/kg of the subject's body weight. In some embodiments of the various methods provided herein, including those methods requiring a first and a second dose, the second dose of the ADC is a dose of 1 to 1.25 mg/kg of the subject's body weight. In some embodiments of the various methods provided herein, including those methods requiring a first and a second dose, the second dose of the ADC is a dose of 0.25 mg/kg of the subject's body weight. In some embodiments of the various methods provided herein, including those methods requiring a first and a second dose, the second dose of the ADC is a dose of 0.5 mg/kg of the subject's body weight. In some embodiments of the various methods provided herein, including those methods requiring a first and a second dose, the second dose of the ADC is a dose of 0.75 mg/kg of the subject's body weight. In some embodiments of the various methods provided herein, including those methods requiring a first and a second dose, the second dose of the ADC is a dose of 1.0 mg/kg of the subject's body weight. In some embodiments of the various methods provided herein, including those methods requiring a first and a second dose, the second dose of the ADC is a dose of 1.25 mg/kg of the subject's body weight. In some embodiments of the various methods provided herein, including those methods requiring a first and a second dose, the second dose of the ADC is a dose of 1.5 mg/kg of the subject's body weight. In some embodiments of the various methods provided herein, including those methods requiring a first and a second dose, the second dose of the ADC is a dose of 1.75 mg/kg of the subject's body weight. In some embodiments of the various methods provided herein, including those methods requiring a first and a second dose, the second dose of the ADC is a dose of 2.0 mg/kg of the subject's body weight. In some embodiments of the various methods provided herein, including those methods requiring a first and a second dose, the second dose of the ADC is a dose of 2.25 mg/kg of the subject's body weight. In some embodiments of the various methods provided herein, including those methods requiring a first and a second dose, the second dose of the ADC is a dose of 2.5 mg/kg of the subject's body weight.

In certain embodiments of the various methods provided herein, including those methods requiring a first and a second dose, the second dose of the ADC is identical to the first dose of the ADC.

In some embodiments of the methods provided herein, the ADC is administered by an intravenous (IV) injection or infusion. In one embodiment, the first dose of the ADC is administered by an IV injection. In another embodiment, the first dose of the ADC is administered by an IV infusion. In yet another embodiment, the second dose of the ADC is administered by an IV injection. In yet another embodiment, the second dose of the ADC is administered by an IV injection infusion. In one embodiment, the first dose of the ADC is administered by an IV injection and the second dose of the ADC is administered by an IV injection. In another embodiment, the first dose of the ADC is administered by an IV infusion and the second dose of the ADC is administered by an IV injection. In yet another embodiment, the second dose of the ADC is administered by an IV injection and the second dose of the ADC is administered by an IV injection infusion. In yet another embodiment, the second dose of the ADC is administered by an IV injection infusion and the second dose of the ADC is administered by an IV injection infusion. In some embodiments, the ADC of the methods for which the various dosages are described in this paragraph is enfortumab vedotin (EV).

In certain embodiments of the methods provided herein, the ADC is administered by an IV injection or infusion three times every four-week cycle. In some embodiments of the methods provided herein, the first dose of the ADC is administered by an IV injection or infusion three times every four-week cycle. In some embodiments of the methods provided herein, the second dose of the ADC is administered by an IV injection or infusion three times every four-week cycle. In some embodiments of the methods provided herein, the first dose of the ADC is administered by an IV injection or infusion three times every four-week cycle and the second dose of the ADC is administered by an IV injection or infusion three times every four-week cycle. In some embodiments, the ADC of the methods for which the various dosages are described in this paragraph is enfortumab vedotin (EV).

In some embodiments of the methods provided herein, the ADC is administered by an IV injection or infusion on Days 1, 8 and 15 of every four-week cycle. In some embodiments, the first dose of ADC is administered by an IV injection or infusion on Days 1, 8 and 15 of every four-week cycle. In some embodiments, the second dose of ADC is administered by an IV injection or infusion on Days 1, 8 and 15 of every four-week cycle. In some embodiments, the first dose of ADC is administered by an IV injection or infusion on Days 1, 8 and 15 of every four-week cycle and the second dose of ADC is administered by an IV injection or infusion on Days 1, 8 and 15 of every four-week cycle. In some embodiments, the ADC of the methods for which the various dosages are described in this paragraph is enfortumab vedotin (EV).

In certain embodiments of the methods provided herein, the ADC is administered by an IV injection or infusion over about 30 minutes three times every four-week cycle. In some embodiments, the first dose of the ADC is administered by an IV injection or infusion over about 30 minutes three times every four-week cycle. In some embodiments, the second dose of the ADC is administered by an IV injection or infusion over about 30 minutes three times every four-week cycle. In some embodiments, the first dose of the ADC is administered by an IV injection or infusion over about 30 minutes three times every four-week cycle and the second dose of the ADC is administered by an IV injection or infusion over about 30 minutes three times every four-week cycle. In some embodiments, the ADC of the methods for which the various dosages are described in this paragraph is enfortumab vedotin (EV).

In some embodiments of the methods provided herein, the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle. In some embodiments of the methods provided herein, the second dose of the ADC is administered by an IV injection or infusion over about 30 minutes on Days 1, 8 and 15 of every four-week cycle. In some embodiments of the methods provided herein, the first dose of the ADC is administered by an IV injection or infusion over about 30 minutes on Days 1, 8 and 15 of every four-week cycle and the second dose of the ADC is administered by an IV injection or infusion over about 30 minutes on Days 1, 8 and 15 of every four-week cycle. In some embodiments, the ADC of the methods for which the various dosages are described in this paragraph is enfortumab vedotin (EV).

In other more specific embodiments, the antibody drug conjugate formulated in the pharmaceutical composition provided herein is administered at a dose of about 1 mg/kg, 1.25 mg/kg, or about 1.5 mg/kg of the subject's body weight by an intravenous (IV) injection or infusion over about 30 minutes three times every 28-day cycle. In some embodiments, the antibody drug conjugate formulated in the pharmaceutical composition is administered by an intravenous (IV) injection or infusion over about 30 minutes on Days 1, 8 and 15 of every 28-day cycle. In some embodiments, the method further comprises administering an immune checkpoint inhibitor by an intravenous (IV) injection or infusion one or more times in each four-week cycle. In some embodiments of the methods provided herein, the ADC is administered three times within a 28 day cycle. In some embodiments of the methods provided herein, the ADC is administered on Days 1, 8 and 15 of a 28 day cycle. In some embodiments, the ADC of the methods for which the various dosages are described in this paragraph is enfortumab vedotin (EV).

In one specific embodiment of the methods provided herein, the ADC has the following structure:

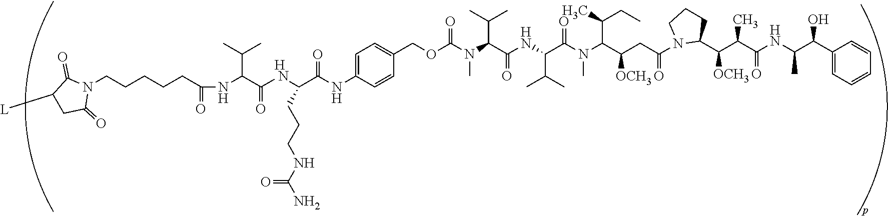

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV injection or infusion over about 30 minutes on Days 1, 8 and 15 of every four-week cycle.

In one specific embodiment of the methods provided herein, the ADC has the following structure:

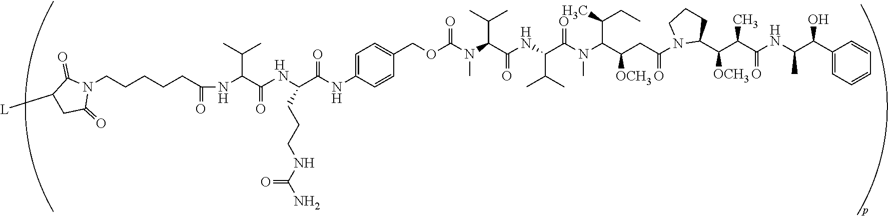

wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV infusion on Days 1 and 8 of every three-week cycle.

### 5.8 Methods for Determining the Biomarkers

The disclosure provides that the expression of any of the markers provided herein can be determined by various methods known in the field. In some embodiments, the expression of the markers can be determined by the amount or relative amount of mRNA transcribed from the marker genes. In one embodiment, the expression of the marker genes can be determined by the amount or relative amount of the protein products encoded by the marker genes. In another embodiment, the expression of the marker genes can be determined by the level of biological or chemical response induced by the protein products encoded by the marker genes. Additionally, in certain embodiments, the expression of the marker genes can be determined by the expression of one or more genes that correlates with the expression of the marker genes.

As described above, levels or amounts of gene transcripts (e.g. mRNA) of the marker genes can be used as a proxy for the expression levels of markers genes. Numerous different PCR or qPCR protocols are known in the art including those exemplified herein. In some embodiments, the various PCR or qPCR methods are applied or adapted for determining the mRNA level of the various marker genes. Quantitative PCR (qPCR) (also referred as real-time PCR) is applied and adapted in some embodiments as it provides not only a quantitative measurement, but also reduced time and contamination. As used herein, “quantitative PCR (or “qPCR”) refers to the direct monitoring of the progress of PCR amplification as it is occurring without the need for repeated sampling of the reaction products. In quantitative PCR, the reaction products can be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. When qPCR is applied to determine mRNA expression level, an extra step of reverse-transcription of mRNA to DNA is performed before the qPCR analysis. Examples of PCR methods can be found in the literature (Wong et al., BioTechniques 39:75-85 (2005); D'haene et al., Methods 50:262-270 (2010)), which is incorporated by reference herein in its entirety. Examples of PCR assays can also be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In one specific embodiment, qPCR can be performed to determine or measure the mRNA levels of the marker genes as follows. Briefly, mean Ct (cycle threshold) values (or referred to herein interchangeably as Cq (quantification cycle)) of replicate qPCR reactions for the marker genes and one or more housekeeping genes are determined. Mean Ct values for the marker genes can be then normalized to the Ct values of the housekeeping genes using the following exemplary formula: marker-gene-ΔCt=(mean Ct of marker gene−mean Ct of housekeeping gene A). The relative marker-gene-ΔCt can then be used to determine relative level of marker gene mRNA, for example by using the formula of mRNA expression=2^{-ΔCt}. For a summary of Ct and Cq values, see MIQE guideline (Bustin et al., The MIQE Guidelines: Minimum Information for Publication of Quantitative Real-Time PCR Experiments, Clinical Chemistry 55:4 (2009)).

Other commonly used methods known in the art can also be used for the quantification of RNA transcripts of the marker genes in a sample as the proxy for the expression of the marker genes, including northern blotting and in situ hybridization (Parker & Barnes, Methods in Molecular Biology 106:247-283 (1999)); RNAse protection assays (Hod, Biotechniques 13:852-854 (1992)); microarrays (Hoheisel et al., Nature Reviews Genetics 7:200-210 (2006); Jaluria et al., Microbial Cell Factories 6:4 (2007)); and polymerase chain reaction (PCR) (Weis et al, Trends in Genetics 8:263-264 (1992)). RNA in situ hybridization (ISH) is a molecular biology technique widely used to measure and localize specific RNA sequences, for example, messenger RNAs (mRNAs), long non-coding RNAs (lncRNAs), and microRNAs (miRNAs) within cells, such as circulating tumor cells (CTCs) or tissue sections, while preserving the cellular and tissue context. ISH is a type of hybridization that uses a directly or indirectly labeled complementary DNA or RNA strand, such as a probe, to bind to and localize a specific nucleic acid, such as DNA or RNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides. The term “fluorescent in situ hybridization” or “FISH” refers to a type of ISH utilizing a fluorescent label. The term “chromogenic in situ hybridization” or “CISH” refers to a type of ISH with a chromogenic label. ISH, FISH and CISH methods are well known to those skilled in the art (see, for example, Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000)). RNA ISH therefore provides for spatial-temporal visualization as well as quantification of gene expression within cells and tissues. It has wide applications in research and in diagnostics (Hu et al., Biomark. Res. 2(1):1-13, doi: 10.1186/2050-7771-2-3 (2014); Ratan et al., Cureus 9(6):e1325. doi: 10.7759/cureus. 1325 (2017); Weier et al., Expert Rev. Mol. Diagn. 2(2):109-119 (2002)). Fluorescent RNA ISH utilizes fluorescent dyes and fluorescent microscopes for RNA labeling and detection, respectively. Fluorescent RNA ISH can provides for multiplexing of four to five target sequences.

Alternatively, RNA transcripts of the marker genes in a sample as the proxy for the expression of the marker genes can be determined by sequencing techniques. Representative methods for sequencing-based gene expression analysis include Serial Analysis of Gene Expression (SAGE), and gene expression analysis by massively parallel signature sequencing (MPSS).

In some embodiments, expression of the marker genes can be determined by the relative abundance of the RNA transcripts (including for example mRNA) of the marker genes in a pool of total transcribed RNA. Such relative abundance of the RNA transcripts of the marker genes can be determined by next generation sequencing, which is known as RNA-seq. In one example of the RNA-seq procedure, RNAs from different sources (blood, tissue, cells) are purified, optionally enriched (e.g. with oligo (dT) primers), converted to cDNA, and fragmented. Millions or even billions of short sequence reads are generated from the randomly fragmented cDNA library. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference. The expression level of each mRNA transcript of the marker genes is determined by the total number of mapped fragments upon normalization, which is directly proportional to its abundance level. A few normalization schemes are known and used to facilitate the use of the abundance of the RNA transcripts as the parameter for determining gene expression, including RPKM (Reads Per Kilobase Million), FPKM (Fragments Per Kilobase Million), and/or TPM (Transcripts Per Kilobase Million). Briefly, RPKM can be calculated as follows: count up the total reads in a sample and divide that number by 1,000,000—which is the “per million” scaling factor; divide the read counts by the “per million” scaling factor, which normalizes for sequencing depth, giving the reads per million (RPM); and divide the RPM values by the length of the gene, in kilobases, which gives RPKM. FPKM is closely related to RPKM except with fragment replacing read. RPKM was made for single-end RNA-seq, where every read corresponded to a single fragment that was sequenced. FPKM was made for paired-end RNA-seq, in which two reads can correspond to a single fragment, or, if one read in the pair did not map, one read can correspond to a single fragment. TPM is very similar to RPKM and FPKM and is calculated as follows: divide the read counts by the length of each gene in kilobases, which gives the reads per kilobase (RPK); count up all the RPK values in a sample and divide this number by 1,000,000, which gives the “per million” scaling factor; divide the RPK values by the “per million” scaling factor, which gives TPM. See Zhao et al. BMC genomics 16: 97 (2015); Zhao et al. Scientific Reports 8: 4781 (2018); Shanrong Zhao et al., RNA, published in advance Apr. 13, 2020, doi: 10.1261/rna.074922.120, all of which are incorporated herein in their entirety by reference.

In one embodiment, the expression of the marker genes is determined by RNA-seq, for example by TPM, RPKM, and/or FPKM. In some embodiments, the expression of the marker genes is determined by TPM. In some embodiments, the expression of the marker genes is determined by RPKM. In some embodiments, the expression of the marker genes is determined by FPKM.

As described earlier, the expression of the marker genes can be determined in a sample from a subject. In some embodiments, the sample is a blood sample, a serum sample, a plasma sample, bodily fluid (e.g. tissue fluid including cancer tissue fluid), or a tissue (e.g. cancer tissue or the tissue surrounding the cancer). In some embodiments, the sample is a tissue sample. In some embodiments, the tissue sample is tissue fractions isolated or extracted from a mammal, in particular a human. In some embodiments, the tissue sample is a population of cells isolated or extracted from a mammal, in particular a human. In some embodiments, the tissue sample is a sample obtained from a biopsy. In certain embodiments, the samples can be obtained from a variety of organs of a subject, including a human subject. In some embodiments, the samples are obtained from organs of a subject having a cancer. In some embodiments, the samples are obtained from organs having a cancer in a subject having a cancer. In other embodiments, the samples, for example reference samples, are obtained from normal organs from the patient or from a second human subject.

In certain embodiments of the methods provided herein, the tissue includes a tissue from bladder, ureter, breast, lung, colon, rectum, ovary, Fallopian tube, esophagus, cervix, uterine endometrium, skin, larynx, bone marrow, salivary gland, kidney, prostate, brain, spinal cord, placenta, adrenal, pancreas, parathyroid, hypophysis, testis, thyroid, spleen, tonsil, thymus, heart, stomach, small intestine, liver, skeletal muscle, peripheral nerve, mesothelium, or eye.

In further embodiments of the methods provided herein, the expression of the various marker genes can be detected by a variety of immunoassays known in the art, including an immunohistochemistry (IHC) assay, an immunoblotting assay, a FACS assay, and an ELISA.

The expression of the various marker genes can be detected by antibodies against the protein products encoded by the marker genes in a variety of IHC assays. IHC staining of tissue sections has been shown to be a reliable method of assessing or detecting the presence of proteins in a sample. IHC techniques utilize an antibody to probe and visualize cellular antigens in situ, generally by chromogenic or fluorescent methods. Primary antibodies or antisera, such as polyclonal antisera and monoclonal antibodies that specifically target the protein products encoded by the marker genes, can be used to detect expression of the marker genes in an IHC assay. In some embodiments, the tissue sample is contacted with a primary antibody for a specific target for a period of time sufficient for the antibody-target binding to occur. As discussed in detail earlier, the antibodies can be detected by direct labels on the antibodies themselves, for example, radioactive labels, fluorescent labels, hapten labels such as biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Leica BOND Autostainer and Leica Bond Refine Detection system is an example of such an automated system.

In some embodiments, an IHC assay is performed with an unlabeled primary antibody in conjunction with a labeled secondary antibody in an indirect assay. The indirect assay utilizes two antibodies for the detection of the protein products encoded by the marker genes in a tissue sample. First, an unconjugated primary antibody was applied to the tissue (first layer), which reacts with the target antigen in the tissue sample. Next, an enzyme-labeled secondary antibody is applied, which specifically recognize the antibody isotype of the primary antibody (second layer). The secondary antibody reacts with the primary antibody, followed by substrate-chromogen application. The second-layer antibody can be labeled with an enzyme such as a peroxidase, which reacts with the chromogen 3,3′-diaminobenzidine (DAB) to produce brown precipitate at the reaction site. This method is sensitive and versatile due to the potential signal amplification through a signal amplification system.

In certain embodiments to increase the sensitivity of the detection, a signal amplification system can be used. “A signal amplification system”, as used herein, means a system of reagents and methods that can be used to increase the signal from detecting the bound primary or the secondary antibody. A signal amplification system increases the sensitivity of the target protein detection, increases the detected signal, and decreases the lower boundary of the detection limits. There are several types of signal amplification systems including an enzyme labeling system and macrolabeling system. These systems/approaches are not mutually exclusive and can be used in combination for additive effect.

Macrolabels or macrolabeling system are collections of labels numbering in the tens (e.g. phycobiliproteins) to millions (e.g. fluorescent microspheres) attached to or incorporated in a common scaffold. The scaffold can be coupled to a target-specific affinity reagent such as an antibody, and the incorporated labels are thereby collectively associated with the target upon binding. The labels in the macrolabels can be any of the labels described herein such as fluorophores, haptens, enzymes, and/or radioisotopes. In one embodiment of the signal amplification system, a labeled chain polymer-conjugated secondary antibody was used. The polymer technology utilized an HRP enzyme-labeled inert “spine” molecule of dextran to which 1, 2, 3, 4, 5, 6, 7, 8, 9 10, 15, 20, 25, 30, 50 or more molecules of secondary antibodies can be attached, making the system even more sensitive.

Signal amplification system based on an enzyme labeling system utilizes the catalytic activity of enzymes, such as horseradish peroxidase (HRP) or alkaline phosphatase to generate high-density labeling of a target protein or nucleic acid sequence in situ. In one embodiment, tyramide can be used to increase the signal of HRP. In such a system, HRP enzymatically converts the labeled tyramide derivative into highly reactive, short-lived tyramide radicals. The labeled active tyramide radicals then covalently couple to residues (principally the phenol moiety of protein tyrosine residues) in the vicinity of the HRP-antibody-target interaction site, resulting amplification of the number of labels at the site with minimal diffusion-related loss of signal localization. Consequently, the signal can be amplified 1, 2, 3, 4, 5, 6, 7, 8, 9 10, 15, 20, 25, 30, 50, 75, or 100 folds. As known to a person skilled in the art, the labels on the tyramide can be any labels described herein, including fluorophores, enzymes, haptens, radioisotopes, and/or photophores. Other enzyme-based reactions can be utilized to create signal amplification as well. For example, Enzyme-Labeled Fluorescence (ELF) signal amplification is available for alkaline phosphatase, wherein the alkaline phosphatase enzymatically cleaves a weakly blue-fluorescent substrate (ELF 97 phosphate) and converts it into a bright yellow-green-fluorescent precipitate that exhibits an unusually large Stokes shift and excellent photostability. Both tyramide-based signal amplification system and ELF signal amplification are available commercially, for example from ThermoFisher Scientific (Waltham, MA USA 02451).

Thus in some embodiments of the methods provided herein, the expression level of the marker genes is detected with IHC using a signal amplification system. In some embodiments, the specimen is then counterstained to identify cellular and subcellular elements.

In some embodiments, the expression level of the protein products encoded by the marker genes can also be detected with antibodies against the protein products encoded by the marker genes using an immunoblotting assay. In some embodiments of an immunoblotting assay, proteins are often (but do not have to be) separated by electrophoresis and transferred onto membranes (usually nitrocellulose or PVDF membrane). Similar to the IHC assays, primary antibodies or antisera, such as polyclonal antisera and monoclonal antibodies that specifically target the protein products encoded by the marker genes, can be used to detect expression of the marker genes. In some embodiments, the membrane is contacted with a primary antibody for a specific target for a period of time sufficient for the antibody-antigen binding to occur and the bound antibodies can be detected by direct labels on the primary antibodies themselves, e.g. with radioactive labels, fluorescent labels, hapten labels such as biotin, or enzymes such as horseradish peroxidase or alkaline phosphatase. In other embodiments, unlabeled primary antibody is used in an indirect assay as described above in conjunction with a labeled secondary antibody specific for the primary antibody. As described herein, the secondary antibodies can be labeled, for example, with enzymes or other detectable labels such as fluorescent labels, luminescent labels, colorimetric labels, or radioisotopes. Immunoblotting protocols and kits are well known in the art and are commercially available. Automated systems for immunoblotting, e.g. iBind Western Systems for Western blotting (ThermoFisher, Waltham, MA USA 02451), are available commercially. Immunoblotting includes, but is not limited to, Western blot, in-cell Western blot, and dot blot. Dot blot is a simplified procedure in which protein samples are not separated by electrophoresis but are spotted directly onto a membrane. In cell Western blot involves seeding cells in microtiter plates, fixing/permeabilizing the cells, and subsequent detection with a primary labeled primary antibody or unlabelled primary antibody followed by labeled secondary antibody as described herein.

In other embodiments, the expression levels of the protein products encoded by the marker genes can also be detected with the antibodies described herein in a flow cytometry assay, including a fluorescence-activated cell sorting (FACS) assay. Similar to the IHC or immunoblotting assays, primary antibodies or antisera, such as polyclonal antisera and monoclonal antibodies that specifically target the protein products encoded by the marker genes, can be used to detect protein expression in a FACS assay. In some embodiments, cells are stained with primary antibodies against specific target protein for a period of time sufficient for the antibody-antigen binding to occur and the bound antibodies can be detected by direct labels on the primary antibodies, for example, fluorescent labels or hapten labels such as biotin on the primary antibodies. In other embodiments, unlabeled primary antibody is used in an indirect assay as described above in conjunction with a fluorescently labeled secondary antibody specific for the primary antibody. FACS provides a method for sorting or analyzing a mixture of fluorescently labeled biological cells, one cell at a time, based upon the specific light scattering and fluorescent characteristics of each cell. The flow cytometer thus detects and reports the intensity of the fluorichrome-tagged antibody, which indicates the expression level of the target protein. Therefore, the expression level of the protein products encoded by the marker genes can be detected using antibodies against such protein products. Non-fluorescent cytoplasmic proteins can also be observed by staining permeablized cells. Methods for performing FACS staining and analyses are well known to a person skilled in the art and are described by Teresa S. Hawley and Robert G. Hawley in Flow Cytometry Protocols, Humana Press, 2011 (ISBN 1617379506, 9781617379505).

In other embodiments, the expression levels of the protein products encoded by the marker genes can also be detected using immunoassays such as an Enzyme Immune Assay (EIA) or an ELISA. Both EIA and ELISA assays are known in the art, e.g. for assaying a wide variety of tissues and samples, including blood, plasma, serum or bone marrow. A wide range of ELISA assay formats are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target protein. Sandwich assays are commonly used assay format. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results can either be qualitative, by simple observation of the visible signal, or can be quantitated by comparing with a control sample containing known amounts of target protein.

In some embodiments of the EIA or ELISA assays, an enzyme is conjugated to the second antibody. In other embodiments, fluorescently labeled secondary antibodies can be used in lieu of the enzyme-labeled secondary antibody to produce a detectable signal an ELISA assay format. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA and ELISA, the fluorescent labeled antibody is allowed to bind to the first antibody-target protein complex. After washing off the unbound reagent, the remaining tertiary complex is then exposed to the light of the appropriate wavelength; the fluorescence observed indicates the presence of the target protein of interest. Immunofluorescence and EIA techniques are both very well established in the art and are disclosed herein.

For the immunoassays described herein, any of a number of enzymes or non-enzyme labels can be utilized so long as the enzymatic activity or non-enzyme label, respectively, can be detected. The enzyme thereby produces a detectable signal, which can be utilized to detect a target protein. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galatoside 6-phosphogalactohydrolase, β-gluosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods of the present disclosure.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis(2-amino-2-methyl-1,3-propanediol), 4-Methylumbelliferyl phosphate bis (cyclohexylammonium) and 4-Methylumbelliferyl phosphate for phosphatases; QuantaBlu™ and QuantaRed™ for horseradish peroxidase; 4-Methylumbelliferyl β-D-galactopyranoside, Fluorescein di(3-D-galactopyranoside) and Naphthofluorescein di-(β-D-galactopyranoside) for β-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford IL; Santa Cruz Biotechnology, Dallas TX; Invitrogen, Carlsbad CA; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord CA; biocare.net/products/detection/chromogens).

In some embodiments of the immunoassays, a detectable label can be directly coupled to either the primary antibody or the secondary antibody that detects the unlabeled primary antibody can have. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene Oreg.); pyrenes and sulfonated pyrenes such as Cascade Blue™ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof, CyDye™ fluorescent dyes (Amersham/GE Healthcare Life Sciences; Piscataway NJ), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

Methods well known to a person skilled in the art such as microscopy or spectroscopy can be utilized to visualize chromogenic or fluorescent detectable signals associated with the bound primary or secondary antibodies.

The methods provided in this Section (Section 5.8) can be used with various cancer models known in the art. In one embodiment, mouse xenograft cancer models are used. Briefly, T-24 and UM-UC-3 cells are purchased from ATCC and cultured using the recommended media conditions. The T-24 hNectin-4 (human nectin-4) and the UM-UC-3 Nectin-4 cells are generated by transducing parental cells with lentivirus containing the human Nectin-4 using the pRCDCMEP-CMV-hNectin-4 EF1-Puro construct and selected using puromycin. The T-24 Nectin-4 (clone 1A9) cells are implanted into nude mice and passaged via trocar, allowed to reach approximately 200 mm³ tumor volume, and subsequently treated with a single intraperitoneal (IP) dose of enfortumab vedotin (3 mg/kg) or non-binding ADC (3 mg/kg) with 7 animals per treatment group. Follow-up ICD studies with this model involve collecting tumors 5 days post treatment for downstream analysis by RNA-seq, flow, immunohistochemistry (IHC), and Luminex. Tumors are fixed in formalin and prepared as FFPE tissue blocks. Blocks are cut at 4 m and immunohistochemistry is performed using F4/80, CD11c. The immunohistochemically stained slides sections are scanned with a Leica AT2 digital whole slide scanner, and the images are analyzed with Visiopharm software by use of custom-made algorithms for Nectin 4, CD11c and F4/80 staining. The algorithms are optimized on the basis of staining intensity and background staining. Percent positive staining is calculated for Nectin 4 and positive cells per mm² is calculated for F480 and CD11c.

Sections of tumor are lysed in Cell Lysis Buffer 2 (R&D Systems®, Catalog #895347). The cytokines and chemokines from the tumor samples are measured using the MILLIPLEX MAP mouse cytokine/chemokine magnetic bead panel (Millipore) and read on the LUMINEX MAGPIX system.

For the RNA-seq analysis RNA from flash frozen tumors is isolated using the TRIZOL Plus RNA Purification Kit (Life Technologies) according to the manufacturer's protocol yielding high quality RNA (average RNA integrity number >8). RNA selection method is using Poly(A) selection and the mRNA Library Prep Kit from Illumina and read on the Hi-Seq 2×150 bp, single index (Illumina). The sequence reads are mapped to the human and mouse transcriptome and total reads per million were determined.

The disclosure is generally provided using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

Particular embodiments of this disclosure are described herein, including the best mode known to the inventors for carrying out the disclosure. Upon reading the foregoing description, variations of the disclosed embodiments can become apparent to individuals working in the art, and it is expected that those skilled artisans can employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference in their entireties as if each individual publication or patent application were specifically and individually indicated to be incorporated by reference.

A number of embodiments of the disclosure have been described. Nevertheless, it will be understood that various modifications can be made without departing from the spirit and scope of the disclosure.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### 6.1 Example 1—a Phase 1b/2 Study of Neoadjuvant Enfortumab Vedotin (ASG-22CE) Monotherapy Followed by Radical Cystectomy and Pelvic Lymph Node Dissection (RC+PLND) for Treatment of Cisplatin-Ineligible Patients with Muscle Invasive Urothelial Cancer (MIUC). (Cohort H)

### 6.1.1 Drug Used in the Clinical Study

Enfortumab vedotin is a Nectin-4 targeted monoclonal antibody (AGS-22C3) covalently linked to the microtubule-disrupting agent monomethyl auristatin E (MMAE). Enfortumab vedotin consists of three functional subunits:
A fully human IgG1K antibody (AGS-22C3);
The microtubule-disrupting agent MMAE;
A protease-cleavable maleimidocaproyl-valine-citrulline (vc) linker that covalently attaches MMAE to AGS-22C3.

Enfortumab vedotin binds the V domain of Nectin-4 (Challita-Eid et al., Cancer Res (2016); 76(10): 3003-13.). In the presumed mechanism of action, the drug binds Nectin-4 protein on the cell surface and is internalized, causing proteolytic cleavage of the vc linker and intracellular release of MMAE. Free MMAE subsequently disrupts tubulin polymerization and leads to mitotic arrest.

### 6.1.2 Summary of the Study

### 6.1.2.1 Synopsis

### (i) Name of Study Drug

Enfortumab Vedotin (ASG-22CE)

### (ii) Phase of Development

Phase 1b/2

(iii) Title of Study

A Phase 1b/2 study of neoadjuvant enfortumab vedotin (ASG-22CE) as monotherapy followed by radical cystectomy and pelvic lymph node dissection (RC+PLND) for the treatment of cisplatin-ineligible patients with muscle invasive urothelial cancer (MIUC).

### (iv) Study Objective(s)

### Primary

To assess the antitumor activity of neoadjuvant enfortumab vedotin monotherapy as measured by the pathological complete response (pCR) rate, defined as the absence of viable tumor (pT0N0) in examined tissue from radical cystectomy (RC) and pelvic lymph node dissection (PLND) by central pathology review.

### Secondary

To assess the Event-Free Survival (EFS) on study therapy by investigator
To assess the pathologic downstaging (pDS) rate, defined as patients with tumors <pT2 (includes pT0, pTis, pTa, pT1) and N0 in examined tissue from RC+PLND by central pathology review
To assess the disease free survival (DFS) by investigator
To assess overall survival (OS)
To assess the safety and tolerability of enfortumab vedotin monotherapy
To assess the percentage of planned RC+PLND delayed due to treatment-related adverse events (AEs)

### Additional

To assess Nectin-4 and PD-L1 expression levels
To assess biomarkers of biological activity and disease resistance, and their potential associations with clinical outcome measures
To assess PK and the incidence of ATA
To assess patient-reported experience and patient-reported tolerability of treatment

### (v) Study Population

Eligible patients include patients ≥18 years of age with histologically confirmed muscle invasive bladder cancer (MIBC) (predominant urothelial type [ie, >50%]) with an ECOG performance status of 0, 1, or 2, who are deemed eligible for, and agree to undergo, curative intent RC+PLND.

Eligible patients should have a clinical stage of cT2 T4aN0M0 by review of pathology and imaging. Patients require imaging using computed tomography (CT) with intravenous (IV) contrast of the chest and a CT urogram of the abdomen and pelvis at screening.

Imaging (as described in the preceding sentence) should be done ≤28 days before enrollment [American Joint Commission on Cancer, sixth edition]. Tumor samples with an associated pathology report from the diagnostic transurethral resection of a bladder tumor (TURBT) must be available prior to enrollment and determined to be sufficient for pathology review and biomarker analysis. Patients should have adequate hematologic and organ function tests.

Eligible patients must be ineligible for cisplatin based chemotherapy at the time of enrollment due to at least 1 of the following criteria: GFR<60 mL/min but ≥30 mL/min, ECOG performance status of 2, NCI CTCAE Version 4.03 Grade ≥2 hearing loss, or NYHA Class III heart failure. Patients must not have received prior systemic treatment, chemoradiation, or radiation therapy for MIBC. Patients may have received prior intravesical Bacillus Calmette-Guérin (BCG) or intravesical chemotherapy for non-muscle invasive bladder cancer (NMIBC).

### (vi) Number of Planned Patients

Approximately 20 patients are enrolled in this study.

(vii) Study Design

This study is designed to evaluate the safety and antitumor activity of enfortumab vedotin as monotherapy for the treatment of cisplatin ineligible patients with MIBC in the neoadjuvant and perioperative settings. The study design is depicted in FIG. 2.

Treatment with enfortumab vedotin monotherapy is evaluated in patients with MIBC. This study enrolls approximately 20 patients with cT2-T4aN0M0 MIBC. All patients are treated with neoadjuvant enfortumab vedotin (1.25 mg/kg) administered as an IV infusion on Days 1 and 8 of every 3 week cycle for 3 cycles prior to RC+PLND.

Safety is monitored by the SMC on an ongoing basis. Step-down dose levels (up to 2 levels) for enfortumab vedotin below the dose of 1.25 mg/kg will be allowed if recommended by the SMC and upon approval by the study sponsor.

All patients should have a transurethral resection of a bladder tumor (TURBT) within 90 days prior to the first treatment dose, and tissue from the diagnostic TURBT must be confirmed to be available prior to enrollment and be deemed sufficient for pathology review and biomarker analysis. All patients undergo baseline post-TURBT radiographic screening assessment using CT with IV contrast of the chest and a CT urogram of the abdomen and pelvis for clinical staging.

Following neoadjuvant treatment, restaging is done via the same radiologic modalities ≤4 weeks prior to RC+PLND to exclude disease progression, which would preclude curative surgery. Surgery consists of RC+PLND with curative intent in accordance with the American Urological Association (AUA)/American Society of Clinical Oncology (ASCO)/American Society for Radiation Oncology (ASTRO)/Society of Urologic Oncology (SUO) guidelines (Chang 2017). RC+PLND with curative intent must occur between 4 to 12 weeks after the last dose of neoadjuvant treatment.

Any patient refusing RC+PLND is followed observationally with disease assessments per schedule of events.

pCR is the primary endpoint and is defined as the absence of viable tumor (pT0N0) in examined tissue from RC+PLND. pCR is assessed by central pathology review after RC+PLND with curative intent. Post-RC restaging is performed via the same radiologic modalities at the first follow-up disease assessment scan (12 weeks after RC+PLND). During the first 2 years on study, follow-up is performed every 12 weeks (±14 days) from RC+PLND until disease progression/recurrence, patient death study closure, or withdrawal of consent, whichever occurs first. After 2 years on study, the frequency of visits including response assessments is reduced to every 24 weeks (±14 days). Tumor imaging should also be performed whenever disease progression is suspected.

Radiographic disease progression precluding a curative intent surgery noted prior to RC+PLND, failure to undergo RC+PLND for patients with residual muscle invasive disease and/or any radiographic disease present, gross residual disease left behind at time of RC+PLND, local or distant recurrence post-RC as assessed radiographically and/or confirmed with biopsy (local pathology review), or death from any cause are considered EFS events. Patients who are found to have EFS events but are still alive are to transition into long-term follow-up.

After RC+PLND, patients with evidence of residual disease and/or pathologically node-positive disease are managed according to local guidelines or standard of care (SOC). For patients that do not have an EFS event, but start a new anticancer treatment after RC+PNLD, imaging assessments continue according to the protocol-defined schedule until disease progression/recurrence, patient death, study closure, or withdrawal of consent, whichever occurs first.

During long-term follow up, patients are contacted every 24 weeks (±14 days) for survival status and collection of subsequent anticancer therapy information until death, study closure, or withdrawal of consent, whichever occurs first.

Results of any additional investigative imaging (eg, urograms) should be reported in the electronic CRFs (eCRFs).

(viii) Test Product, Dose, and Mode of Administration

Enfortumab vedotin is administered as an IV infusion at 1.25 mg/kg over approximately 30 minutes on Days 1 and 8 of every 3-week cycle for 3 cycles prior to RC+PLND.

In the absence of infusion-related reactions (IRRs), the infusion rate for all patients should be calculated in order to achieve an approximate 30-minute infusion period. Enfortumab vedotin must not be administered as an IV push or bolus. Enfortumab vedotin should not be mixed with other medications. At least 1 week (7 days) must elapse between doses of enfortumab vedotin.

Enfortumab vedotin doses are calculated on the basis of a patient's actual body weight at baseline. Doses should be recalculated when a patient's body weight changes by ≥10% of baseline or the previous cycle, or when dose adjustment criteria are met. Actual weight is used except for patients weighing >100 kg; in such cases, the dose will be calculated based on a weight of 100 kg. The maximum dose permitted in this study is 125 mg.

Table 6 shows the Enfortumab vedotin step-down dose levels for muscle invasive bladder cancer.

| Dose level | Enfortumab Vedotin Dose (mg/kg) |
|---|---|
| 1 | 1.25 |
| −1 | 1 |
| −2 | 0.75 |

### (ix) Duration of Treatment

Patients receive 3 cycles of enfortumab vedotin monotherapy, followed by RC+PLND. Surgery consists of RC+bilateral PLND with curative intent in accordance with the AUA/ASCO/ASTRO/SUO guidelines.

The study closes 5 years after enrollment of the last patient, or when no patients remain in long-term follow-up, whichever occurs first. Additionally, the sponsor may terminate the study at any time.

### (x) Efficacy Assessments

Initial staging is determined primarily from pathologic findings from the diagnostic TURBT and reviewed by a local laboratory, supplemented with staging imaging from radiographic studies per RECIST Version 1.1. Patients require imaging using CT with IV contrast of the chest and a CT urogram of the abdomen and pelvis at screening (≤28 days before enrollment) for clinical staging. Patients must receive the same imaging modality throughout the study for response assessments.

Tumor tissue samples collected from the patient's diagnostic TURBT within 90 days prior to the first dose of study treatment are sent for central pathology review. Tissue samples from RC+PLND will also be sent for central pathology review of pathologic response assessment. Tumor response is assessed after RC+PLND with curative intent by pathology review analysis. Pathologic staging information includes TNM classification, histology, lymph node counts, and surgical margins.

Patients are also be followed for disease recurrence using serial imaging as outlined in the Study Design subsection of the Synopsis. Disease recurrence is determined by radiologically confirmed disease progression per RECIST Version 1.1 (investigator assessment). Optional tumor biopsies at the time of disease progression, if available, may also be used to document disease progression.

Delay of planned RC+PLND with curative intent (defined as >12 weeks after the last dose of study treatment) due to treatment-related AEs is documented.

### (xi) Pharmacokinetic and ATA Assessments

Blood samples for PK and ATA are collected throughout the study at specific time points. Validated or qualified assays are used to measure the concentrations of enfortumab vedotin ADC, total antibody (TAb), and MMAE in serum or plasma. PK samples are collected and archived for possible analysis of concomitant drug levels or other enfortumab vedotin-related species, such as circulating metabolites of MMAE.

(xii) Biomarker Assessments

Peripheral blood, urine, and tumor biopsies are collected at protocol specified time points. Exploratory, predictive, and prognostic biomarkers associated with response, resistance, or safety observations are monitored before and during study treatment. Tumor samples obtained at RC+PLND are used to characterize the clinical mechanisms of action and resistance.

Tumor tissue from diagnostic TURBT specimens and RC+PLND is required (fine needle aspiration is not adequate) to identify novel biomarkers. If additional post-treatment biopsies are done as part of SOC, the samples may also be used to further identify biomarkers of response and mechanism of action and resistance to treatment.

Biomarker assessments in tumor tissue may include, but may not be limited to, measurement of gene expression (GE) and mutation burden, characterization of the tumor microenvironment (TME) and tumor subtype, and drug effects. Assays may include, but may not be limited to, immunohistochemistry (IHC) for Nectin-4 and PD-L1, and Next Generation Sequencing (NGS) of RNA and DNA. Biomarker assessments in blood samples may include, but may not be limited to, measurement of baseline and drug induced changes in circulating blood cell subpopulations, immunoassays, and circulating disease markers. Blood and urine assays may include, but may not be limited to, circulating tumor DNA, proteomic methodologies such as enzyme-linked immunosorbent assay (ELISA), immunoassays as a marker of tumor response or therapy resistance, and markers of immune function, including abundance of immune cell subsets and cytokines.

Other tissue (eg, skin) from optional tissue sample submissions may be analyzed for biomarkers including tissue levels of drug and drug products, nucleic acids, and protein to investigate possible associations with mechanisms of resistance or sensitivity to treatments as well as dynamic changes associated with treatments. Methods of analysis include IHC, NGS of DNA and RNA, T cell receptor beta chain sequencing, polymerase chain reaction, flow cytometry, and immunoassays.

(xiii) Safety Assessments

Safety assessments are based on the information collected through the safety surveillance process and will include the data from recorded AEs, including serious adverse events (SAEs), concomitant medications, physical examination findings, cardiac monitoring, and laboratory tests. Safety is monitored over the course of the study by the SMC.

(xiv) Statistical Methods

### (a) Analysis for the Key Safety Endpoint

The safety analysis evaluates the type, incidence, severity, seriousness, and relatedness of AEs, and the type, incidence, and severity of laboratory abnormalities. The incidence, duration, and resolution of AEs of special interest (AESIs) are summarized.

### (b) Analysis for the Key Efficacy Endpoint

The observed pathological complete response rate (pCRR) and the 95% CIs will be provided using Clopper-Pearson methodology, by cohorts.

### (c) Sample Size

Approximately 20 patients may be enrolled.

The sample size is not based on power calculations for formal hypothesis testing, but is selected based on the precision of the estimate for pCRR as characterized by the 95% CIs.

For illustration purposes, Table 7 below is a summary of the 2-sided 95% CIs, assuming a pCRR of 30% or 40%, and for a sample size of 20 patients:

TABLE 7

| pCRR | 95% Exact CI (n = 20) | 95% Exact CI (n = 50) |
|---|---|---|
| 30% | (12%, 54%) | (18%, 45%) |
| 40% | (19%, 64%) | (26%, 55%) |

### 6.1.3 Objectives

### Primary Objective

The primary Objective is to assess the antitumor activity of neoadjuvant and perioperative enfortumab vedotin monotherapy or neoadjuvant enfortumab vedotin as measured by the pCR rate, defined as the absence of viable tumor (pT0N0) in examined tissue from RC and PLND by central pathology review.

### Secondary Objectives Include:

To assess the EFS on study therapy by investigator,
To assess the pDS rate, defined as patients with tumors <pT2 (includes pT0, pTis, pTa, pT1) and N0 in examined tissue from RC+PLND by central pathology review,
To assess the DFS by investigator,
To assess OS,
To assess the safety and tolerability of enfortumab vedotin monotherapy, and
To assess the percentage of planned RC+PLND delayed due to treatment-related AEs.

### Additional Objectives Include:

To assess Nectin-4 and PD-L1 expression levels,
To assess biomarkers of biological activity and disease resistance, and their potential associations with clinical outcome measures,
To assess PK and the incidence of ATA, and
To assess patient-reported experience and patient reported tolerability of treatment.

### Endpoints

### Primary Endpoint

The primary efficacy endpoint of this study is pCR rate by central pathology review.

### Secondary Endpoints

EFS by investigator assessment,
pDS rate by central pathology review
DFS by investigator assessment,
OS,
Type, incidence, severity, seriousness, and relatedness of AEs,
Type, incidence, and severity of laboratory abnormalities, and
Percentage of planned RC+PLND delayed (>12 weeks after the last dose of treatment) due to treatment-related AEs.

### Additional Endpoints

Exploratory biomarkers of clinical activity, including relationship of Nectin-4 expression status to response
Selected plasma or serum PK parameters of enfortumab vedotin, MMAE, and TAb
Incidence of ATA to enfortumab vedotin
Patient experience as assessed by exit interviews

### 6.1.4 Study Plan

### 6.1.4.1 Summary of Study Design

This study is designed to evaluate the safety and antitumor activity of enfortumab vedotin as monotherapy for the treatment of cisplatin-ineligible patients with MIBC in the neoadjuvant and perioperative settings.

Treatment with enfortumab vedotin monotherapy is evaluated in patients with MIBC. The study enrolls approximately 20 patients with cT2-T4aN0M0 MIBC. All patients are treated with neoadjuvant enfortumab vedotin (1.25 mg/kg) administered as an IV infusion on Days 1 and 8 of every 3-week cycle for 3 cycles prior to RC+PLND.

Safety is monitored by the SMC on an ongoing basis. Step-down dose levels (up to 2 levels) for enfortumab vedotin below the dose of 1.25 mg/kg is allowed if recommended by the SMC and approved by the study sponsor. Enfortumab vedotin step-down dose levels for muscle invasive bladder cancer are illustrated in Table 8, below.

TABLE 8

| Dose Level | Enfortumab Vedotin Dose (mg/kg) |
|---|---|
| 1 | 1.25 |
| −1 | 1 |
| −2 | 0.75 |

All patients should have a TURBT within 90 days prior to the first treatment dose, and tissue from the diagnostic TURBT must be confirmed to be available prior to enrollment and be deemed sufficient for pathology review and biomarker analysis. All patients undergo baseline post-TURBT radiographic screening assessment using CT with IV contrast of the chest and a CT urogram of the abdomen and pelvis for clinical staging. CT urogram or MRI urogram are acceptable imaging methods to satisfy the abdominal and pelvic scanning requirements for MIBC patients specified in the protocol.

Following neoadjuvant treatment, restaging is done via the same radiologic modalities ≤4 weeks prior to RC+PLND to exclude disease progression, which would preclude curative surgery. Surgery consists of RC+PLND with curative intent in accordance with the AUA/ASCO/ASTRO/SUO guidelines (Chang 2017). RC+PLND with curative intent must occur between 4 to 12 weeks after the last dose of neoadjuvant treatment.

Any patient refusing RC+PLND is followed observationally with disease assessments per schedule of assessments.

pCR is the primary endpoint and is defined as the absence of viable tumor (pT0N0) in examined tissue from RC+PLND. pCR is assessed by central pathology review after RC+PLND with curative intent. Post-RC restaging is performed via the same radiologic modalities at the first follow-up disease assessment scan (12 weeks after RC+PLND). During the first 2 years on study, follow-up is performed every 12 weeks (±14 days) from RC+PLND until disease progression/recurrence, patient death, study closure, or withdrawal of consent, whichever occurs first. After 2 years on study, the frequency of visits including response assessments is reduced to every 24 weeks (±14 days). Tumor imaging should also be performed whenever disease progression is suspected.

Radiographic disease progression precluding a curative intent surgery noted prior to RC+PLND, failure to undergo RC+PLND for patients with residual muscle invasive disease and/or any radiographic disease present, gross residual disease left behind at time of RC+PLND, local or distant recurrence post-RC as assessed radiographically and/or confirmed with biopsy (local pathology review), or death from any cause is considered to be EFS events. Patients who are found to have EFS events but are still alive transition into long-term follow-up.

After RC+PLND, patients with evidence of residual disease and/or pathologically node-positive disease are managed according to local guidelines or standard of care (SOC). For patients that do not have an EFS event, but start a new anticancer treatment after RC+PNLD, imaging assessments continue according to the protocol-defined schedule until disease progression/recurrence, patient death, study closure, or withdrawal of consent, whichever occurs first.

During long-term follow up, patients are contacted every 24 weeks (±14 days) for survival status and collection of subsequent anticancer therapy information until death, study closure, or withdrawal of consent, whichever occurs first.

Results of any additional investigative imaging (eg, urograms) should be reported in the electronic CRFs (eCRFs).

### 6.1.4.2 Discussion and Rationale for Study Design

This study provides safety and efficacy data for enfortumab vedotin monotherapy in the neoadjuvant MIBC setting. A dose level of enfortumab vedotin 1.25 mg/kg IV on Days 1 and 8 of every 3-week cycle has been determined for these patients. Dose de-escalation is possible if recommended by the SMC and agreed to by the sponsor.

An SMC monitors the safety throughout the study. The safety endpoints are appropriate for evaluating the safety of the treatment regimens. The key efficacy endpoint of this component of the study (pCR rate by central review) is a direct measure of antitumor activity. This is complemented by contrast-enhanced imaging at baseline, pre-RC as well as in the adjuvant and post-RC stages. To further assess the significance of pCR rate in this study, EFS, DFS, and OS are evaluated as secondary endpoints.

### (i) Method of Assigning Patients to Treatment Groups

This study enrolls patients sequentially.

### (ii) Blinding

This is an open-label, single-arm study.

### 6.1.5 Study Population

### 6.1.5.1 Inclusion Criteria

Patients must have histologically confirmed MIBC with predominant >50% urothelial histology (previously known as transitional cell carcinoma).
Clinical stage cT2-T4aN0M0 determined by TURBT within 90 days prior to the first dose of treatment and by CT with IV contrast of the chest and a CT urogram of the abdomen and pelvis ≤28 days of enrollment.
Mixed cell types are eligible as long as urothelial cancer is predominant: >50%.
Patients with plasmacytoid and/or neuroendocrine tumors are ineligible regardless of component percentage.
Urothelial tumors not originating in the bladder (e.g., upper tract tumors, urethral tumors) are ineligible.
Lymph nodes <10 mm in the short axis are considered normal.

Patients must be cisplatin-ineligible. Patients will be considered cisplatin-ineligible if they meet at least 1 of the following criteria:
GFR<60 mL/min but ≥30 mL/min (measured by the Cockcroft-Gault formula, MDRD or 24-hour urine).
ECOG performance status of 2.
NCI CTCAE Version 4.03 Grade ≥2 hearing loss.
NYHA Class III heart failure.
Patients eligible for enrollment must not have received prior systemic treatment, chemoradiation, or radiation therapy for MIBC. Patients may have received prior intravesical Bacillus Calmette-Guérin (BCG) or intravesical chemotherapy for NMIBC.

Minimum age of 18 years.

An ECOG performance status of 0, 1, or 2.

Anticipated life expectancy of ≥3 months as assessed by the investigator.

Tumor samples with an associated pathology report from the diagnostic TURBT done 90 days prior to the first dose of study treatment must be available prior to enrollment and determined to be sufficient for pathology review and biomarker analysis. Note: Submitted TURBT biopsy must contain the detrusor muscle for confirmation of disease stage.

Patients must be deemed eligible for RC+PLND by his/her urologist and/or oncologist and agree to undergo curative intent standard RC+PLND (including prostatectomy if applicable) as per AUA/ASTRO/ASCO/SUO guidelines.

Have adequate organ function as defined in Table 9, below. Specimens must be collected within 7 days prior to the start of study treatment.

TABLE 9

| System | Laboratory Value |
|---|---|
| Hematological: | |
| ANC | ≥1500/uL |
| Platelets | ≥100,000/uL |
| Hemoglobin | ≥9.0 g/dL or ≥5.6 mmol/L^{a} |
| Renal: | |
| Measured or calculated^{b} CrCl | ≥30 mL/min for participants with |
| AND/OR creatinine ≤1.5 × ULN. | creatinine levels |
| Both creatinine level and creatinine | >1.5 × institutional ULN, AND/OR |
| clearance are applicable when local | creatinine levels |
| guidelines require both assessments | ≤1.5 × upper limit of normal (ULN) |
| (GFR can also be used in place of | |
| CrCl) | |
| Hepatic: | |
| Serum Total bilirubin | ≤1.5 × ULN OR direct bilirubin ≤ |
| | ULN for participants with total |
| | bilirubin levels >1. × ULN |
| | ≤3 × ULN for patients with Gilbert's |
| | disease |
| AST (SGOT) and ALT (SGPT) | ≤3 × ULN |
| Coagulation: | |
| INR OR PT | ≤1.5 × ULN unless participant |
| aPTT OR PTT | is receiving anticoagulant therapy |
| | as long as PT or aPTT is within |
| | therapeutic range of intended use |
| | of anticoagulants |

Note:
Table 9 includes eligibility-defining laboratory value requirements for treatment; laboratory value requirements should be adapted according to local regulations and guidelines for the administration of specific chemotherapies.
ALT (SGPT) = alanine aminotransferase (serum glutamic pyruvic transaminase);
ANC = absolute neutrophil count;
aPTT = activated partial thromboplastin time;
AST (SGOT) = aspartate aminotransferase (serum glutamic oxaloacetic transaminase);
CrCl = creatinine clearance;
GFR = glomerular filtration rate
^{a}Criteria must be met without erythropoietin dependency and without packed red blood cell (pRBC) transfusion within last 2 weeks.
^{b}Creatinine clearance (CrCl) should be calculated per institutional standards using the Cockcroft-Gault method, Modification of Diet in Renal Disease equations (MDRD), or by 24 hour urine collection.

A female subject of childbearing potential is anyone born female who has experienced menarche and who has not undergone surgical sterilization (eg, hysterectomy, bilateral salpingectomy, bilateral oophorectomy) or has not completed menopause. Menopause is defined clinically as 12 months of amenorrhea in a person over age 45 in the absence of other biological, physiological, or pharmacological causes. Female subjects of childbearing potential must meet the following conditions:
Agree not to try to become pregnant during the study and for at least 6 months after the final dose of study drug.
Must have a negative urine or serum pregnancy test (minimum sensitivity of 25 mIU/mL or equivalent units of beta human chorionic gonadotropin [β-hCG]) within 3 days prior to Day 1. Female subjects with false positive results and documented verification of negative pregnancy status are eligible for participation.
If heterosexually active, must consistently use highly effective methods of birth control, with a failure rate of less than 1% starting at screening, throughout the study period, and for at least 6 months after the final dose of study drug.
Female subjects must agree not to breastfeed or donate ova starting at screening and throughout the study period, and for at least 6 months after the final dose of study drug.

A male subject who can father children is anyone born male who has testes and who has not undergone surgical sterilization (eg, vasectomy followed by a clinical test proving that the procedure was effective). Male subjects who can father children, must meet the following conditions:
Must not donate sperm starting at screening and throughout the study period, and for at least 6 months after the final dose of study drug. Male subjects will be informed about the negative risk to reproductive function and fertility from the study treatment. Prior to treatment male subjects should be advised to seek information on fertility preservation and sperm cryoconservation.
Must consistently use highly effective methods of birth control, with a failure rate of less than 1% starting at screening and continue throughout study period and for at least 6 months after the final dose of study drug.
Male subjects with a pregnant or breastfeeding partner(s) must consistently use one of 2 contraception options for preventing secondary exposure to seminal fluid for the duration of the pregnancy or time partner is breastfeeding throughout the study period and for at least 6 months after the final dose of study drug.

The patient must provide written informed consent.

### 6.1.5.2 Exclusion Criteria

Received prior systemic treatment, chemoradiation, and/or radiation therapy for MIBC.

Received any prior treatment with a CPI. A CPI is defined as a PD-1 inhibitor, PD-L1 inhibitor, or PD-L2 inhibitor (including, but not limited to, atezolizumab, pembrolizumab, nivolumab, durvalumab, or avelumab).

Received any prior treatment with an agent directed to another stimulatory or co inhibitory T-cell receptor (including but not limited to CD137 agonists, CTLA 4 inhibitors, or OX-40 agonists).

Evidence of nodal disease on imaging.

Evidence of nodal disease on imaging.

Has undergone partial cystectomy of the bladder to remove any NMIBC or MIBC.

Ongoing sensory or motor neuropathy Grade 2 or higher.

Patients with conditions requiring high doses of steroids (>10 mg/day of prednisone or equivalent) or other immunosuppressive medications are excluded. Inhaled or topical steroids are permitted in the absence of active autoimmune disease.

Prior treatment with enfortumab vedotin or other MMAE-based ADCs for urothelial cancer.

Subjects with a history of another invasive malignancy within 3 years before the first dose of study drug, or any evidence of residual disease from a previously diagnosed malignancy. Subjects with nonmelanoma skin cancer or carcinoma in situ of any type (if complete resection was performed) are allowed.

A history of prostate cancer (T2NXMX or lower with Gleason score ≤7) treated with definitive intent (surgically or with radiation therapy) at least 1 year prior to study entry is acceptable, provided that the subject is considered prostate cancer-free and the following criteria are met:
Participants who have undergone radical prostatectomy must have undetectable PSA for >1 year and at screening.
Participants who have had radiation must have a PSA doubling time >1 year (based on at least 3 values determined >1 month apart) and a total PSA value that does not meet Phoenix criteria for biochemical recurrence (i.e., <2.0 ng/mL above nadir).

Participants with untreated low-risk prostate cancer (Gleason score ≤6) on active surveillance with PSA doubling time >1 year (based on at least 3 values determined >1 month apart) are also eligible.

Currently receiving systemic antimicrobial treatment for active infection (viral, bacterial, or fungal) at the time of first dose of enfortumab vedotin. Routine antimicrobial prophylaxis is permitted.

Patients with a positive hepatitis B surface antigen and/or antihepatitis B core antibody; patients with a negative PCR assay are permitted with either universal prophylaxis or the use of a pre-emptive approach. The approach is selected in accordance with regional or national guidelines for patients who receive anticancer therapies.

Active hepatitis C infection or known HIV infection. Patients who have been curatively treated for hepatitis C infection are permitted if they have documented sustained virologic response of 12 weeks. No HIV testing is required unless mandated by local health authority.

Patients with active tuberculosis.

Documented history of a cerebral vascular event (stroke or transient ischemic attack), unstable angina, myocardial infarction, or cardiac symptoms (including congestive heart failure) consistent with NYHA Class IV (see Appendix E) within 6 months prior to the first dose of enfortumab vedotin.

Patients with active keratitis or corneal ulcerations. Patients with superficial punctate keratitis are allowed if the disorder is being adequately treated in the opinion of the investigator.

Has an active autoimmune disease that has required systemic treatment in past 2 years (ie, use of disease modifying agents, corticosteroids, or immunosuppressive drugs). Replacement therapy (eg, thyroxine, insulin, or physiologic corticosteroid replacement therapy for adrenal or pituitary insufficiency) is not considered a form of systemic treatment and is allowed.

History of idiopathic pulmonary fibrosis; organizing pneumonia, drug-induced pneumonitis, idiopathic pneumonitis, or evidence of active pneumonitis on screening chest CT scan.

Prior allogeneic stem cell or solid organ transplant.

Administration of a live, attenuated vaccine within 30 days prior to first dose of study drug. Examples of live vaccines include, but are not limited to, the following: measles, mumps, rubella, varicella/zoster (chicken pox), yellow fever, rabies, BCG, and typhoid vaccine. Seasonal influenza vaccines for injection are generally killed virus vaccines and are allowed; however, intranasal influenza vaccines (eg, FluMist®) are live attenuated vaccines and are not allowed.

Other underlying medical condition that, in the opinion of the investigator, would impair the ability of the patient to receive or tolerate the planned treatment and follow-up; any known psychiatric or substance abuse disorders that would interfere with cooperating with the requirements of the study.

Patients with uncontrolled diabetes. Uncontrolled diabetes is defined as HbA1c≥8% or HbA1c 7% to <8% with associated diabetes symptoms (polyuria or polydipsia) that are not otherwise explained.

### 6.1.6 Treatments

### 6.1.6.1 Treatments Administered

Patients in this study will receive enfortumab vedotin 1.25 mg/kg administered as an IV infusion on Days 1 and 8 of every 3-week cycle during 3 cycles prior to RC+PLND. Surgery will consist of RC+bilateral PLND with curative intent in accordance with the AUA/ASCO/ASTRO/SUO guidelines.

Step down dose levels (up to 2 levels) for enfortumab vedotin below 1.25 mg/kg will be allowed if recommended by the SMC and approved by the study sponsor.

### 6.1.6.2 Investigational Study Drug

### (i) Description

Enfortumab vedotin is generated by conjugation of a chemical intermediate that contains both the MMAE and linker subunits to cysteine residues of the antibody. The resulting ADC contains an average of 3.8 drug molecules per antibody. The enfortumab vedotin drug product is a sterile, preservative free, white to off-white lyophilized powder to be reconstituted for IV administration. Enfortumab vedotin is supplied in 30 mg single-dose vials.

### (ii) Dose and Administration

Enfortumab vedotin will be administered as an IV infusion at 1.25 mg/kg over approximately 30 minutes on Days 1 and 8 of every 3-week cycle for 3 cycles prior to RC+PLND.

In the absence of IRRs, the infusion rate for all patients should be calculated in order to achieve an approximate 30-minute infusion period. Enfortumab vedotin must not be administered as an IV push or bolus. Enfortumab vedotin should not be mixed with other medications. At least 1 week (7 days) must elapse between doses of enfortumab vedotin.

Enfortumab vedotin doses are calculated on the basis of a patients actual body weight at baseline. Doses should be recalculated when a patients body weight changes by ≥10% of baseline or the previous cycle, or when dose adjustment criteria are met. Actual weight is used except for patients weighing >100 kg; in such cases, the dose will be calculated based on a weight of 100 kg. The maximum dose permitted in this study is 125 mg.

The patient should be observed during enfortumab vedotin administration and for at least 60 minutes following the infusion during the first 3 cycles. All supportive measures consistent with optimal patient care should be given throughout the study according to institutional standards.

The infusion site should be monitored closely for redness, swelling, pain, and infection during and at any time after administration. Patients should be advised to report redness or discomfort promptly at the time of administration or after infusion. Institutional guidelines will be followed for the administration of chemotherapy agents and precautions taken to prevent extravasation per institutional standards and as described in “Chemotherapy and Biotherapy Guidelines and Recommendations for Practice” (Polovich 2014) and “Management of Chemotherapy Extravasation: ESMO-EONS Clinical Practice Guidelines” (Perez Fidalgo 2012). In case of enfortumab vedotin extravasation, the combination drug(s) should be held until consultation and further discussion with the medical monitor/sponsor.

(iii) Dose Modifications for Enfortumab Vedotin

Intrapatient dose reduction by 1 or 2 dose levels (see Table 8) will be allowed depending on the type and severity of toxicity. Patients requiring a dose reduction may be re-escalated by 1 dose level (i.e., patients reduced to 0.75 mg/kg may only be re-escalated to 1 mg/kg) provided the toxicity does not require study drug discontinuation and has returned to baseline or ≤Grade 1. If the toxicity recurs, re escalation will not be permitted. Patients with ≥Grade 2 corneal AEs will not be permitted to dose re-escalate.

Dose modification recommendations for enfortumab vedotin-associated toxicity are presented in Table 10 and Table 11.

Intrapatient dose reduction or interruption for other enfortumab vedotin-associated toxicity is permitted at the discretion of the medical monitor and site investigator. On a per-patient basis, dose reductions for toxicity, including DLT, may be allowed. The DLT-evaluation period is the first treatment cycle. Patients who experience a DLT in the DLT-evaluation period should not receive further treatment unless clinical benefit is demonstrated with adequately managed toxicity, and there is approval from the medical monitor. The subsequent dose level will be defined by the medical monitor in discussion with the site investigator; the type and severity of the AE observed will be taken into consideration to inform the decision.

Dose interruptions for patients without prior dose reductions and who are responding to treatment may be dose interrupted beyond 3 weeks with approval of the medical monitor, if the patient's toxicity does not otherwise require permanent discontinuation. Patients may not receive other investigational drugs, radiotherapy (except palliative radiotherapy of symptomatic and nonprogressing nontarget bone lesions), or systemic antineoplastic therapy during dose delays. If toxicities warranting a dose delay occur after Day 1 dosing and are not resolved prior to Day 8 dosing (up to Day 10), Day 8 enfortumab vedotin administration must be skipped rather than delayed. If a patient is dose-reduced due to toxicity that subsequently resolves, the patient may resume treatment at the original dose at the discretion of the medical monitor and site investigator. If there is a dose interruption, the schedule for response assessments will not be adjusted and should still be calculated from Cycle 1 Day 1.

Dose interruptions may be allowed for situations other than treatment-related AEs such as medical/surgical events or logistical reasons not related to study therapy. Participants should be placed back on study therapy within 3 weeks of the scheduled interruption, unless otherwise discussed with the sponsor. The reason for interruption should be documented in the patient's study record.

TABLE 10
Recommended dose modifications for enfortumab
vedotin-associated hematologic toxicity

| Grade 1 | Grade 2 | Grade 3 | Grade 4 |
|---|---|---|---|
| Continue at same | Continue at same | Withhold dose until | Withhold dose until |
| dose level. | dose level. | toxicity is ≤ Grade 1 | toxicity is ≤ Grade 1 |
| | For Grade 2 | or has returned to | or has returned to |
| | thrombocytopenia, | baseline, then | baseline, then reduce |
| | withhold dose until | resume treatment at | dose by 1 dose level |
| | toxicity is ≤ Grade 1 | the same dose level | and resume |
| | or has returned to | or consider dose | treatment, or |
| | baseline, then resume | reduction by 1 dose | discontinue at the |
| | treatment at the same | level. | discretion of the |
| | dose level. | Transfusions or | investigator. |
| | | growth factors may | Transfusions or |
| | | be used as indicated | growth factors may |
| | | per institutional | be used as indicated |
| | | guidelines. | per institutional |
| | | | guidelines. |
| | | | For anemia, |
| | | | treatment |
| | | | discontinuation |
| | | | should be strongly |
| | | | considered. |

Note:
hematologic toxicity refers to anemia, thrombocytopenia, neutropenia, and febrile neutropenia.

TABLE 11
Recommended dose modifications for enfortumab
vedotin-associated nonhematologic toxicity

| Grade 1 | Grade 2 | Grade 3 | Grade 4 |
|---|---|---|---|
| Continue at same | Continue at same | Withhold dose until | For Grade 4 AEs, |
| dose level. | dose level, except in | toxicity is ≤ Grade 1 or | discontinue |
| For Grade 1 rash or | the event of Grade 2 | has returned to | treatment.^{a} For |
| skin reactions, may | neuropathy or | baseline, then resume | confirmed SJS or |
| continue at same | corneal AEs. | treatment at the same | TEN, or Grade 4 |
| dose level. | For worsening rash | dose level or consider | rash, permanently |
| If ocular symptoms | or skin reactions, | dose reduction by 1 | discontinue treatment. |
| and/or changes in | consider withholding | dose level^{.a} | Grade 4 vomiting |
| vision are | enfortumab vedotin | For Grade 3 rash or | and/or diarrhea that |
| identified, the | until toxicity is ≤ | skin reactions withhold | improves to ≤ |
| patient should be | Grade 1 or has | enfortumab vedotin | Grade 2 within 72 |
| evaluated by a | returned to baseline, | until toxicity is ≤ | hours with supportive |
| qualified | then resume | Grade 1 or has returned | management does not |
| optometrist or | treatment at the | to baseline, then | require discontinuation. |
| ophthalmologist. | same dose level or | resume treatment at the | For Grade 4 |
| | consider dose | same dose level or | hyperglycemia or |
| | reduction by 1 | consider dose reduction | blood glucose >500 |
| | level. | by 1 level. | mg/dL, withhold |
| | For Grade 2 | For suspected Stevens | enfortumab vedotin |
| | neuropathy or | Johnson Syndrome | treatment and |
| | corneal AEs, | (SJS) or suspected | undertake a full |
| | withhold dose until | toxic epidermal | evaluation of the |
| | toxicity is ≤ Grade | necrolysis (TEN) | hyperglycemia to |
| | 1 or has returned to | withhold enfortumab | determine the |
| | baseline, then | vedotin. Consider | underlying diagnosis. |
| | resume treatment at | referral of the subject | Once the blood glucose |
| | the same dose level. | to a dermatologist/ | is ≤250 mg/dL and |
| | For the second | specialist for diagnosis | patient is clinically |
| | occurrence of Grade | and specialized care. | and metabolically |
| | 2 neuropathy or | Subjects who have | stable, dosing may |
| | corneal AEs, | confirmed SJS or | be resumed with |
| | withhold dose until | recurrent Grade 3 rash | close monitoring |
| | toxicity is ≤ Grade | events should have | after consultation |
| | 1, then reduce the | therapy permanently | with medical monitor. |
| | dose by 1 dose level | discontinued. | |
| | and resume treatment. | For Grade 3 neuropathy | |
| | If ocular symptoms | or corneal AEs, | |
| | and/or changes in | discontinue treatment. | |
| | vision are identified, | For Grade 3 | |
| | the patient should | hyperglycemia or | |
| | be evaluated by a | blood glucose >250 | |
| | qualified optometrist | mg/dL, withhold | |
| | or ophthalmologist. | enfortumab vedotin | |
| | | treatment. Resume | |
| | | treatment once | |
| | | hyperglycemia/blood | |
| | | glucose is ≤250 mg/dL | |
| | | and patient is | |
| | | clinically and | |
| | | metabolically stable. | |
| | | If ocular symptoms | |
| | | and/or changes in | |
| | | vision are identified, | |
| | | the patient should be | |
| | | evaluated by a | |
| | | qualified optometrist or | |
| | | ophthalmologist. | |

^{a}Grade 3/4 electrolyte imbalances/laboratory abnormalities, that are not associated with clinical sequelae or are corrected with supplementation/appropriate management within 72 hours of their onset do not require discontinuation (eg, Grade 4 hyperuricemia). Grade 4 amylase/lipase elevation that is not associated with clinical sequalae does not require discontinuation; withhold dose until toxicity is ≤ Grade 3 or has returned to baseline, then resume treatment at the same dose level. Grade 3 amylase/lipase elevation that is not associated with clinical sequelae may continue treatment at the same dose level. Hold therapy for ≥ Grade 3 amylase/lipase with clinical sequelae until evaluation is complete.

See Section 6.1.6.4(i) for recommended management of infusion reactions. See Section 6.1.6.4(ii) for recommended management of hyperglycemia. See Section 6.1.6.4(iii) for recommended management of rash.

Patients who experience unacceptable toxicity that is attributable only to enfortumab vedotin should be discontinued from study drug.

### (a) Treatment Discontinuation Recommendations Related to Liver Safety

In the absence of an explanation for increased liver function tests (LFTs), such as viral hepatitis, preexisting or acute liver disease, or exposure to other agents associated with liver injury, the patient may be discontinued from the study treatment. The investigator may determine that it is not in the patient's best interest to continue study treatment.

Discontinuation of treatment should be considered if:
ALT or AST>8×upper limit of normal (ULN)
ALT or AST>5×ULN for more than 2 weeks
ALT or AST>3×ULN and total bilirubin >2×ULN or international normalized ratio (INR)>1.5 (if INR testing is applicable/evaluated)
ALT or AST>3×ULN with the appearance of symptoms suggestive of liver injury (e.g., right upper quadrant pain or tenderness) and/or eosinophilia (>5%)

These treatment discontinuation recommendations are based on the FDA Guidance for Industry (Drug-Induced Liver Injury: Premarketing Clinical Evaluation, July 2009). The recommendations are a basic guide to the investigator based on accumulated clinical experience with drugs in development, and are not specific to clinical experience with enfortumab vedotin.

See Section 6.1.11 for recommended liver safety monitoring and assessment criteria in patients with Grade 2 or greater elevations in ALT, AST, or bilirubin.

### 6.1.6.3 Concomitant Therapy

All concomitant medications and blood products administered are recorded from Day 1 (predose) through the safety reporting period (EOT visit or 30 days after the last study treatment, whichever is later). Any concomitant medication given for a study protocol-related AE should be recorded from the time of informed consent.

### (i) Required Concomitant Therapy

There are no required concomitant therapies.

### (ii) Allowed Concomitant Therapy

The use of anti-emetics is permitted. The use of insulin is permitted as part of SOC. Premedications for IRRs per Section 6.1.6.4(i) are permitted; however, prophylactic premedication prior to study treatment on Cycle 1 Day 1 for prevention of IRRs may not be administered.

Therapy to manage enfortumab vedotin-associated toxicity as recommended in Section 6.1.6.2(iii) is permitted, including hematopoietic growth factors and transfusions.

Patients who are receiving strong CYP3A4 inhibitors or P-gp inhibitors concomitantly with enfortumab vedotin should be monitored for adverse reactions.

Routine prophylaxis with vaccines is permitted; however, patients may not be treated with a live, attenuated vaccine during the study.

Steroids may be used as clinically indicated.

(iii) Prohibited Concomitant Therapy

Medications or vaccinations specifically prohibited in the exclusion criteria are not allowed during the ongoing trial. If there is a clinical indication for any medication or vaccination specifically prohibited during the trial, discontinuation from trial therapy or vaccination may be required.

Listed below are specific restrictions for concomitant therapy or vaccination during the course of the study:
Antineoplastic systemic chemotherapy or biological therapy.
Immunotherapy not specified in this protocol.
Chemotherapy not specified in this protocol.
Investigational agents other than enfortumab vedotin.
Radiation therapy.
Live vaccines within 30 days prior to the first dose of study treatment and while participating in the study. Examples of live vaccines include, but are not limited to, the following: measles, mumps, rubella, varicella/zoster, yellow fever, rabies, BCG, and typhoid vaccine. Seasonal influenza vaccines for injection are generally killed virus vaccines and are allowed; however, intranasal influenza vaccines (eg, FluMist) are live attenuated vaccines and are not allowed.
Pioglitazone.

Participants who require the use of any of the aforementioned treatments for clinical management should be removed from the study.

### 6.1.6.4 Management of Adverse Reactions

### (i) Management of Infusion Reactions

An infusion-related reaction (IRR) may occur during the infusion of study treatment. The infusion should be administered at a site properly equipped and staffed to manage anaphylaxis should it occur. All supportive measures consistent with optimal patient care should be given throughout the study according to institutional standards. Supportive measures may include administering medications for IRRs.

Premedications for IRRs are permitted as described below; however, prophylactic premedication prior to study treatment on Cycle 1 Day 1 for prevention of IRRs may not be administered. Patients who have experienced an IRR may be premedicated for subsequent infusions. Premedication may include pain medication (eg, acetaminophen or equivalent), an antihistamine (eg, diphenhydramine hydrochloride), and a corticosteroid administered approximately 30 to 60 minutes prior to each enfortumab vedotin infusion or according to institutional standards. Should a patient experience IRRs in the setting of premedication, continued treatment must be discussed with the medical monitor prior to the next planned dose.

If anaphylaxis occurs, study treatment administration should be immediately and permanently discontinued.

If a patient experiences an IRR after receiving study treatments and a single cause of the IRR cannot be determined, appropriate sets of guidelines for IRRs must be followed.

### (ii) Management of Hyperglycemia

Investigators should monitor blood glucose levels and are advised to perform additional assessments if any symptoms of hyperglycemia are observed, including a thorough evaluation for infection. In addition, if steroids are used to treat any other condition, blood glucose levels may require additional monitoring. If elevated blood glucose levels are observed, patients should be treated according to local SOC and referral to endocrinology may be considered.

Patients, especially those with a history of or ongoing diabetes mellitus or hyperglycemia, should be advised to immediately notify their physician if their glucose level becomes difficult to control or if they experience symptoms suggestive of hyperglycemia such as frequent urination, increased thirst, blurred vision, fatigue, and headache.

Patients who enter the study with an elevated HbA1c (≥6.5%) at baseline should be referred to an appropriate provider during Cycle 1 for glucose management. Blood glucose should be checked prior to each dosing and Dose should be withheld for blood glucose >250 mg/dL. Dosing may continue once the patient's blood glucose has improved to ≤250 mg/dL and patient is clinically and metabolically stable. The use of insulin is permitted as part of SOC. Blood glucose >500 mg/dL considered related to enfortumab vedotin requires drug interruption and a full evaluation of the hyperglycemia to determine the underlying diagnosis. Once hyperglycemia/elevated blood glucose has improved to ≤250 mg/dL, dosing may resume with close monitoring after consultation with medical monitor. If a patient experiences new onset of diabetes mellitus, evaluate with a metabolic panel, urine ketones, HbA1c, C-peptide, to assess new onset of type 1 diabetes in the setting of combination with CPI.

(iii) Management of Rash

Enfortumab vedotin is a Nectin-4 directed antibody drug conjugate. Nectin-4 is a cell adhesion molecule that is highly expressed in urothelial carcinoma. Low to moderate levels of Nectin-4 are also expressed on normal tissues, including skin keratinocytes, sweat glands and hair follicles; thus, skin reactions are anticipated events. As such, skin reactions are AEs of interest in all clinical studies with enfortumab vedotin.

Reports of severe cutaneous adverse reactions were identified in 15 patients receiving enfortumab vedotin, some of whom had fatal outcomes. These reactions occurred predominantly during the first cycle of treatment. AEs reported in these cases included Stevens-Johnson Syndrome (SJS) (5 cases), blister (3 cases), dermatitis bullous (3 cases), symmetrical drug-related intertriginous and flexural exanthema (SDRIFE; 2 cases), and 1 case each of dermatitis exfoliative, exfoliative rash, epidermal necrosis, oropharyngeal blistering, stomatitis, and toxic epidermal necrolysis (TEN).

In enfortumab vedotin monotherapy studies of urothelial carcinoma, SAEs of severe cutaneous adverse reactions were reported in 11 of 749 subjects (1.5%) and included dermatitis bullous (0.4%), drug eruption (0.4%), blister (0.1%), conjunctivitis (0.1%), SJS (0.1%), stomatitis (0.1%), and toxic skin eruption (0.1%).

Subjects should be advised to contact the Investigator immediately if they have signs and symptoms of skin reactions, oral mucosal and ocular abnormalities including mucositis or conjunctivitis. Starting in the first cycle and throughout treatment, closely monitor subjects for skin reactions. For mild to moderate skin reactions, consider appropriate treatment, such as topical corticosteroids and antihistamines as clinically indicated. For worsening Grade 2 rash or skin reactions, consider withholding enfortumab vedotin. For severe (Grade 3) rash or skin reactions or suspected SJS or TEN, withhold enfortumab vedotin and consider referral for specialized care. Permanently discontinue enfortumab vedotin in patients with confirmed SJS or TEN, or Grade 4 or recurrent Grade 3 skin reactions.

### 6.1.6.5 Treatment Compliance

Study drug administration will be performed by study site staff and documented in source documents and the CRF.

### 6.1.7 Study Assessments

### 6.1.7.1 Screening Baseline Assessments

Only patients who meet all inclusion and exclusion criteria specified in Section 6.1.5 will be enrolled in this study. Enrollment status and date will be recorded in CRF.

Patient medical history includes a thorough review of significant past medical history, current conditions, any treatment for prior malignancies and response to prior treatment, and any concomitant medications.

Acquisition of a fresh tumor specimen from TURBT (tissue must be collected within 90 days of planned Cycle 1 Day 1), a complete eye examination, brain scan (if clinically indicated) (MRI with gadolinium contrast preferred), bone scan (if clinically indicated), CT with IV contrast of the chest and a CT urogram of the abdomen and pelvis for baseline tumor imaging, serology for hepatitis B surface antigen and antihepatitis B core antibody, serology for antihepatitis C antibody, HbA1c, urinalysis with reflexive microscopic analysis, and thyroid function tests are required for all patients at screening.

INR/PT/PTT, a pregnancy test (either urine or serum, for females of childbearing potential), physical examination (including weight), collection of height, vital signs, CBC with differential, serum chemistry panel, CrCl, ECOG performance status assessment, and ECG are required at baseline. An ECHO is required at baseline for subjects with NYHA Class III heart failure or a history of coronary heart disease, arrhythmia or other significant heart disease.

### 6.1.7.2 Response Efficacy Assessments

Initial staging will be determined primarily from pathologic findings from the diagnostic TURBT and reviewed by local laboratory, supplemented with staging imaging from radiographic studies as per RECIST Version 1.1. CT with IV contrast of the chest and a CT urogram of the abdomen and pelvis will be used ≤28 days before enrollment for initial clinical staging. Patients must receive the same imaging modality throughout the study for response assessments.

Pre-RC scans should be completed ≤28 days prior to RC+PLND, to exclude disease progression, which would preclude curative surgery (RC+PLND must occur between 4 to 12 weeks after the last dose of neoadjuvant treatment). Pre-RC scans should be reviewed prior to RC+PLND. The post-RC scans should be performed every 12 weeks (±14 days) (based on the date of RC+PLND) for the first 2 years and then every 24 weeks (±14 days) until disease progression/recurrence, patient death, withdrawal of consent, or study closure, whichever occurs first. Tumor imaging should also be performed (using RECIST Version 1.1 criteria) whenever disease progression is suspected.

For patients that do not have an EFS event, but start a new anticancer treatment after RC+PNLD, imaging assessments continue at post-treatment follow-up visits until disease progression/recurrence, patient death, withdrawal of consent, or study closure, whichever occurs first.

Tumor tissue samples collected from the patient's diagnostic TURBT within 90 days prior to the first dose of study treatment are sent for central pathology review. Tissue samples from RC+PLND are also be sent for central pathology review of pathologic response assessment. Tumor response is assessed after RC+PLND with curative intent by pathology review analysis. Pathologic staging information will include TNM classification, histology, lymph node counts, and surgical margins. pCR as the primary endpoint is evaluated by central pathology review.

Patients will also be followed for disease recurrence using serial imaging. Disease recurrence will be determined by radiologically confirmed disease progression per RECIST Version 1.1 (investigator assessment). Optional tumor biopsies at the time of disease progression, if available, may also be used to document disease progression.

Delayed planned RC+PLND with curative intent (defined as >12 weeks after the last dose of study treatment) due to treatment-related AEs is documented.

See Section 6.1.8.2 for definitions of study endpoints.

### 6.1.7.3 Pharmacokinetic and ATA Assessments

Blood samples for PK and ATA are collected throughout the study per the sample collection schedule provided in Table 12. Validated or qualified assays is used to measure the concentrations of enfortumab vedotin ADC, TAb, and MMAE in serum or plasma. PK samples are collected and archived for possible analysis of concomitant drug levels or other enfortumab vedotin-related species, such as circulating metabolites of MMAE. A validated assay is used to determine the levels of ATA for enfortumab vedotin in plasma. If enfortumab vedotin infusions are discontinued, the corresponding research samples no longer needs to be collected. If at some point prospective PK blood sample collection is no longer required, sites are notified.

### 6.1.7.4 Biomarker Studies

Samples for exploratory biomarkers are collected at protocol-specified timepoints (see Table 12). Biomarker assessments are not used for patient selection.

Methods of analysis may include: immunohistochemistry (IHC), Next Generation Sequencing (NGS) of DNA and RNA, T cell receptor beta chain sequencing, PCR, flow cytometry, and immunoassays.

All patients should provide tissue for biomarker analysis from an archival tissue sample or newly obtained core or excisional biopsy of a tumor lesion. A formalin-fixed paraffin embedded tumor tissue block is requested. Core needle and excisional biopsies from tumor tissue (non-bone sites) are preferred. A fresh baseline core needle biopsy of a tumor lesion may be submitted, if feasible. In the event a biopsy is clinically required as part of SOC, tissue should be made available for biomarker assessment.

Exploratory, predictive, and prognostic biomarkers associated with response, resistance, or safety observations are monitored before and during treatment with enfortumab vedotin. Biomarker assessments in tumor tissue may include, but may not be limited to, measurements of enfortumab vedotin and its metabolites, as well as characterization of the tumor microenvironment (TME) and drug effects. Assays may include, but may not be limited to, IHC and next generation sequencing of RNA and DNA.

### (i) Biomarkers in Blood

The primary effects of enfortumab vedotin on tumor cells may lead to changes in the activation state of local, tumor-associated, and peripheral immune cells. Biomarker assessments in blood and urine samples may include, but may not be limited to, circulating tumor DNA, proteomic methodologies such as enzyme-linked immunosorbent assay (ELISA), immunoassays as a marker of tumor response or therapy resistance, and markers of immune function, including abundance of immune cell subsets and cytokines. These may provide insight into treatment-related changes associated with enfortumab vedotin.

TABLE 12
Pharmacokinetic, immunogenicity, and biomarker sample collection time points

| | | | | | Pharmacokinetics | | | | | |
|---|---|---|---|---|---|---|---|---|---|---|
| | Study | | | | and Immunogenicity | | Blood Biomarkers | | | |
| | Day | Time | Window | Relative Time | EV PK | ATA EV | PBMC-1^{H} | PBMC-2^{H} | Serum | Plasma-1 |
| Screening | Days −28 | N/A | N/A | N/A | | | | | | |
| | to 1 | | | | | | | | | |
| Cycles 1 | Day 1 | Predose | Within | Start of EV | X | X | X | X | X | X |
| and 2 | | | 24 h | infusion | | | | | | |
| | | End of EV | Within | End of EV | X | | | | | |
| | | IV infusion | 15 min | infusion | | | | | | |
| | Day 8 | Predose | Within | Start of EV | X | | X | X | X | X |
| | | | 4 h | infusion | | | | | | |
| | | End of EV | Within | End of EV | X | | | | | |
| | | IV infusion | 15 min | infusion | | | | | | |
| Cycle 3 | Day 1 | Predose | Within | Start of | X | X | X | X | X | X |
| | | | 24 h | infusion | | | | | | |
| Radical Cystectomy^{C} | | | | | | X | X^{F} | | X^{F} | X^{F} |
| EOT occurs 30-37 Days Post Radical Cystectomy | | | | | | | | | X | X |
| Follow-up | Every 12 | N/A | ±7 days | N/A | | | | | X | X |
| | weeks | | | | | | | | | |
| | (±14 d) in | | | | | | | | | |
| | the first 2 | | | | | | | | | |
| | years, and | | | | | | | | | |
| | subsequently | | | | | | | | | |
| | every | | | | | | | | | |
| | 24 weeks | | | | | | | | | |
| | (±14 d) | | | | | | | | | |

| | | | | | | | Urine | Tissue |
|---|---|---|---|---|---|---|---|---|
| | Study | | | | Blood Biomarkers | | Biomarkers | Biomarkers |
| | Day | Time | Window | Relative Time | Plasma-2^{E} | Plasma-3 | Urine | Tumor Tissue^{A} |
| Screening | Days −28 | N/A | N/A | N/A | | | | X^{B} |
| | to 1 | | | | | | | |
| Cycles 1 | Day 1 | Predose | Within | Start of EV | X | X | X | |
| and 2 | | | 24 h | infusion | | | | |
| | | End of EV | Within | End of EV | | | | |
| | | IV infusion | 15 min | infusion | | | | |
| | Day 8 | Predose | Within | Start of EV | X | X | X | |
| | | | 4 h | infusion | | | | |
| | | End of EV | Within | End of EV | | | | |
| | | IV infusion | 15 min | infusion | | | | |
| Cycle 3 | Day 1 | Predose | Within | Start of | X | X | X | |
| | | | 24 h | infusion | | | | |
| Radical Cystectomy^{C} | | | | | X^{F} | X^{F} | X^{F} | X^{D} |
| EOT occurs 30-37 Days Post Radical Cystectomy | | | | | X | X | X | |
| Follow-up | Every 12 | N/A | ±7 days | N/A | X | X | X | X^{G} |
| | weeks | | | | | | | |
| | (±14 d) in | | | | | | | |
| | the first 2 | | | | | | | |
| | years, and | | | | | | | |
| | subsequently | | | | | | | |
| | every | | | | | | | |
| | 24 weeks | | | | | | | |
| | (±14 d) | | | | | | | |

ATA = antitherapeutic antibody;
EV = Enfortumab vedotin;
IV = Intravenous;
N/A = not applicable;
PBMC = peripheral blood mononuclear cells;
PK = pharmacokinetics.
Note:
For subjects who permanently discontinue enfortumab vedotin, no PK samples will be collected. If at some point prospective PK blood sample collection is no longer required, sites are notified.
^{A}If nonprotocol mandated tumor tissue is collected as part of standard care, tissue may be submitted for further biomarker analysis.
^{B}Obtain tissue samples from TURBT (tissue must have been collected via TURBT within 90 days of planned Cycle 1 Day 1)
^{C}Radical cystectomy + bilateral PLND with curative intent will be done between 4 to 12 weeks post-last dose of neoadjuvant treatment. All samples except for tumor tissue should be collected within 7 days prior to RC. Tumor tissue should be obtained on the day of RC.
^{D}See Section 6.1.7.4 for tissue collection requirements for RC.
^{E}Cell Pellet will be generated from the Plasma-2 collection tube.
^{F}Blood and urine samples to be taken within 7 days prior to RC.
^{G}Submit a biopsy in the event that non-protocol-mandated tissue is collected at the follow-up visit.
^{H}PBMC specimen collection is no longer required for patients who consent to Amendment 7 and subsequent amendments on the SGN22E-002 Protocol.

### (ii) Biomarkers in Tumor Tissue

To understand the relationship between the biological characteristics of tumors before treatment and patient outcomes, tissue from the TURBT (tumor biopsies) is examined. Biopsies are assessed for specific pharmacodynamic, predictive, and prognostic biomarkers in the tumor. If tissue is available from a standard-of-care biopsy collected after enrollment (until disease progression after treatment with enfortumab vedotin), it may also be examined.

Tumor tissue from diagnostic TURBT specimens and RC+PLND is required (fine needle aspiration is not adequate) to identify novel biomarkers. If additional post-treatment biopsies are done as part of SOC, the samples may also be used to further identify biomarkers of response and mechanism of action and resistance to treatment.

Biomarker assessments in tumor tissue may include, but are not limited to, central assessment of Nectin-4 expression by IHC and next generation sequencing, tumor subtyping, tumor microenvironment analyses, and profiling of somatic mutations or alterations in genes or RNA commonly altered in cancer.

Other tissue (eg, skin) may be analyzed for biomarkers including tissue levels of drug and drug products, nucleic acids, and protein to investigate possible associations with mechanisms of resistance or sensitivity to treatments as well as dynamic changes associated with treatments. If nonprotocol mandated biopsies of other tissue are conducted as part of standard care, tissue may be submitted for further biomarker analysis.

### 6.1.7.5 Biospecimen Repository

For patients in the US who provide additional consent, remaining de-identified unused blood, urine and/or tissue will be retained by the sponsors and used for future research, including but not limited to the evaluation of targets for novel therapeutic agents, the biology of ADC sensitivity and resistance mechanisms, and the identification of biomarkers of ADCs. Blood, urine, and tissue samples donated for future research will be retained for a period of up to 25 years. If additional consent is not provided, any remaining biological samples will be destroyed following study completion.

### (i) EORTC Core Quality of Life Questionnaire, QLQ-C30

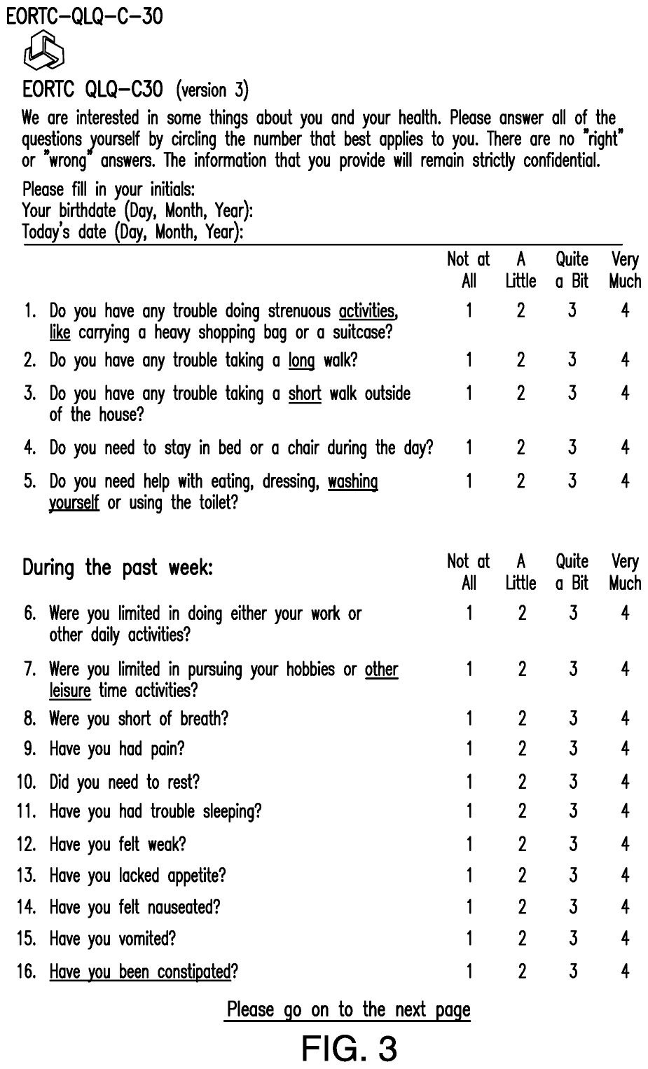

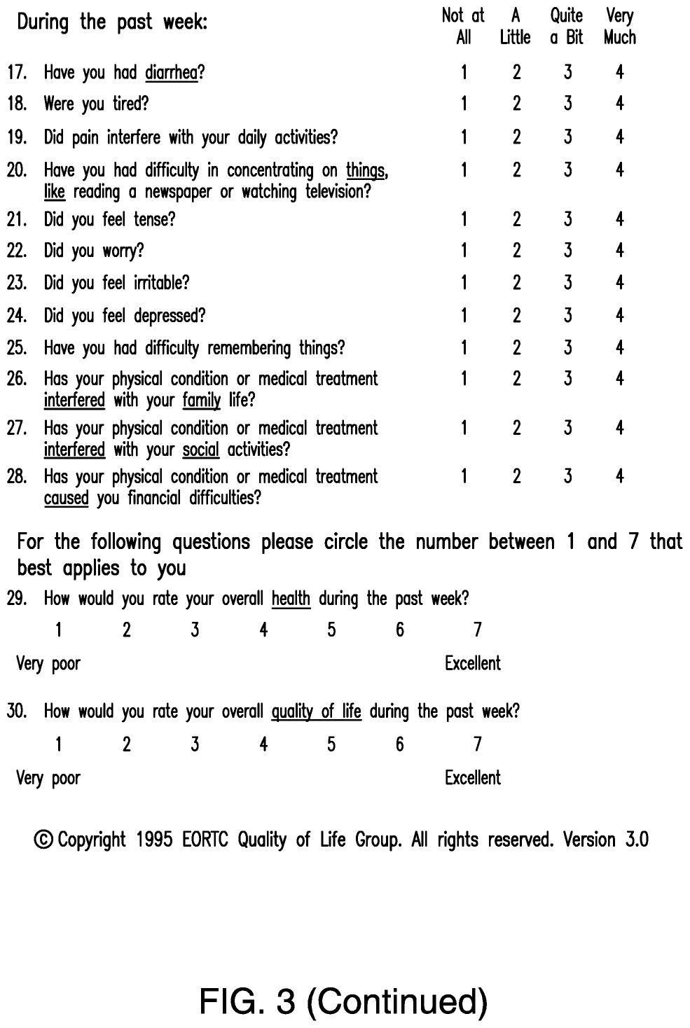

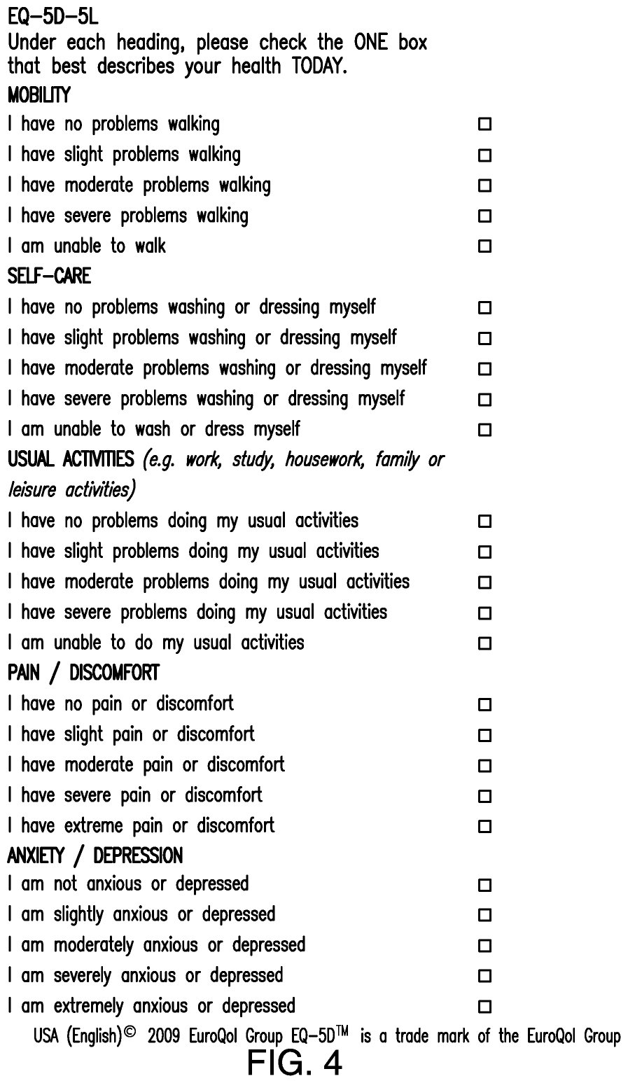

The EORTC Core Quality of Life (QLQ-C-30) assessment was developed to measure aspects of QoL pertinent to patients with a broad range of cancers who are participating in clinical trials (Aaronson 1993); (Sneeuw 1998). The current version of the core instrument (QLQ-C30, Version 3) is depicted in FIG. 3 and is a 30-item assessment consisting of the following:
5 functional domains (physical, role, cognitive, emotional, and social);
3 symptoms scales (fatigue, pain, nausea and vomiting);
Single items for symptoms (shortness of breath, loss of appetite, sleep disturbance, constipation, diarrhea) and financial impact of the disease, and;
2 global items (health, overall QoL)

Each domain is scored from 0 to 100. For the global health status/QoL and functional domain scores, higher scores represent better QoL and functioning, respectively. For symptom scales, higher scores represent greater symptomatology.

### (ii) EuroQol-5 Dimensions (EQ-5D-5L)

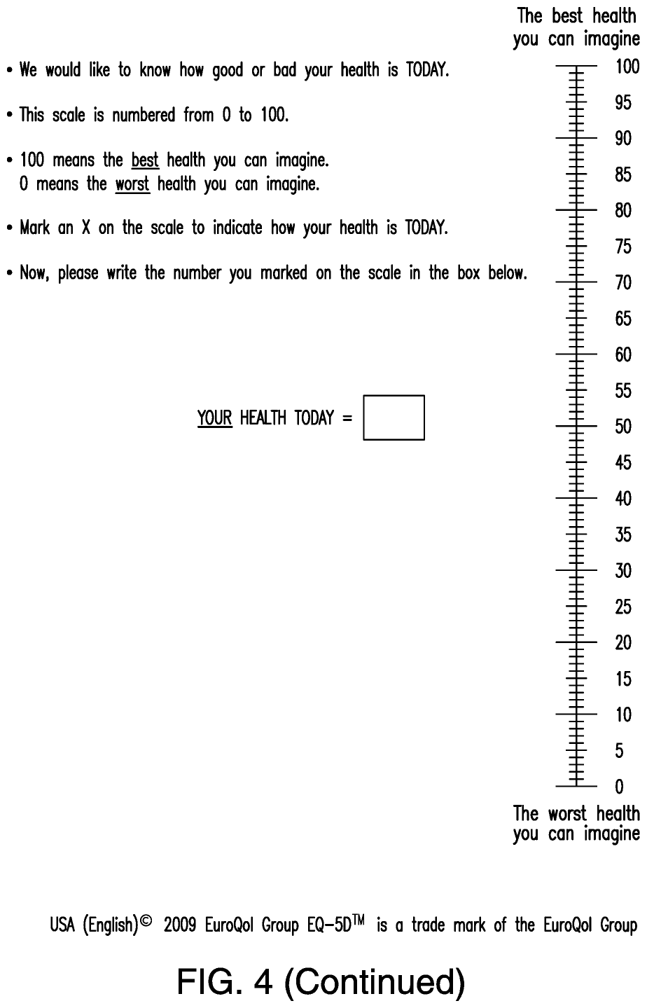

The EQ-5D is a standardized instrument developed by the EuroQol Group for use as a generic, preference-based measure of health outcomes. It is applicable to a wide range of health conditions and treatments and provides a simple descriptive profile and a single index value for health status. The EQ-5D is a 5-item self-reported measure of functioning and well being, which assesses 5 dimensions of health, including mobility, self-care, usual activities, pain/discomfort, and anxiety/depression (see FIG. 4). Each dimension comprises 3 levels (no problems, some/moderate problems, extreme problems). A unique EQ-5D health state is defined by combining 1 level from each of the 5 dimensions. Responses to the 5 items are then converted to a weighted health state index (utility score) based on values derived from general population samples (Herdman 2011). The health utility score is between 0 and 1, where 0 is death and 1 is perfect health. In addition to the utility score, this assessment also records the respondent's self-rated health status on a vertical graduated (0 to 100) visual analogue scale.

### 6.1.7.6 Safety Assessments

The assessment of safety during the course of this study will consist of the surveillance and recording of AEs including SAEs, recording of concomitant medication, and measurements of protocol-specified physical examination findings, cardiac monitoring, and laboratory tests.

Safety will be monitored over the course of the study by the SMC as described in Section 6.1.4.1.

### (i) Adverse Events

### (a) About Adverse Events

### Adverse Event

According to the International Council for Harmonisation (ICH) E2A guideline Definitions and Standards for Expedited Reporting, and 21 Code of Federal Regulations (CFR) 312.32, Investigational New Drug (IND) Safety Reporting, an AE is any untoward medical occurrence in a patient or clinical investigational subject administered a medicinal product and which does not necessarily have a causal relationship with this treatment.

The following information should be considered when determining whether or not to record a test result, medical condition, or other incident on the AEs and Pre-existing Conditions CRF:
From the time of informed consent through the day prior to study Day 1, only study protocol related AEs should be recorded. A protocol-related AE is defined as an untoward medical event occurring as a result of a protocol mandated procedure.
All medical conditions present or ongoing predose on study Day 1 should be recorded.
All AEs (regardless of relationship to study drug) should be recorded from study Day 1 (predose) through the end of the safety reporting period (see Section 6.1.7.6(i)(c)). Complications that occur in association with any procedure (eg, biopsy) should be recorded as AEs whether or not the procedure was protocol mandated.
Changes in medical conditions and AEs, including changes in severity, frequency, or character, during the safety reporting period should be recorded.
In general, an abnormal laboratory value should not be recorded as an AE unless it is associated with clinical signs or symptoms, requires an intervention, results in a SAE, or results in study termination or interruption/discontinuation of study treatment. When recording an AE resulting from a laboratory abnormality, the resulting medical condition rather than the abnormality itself should be recorded (eg, record “anemia” rather than “low hemoglobin”).

### Serious Adverse Events

An AE should be classified as an SAE if it meets one of the following criteria:
Fatal: AE results in death.
Life threatening: The AEs places the patient at immediate risk of death. This classification does not apply to an AE that hypothetically might cause death if it were more severe.
Hospitalization: The AE results in hospitalization or prolonged an existing inpatient hospitalization. Hospitalizations for elective medical or surgical procedures or treatments planned before the signing of informed consent in the study or routine check-ups are not SAEs by this criterion. Admission to a palliative unit or hospice care facility is not considered to be a hospitalization. Pre-planned hospitalizations for therapeutic, diagnostic, or surgical procedures of the underlying cancer or study target disease that do not worsen during the clinical trial need not be captured as SAEs.
Disabling/incapacitating: An AE that results in a persistent or significant incapacity or substantial disruption of the patient's ability to conduct normal life functions.
Congenital anomaly or birth defect: An adverse outcome in a child or fetus of a patient exposed to the molecule or study treatment regimen before conception or during pregnancy.
Medically significant: The AE did not meet any of the above criteria, but could have jeopardized the patient and might have required medical or surgical intervention to prevent one of the outcomes listed above or involves suspected transmission via a medicinal product of an infectious agent.

### Adverse Event Severity

AE severity should be graded using the National Cancer Institute's Common Terminology Criteria for Adverse Events (NCI CTCAE), Version 4.03.

AE severity and seriousness are assessed independently. ‘Severity’ characterizes the intensity of an AE. ‘Serious’ is a regulatory definition and serves as a guide to the sponsor for defining regulatory reporting obligations (see definition for SAEs, above).

### Relationship of the Adverse Event to Study Treatment

The relationship of each AE to enfortumab vedotin should be evaluated by the investigator using the following criteria:
Related: There is evidence to suggest a causal relationship between the drug and the AE, such as:
   A single occurrence of an event that is uncommon and known to be strongly associated with drug exposure (e.g., angioedema, hepatic injury, Stevens-Johnson Syndrome)
   One or more occurrences of an event that is not commonly associated with drug exposure, but is otherwise uncommon in the population exposed to the drug (e.g., tendon rupture)
Unrelated: Another cause of the AE is more plausible (e.g., due to underlying disease or occurs commonly in the study population), or a temporal sequence cannot be established with the onset of the AE and administration of the study treatment, or a causal relationship is considered biologically implausible.

### (b) Procedures for Eliciting and Recording Adverse Events

Investigator and study personnel report all AEs and SAEs whether elicited during patient questioning, discovered during physical examination, laboratory testing and/or other means by recording them on the CRF and/or SAE form, as appropriate.

### Eliciting Adverse Events

An open-ended or non-directed method of questioning should be used at each study visit to elicit the reporting of AEs.

### Recording Adverse Events

The following information should be recorded on the AEs and Pre-existing Conditions CRF:
Description including onset and resolution dates
Whether it met SAE criteria
Severity
Relationship to study treatment or other causality
Outcome
   Diagnosis Vs. Signs or Symptoms

In general, the use of a unifying diagnosis is preferred to the listing out of individual symptoms. Grouping of symptoms into a diagnosis should only be done if each component sign and/or symptom is a medically confirmed component of a diagnosis as evidenced by standard medical textbooks. If any aspect of a sign or symptom does not fit into a classic pattern of the diagnosis, report the individual symptom as a separate AE.

Important exceptions for this study are adverse reactions associated with the infusion of study drug. For IRRs, record the NCI CTCAE term of ‘infusion related reaction’ with an overall level of severity (per NCI CTCAE). In addition, record each sign or symptom of the reaction as an individual AE. If multiple signs or symptoms occur with a given infusion related event, each sign or symptom should be recorded separately with its level of severity.

### Recording Serious Adverse Events

For SAEs, record the event(s) on both the CRF and an SAE form.

The following should be considered when recording SAEs:
Death is an outcome of an event. The event that resulted in the death should be recorded and reported on both an SAE form and CRF.
For hospitalizations, surgical, or diagnostic procedures, the illness leading to the surgical or diagnostic procedure should be recorded as the SAE, not the procedure itself. The procedure should be captured in the narrative as part of the action taken in response to the illness.

### Progression of the Underlying Cancer

Since progression of the underlying malignancy is being assessed as an efficacy variable, it should not be reported as an AE or SAE. Radiographic signs of disease progression (eg, ‘tumor progression’ or ‘metastases’) should not be reported as AEs or SAEs (these data are captured in the efficacy assessment). The clinical symptoms and signs of disease progression (e.g., ‘fatigue’, ‘dyspnea’) may be reported as AEs or SAEs if the symptom cannot be determined as being exclusively due to progression of the underlying malignancy or does not fit the expected pattern of progression for the disease under study; do not report disease progression as the AE term. In addition, complications from progression of the underlying malignancy should be reported as AEs or SAEs.

Death is an outcome of an event. The event that resulted in the death should be recorded and reported on both an SAE form and CRF.

### Pregnancy

Notification to Drug Safety: Complete a Pregnancy Report Form for all pregnancies that occur from the time of first study drug dose until 6 months after the last dose of study drug(s) including any pregnancies that occur in the partner of a male study patient. Only report pregnancies that occur in a male patient's partner if the estimated date of conception is after the male patient's first study drug dose. Email or fax to the Drug Safety Department within 48 hours of becoming aware of a pregnancy (see e-mail or fax number specified on the SAE report form). All pregnancies are monitored for the full duration; all perinatal and neonatal outcomes should be reported. Infants should be followed for a minimum of 8 weeks.

Collection of data on the CRF: All pregnancies (as described above) that occur within 30 days of the last dose of study drug(s) are also be recorded on the AEs and Pre Existing Conditions CRF.

Abortion, whether accidental, therapeutic, or spontaneous, should be reported as an SAE. Congenital anomalies or birth defects, as defined by the ‘serious’ criterion above (see definitions Section 6.1.7.6(i)(a)) should be reported as SAEs.

### Corneal Adverse Events

Corneal ulcer or keratitis AEs≥Grade 2 should be graded within their respective NCI CTCAE categories. Grade 1 corneal ulcer or keratitis AEs should be graded per “Eye disorders—Other, specify” criteria. Other corneal AEs should be recorded and graded per “Eye disorders—Other, specify” criteria.

### Diabetes and Hyperglycemia

Grading for diabetes should be based on the NCI CTCAE v4.03 event term of glucose intolerance. Grading for hyperglycemia should be based on the NCI CTCAE v4.03 event term of hyperglycemia.

### Adverse Events of Possible Hepatic Origin

If an AE is accompanied by increases in LFT values (eg, AST, ALT, bilirubin, etc.) or is suspected to be due to hepatic dysfunction, see Section 6.1.11 for detailed information on recommended monitoring and assessment of liver abnormalities. See Section 6.1.6.2(iii)(a) for treatment discontinuation recommendations related to hepatic safety.

Patients with AEs of hepatic origin accompanied by LFT abnormalities should be carefully monitored.

### (c) Reporting Periods for Adverse Events and Serious Adverse Events

The safety reporting period for all AEs and SAEs, for all cohorts is from study Day 1 (predose) through the EOT visit or 30 days after the last study treatment, whichever is later. All study protocol-related AEs are recorded from the time of informed consent. All SAEs that occur after the safety reporting period and are considered study treatment-related in the opinion of the investigator should also be reported to the sponsor. The investigator will make every attempt to follow all patients with non serious AE for outcome.

The reporting timeframe for AE meeting any serious criteria is described in Section 6.1.7.7.(i)(d).

AEs and SAEs should be reported from study Day 1 through RC+PLND, through 90 days post last dose of neoadjuvant cycle treatment or RC+PLND (if delayed) whichever is last, at treatment discontinuation or through the EOT visit if the participant initiates new anticancer therapy, whichever is later. From day of RC+PLND through the EOT visit, all related Grade 3 events or related SAEs are reported. SAEs that occur after the safety reporting period, assessed as related to RC+PLND are reported.

AEs and SAEs for neoadjuvant should be reported from study Day 1 through EOT or 30 days post-study intervention, whichever is later. SAEs that occur after the safety reporting period that are assessed as related to enfortumab vedotin or RC+PLND are reported.

### (d) Serious Adverse Events Require Immediate Reporting

Within 24 hours of observing or learning of an SAE, investigators report the event to the sponsor, regardless of the relationship of the event to the study treatment regimen.

For initial SAE reports, available case details are recorded on an SAE form. At a minimum, the following should be included:
Patient number
Date of event onset
Description of the event
Investigator's causality assessment
Study treatment, if known

The completed SAE form and SAE Fax Cover Sheet are emailed or faxed to the sponsor's Drug Safety Department within 24 hours.

Relevant follow-up information is submitted to the study sponsor as soon as it becomes available.

### (ii) Clinical Laboratory Tests

Samples are drawn for central and local labs.

Local laboratory testing includes institutional standard tests for study eligibility, evaluating safety, and making clinical decisions. All local laboratory results must be reviewed prior to study drug administration in order to determine whether to proceed with dosing or whether dose modification is required.

The following laboratory assessments will be performed by the local lab to evaluate safety at scheduled time points (see Table 14) during the course of the study:
The serum chemistry panel includes the following tests: albumin, alkaline phosphatase, ALT, AST, bicarbonate, blood urea nitrogen, calcium, creatinine, chloride, glucose, lactate dehydrogenase, phosphorus, potassium, sodium, total bilirubin, amylase, lipase, uric acid, and GFR.
   Verify blood glucose is <250 mg/dL prior to dosing (see Section 6.1.6.4). Patients with diabetes must be tested in the clinic and blood glucose must be <250 mg/dL prior to dosing. Use of insulin is permitted as part of the SOC.
The CBC with differential includes the following tests: white blood cell count with five part differential (neutrophils, lymphocytes, monocytes, eosinophils, and basophils), platelet count, hemoglobin, and hematocrit.
CrCl at baseline as determined per institutional standards.
Thyroid function tests, including:
   Triiodothyronine or free triiodothyronine
   Free thyroxine
   Thyroid-stimulating hormone
Standard urinalysis (with reflexive microscopy).
INR/PT/PTT.
Serology for hepatitis B surface antigen and antihepatitis B core antibody.
Serology for antihepatitis C antibody. If positive, follow-up with polymerase chain reaction/viral load testing.
A serum or urine beta human chorionic gonadotropin pregnancy test for females of childbearing potential.
HbA1c. If HbA1c is elevated (≥6.5%), refer patient to appropriate provider during Cycle 1 for glucose management.
   (iii) Physical Examination Including Weight

Physical examinations should include assessments of the following body parts/systems: skin, abdomen, extremities, head, heart, lungs, neck, and neurological. Height will only be collected at the Baseline visit. Weight is collected at specified time points (see Table 14), and additionally per institutional standards, if applicable, but does not need to be collected at visits following EOT.

### (iv) ECOG Performance Status

ECOG performance status (Table 16) is evaluated at protocol-specified timepoints (see Table 14).

### (v) Cardiac Monitoring

ECGs are conducted at baseline and at the EOT visit. Additional ECGs should be conducted if clinically indicated. Routine 12-lead ECGs are performed after the patient has been in a supine position for at least 5 minutes. The ECG assessments should be performed prior to obtaining the PK and biomarker samples, if possible. For subjects with NYHA Class III heart failure (see Section 6.1.10) or a history of coronary heart disease, arrhythmia or other significant heart disease, a transthoracic ECHO is required at baseline. If clinically indicated, future testing should use the same modality.

### (vi) Complete Eye Examination

Patients have a complete eye examination at screening performed by a qualified optometrist or ophthalmologist, including but not limited to: visual acuity, slit lamp, tonometry examination, and dilated fundus examination. Subsequent eye examinations are conducted as clinically indicated. EOT slit lamp examinations are required for patients who experience corneal AEs during the study. EOT slit lamp examinations (only required for patients who experience corneal AEs during the study) must be performed at least 4 weeks from last dose.

### 6.1.7.7 Post-treatment Assessments

### (i) Follow-Up Assessments

The assessments listed below are done every 12 weeks (±14 days) in the first 2 years after the previous response assessment scan and subsequently every 24 weeks (±14 days) until disease progression/recurrence, patient death, withdrawal of consent, or study closure, whichever occurs first.
Physical exam (including weight)
ECOG performance status
CT with IV contrast of the chest and a urogram of the abdomen and pelvis. If contrast media are contraindicated, please refer to the current Image Acquisition Guidelines (Section 6.1.12) for preferred scanning and contrast options. Subsequent scans are performed via the same radiologic modalities.
QoL PRO assessment (EQ-5D-5L)
Brain scan (if clinically indicated)
Bone scan (if clinically indicated)
   (ii) Long-term follow-up Assessments

During long term follow up, patients are contacted every 24 weeks (±14 days) for survival status and collection of subsequent anticancer therapy information until death, study closure, or withdrawal of consent, whichever occurs first.

### 6.1.7.8 Appropriateness of Measurements

The safety measures that are used in this trial are considered standard procedures for evaluating the potential adverse effects of study medications.

The determination of antitumor activity will be based on confirmed objective response assessments as defined by RECIST Version 1.1 (Eisenhauer 2009) (see Table 15) (Eisenhauer et al., Eur J Cancer (2009); 45(2): 228-47.) and treatment decisions by the investigator will be based on RECIST Version 1.1. RECIST criteria are considered standard in oncological practice for this type of neoplasm, and the intervals of evaluation in this protocol are appropriate for disease management.

Immunogenicity is commonly assessed for biologics; therefore, standard tests are performed to detect the possible presence of specific antibodies to enfortumab vedotin.

Pharmacokinetic assessments are also common in clinical studies to help characterize dose exposure response relationships.

Exploratory biomarker measurements in peripheral blood samples enable correlation with PK assessments and are common in clinical studies. Assessments conducted on pre-treatment tumor tissue are similarly common. Both peripheral blood and tumor biomarker samples are assessed using commonly employed, standard tests.

RC+PLND with curative intent usually occurs 8 to 12 weeks after MIBC diagnosis, offering a unique opportunity to test new perioperative therapeutic approaches.

Pathologic downstaging (≤pT1) to non-muscle-invasive disease in response to NAC is a well-recognized biomarker of improved OS and would be considered a suitable endpoint for neoadjuvant trials (Sonpavde 2009; Chism 2013).

### 6.1.8 Data Analysis Methods

### 6.1.8.1 Determination of Sample Size

Approximately 20 patients may be enrolled in this study.

The sample size is not based on power calculations for formal hypothesis testing, but is selected based on the precision of the estimate for pCRR as characterized by the 95% CIs.

For a sample size of 20 patients, and assuming a pCRR of 30% or 40%, the 2-sided 95% CIs are summarized below in Table 13.

TABLE 13

| pCRR | 95% Exact CI (n = 20) | 95% Exact CI (n = 50) |
|---|---|---|
| 30% | (12%, 54%) | (18%, 45%) |
| 40% | (19%, 64%) | (26%, 55%) |

### 6.1.8.2 Study Endpoint Definitions

(i) Pathological Complete Response Rate (pCRR)

The pCRR is defined as the proportion of patients having pCR. pCR is defined as the absence of viable tumor (pT0N0) in examined tissue from RC+PLND.

(ii) Pathological Downstaging Rate (pDSR)

pDSR is defined as the proportion of patients having pDS. pDS is defined as patients with <pT2 (includes pT0, pTis, pTa, pT1) and N0 in examined tissue from RC+PLND.

(iii) Disease Free Survival (DFS)

DFS is defined as the time from post-RC baseline scan to the first occurrence of either:
Local or distant recurrence as assessed by CT or MRI and/or biopsy; or
Death due to any cause.

DFS is only be calculated for the patients who are confirmed to be disease free at the post-RC baseline scan.

### (iv) Event-Free Survival (EFS)

EFS is defined as the time from the start of study treatment to the first occurrence of any of the following events:
Radiographic disease progression precluding a curative intent surgery prior to RC+PLND
Failure to undergo RC+PLND for participants with residual muscle-invasive disease and/or any radiographic disease present
Gross residual disease left behind at time of RC+PLND (surgeon unable to complete curative intent surgery due to unresectable tumor or newly discovered metastatic disease)
Local or distant recurrence post-RC as assessed by CT or MRI and/or biopsy. If biopsy is not feasible due to participant safety, CT/MRI alone is sufficient
Death from any cause

NOTE: A non-urothelial second primary malignancy is not considered an event.

EFS data is censored as described below, for the primary analysis of EFS:
Patients who do not refuse surgery and do not have any EFS event are censored at the date of the last disease assessment;
Patients with no disease, with NMIBC or incomplete evaluation who refuse surgery are censored at the last disease assessment prior to refusal of surgery;
Patients with residual MIBC or progressive disease who refuse surgery are counted as events. Patients who refuse surgery but without a post-screening scan are censored at study day 1.

### (v) Overall Survival

OS is defined as the time from start of study treatment to date of death due to any cause. In the absence of confirmation of death, OS is censored at the last date the patient is known to be alive.

### 6.1.8.3 Statistical and Analytical Plans

The statistical and analytical plans presented below summarize the more complete plans to be detailed in the statistical analytical plan (SAP). A change to the data analysis methods described in the protocol requires a protocol amendment only if it alters a principal feature of the protocol. The SAP is finalized prior to database lock. Any changes to the methods described in the final SAP are described and justified in the clinical study report.

### (i) General Considerations

Disease response is summarized based on the pathological tumor response at the time of RC+PLND by central pathology reviews.

In general, descriptive statistics are presented that include the number of observations, mean, standard deviation, median, minimum and maximum for continuous variables, and the number and percentages (of non-missing) per category for categorical variables.

Unless otherwise specified, CI will be calculated at two-sided 95% level.

The two-sided 95% exact CI using Clopper-Pearson methodology (Clopper 1934) is calculated for the response rates where applicable (e.g., ORR, DCR, pCRR, and pDSR).

For time-to-event endpoints, the median survival time is estimated using the Kaplan Meier method; the associated 95% CI is calculated based on the complementary log-log transformation (Collett 1994).

### Data Transformations and Derivations

Time variables based on 2 dates, eg, Start Date and End Date, are calculated as (End Date−Start Date+1) (in days) unless otherwise specified in the planned analysis section.

Unless otherwise specified in the analysis plan, baseline values used in all analyses are the most recent non-missing measurement prior to the first dose of study drug.

### Analysis Sets

The safety analysis set included all patients who receive any amount of study drug. The safety analysis set is used for all safety analyses.

The full analysis set (FAS) includes all patients who are enrolled in the study and received any amount of study drug (enfortumab vedotin or combination agent). The FAS is used for the analysis of efficacy endpoints. Note that the analysis population for pCR includes all patients in the FAS, with the exception of those with no disease who refuse surgery. The analysis population for pDS includes all patients in the FAS who undergo surgery and have a tissue sample examined from RC+PLND. Patient demographics and baseline disease characteristics are summarized based on the FAS.

The efficacy-evaluable analysis set includes all patients in the FAS who had at least 2 post-baseline response assessments or discontinued from treatment for any reason. The efficacy-evaluable analysis set is used for additional analyses of efficacy endpoints.

The PK analysis set includes all patients who received enfortumab vedotin and from whom at least 1 blood sample was collected and assayed for enfortumab vedotin, MMAE, or TAb concentration. Corresponding records of the time of dosing and sample collection must also be available for all enfortumab vedotin, MMAE, and TAb concentration. The PK analysis set is used for analyses of PK parameters.

Additional analysis sets of patients may be defined in the SAP.

### Examination of Subgroups

As exploratory analyses, subgroup analyses may be conducted for selected endpoints.

### Timing of Analyses

The primary analyses are conducted after all treated patients have completed their assigned study interventions and have had adequate follow-up on the study.

Additional cutoff dates may be defined and corresponding database locks may occur to allow for more precise estimates of time-to-event endpoints.

### (ii) Patient Disposition

An accounting of study patients by disposition is tabulated and the number of patients in each analysis set are summarized, by cohorts and overall. Patients who discontinue study treatment and patients who withdraw from the study are summarized with reason for discontinuation or withdrawal for all enrolled patients.

(iii) Patient Characteristics

Demographics and other baseline characteristics are summarized using FAS, by cohorts and overall. Details are provided in the SAP.

### (iv) Efficacy Analyses

The primary analysis of efficacy endpoints is analyzed using the FAS as defined in Section 6.1.8.3. Supplemental analyses of efficacy endpoints may be presented using the efficacy-evaluable analysis sets.

Analyses of pCRR, pDSR, DFS, EFS, and OS are performed.

The key efficacy endpoint of this component of the study is pCRR by central pathology review. The observed pCRR and the 95% CIs is provided using Clopper-Pearson methodology, by cohorts.

pDSR by central pathology review is a secondary endpoint and is analyzed similarly as mentioned above.

Other secondary endpoints, such as DFS by the investigator, EFS by the investigator, and OS, are time-to-event endpoints, and they are analyzed using Kaplan-Meier methodology and Kaplan-Meier plots are provided, by cohorts. Details on the censoring algorithm are provided in the SAP.

### (v) Pharmacokinetic and ATA Analyses

Enfortumab vedotin ADC, MMAE, and TAb concentrations are summarized with descriptive statistics at each PK sampling time point. PK parameters, including, but not limited to AUC, Cmax, and Tmax, are estimated by noncompartmental analyses and summarized by descriptive statistics. The relationship between PK and pharmacodynamics endpoints, safety, or efficacy may be explored.

The incidence of ATA will be summarized by visit and cohort using the safety analysis set.

### (vi) Patient Reported Outcomes Analyses

Completion and compliance rates for each assessment are summarized. PRO analyses is performed on the FAS population. If any additional analysis is planned, it is detailed in a supplemental SAP.

(vii) Biomarker Analyses

Relationships of biomarker parameters (e.g., pre-treatment values, absolute and relative changes from pre-treatment) to efficacy, safety, and PK parameters are explored. Relationships and associated data that are determined to be of interest are summarized. Details of these analyses are described separately.

(viii) Safety Analyses

The safety analysis set will be used to summarize all safety endpoints.

### (a) Extent of Exposure

Duration of treatment, number of cycles, total dose and dose intensity is summarized. Dose modifications are also summarized in similar manner. Details are provided in the SAP.

### (b) Adverse Events

An overview of AEs provide a tabulation, by cohort and overall, of the incidence of all AEs, TEAEs, treatment-related AEs, Grade 3 and higher AEs, SAEs, treatment-related SAEs, deaths, and AEs leading to study treatment discontinuation. AEs are defined as treatment emergent if they are newly occurring or worsen following study treatment.

AEs are listed and summarized by Medical Dictionary for Regulatory Activities (MedDRA), preferred term, severity, and relationship to study drug. In the event of multiple occurrences of the same AE with the same preferred term in 1 patient, the AE are counted once as the occurrence. The incidence of AEs are tabulated by preferred term and cohorts. AEs leading to premature discontinuation of study drug are summarized and listed in the same manner.

### (c) Deaths and Serious Adverse Events

SAEs are listed and summarized in the same manner as all AEs. Events with a fatal outcome are listed.

### (d) Clinical Laboratory Results

Laboratory values (e.g., chemistry, hematology, and urinalysis with reflexive microscopic analysis) may be presented graphically by visit. Summary statistics may be tabulated as appropriate by scheduled visit. Laboratory values are listed with grade per NCI CTCAE Version 4.03 and flagged when values are outside the normal reference range.

### (e) Other Safety Analyses

### Vital Signs

Summary statistics and change from baseline and/or predose to postdose may be tabulated where appropriate.

### ECOG Status

ECOG status is summarized for each visit. Shifts from baseline to the best and worst post-baseline score may be tabulated, by cohort and overall.

### ECG

ECG status (normal, abnormal clinically significant, or abnormal not clinically significant) may be summarized for each scheduled and unscheduled ECG, and shifts from baseline may be tabulated, by cohort and overall.

### (ix) Interim Analyses

No formal interim analysis is planned.

### 6.1.9 List of Abbreviations and Descriptions of Terms

ACS American Cancer Society
ADC antibody-drug conjugate
AE adverse event
AESI adverse event of special interest
ALT alanine aminotransferase
ANC absolute neutrophil count
aPTT activated partial thromboplastin time
ASCO American Society of Clinical Oncology
AST aspartate aminotransferase
ASTRO American Society for Radiation Oncology
ATA antitherapeutic antibodies
AUA American Urological Association
AUC area under the curve
BCG Bacillus Calmette-Guérin
BICR Blinded independent central review
BPI-SF Brief Pain Inventory Short Form
BSA body surface area
carboplatin cis-Diammine (cyclobutane 1,1 dicarboxylato) platinum
CBC complete blood count
cCR clinical complete response
CFR Code of Federal Regulations
CI confidence interval
cisplatin cis-diamminedichloroplatinum (II)
CNS central nervous system
CPI checkpoint inhibitor
CR complete response
CrCl creatinine clearance
CRF case report form
CT computed tomography
CTLA-4 cytotoxic T-lymphocyte-associated protein 4
DE DLT-evaluable
DCR disease control rate
DFS disease free survival
DLT dose-limiting toxicity
DOR duration of response
ECD extracellular domain
ECG electrocardiogram
ECHO transthoracic echocardiogram
ECOG Eastern Cooperative Oncology Group
eCRF electronic case report form
EFS event-free survival
EORTC European Organization for the Research and Treatment of Cancer
EOT end of treatment
EQ-5D EuroQol 5-dimensions
EV Enfortumab vedotin
EV Mono Enfortumab vedotin monotherapy
FAS full analysis set
HbA1c hemoglobin A1c
HIV human immunodeficiency virus
HRU health resource utilization
ICH International Council for Harmonisation
iCPD confirmed progressive disease based on iRECIST guidelines
IEC independent ethics committee
Ig immunoglobulin
IHC immunohistochemistry
imAE immune-mediated adverse event
IND investigational new drug
INR international normalized ratio
IRB institutional review board
iRECIST modified RECIST 1.1 for immune-based therapeutics
IRR infusion-related reaction
iUPD unconfirmed progressive disease (based on iRECIST guidelines)
IV intravenous
LFT liver function test
M-CAVI methotrexate/carboplatin/vinblastine
MDRD Modification of Diet in Renal Disease
MIBC muscle invasive bladder cancer
MMAE monomethyl auristatin E
MRI magnetic resonance imaging
MTD maximum tolerated dose
MVAC methotrexate, vinblastine, doxorubicin, and cisplatin
NAC neoadjuvant chemotherapy
NCI CTCAE National Cancer Institute's Common Terminology Criteria for Adverse Events
NGS Next Generation Sequencing
NMIBC non-muscle invasive bladder cancer
NYHA New York Heart Association
ORR objective response rate
OS overall survival
PBMC peripheral blood mononuclear cell
pCR pathological complete response
pCRR pathological complete response rate
PD progressive disease
PD-1 programmed cell death 1
PD-L1 programmed death-ligand 1
PD-L2 programmed death-ligand 2
pDS pathological downstaging
pDSR pathological downstaging rate
Pembro pembrolizumab
PFS progression-free survival
PFS2 progression-free survival on subsequent therapy
PK pharmacokinetics
PLND pelvic lymph node dissection
PR partial response
PRO patient reported outcomes
PSA prostate specific antigen
PT prothrombin time
PTT partial thromboplastin time
QLQ-C30 Core QoL Assessment
QoL quality of life
Q2W every 2 weeks
Q3W every 3 weeks
RC radical cystectomy
RC+PLND radical cystectomy and pelvic lymph node dissection
RECIST Response Evaluation Criteria in Solid Tumors Version 1.1
SAE serious adverse event
SAP statistical analysis plan
SD stable disease
SDRIFE symmetrical drug-related intertriginous and flexural exanthema
SJS Stevens-Johnson Syndrome
SMC Safety Monitoring Committee
SOC standard of care
SUO Society of Urologic Oncology
TAb total antibody
T1DM Type 1 diabetes mellitus
TEAE treatment-emergent adverse event
TEN toxic epidermal necrolysis
TME tumor microenvironment
TURBT transurethral resection of a bladder tumor
ULN upper limit of normal
USP United States Pharmacopeia

TABLE 14
Schedule of Events

| | | Screening/ | | | | | |
|---|---|---|---|---|---|---|---|
| | | Baseline | | Enrollment | Every 3-Week Cycle | | |
| | | Day | | | | | |
| | | | | Within 1 | | | |
| | | D −28 | D −7 | to 2 d of | | | |
| | | to 1 | to 1 | First Dose | D 1 | D 8 | D 15 |
| | | Visit Window | | | | | |
| | | | | | ±2 d^{C} | +1 d | ±1 d |
| Screening/ | Inclusion/Exclusion, medical history | X | | Submit | | | |
| Baseline | Informed consent | X | | confirmation | | | |
| Assessments | Initiate collection of tumor specimen | X | | prior to | | | |
| | from TURBT^{D,E} | | | treatment | | | |
| | Complete eye exam | X | | | | | |
| | Brain scan if clinically indicated^{F} | X | | | | | |
| | Bone scanif clinically indicated^{F} | X | | | | | |
| | INR/PT/PTT | | X | | | | |
| | Serology for hepatitis B surface antigen and | X | | | | | |
| | antihepatitis B core antibody | | | | | | |
| | Serology for antihepatitis C antibody | X | | | | | |
| | HbA1c^{G} | X | | | | | |
| | Urinalysis with reflexive microscopic | X | | | | | |
| | analysis | | | | | | |
| | Pregnancy test (urine or serum; only for | | X | | X^{J} | | |
| | females of childbearing potential)^{H} | | | | | | |
| Safety | Physical exam (including weight) | | | | X^{I,J} | | |
| Assessments | Height | | X | | | | |
| | Vital signs | | X | | X^{I,J} | X^{J} | |
| | CBC with differential^{L,M} | | X | | X^{I,J} | X^{J} | X |
| | Serum chemistry panel^{L,M} | | X | | X^{I,J,N} | X^{J,N} | X |
| | CrCl | | X | | | | |
| | Thyroid function tests | X | | | X^{I,J} | | |
| | ECOG performance status | | X | | X^{I,J} | | |
| | ECG^{O} | | X | | | | |
| | Transthoracic ECHO^{Z} | | X | | | | |
| | Slit lamp examination | | | | | | |
| | Concomitant medications | Related to study procedures^{Q} | | | For AEs: Collect from Day 1 (predose) | | |
| | Adverse event collection | | | | through RC, through 90 days post last | | |
| | | | | | dose, at early discontinuation, or | | |
| | | | | | through the EOT visit, whichever is later. | | |
| | | | | | From day of RC through the EOT visit, | | |
| | | | | | all related Grade 3 events (study drug) | | |
| | | | | | or related SAEs are to be reported. SAEs | | |
| | | | | | that occur after the safety reporting | | |
| | | | | | period, assessed as related to | | |
| | | | | | investigational agent or RC + PLND are to be | | |
| | | | | | reported. Certain non-serious AEs of | | |
| | | | | | clinical interest (AECI) may be followed | | |
| | | | | | until resolution, return to baseline, | | |
| | | | | | or study closure, whichever is later. | | |
| Treatment^{Y} | Enfortumab vedotin administration^{R} | | | | X | X | |
| PK/Immunogenicity/ | Blood, urine, tissue sample collection | See PK, ATA, and biomarker tables for | | | | | |
| Biomarker | | sample collection details^{A} | | | | | |
| Response Assessment | Imaging^{S} | X | | | | | |
| | Pathologic assessment at time of RC | | | | | | |
| | Survival status^{T} | | | | | | |
| Other Assessments | Patient Exit Interviews | | | | | | |

| | | | | | | Long-term |
|---|---|---|---|---|---|---|
| | | Every 3-Week | | | | Follow- |
| | | Cycle | RC | EOT | Follow-up^{W} | up^{X} |
| | | Day | | | | |
| | | | | | Every 12 weeks in | |
| | | | | | the first 2 years, | |
| | | | | 30-37 Days | and subsequently | Every 24 |
| | | Pre-RC | | Post-RC^{B} | every 24 weeks | Weeks |
| | | Visit Window | | | | |
| | | ≤4 wk prior | | | | |
| | | to RC | | | ±14 d | ±14 d |
| Screening/ | Inclusion/Exclusion, medical history | | | | | |
| Baseline | Informed consent | | | | | |
| Assessments | Initiate collection of tumor specimen | | | | | |
| | from TURBTD^{E} | | | | | |
| | Complete eye exam | | | | | |
| | Brain scan if clinically indicated^{F} | X | | | X | |
| | Bone scanif clinically indicated^{F} | X | | | X | |
| | INR/PT/PTT | | | | | |
| | Serology for hepatitis B surface antigen and | | | | | |
| | antihepatitis B core antibody | | | | | |
| | Serology for antihepatitis C antibody | | | | | |
| | HbA1c^{G} | | | | | |
| | Urinalysis with reflexive microscopic | | | | | |
| | analysis | | | | | |
| | Pregnancy test (urine or serum; only for | | | X | | |
| | females of childbearing potential)^{H} | | | | | |
| Safety | Physical exam (including weight) | | | X | X^{K} | |
| Assessments | Height | | | | | |
| | Vital signs | | | X | | |
| | CBC with differential^{L,M} | | | X | | |
| | Serum chemistry panel^{L,M} | | | X | | |
| | CrCl | | | | | |
| | Thyroid function tests | | | X | | |
| | ECOG performance status | | | X | X | |
| | ECG^{O} | | | X | | |
| | Transthoracic ECHO^{Z} | | | | | |
| | Slit lamp examination | | | X^{P} | | |
| | Concomitant medications | For AEs: Collect from Day 1 (predose) | | | | |
| | Adverse event collection | through RC, through 90 days post last | | | | |
| | | dose, at early discontinuation, or | | | | |
| | | through the EOT visit, whichever is later. | | | | |
| | | From day of RC through the EOT visit, | | | | |
| | | all related Grade 3 events (study drug) | | | | |
| | | or related SAEs are to be reported. SAEs | | | | |
| | | that occur after the safety reporting | | | | |
| | | period, assessed as related to | | | | |
| | | investigational agent or RC + PLND are to be | | | | |
| | | reported. Certain non-serious AEs of | | | | |
| | | clinical interest (AECI) may be followed | | | | |
| | | until resolution, return to baseline, | | | | |
| | | or study closure, whichever is later. | | | | |
| Treatment^{Y} | Enfortumab vedotin administration^{R} | | | | | |
| PK/Immunogenicity/ | Blood, urine, tissue sample collection | See PK, ATA, and biomarker tables for | | | | |
| Biomarker | | sample collection details^{A} | | | | |
| Response Assessment | Imaging^{S} | X^{S} | | X^{AA} | X^{S} | |
| | Pathologic assessment at time of RC | | X | | | |
| | Survival status^{T} | | | | | X^{T} |
| Other Assessments | Patient Exit Interviews | X^{U} | | X^{V} | | |

^{A}See Table 19 for PK, ATA, and biomarker sampling.
^{B}EOT visits should occur within 30-37 days after patient completes all required study intervention (study intervention includes RC + PLND) or discontinues prior to completion of all required study intervention. EOT evaluations must be performed before initiation of a new therapy for cancer, with the exception of the slit lamp examination (slit lamp examinations must be performed ≥4 weeks after last dose). If EOT evaluations are completed before 30 days after last study intervention, the patient will be contacted by telephone 30 to 37 days following the last date of study intervention to complete safety follow up and assess for AEs.
^{C}Window applies to Cycle 2+.
^{D}Within 90 days before Cycle 1 Day 1. This tissue will also be sent for PD-L1 determination by immunohistochemistry (IHC).
^{E}Invasive tumor tissue obtained at the time of the patient's original diagnosis for biomarker studies. Samples must be available for participation in the study. Tumor block is preferred. If tumor block is not available, slides are acceptable.
^{F}Repeated as clinically indicated throughout the study.
^{G}If HbA1c is elevated (≥6.5%), refer patient to appropriate provider during Cycle 1 for glucose management.
^{H}Either serum or urine pregnancy test for females of childbearing potential. Baseline assessment Day −3 to Day 1. Either serum or urine pregnancy test. Not required for Cycle 1 if baseline assessment performed within 7 days. Repeat every month (±1 week) for 6 months after EOT. The pregnancy test may be performed within 1 day prior to administration of study drug.
^{I}Not required for Cycle 1 if baseline assessments performed within 1 day.
^{J}Procedures should be conducted pre-dose. Day 8 vital signs will not be collected for patients who discontinue treatment with enfortumab vedotin permanently.
^{K}Follow-up physical exam will not include weight measurement.
^{L}Abnormal lab values that are clinically significant will be confirmed within 1 day during Cycle 1, as clinically indicated.
^{M}Safety labs may be collected up to 1 day prior to dosing.
^{N}Verify blood glucose is <250 mg/dL prior to dosing. Patients with diabetes must be tested in the clinic and blood glucose must be <250 mg/dL prior to dosing. Use of insulin is permitted as part of the standard of care.
^{O}Standard Baseline/EOT ECG assessments will be performed for all patients.
^{P}Only required for patients who experience corneal AEs during the study; must be performed ≥4 weeks after last dose.
^{Q}From time of informed consent.
^{R}At least 1 week must elapse between doses of enfortumab vedotin.
^{S}Baseline radiographic screening assessment will be done using CT with IV contrast of the chest and a CT urogram of the abdomen and pelvis. If contrast media are contraindicated, please refer to the current Image Acquisition Guidelines (Section 6.1.12) for preferred scanning and contrast options. Restaging will be done via the same radiologic modalities ≤4 weeks prior to RC. Pre-RC scans should be reviewed prior to RC + PLND. RC + PLND with curative intent must occur between 4 to 12 weeks post last dose of study treatment. Subsequent scans should be performed every 12 weeks (±14 days) (based on date of RC + PLND) for the first 2 years and then every 24 weeks (±14 days) until disease progression/recurrence, patient death, withdrawal of consent, or study closure, whichever occurs first.
^{T}Contact patient for survival status, disease status, and collection of subsequent anticancer treatment information until death, study closure, or patient withdraws consent, whichever occurs first.
^{U}Interview (optional) will be conducted when the patient finishes the last cycle but before surgery.
^{V}Interview (optional) to be conducted at EOT or time of early discontinuation, whichever is earlier.
^{W}Follow-up will be done every 12 weeks (±14 days) in the first 2 years (based on date of RC + PLND) until disease progression/recurrence, patient death, study closure, or withdrawal of consent, whichever occurs first. After 2 years on study, the frequency of visits including response assessments will be reduced to every 24 weeks (±14 days). Patients will be contacted every 24 weeks (±14 days) after RC + PLND for survival status and collection of subsequent anticancer therapy information until death, study closure, or withdrawal of consent, whichever occurs first.
^{X}Patients will be contacted every 24 weeks (±14 days) after RC + PLND for survival status and collection of subsequent anticancer therapy information until death, study closure, or withdrawal of consent, whichever occurs first.
^{Y}Review the results of all local laboratory tests prior to study drug administration in order to determine whether to proceed with dosing or whether dose modification is required. If there are AEs preventing dosing on Day 1 of any Cycle (Cycle 2+), dosing should be delayed and not eliminated. Unless a drug is permanently discontinued, if one drug is held all need to be held. If there are AEs that do not resolve within 2 days of planned dose for Day 8 of any cycle then that dose should be eliminated. If Day 8 dose is eliminated, PK samples should still be drawn (for the applicable cycles). Day 8 vital signs will not be collected for patients who discontinue treatment with enfortumab vedotin permanently.
^{Z}A transthoracic echocardiogram is required for subjects with NYHA Class III heart failure or a history of coronary heart disease, arrhythmia or other significant heart disease.
^{AA}EOT Imaging for patients that do not enter follow up after RC + PLND (but enter long term follow up) should be done within 4 weeks of the last study intervention. If EOT Imaging is completed, subsequent scans should be based on the protocol-specified imaging schedule.

TABLE 15
RECIST Criteria Summary (Version 1.1)
Response Evaluation Criteria in Solid Tumors

| Term | Definition |
|---|---|
| Complete response (CR) | Disappearance of all target lesions. Any pathological lymph nodes |
| | must have reduction in short axis to <10 mm. |
| Partial response (PR) | A ≥30% decrease in the sum of diameters of target lesions, taking |
| | as reference the baseline sum diameters. |
| Progressive disease (PD) | At least a 20% increase in the sum of diameters of target lesions, |
| | taking as reference the smallest sum of diameters on study (this |
| | includes the baseline sum if that is the smallest on study). In |
| | addition to the relative increase of 20%, the sum must also |
| | demonstrate an absolute increase of at least 5 mm. The appearance |
| | of one or more new lesions is also considered progression. |
| Stable disease (SD) | Neither sufficient shrinkage to qualify for PR nor sufficient |
| | increase to qualify for PD, taking as reference the smallest sum of |
| | diameters while on study. |
| Measurable lesion(s) | Must be accurately measured in at least 1 dimension (longest |
| | diameter in the plane of measurement is to be recorded) with a |
| | minimum size of 10 mm by CT/MRI scan (CT slice thickness no |
| | greater than 5 mm) (maximum of 5 lesions, no more than 2 per |
| | site, at least 10 mm in long axis [or 15 mm in short axis for nodal |
| | lesions]). |
| | A lymph node must be ≥15 mm in short axis when assessed by |
| | CT/MRI scan to be considered measurable. |

From RECIST Version 1.1 (Eisenhauer 2009).

A response (CR or PR) is considered confirmed if the following disease assessment (at least 4-5 weeks after the initial response) still shows response (CR or PR). In cases where the initial response is followed by SD, it is considered as confirmed if the SD is later followed by PR or CR. For example, if a patient had PR in Week 9, SD in Week 14, and PR in Week 18, this PR is considered as confirmed.

### Baseline Lesion Identification

When more than one measurable lesion is present at baseline all lesions up to a maximum of five lesions total (and a maximum of two lesions per organ) representative of all involved organs should be identified as target lesions and is recorded and measured at baseline. Target lesions should be selected on the basis of their size (lesions with the longest diameter), be representative of all involved but in addition should be those that lend themselves to reproducible repeated measurements (bone lesions should not be selected as a target lesion). It may be the case that, on occasion, the largest lesion does not lend itself to reproducible measurement in which circumstance the next largest lesion which can be measured reproducibly should be selected. Lesions in the urinary bladder that are detected on CT/MRI scans may be selected as target lesions, provided the radiologist considers the lesion(s) lend to reproducible measurements at post-baseline timepoints and lesions have been measured with adequate bladder distension. The same imaging modality should be used throughout the study treatment.

Pathological lymph nodes which are defined as measurable and may be identified as target lesions must meet the criterion of a short axis of ≥15 mm by CT or MRI scan. All other pathological nodes (those with short axis ≥10 mm but <15 mm) should be considered nontarget lesions. Lymph nodes that have a short axis <10 mm are considered non-pathological and should not be recorded or followed. All other lesions (or sites of disease) including pathological lymph nodes should be identified as nontarget lesions and should also be recorded at baseline.

TABLE 16
Performance Status Scales Conversion

| Karnofsky | | Lansky | | ECOG | |
|---|---|---|---|---|---|
| Percent | Description | Percent | Description | Score | Description |
| 100 | Normal, no complaints, | 100 | Fully active, | 0 | Normal activity. Fully |
| | no evidence of disease. | | normal. | | active, able to carry on |
| 90 | Able to carry on normal | 90 | Minor restrictions in | | all pre-disease |
| | activity; minor signs or | | physically strenuous | | performance without |
| | symptoms of disease. | | activity. | | restriction. |
| 80 | Normal activity with | 80 | Active, but | 1 | Symptoms, but |
| | effort; some signs or | | tires more | | ambulatory. Restricted |
| | symptoms of disease. | | quickly. | | in physically strenuous |
| 70 | Cares for self, unable to | 70 | Both greater restriction | | activity, but ambulatory |
| | carry on normal activity | | of, and less time spent | | and able to carry out |
| | or to do active work. | | in, play activity. | | work of a light or |
| | | | | | sedentary nature (eg, |
| | | | | | light housework, office |
| | | | | | work). |
| 60 | Requires occasional | 60 | Up and around, but | 2 | In bed <50% of the |
| | assistance, but is able to | | minimal active play; | | time. Ambulatory and |
| | care for most of his/her | | keeps busy with quieter | | capable of all self-care, |
| | needs. | | activities. | | but unable to carry out |
| 50 | Requires considerable | 50 | Gets dressed, but lies | | any work activities. Up |
| | assistance and frequent | | around much of the | | and about more than |
| | medical care. | | day; no active play; | | 50% of waking hours. |
| | | | able to participate in all | | |
| | | | quiet active play and | | |
| | | | activities. | | |
| 40 | Disabled, requires | 40 | Mostly in bed, | 3 | In bed >50% of the |
| | special care and | | participates in quiet | | time. Capable of only |
| | assistance. | | activities. | | limited self-care, |
| 30 | Severely disabled, | 30 | In bed, needs assistance | | confined to bed or chair |
| | hospitalization | | even for quiet play. | | more than 50% of |
| | indicated. Death not | | | | waking hours. |
| | imminent. | | | | |
| 20 | Very sick, | 20 | Often sleeping, | 4 | 100% bedridden. |
| | hospitalization | | play entirely limited | | Completely disabled. |
| | indicated. Death not | | to very passive | | Cannot carry on any |
| | imminent. | | activities. | | self-care. Totally |
| 10 | Moribund, fatal | 10 | No play, does not get | | confined to bed or chair. |
| | processes progressing | | out of bed. | | |
| | rapidly. | | | | |
| 0 | Dead. | 0 | Dead. | 5 | Dead. |

### 6.1.10 New York Heart Association Classification

A Functional and Therapeutic Classification for Prescription of Physical Activity for Cardiac Patients
Class I: patients with no limitation of activities; they suffer no symptoms from ordinary activities.
Class II: patients with slight, mild limitation of activity; they are comfortable with rest or with mild exertion.
Class III: patients with marked limitation of activity; they are comfortable only at rest.
Class IV: patients who should be at complete rest, confined to bed or chair; any physical activity brings on discomfort and symptoms occur at rest.

On-line source: On-line source: http://www.heart.org/HEARTORG/Conditions/HeartFailure/AboutHeartFailure/Classes-of-Heart-Failure UCM_306328_Article.jsp

### 6.1.11 Liver Safety Monitoring and Assessment

The following recommendations are from the Food and Drug Administration (FDA) Guidance for Industry titled “Drug-Induced Liver Injury: Premarketing Clinical Evaluation” issued July 2009.

Any patient with an increase of serum aminotransferases to >3×upper limit of normal (ULN) or bilirubin >2×ULN should undergo detailed testing for liver enzymes (including at least alanine aminotransferase (ALT), aspartate aminotransferase (AST), alkaline phosphatase (ALP), and total bilirubin (TBL)). To confirm the abnormality, testing should be repeated within 72 hours of notification of the test results.

### Description of Liver Abnormalities:

Confirmed liver abnormalities will be characterized as Moderate and Severe:

### Moderate:

ALT or AST>3×ULN OR Total Bilirubin >2×ULN

### Severe:

ALT or AST>3×ULN AND Total Bilirubin >2×ULN (*See description of Hy's Law below)
ALT or AST>8×ULN
ALT or AST>5×ULN for more than 2 weeks
ALT or AST>3×ULN and International Normalized Ratio (INR)>1.5 (if INR testing is applicable/evaluated)
ALT or AST>3×ULN with the appearance of symptoms suggestive of liver injury (e.g., right upper quadrant pain or tenderness) and/or eosinophilia (>5%)

The investigator may determine that abnormal liver function results, other than as described above, may qualify as moderate or severe abnormalities and require additional monitoring and follow-up.

*Hy's Law: Drug-induced jaundice caused by hepatocellular injury, without a significant obstructive component, has a high rate of bad outcomes, from 10 to 50% mortality (or transplant). The 2 “requirements” for Hy's Law are: 1) Evidence that a drug can cause hepatocellular-type injury, generally shown by an increase in transaminase elevations higher 3×ULN (“2×ULN elevations are too common in treated and untreated patients to be discriminating”). 2) Cases of increased bilirubin (at least 2×ULN) with concurrent transaminase elevations at least 3×ULN and no evidence of intra- or extra-hepatic bilirubin obstruction (elevated ALP) or Gilbert's syndrome (Temple et al., Pharmacoepidemiol Drug Saf (2006); 15(4): 241-3.).

### Follow-Up Procedures

Confirmed moderate and severe abnormalities in hepatic functions should be thoroughly characterized by obtaining appropriate expert consultations, detailed pertinent history, physical examination and laboratory tests. Patients with confirmed abnormal liver function testing should be followed as described below.

Confirmed moderately abnormal liver function tests (LFTs) should be repeated 2 to 3 times weekly then weekly or less if abnormalities stabilize or the study drug has been discontinued and the patient is asymptomatic.

Severe hepatic liver function abnormalities as defined above, in the absence of another etiology, may be considered an important medical event and may be reported as a SAE. The sponsor should be contacted and informed of all patients for whom severe hepatic liver function abnormalities possibly attributable to study drug are observed.

To further assess abnormal hepatic laboratory finding, it is recommended that the investigator:
Obtain a more detailed history of symptoms and prior or concurrent diseases. Illnesses and conditions such as hypotensive events, and decompensated cardiac disease that may lead to secondary liver abnormalities should be noted. Nonalcoholic steatohepatitis is seen in obese hyperlipoproteinemic and/or diabetic patients, and may be associated with fluctuating aminotransferase levels.
Obtain a history of concomitant drug use (including nonprescription medication, complementary and alternative medications), alcohol use, recreational drug use and special diets
Obtain a history of exposure to environmental chemical agents.
Based on the patient's history, other testing may be appropriate including:
   Acute viral hepatitis (A, B, C, D, E or other infectious agents),
   Ultrasound or other imaging to assess biliary tract disease,
   Other laboratory tests including INR, direct bilirubin.
Consider gastroenterology or hepatology consultations.

Conduct additional testing as determined by the investigator to further evaluate possible etiology. See Section 6.1.6.2(iii) for treatment discontinuation recommendations related to hepatic safety.

### 6.1.12 Image Acquisition Guidelines

### Screening Scans in Decreasing Order of Preference of the Following:

1. CT Chest and CT urogram of abdomen and pelvis with IV contrast
2. CT Chest without IV contrast and MRI urogram of abdomen and pelvis with IV gadolinium contrast (if iodinated contrast is medically contraindicated)

### Post-Cystectomy Scans in Decreasing Order of Preference of the Following:

1. CT Chest-Abdomen-Pelvis with oral and IV contrast
2. CT Chest without IV contrast and MRI Abdomen-Pelvis with IV gadolinium contrast (if iodinated contrast is medically contraindicated)
3. CT chest without IV contrast and MRI Abdomen-Pelvis without IV gadolinium contrast (if iodinated contrast and gadolinium contrast medically contraindicated)
4. MRI Chest-Abdomen and Pelvis with IV gadolinium contrast
5. Chest-Abdomen-Pelvis CT without IV contrast (oral contrast is recommended) (if patient has contraindication to iodinated contrast and MRI scan)

### Brain Scan (if Clinically Indicated) in Decreasing Order of Preference of the Following:

1. Brain MRI with IV gadolinium
2. Brain CT with IV contrast (if gadolinium and/or MRI is medically contraindicated)
3. Brain MRI without IV gadolinium (if gadolinium is medically contraindicated)
4. Brain CT without IV contrast (if gadolinium is medically contraindicated)

Imaging modality, anatomical coverage, and acquisition parameters should remain consistent across all imaging visits for each patient.

### 6.1.13 Surgery

### Surgical Guidelines:

Surgical approach for RC+PLND (e.g., open, laparoscopic with robot-assistance) and urinary diversion (e.g., ileal conduit, neobladder) is per discretion of the treating urologist and the participant.

RC+PLND are done in accordance with the AUA/ASCO/ASTRO/SUO guidelines*, as follows:

“When performing a standard radical cystectomy, clinicians should remove the bladder, prostate, and seminal vesicles in males and should remove the bladder, uterus, fallopian tubes, ovaries, and anterior vaginal wall in females. When performing bilateral pelvic lymphadenectomy, clinicians should remove, at a minimum, the external and internal iliac and obturator lymph nodes (standard lymphadenectomy).” In the case of neobladder reconstruction, sparing of female sex organs may be allowed based on standard of care and the treating urologist's judgment. Any deviation from this guidance should be documented in the medical record and eCRFs.

All postoperative complications are reported and graded, if applicable, in the same way as other AEs (CTCAE version 4.0) and are identified by the investigator and/or surgeon as post-surgical complications.

*https://www.auanet.org/guidelines/bladder-cancer-non-metastatic-muscle-invasive-(2017).

### 6.1.14 Guidance on Contraception

For the purposes of this guidance, complete abstinence, if consistent with the patient's preferred lifestyle, is an acceptable form of contraception. Complete abstinence is defined as abstinence starting from the time of informed consent and continuing throughout the study and until the end of systemic exposure (at least 6 months after the final dose of study drug).

### Acceptable Methods for Highly Effective Birth Control (Preventing Conception)

Patients who are of childbearing potentiala or whose partners are of childbearing potentiala and who are sexually active in a way that could lead to pregnancy may choose any TWO of the following methods (please see acceptable combinations below):
Hormonal methods of contraception (excluding progestin-only pills; method must be associated with inhibition of ovulation), unless contraindicated
Intrauterine device with failure rate <1%
Tubal ligation
Vasectomy (at least 90 days from the date of surgery with a semen analysis documenting azoospermia)
Barrier method (male or female condom with or without spermicide, cervical cap with or without spermicide, diaphragm with or without spermicide)
A person of childbearing potential is defined as anyone born female who has experienced menarche and who has not undergone surgical sterilization (eg, hysterectomy, bilateral salpingectomy, bilateral oophorectomy) or has not completed menopause. Menopause is defined clinically as 12 months of amenorrhea in a person born female over age 45 in the absence of other biological, physiological, or pharmacological causes.
A barrier method should only be used with a highly effective birth control method that is not a barrier method. Barrier methods alone, including a double-barrier method, are not considered highly effective contraceptive measures (see unacceptable methods of contraception).

Acceptable combinations of contraceptive methods include hormonal method and vasectomy, hormonal method and barrier method, intrauterine device and vasectomy, intrauterine device and barrier method, tubal ligation and vasectomy, and tubal ligation and barrier method.

Acceptable methods for preventing secondary exposure to seminal fluid are as follows: option 1—male condom (with or without spermicide) and cervical cap and option 2—male condom (with or without spermicide) and diaphragm. Subjects born male and who are sexually active with a pregnant or breastfeeding person must use the contraceptives in Options 1 or 2.

Unacceptable methods of contraception include periodic abstinence, spermicide only, no method, progestin-only pills, withdrawal, concomitant use of female and male condoms, rhythm, and barrier methods alone (including double-barrier methods).

### 6.2 Example 2—Antitumor Activity of Neoadjuvant Enfortumab Vedotin (EV) Monotherapy in Cisplatin-Ineligible Patients with Muscle Invasive Urothelial Cancer (MIUC) in a Phase 1B/2 Trial (Cohort H)

### 6.2.1 Background

Up to 25% of all patients diagnosed with urothelial cancer present with muscle-invasive disease, for whom the risk of progression or metastasis is substantial. Neoadjuvant chemotherapy prior to radical cystectomy and pelvic lymph node dissection (RC+PLND) has been shown to prolong overall survival for patients who are cisplatin (cis) eligible. The standard of care (SOC) for cis-ineligible patients undergoing surgery does not include neoadjuvant therapy. Therefore, safe, and effective neoadjuvant therapies are an unmet need for cis-ineligible patients with Muscle Invasive Urothelial Cancer (MIBC). Enfortumab vedotin (EV) is an antibody-drug conjugate directed to Nectin-4, which is highly expressed in urothelial cancer, and has been shown to benefit locally advanced or metastatic urothelial cancer patients in Phase II and III trials, including cis-ineligible patients.

### 6.2.2 Methods

The methods for this Section 6.2 (Example 2) were as described in Section 6.1 (Example 1). Briefly, as described in Section 6.1 (Example 1), patients with cis-ineligible cT2-T4aN0M0 MIBC who were eligible for RC+PLND and had an ECOG of 0-2 were enrolled in the phase 1b/2 trial. Patients received 3 cycles of neoadjuvant EV (1.25 mg/kg) on Days 1 and 8 of every 3-week cycle prior to RC+PLND. The primary endpoint of the study was pathological complete response rate (pCRR; ypT0N0) by central review. Key secondary endpoints included pathological downstaging (pDS) rate (yp T0, Tis, Ta, T1,N0) and safety.

### 6.2.3 Results

The results are from a preliminary analysis of 22 patients. The disposition of subjects in the study is shown in Table 17.

TABLE 17
Disposition of subjects^{a}

| | EV Mono (N = 22) | |
|---|---|---|
| | N (%) | |
| Number of subjects that completed RC + PLND | 21 | (95.5) |
| Number of subjects on study | 19 | (86) |
| Subjects in follow up | 14 | (64) |
| Subjects in long term follow up^{b} | 5 | (23) |
| Number of subjects off study | 3 | (14) |
| Reason for study discontinuation | | |
| Death | 3 | (14) |

^{a}Study treatment included enfortumab vedotin and/or RC + PLND.
^{b}One (1) subject had a partial cystectomy.
c. Subjects who have disease recurrence or progression or started subsequent anti-cancer therapy.

The patient demographics are shown in Table 18.

TABLE 18
Demographics

EV Mono (N = 22)
n (%)

Age Group

| | | |
|---|---|---|
| <65 years | 1 | (5) |
| ≥65 and <75 years | 10 | (45) |
| ≥75 years | 11 | (50) |

Sex

| | | |
|---|---|---|
| Female | 2 | (9) |
| Male | 20 | (91) |

ECOG performance status

| | | |
|---|---|---|
| 0 | 13 | (59) |
| 1 | 8 | (36) |
| 2 | 1 | (5) |
| BMI ≥30 kg/m² | 6 | (27) |
| HbA1c ≥6.5% | 1 | (5) |

| | |
|---|---|
| Hemoglobin <10 g/dL | 0 |

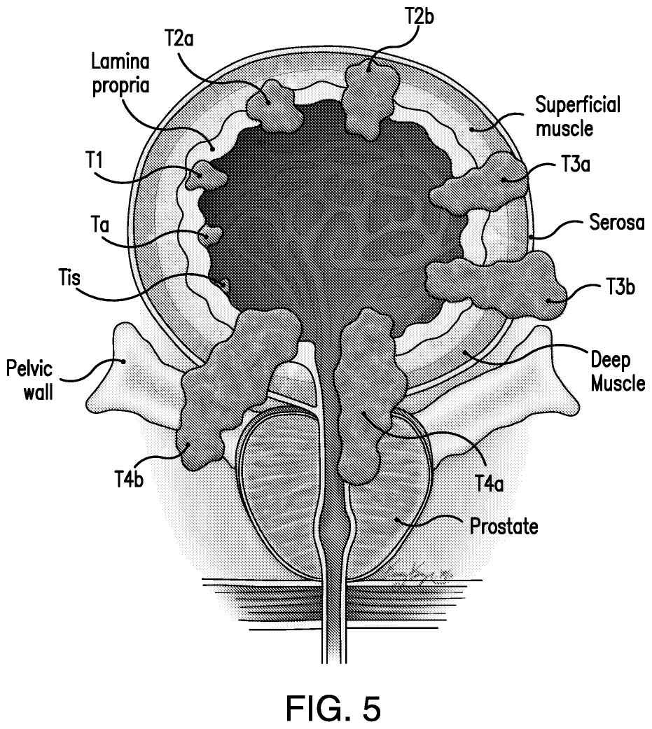

Disease characteristics of patients at baseline are shown in Table 19. In particular, patients had T2 (68.2%), T3 (27.3%), and T4 (4.5%) staging. 68.2% of patients had predominant urothelial cancer and 31.8% of patients had a mixed histology. FIG. 5 depicts the MIUC disease stages (shown in FIG. 5 are Tᵢₛ, Tₐ, T1, T2a, T2b, T3a, T3b, T4a, and T4b) relative to the anatomy of the bladder and pelvic region (See, e.g., www.cancer.org/cancer/bladder-cancer/detection-diagnosis-staging/staging.html, which describes the MIUC disease stages).

TABLE 19
Disease characteristics at baseline

EV Mono (N = 22)
n (%)

Extent of disease

| | | |
|---|---|---|
| Multifocal (multiple lesions) | 8 | (36) |
| Unifocal (single lesion) | 14 | (64) |

Clinical stage^{a}

| | | |
|---|---|---|
| T2 | 13 | (59) |
| T2a | 1 | (4.5) |
| T2b | 1 | (4.5) |
| T3a | 2 | (9) |
| T3b | 4 | (18) |
| T4a | 1 | (4.5) |

Histology

| | | |
|---|---|---|
| Transitional Cell Carcinoma (TCC) | 15 | (68) |
| TCC + squamous | 3 | (14) |
| TCC + micropapillary | 2 | (9) |
| TCC + adenocarcinoma | 1 | (4.5) |
| TCC + sarcomatoid | 1 | (4.5) |

^{a}Subjects summarized using the most recent stage prior to study

A summary of the study drug exposure in patients is provided in Table 20. In particular, 19 patients completed all 3 cycles of EV. 21 patients underwent RC+PLND and 1 patient had a partial cystectomy.

TABLE 20
Study Drug Exposure
Duration of neoadjuvant treatment^{a} (months)

| | |
|---|---|
| Median | 2.07 |
| Min, Max | 0.7, 2.3 |

Number of cycles^{b}

| | |
|---|---|
| Subjects completing 3 EV cycles | 19 |
| Subjects completing 2 EV cycles | 1 |
| Subjects completing 1 EV cycle | 2 |

Time from end of neoadjuvant EV to RC + PLND^{c} (weeks)

| | |
|---|---|
| Median | 8 |
| Min, Max | 4.1, 11.6 |

^{a}Duration of treatment is the time from the first dose of study drug to the earliest of the following: Day 21 of the last treatment cycle, date of death, start of subsequent anti-cancer therapy, or analysis data Snapshot date if the subject is still on treatment at the time of the analysis.
^{b}Cycle with any amount (>0) of enfortumab vedotin or combination agent(s) received.
^{c}Duration from the end of neoadjuvant EV to surgery is the time from the last dose of neoadjuvant EV to the date of surgery.

Results relating to the pathological complete response (pCR) and pathological downstaging (pDS) are as follows. In particular, 36% of patients had a pathological complete response (pCR). Pathological downstaging (pDS) (defined as presence of ypT0, ypTis, ypTa, and ypT1 and N0) was observed in 50% patients (n=11).

In this study, residual disease is defined as subjects with residual muscle invasive UC in the surgical specimens (RC+PLND). Patients with residual disease are summarized in Table 21.

TABLE 21
Summary of Patients with Residual Disease

| Residual Disease* | N(%) | |
|---|---|---|
| ypT2N0 | 3 | (14%) |
| ypT3-4N0 | 2 | (9%) |
| ypTany N+ | 6 | (27%) |

Pathological Response Rates With other Treatments in MIUC are summarized in Table 22.

TABLE 22
Summary of Pathological Response Rates With other Treatments in MIUC

| | | | | | pDS |
|---|---|---|---|---|---|
| | | | | PCR | (non-muscle |
| | | | | (no viable | invasive |
| | Study | | Sample | tumor at | disease at |
| Trial | Population | Regimen | Size | surgery) | surgery) |
| Present study | Cisplatin | EV | 22 | 32% (ypT0 | 50% (includes |
| | ineligible | monotherapy × | | only) | pCR) |
| | | 3 cycles | | 45.5% (ypT0 | |
| | | | | and ypTis) | |
| Retrospective | Cisplatin | Gem + Cis × 4 | 42 | 26% | 36% |
| (Dash et al | eligible | cycles | | | |
| 2008) | | | | | |
| MVAC vs RC | Cisplatin | MVAC | 153 | 38% | |
| (Grossman, | eligible | | | | |
| 2003) | | | | | |
| Meta-analysis | Cisplatin | GC | 1067 | 25.7% | 44.8% |
| (Yin et al 2016) | eligible | | | | |
| Meta-analysis | Cisplatin | MVAC | 667 | 24.3% | 46.5% |
| (Yin et al 2016) | eligible | | | | |
| PURE-01 | Cisplatin | Pembrolizumab × 3 | 50 | 42% | 54% (includes |
| Study | eligible | | | | pCR) |
| | (n = 42) and | | | | |
| | ineligible (n = 8) | | | | |
| ABACUS | Cisplatin | Atezolizumab × 2 | 88 | 31% (ypT0 + | |
| | ineligible | | | ypTis) | |
| BLASST-1 | Cisplatin | Nivo + Gem + | 41 | 49% | 66% |
| | eligible | Cis × 4 | | | |

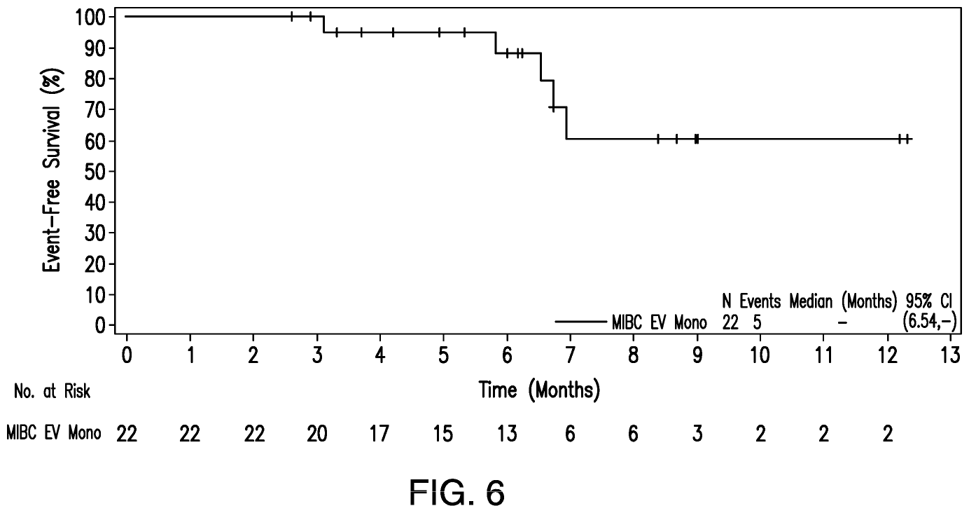

The event-free survival (EFS) observed in the clinical study described herein is shown in FIG. 6. The EFS median was not reached, as shown in FIG. 6. The results from the data shown in FIG. 6 are summarized in Table 23.

TABLE 23
Event-Free Survival (EFS) by Investigator Assessment

| | EV Mono (N = 22) | |
|---|---|---|
| Number of subjects who have recurrence or died^{a} | 5 | (22.7) |
| Event free-survival (EFS) (months) | | |
| Median (95% CI) | — | (6.54, —) |
| 25th, 75th percentile | 6.7, — | |
| Observed min, max | 2.60, 12.32+ | |
| EFS Rate (%) at | | |
| 6 months (95% CI) | 88.2 | (59.86, 96.98) |
| 12 months (95% CI)^{b} | 60.5 | (27.96, 82.01) |

^{a}One PD due to radiographic image scan, 1 PD due to both biopsy and radiographic image scan and 3 deaths.
^{b}One subject was censored at study day 190 (92 days earlier) due to a data issue entered at response assessment at Post-cystectomy Month 6 visit. 12 months EFS rate will be updated as 63.2(31.69, 83.21) after data correction.

The safety of treatment of MIUC patients with EV monotherapy and RC+PLND was also assessed. Table 24 summarizes the observance of treatment emergent adverse events (TEAE's) in the study. In addition, 59.1% patients had Grade 3 or higher TEA's.

TABLE 24
Overview of Treatment Emergent Adverse Events (TEAE's)

| | EV Mono (N = 22) | |
|---|---|---|
| | n (%) | |
| Any TEAEs | 22 | (100) |
| EV-related TEAEs | 22 | (100) |
| RC-related TEAEs | 7 | (32) |
| Any serious TEAEs | 7 | (32) |
| EV-related serious TEAEs | 3 | (13.6) |
| RC-related serious TEAEs | 2 | (9) |

Table 25 summarizes the observance of TEAE's leading to EV discontinuation, bladder surgery not performed, and death.

TABLE 25
Overview of Treatment Emergent Adverse Events (TEAE's)

| | EV Mono | |
|---|---|---|
| | (N = 22) | |
| | n (%) | |
| Any TEAEs leading to EV discontinuation | 3 | (14) |
| EV related TEAEs leading to EV discontinuation | 3 | (14) |
| Any TEAEs leading to bladder surgery not performed | 0 | |
| EV-related TEAEs leading to bladder surgery not performed | 0 | |
| Any TEAEs leading to death^{a} | 1 | (4.5) |

| | |
|---|---|
| EV-related TEAEs leading to death | 0 |
| RC-related TEAEs leading to death | 0 |

^{a}Subjects' deaths occurred after RC + PLND from sudden cardiac arrest not related to EV or RC + PLND.

Table 26 lists specific TEAE's observed in patients in the study.

TABLE 26
TEAE's Observed in Patients in the Study

| | EV Mono (N = 22) | |
|---|---|---|
| Preferred Term | n (%) | |
| Overall | 22 | (100) |
| Fatigue | 12 | (55) |
| Diarrhoea | 9 | (41) |
| Nausea | 8 | (36) |
| Alopecia | 7 | (32) |
| Dysgeusia | 7 | (32) |
| Urinary Tract Infection | 7 | (32) |
| Anemia | 6 | (27) |
| Hypomagnesaemia | 6 | (27) |
| Abdominal pain | 5 | (23) |
| Rash maculo-papular | 5 | (23) |

Table 27 lists specific TEAE's observed in patients in the study.

TABLE 27
TEAE's Observed in Patients in the Study

| | EV Mono (N = 22) | |
|---|---|---|
| Preferred Term | n (%) | |
| Overall | 11 | (50) |
| Hyperglycaemia | 3 | (13.6) |
| Acute kidney injury | 2 | (9.1) |
| Small intestinal obstruction | 2 | (9.1) |
| Urinary tract infection | 2 | (9.1) |
| Anaemia | 1 | (4.5) |
| Asthenia | 1 | (4.5) |
| Atrial fibrillation | 1 | (4.5) |
| Atrial flutter | 1 | (4.5) |
| Blood loss anaemia | 1 | (4.5) |
| Cardiac arrest | 1 | (4.5) |
| Confusional state | 1 | (4.5) |
| Dehydration | 1 | (4.5) |
| Erythema multiforme | 1 | (4.5) |
| Hyperkalaemia | 1 | (4.5) |
| Hypotension | 1 | (4.5) |
| Ileus | 1 | (4.5) |
| Lipase increased | 1 | (4.5) |
| Post procedural urine leak | 1 | (4.5) |
| Rash maculo-papular | 1 | (4.5) |
| Sciatica | 1 | (4.5) |
| Wound infection | 1 | (4.5) |

The most common TEAE's were hyperglycemia (13.6%) (not shown in table above), small intestinal obstruction (9.1%), urinary tract infection (9.1%).

Table 28 lists TEAE's leading to EV discontinuation.

TABLE 28
TEAE's Leading to EV Discontinuation.

| | EV Mono (N = 22) | |
|---|---|---|
| Preferred Term | n (%) | |
| Overall | 3 | (14) |
| Dehydration^{a} | 1 | (5) |
| Erythema multiforme^{b} | 1 | (5) |
| Rash maculo-papular^{c} | 1 | (5) |

^{a}Subject completed Cycle 1. AE onset occurred 6 days after last EV dose. (Gr3)
^{b}Subject completed Cycle 1. AE onset occurred 4 days after last EV dose. (Gr3)
^{c}Subject completed Cycle 2. AE onset occurred 4 days after last EV dose. (Gr3)

Table 29 lists TEAE's leading to dose modification.

TABLE 29
TEAE's leading to dose modification.

| | EV Mono (N = 22) | |
|---|---|---|
| Preferred Term | n (%) | |
| Any TEAEs leading to dose interruption^{a,b} | 3 | (14) |
| EV-related TEAEs leading to dose interruption^{a} | 2 | (9) |
| Any TEAEs leading to dose reduction^{c} | 2 | (9) |
| EV-related TEAEs leading to dose reduction | 2 | (9) |

^{a}Dose interruption includes dose elimination and dose delay as collected on the CRF. Dose elimination is when a scheduled dose is skipped. Dose delay is when a dose did not occur on the scheduled dosing cycle.
^{b}Dose delays were due to Gr2 Fatigue and Gr1 Diarrhea (both related to EV). One unrelated delay was due to inclement weather at site.
^{c}Dose reductions were due to Gr2 Dysgeusia and Gr2 Diarrhea (both related to EV).

Table 30 provides TEAE's leading to death.

TABLE 30
TEAE's Leading to Death

| | EV Mono (N = 22) | |
|---|---|---|
| Preferred Term | n (%) | |
| Overall | 2 | (9) |
| Cardiac arrest^{a} | 1 | (4.5) |

^{a}Death occurred 45 days after last EV dose and 15 days after RC + PLND. Not related to EV or RC + PLND.

Table 31 lists grade 5 adverse events observed in patients in the study. Three (3) patients had grade 5 adverse events while on the study that were related to RC+PLND; in 2 pts these AEs occurred >30 days after the end of study treatment.

TABLE 31
Grade 5 Adverse Events

| | Days from last | Days from | |
|---|---|---|---|
| Grade 5 AE | dose of EV | RC + PLND | Relatedness |
| Cardiac Arrest | 45 | 15 | Unrelated to EV or |
| | | | RC + PLND |
| Acute Kidney Injury | 142 | 62 | Related to |
| | | | RC + PLND |
| Saddle PE | 127 | 55 | Related to |
| | | | RC + PLND |

Table 32 lists TEAE's of special interest.

TABLE 32
TEAE's of Special Interest

| | EV Mono N = 22 | | |
|---|---|---|---|
| AECI: categorized by | | Any Grade | ≥Grade 3 |
| related MedDRA terms | | n (%) | n (%) |

| | | | |
|---|---|---|---|
| Any peripheral neuropathy | 5 | (23) | 0 |
| Any skin reaction | 12 | (55) | 2 (9) |
| Any rash | 12 | (55) | 2 (9) |
| Any SCAR | 1 | (5) | 1 (5) |
| Any hyperglycemia (non-fasting) | 5 | (23) | 3 (7) |
| Any ocular disorder | | | |
| Dry eye | 5 | (23) | 0 |
| Blurred vision | 2 | (9) | 0 |

| | | |
|---|---|---|
| Corneal disorders | 0 | 0 |

| | | | |
|---|---|---|---|
| Any infusion related reactions | 2 | (9) | 0 |

### 6.2.4 Conclusions

The pathological complete response (pCRR) observed in cisplatin-ineligible MIBC patients treated with neoadjuvant EV compares favorably to rates seen with chemotherapy in cisplatin-eligible MIUC patients. The observed safety profile of neoadjuvant EV monotherapy in patients in this study is consistent with the known safety profile of EV. In particular, 3 cycles of neoadjuvant treatment was well-tolerated in patients.

### 6.3 Example 3—A Proposed Phase 1b/2 Study of Neoadjuvant Enfortumab Vedotin (ASG-22CE) Monotherapy Followed by Radical Cystectomy and Pelvic Lymph Node Dissection (RC+PLND) Followed by Adjuvant Enfortumab Vedotin (i.e., in the Perioperative Setting) for Treatment of Cisplatin-Ineligible Patients with Muscle Invasive Bladder Cancer (MIBC) (Cohort L)

### 6.3.1 Drug Used in Clinical Study

This example uses enfortumab vedotin, which is described supra. The protocol described in this Example is effective to treat Cisplatin-Ineligible Patients with Muscle Invasive Bladder Cancer (MIBC).

### 6.3.2 Summary of the Study

### 6.3.2.1 Synopsis

### (i) Name of Study Drug

Enfortumab Vedotin (ASG-22CE)

### (ii) Phase of Development

Phase 1b/2

(iii) Title of Study

A Phase 1b/2 study of neoadjuvant enfortumab vedotin (ASG-22CE) as monotherapy followed by radical cystectomy and pelvic lymph node dissection (RC+PLND) for the treatment of cisplatin-ineligible patients with muscle invasive urothelial cancer (MIUC).

### (iv) Study Objective(s)

### Primary

To assess the antitumor activity of neoadjuvant and perioperative enfortumab vedotin monotherapy as measured by the pathological complete response (pCR) rate, defined as the absence of viable tumor (pT0N0) in examined tissue from radical cystectomy (RC) and pelvic lymph node dissection (PLND) by central pathology review.

### Secondary

To assess the Event-Free Survival (EFS) on study therapy by Blinded independent central review (BICR)
To assess the Event-Free Survival (EFS) on study therapy by investigator
To assess the pathologic downstaging (pDS) rate, defined as patients with tumors <pT2 (includes pT0, pTis, pTa, pT1) and N0 in examined tissue from RC+PLND by central pathology review
To assess the disease free survival (DFS) by BICR
To assess the disease free survival (DFS) by investigator
To assess overall survival (OS)
To assess the safety and tolerability of enfortumab vedotin monotherapy
To assess the percentage of planned RC+PLND delayed due to treatment-related adverse events (AEs)

### Additional

To assess Nectin-4 and PD-L1 expression levels
To assess biomarkers of biological activity and disease resistance, and their potential associations with clinical outcome measures
To assess PK and the incidence of ATA
To assess patient-reported experience and patient-reported tolerability of treatment

### (v) Study Population

Eligible patients include patients ≥18 years of age with histologically confirmed muscle invasive bladder cancer (MIBC) (predominant urothelial type [i.e., >50%]) with an ECOG performance status of 0, 1, or 2, who are deemed eligible for, and agree to undergo, curative intent RC+PLND.

Eligible patients should have a clinical stage of cT2 T4aN0M0 by review of pathology and imaging. Patients require imaging using compound tomography (CT) with intravenous (IV) contrast of the chest and a CT urogram of the abdomen and pelvis at screening.

Imaging (as described in the preceding sentence) should be done ≤28 days before enrollment [American Joint Commission on Cancer, sixth edition]. Tumor samples with an associated pathology report from the diagnostic transurethral resection of a bladder tumor (TURBT) must be available prior to enrollment and determined to be sufficient for pathology review and biomarker analysis. Patients should have adequate hematologic and organ function tests.

Eligible patients must be ineligible for cisplatin-based chemotherapy at the time of enrollment due to at least 1 of the following criteria: GFR<60 mL/min but ≥30 mL/min, ECOG performance status of 2, NCI CTCAE Version 4.03 Grade ≥2 hearing loss, or NYHA Class III heart failure. Patients must not have received prior systemic treatment, chemoradiation, or radiation therapy for MIBC. Patients may have received prior intravesical Bacillus Calmette-Guérin (BCG) or intravesical chemotherapy for non-muscle invasive bladder cancer (NMIBC).

### (vi) Number of Planned Patients

Approximately 50 patients are enrolled in this study.

(vii) Study Design

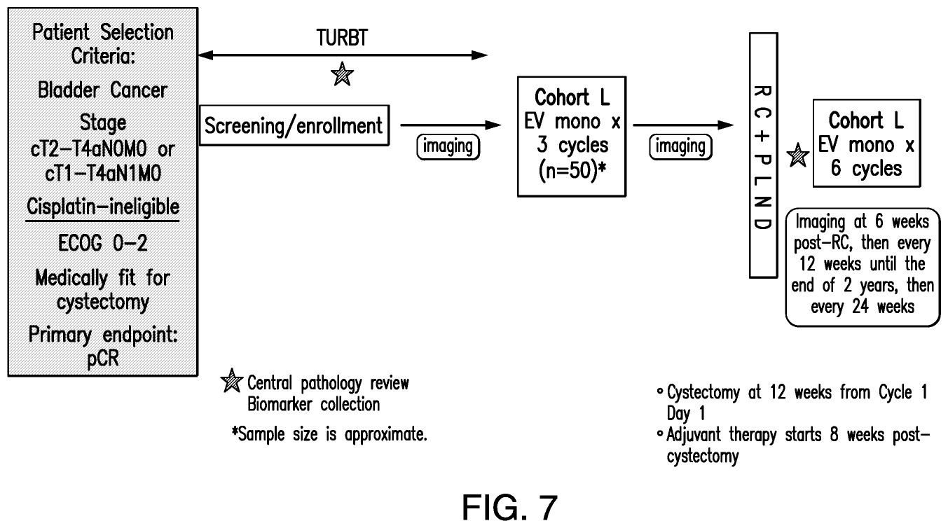

This study is designed to evaluate the safety and antitumor activity of enfortumab vedotin as monotherapy for the treatment of cisplatin ineligible patients with MIBC in the neoadjuvant and perioperative settings. The study design is depicted in FIG. 7.

Treatment with enfortumab vedotin monotherapy is evaluated in patients with MIBC. This study enrolls approximately 50 patients with cT2-T4aN0M0 or cT1-T4aN1M0 MIBC. All patients are treated with neoadjuvant enfortumab vedotin (1.25 mg/kg) on Days 1 and 8 of every 3 week cycle for a total of 3 cycles prior to RC+PLND, followed by adjuvant enfortumab vedotin (1.25 mg/kg) on Days 1 and 8 of every 3 week cycle for a total of 6 cycles starting 8 weeks post-RC.

Safety is monitored by the SMC on an ongoing basis. Step-down dose levels (up to 2 levels) for enfortumab vedotin below the established expansion (1a/mUC cohort) dose of 1.25 mg/kg is allowed for any cohort if recommended by the SMC and upon approval by the study sponsor.

All patients should have a transurethral resection of a bladder tumor (TURBT) within 90 days prior to the first treatment dose, and tissue from the diagnostic TURBT must be confirmed to be available prior to enrollment and be deemed sufficient for pathology review and biomarker analysis. All patients undergo baseline post-TURBT radiographic screening assessment using CT with IV contrast of the chest and a CT urogram of the abdomen and pelvis for clinical staging.

Following neoadjuvant treatment, restaging is done via the same radiologic modalities ≤4 weeks prior to RC+PLND to exclude disease progression, which would preclude curative surgery. Patients who remain radiographically free of distant metastases proceed to RC+PLND at week 12 (84 days±7 days) from the start of study treatment. Patients may be allowed to undergo RC+PLND outside of this time frame if the delay is due to an AE. Patients with delays other than for an AE may be considered with medical monitor consultation. Surgery consists of RC+PLND with curative intent in accordance with the American Urological Association (AUA)/American Society of Clinical Oncology (ASCO)/American Society for Radiation Oncology (ASTRO)/Society of Urologic Oncology (SUO) guidelines (Chang 2017).

Any patient refusing RC+PLND no longer receives any study intervention; patients who refuse RC+PLND who have achieved a clinical complete response (cCR), have been downstaged to non-MIBC, or have incomplete evaluations will enter study follow up, and will be followed observationally with disease assessments according to the protocol-defined schedule until disease progression/recurrence, patient death, study closure, or withdrawal of consent, whichever occurs first.

Pathological CR is assessed after RC+PLND with curative intent by central pathology review as the primary endpoint. Radiographic disease progression precluding a curative intent surgery noted prior to RC+PLND, failure to undergo RC+PLND for patients with residual muscle-invasive disease and/or any radiographic disease present, gross residual disease left behind at time of RC+PLND, local or distant recurrence post-RC as assessed radiographically and/or confirmed with biopsy (local pathology review), or death from any cause is considered events and such patients do not receive further intervention on study, but transition into long-term follow-up.

At 6 weeks (±14 days) after RC+PLND, all patients have repeat imaging (via the same radiologic modalities as their post-RC baseline scan). At this time, patients who are noted to have new recurrent or metastatic disease are considered to have had an event and do not receive further intervention on the study but transition to long-term follow-up. Patients who are radiographically free of disease proceed to the adjuvant phase. Adjuvant therapy should start 8 weeks (±14 days) post-RC and patients receive adjuvant enfortumab vedotin for a total of 6 cycles.

After the post-RC baseline imaging, imaging is performed via the same radiologic modalities every 12 weeks (±7 days) until the end of 2 years from their post-RC baseline scan. After this period on study, the frequency of visits, including response assessments, is reduced to every 24 weeks (±14 days). Tumor imaging should also be performed (using RECIST Version 1.1 criteria) whenever disease progression is suspected. All imaging scans in Cohort L is retrospectively evaluated by blinded independent central review (BICR). Imaging continues until disease progression/recurrence, patient death, study closure, or withdrawal of consent, whichever occurs first. For patients that do not have an EFS event, but start a new anticancer treatment anytime after RC+PNLD, imaging assessments continue according to the protocol-defined schedule until disease progression/recurrence, patient death, study closure, or withdrawal of consent, whichever occurs first.

Imaging should be performed within ±4 weeks of treatment discontinuation or end of treatment (EOT). If imaging was performed within 4 weeks of treatment discontinuation, then imaging at treatment discontinuation is not necessary. Results of any additional investigative imaging (eg, urograms) should be reported in the electronic CRFs (eCRFs) and submitted for central radiology assessment by BICR.

Patients complete the EuroQOL-5 Dimensions (EQ 5D-5L) Patient Reported Outcome (PRO) assessment on an electronic device (preferred medium for reporting). Assessments may be reported on paper or by clinic staff by telephone only if the use of an electronic device is not feasible. (Please see the “Other Assessments” subsection of the Synopsis for details of PRO assessments.)

(viii) Test Product, Dose, and Mode of Administration

Enfortumab vedotin is administered as an IV infusion at 1.25 mg/kg over approximately 30 minutes on Days 1 and 8 of every 3-week cycle for 3 cycles prior to RC+PLND.

Patients receive enfortumab vedotin on Days 1 and 8 of every 3-week cycle for a total of 6 cycles post-RC.

In the absence of infusion-related reactions (IRRs), the infusion rate for all patients should be calculated in order to achieve an approximate 30-minute infusion period. Enfortumab vedotin must not be administered as an IV push or bolus. Enfortumab vedotin should not be mixed with other medications. At least 1 week (7 days) must elapse between doses of enfortumab vedotin.

Enfortumab vedotin doses are calculated on the basis of a patient's actual body weight at baseline. Doses should be recalculated when a patient's body weight changes by ≥10% of baseline or the previous cycle, or when dose adjustment criteria are met. Actual weight is used except for patients weighing >100 kg; in such cases, the dose is calculated based on a weight of 100 kg. The maximum dose permitted in this study is 125 mg.

Table 33 shows the Enfortumab vedotin step-down dose levels for muscle invasive bladder cancer:

TABLE 33

| Dose level | Enfortumab Vedotin Dose (mg/kg) |
|---|---|
| 1 | 1.25 |
| −1 | 1 |
| −2 | 0.75 |

### (ix) Duration of Treatment

Patients receive 3 cycles of enfortumab vedotin monotherapy followed by RC+PLND with curative intent followed by 6 cycles of enfortumab vedotin monotherapy after RC+PLND.

The study closes 5 years after enrollment of the last patient, or when no patients remain in long term follow-up, whichever occurs first. Additionally, the sponsor may terminate the study at any time.

### (x) Efficacy Assessments

Initial staging is determined primarily from pathologic findings from the diagnostic TURBT and reviewed by a local laboratory, supplemented with staging imaging from radiographic studies per RECIST Version 1.1. Patients require imaging using CT with IV contrast of the chest and a CT urogram of the abdomen and pelvis at screening (≤28 days before enrollment) for clinical staging. Patients must receive the same imaging modality throughout the study for response assessments.

Tumor tissue samples collected from the patient's diagnostic TURBT within 90 days prior to the first dose of study treatment are sent for central pathology review. Tissue samples from RC+PLND are also sent for central pathology review of pathologic response assessment. Tumor response is assessed after RC+PLND with curative intent by pathology review analysis. Pathologic staging information includes TNM classification, histology, lymph node counts, and surgical margins.

Patients are also followed for disease recurrence using serial imaging as outlined in the Study Design subsection of the Synopsis. Disease recurrence is determined by radiologically confirmed disease progression per RECIST Version 1.1 (investigator assessment). Optional tumor biopsies at the time of disease progression, if available, may also be used to document disease progression.

Delay of planned RC+PLND with curative intent (defined as >12 weeks after the last dose of study treatment) due to treatment-related AEs is documented.

### (xi) Pharmacokinetic and ATA Assessments

Blood samples for pharmacokinetics (PK) and (antitherapeutic antibodies) ATA are collected throughout the study at specific time points. Validated or qualified assays are used to measure the concentrations of enfortumab vedotin ADC, total antibody (TAb), and MMAE in serum or plasma. PK samples are collected and archived for possible analysis of concomitant drug levels or other enfortumab vedotin-related species, such as circulating metabolites of MMAE.

(xii) Biomarker Assessments

Peripheral blood, urine, and tumor biopsies are collected at protocol specified time points. Exploratory, predictive, and prognostic biomarkers associated with response, resistance, or safety observations are monitored before and during study treatment. Tumor samples obtained at RC+PLND (for the MIBC component) are used to characterize the clinical mechanisms of action and resistance.

Tumor tissue from diagnostic TURBT specimens and RC+PLND is required (fine needle aspiration is not adequate) to identify novel biomarkers. If additional post-treatment biopsies are done as part of SOC, the samples may also be used to further identify biomarkers of response and mechanism of action and resistance to treatment.

Biomarker assessments in tumor tissue may include, but may not be limited to, measurement of gene expression (GE) and mutation burden, characterization of the tumor microenvironment (TME) and tumor subtype, and drug effects. Assays may include, but may not be limited to, immunohistochemistry (IHC) for Nectin-4 and PD-L1, and Next Generation Sequencing (NGS) of RNA and DNA. Biomarker assessments in blood samples may include, but may not be limited to, measurement of baseline and drug induced changes in circulating blood cell subpopulations, immunoassays, and circulating disease markers. Blood and urine assays may include, but may not be limited to, circulating tumor DNA, proteomic methodologies such as enzyme-linked immunosorbent assay (ELISA), immunoassays as a marker of tumor response or therapy resistance, and markers of immune function, including abundance of immune cell subsets and cytokines.

Other tissue (e.g., skin) from optional tissue sample submissions may be analyzed for biomarkers including tissue levels of drug and drug products, nucleic acids, and protein to investigate possible associations with mechanisms of resistance or sensitivity to treatments as well as dynamic changes associated with treatments. Methods of analysis include IHC, NGS of DNA and RNA, T cell receptor beta chain sequencing, polymerase chain reaction, flow cytometry, and immunoassays.

(xiii) Other Assessments

PRO assessments are completed using EQ 5D 5L. Assessments are completed on an electronic device (preferred medium for reporting) within 2 days prior to dosing on neoadjuvant Cycles 1-3 Days 1 and 8. After completion of the neoadjuvant cycles, patients complete ePROs once at RC+PLND (if applicable) and at EOT. Following RC+PLND (if applicable) and EOT, patients complete PRO assessments once every 12 weeks for 2 years (for the Follow-Up period) and once every 24 weeks thereafter (for the Long-Term Follow-Up period). On dosing visit days, assessments should be completed prior to study drug administration. Assessments may be reported on paper or by clinic staff by telephone if the use of an electronic device is not feasible.

(xiv) Safety Assessments

Safety assessments are based on the information collected through the safety surveillance process and include the data from recorded AEs, including serious adverse events (SAEs), concomitant medications, physical examination findings, cardiac monitoring, and laboratory tests. Safety will be monitored over the course of the study by the SMC.

### (xv) Statistical Methods

### (a) Analysis for the Key Safety Endpoint

The safety analysis evaluates the type, incidence, severity, seriousness, and relatedness of AEs, and the type, incidence, and severity of laboratory abnormalities. The incidence, duration, and resolution of AEs of special interest (AESIs) are summarized.

### (b) Analysis for the Key Efficacy Endpoint

The observed pathological complete response rate (pCRR) and the 95% CIs are provided using Clopper-Pearson methodology, by cohorts.

### (c) Sample Size

Approximately 50 patents may be enrolled.

The sample size is not based on power calculations for formal hypothesis testing, but is selected based on the precision of the estimate for pCRR as characterized by the 95% CIs.

For illustration purposes, Table 34 is a summary of the 2-sided 95% CIs, assuming a pCRR of 30% or 40%, and for a sample size of 50 patients:

TABLE 34

| pCRR | 95% Exact CI (n = 20) | 95% Exact CI (n = 50) |
|---|---|---|
| 30% | (12%, 54%) | (18%, 45%) |
| 40% | (19%, 64%) | (26%, 55%) |

### 6.3.3 Objectives

### Primary Objective

The Primary Objective is to assess the antitumor activity of neoadjuvant and perioperative enfortumab vedotin monotherapy or neoadjuvant enfortumab vedotin as measured by the pCR rate, defined as the absence of viable tumor (pT0N0) in examined tissue from RC and PLND by central pathology review

### Secondary Objectives Include:

To Assess the EFS on study therapy by BICR,
To assess the EFS on study therapy by investigator,
To assess the pDS rate, defined as patients with tumors <pT2 (includes pT0, pTis, pTa, pT1) and N0 in examined tissue from RC+PLND by central pathology review,
To assess the DFS by BICR,
To assess the DFS by investigator,
To assess OS,
To assess the safety and tolerability of enfortumab vedotin monotherapy, and
To assess the percentage of planned RC+PLND delayed due to treatment-related AEs.

### Additional Objectives Include:

To assess Nectin-4 and PD-L1 expression levels,
To assess biomarkers of biological activity and disease resistance, and their potential associations with clinical outcome measures,
To assess PK and the incidence of ATA, and
To assess patient-reported experience and patients-reported tolerability of treatment.

### Endpoints

### Primary Endpoint

The primary efficacy endpoint of this study is pCR rate by central pathology review.

### Secondary Endpoints

EFS by BICR,
EFS by investigator assessment,
pDS rate by central pathology review,
DFS by BICR,
DFS by investigator assessment,
Type, incidence, severity seriousness, and relatedness of AEs,
Type, incidence, and severity of laboratory abnormalities, and
Percentage of planned RC+PLND delayed (>12 weeks after the last dose of treatment) due to treatment-related AEs.

### Additional Endpoints

Exploratory biomarkers of clinical activity, including relationship of Nectin-4 expression status to response,
Selected plasma or serum PK parameters of enfortumab vedotin, MMAE, and Tab,
Incidence of ATA to enfortumab vedotin, and
Change from baseline in PRO assessment of the EQ-5D-5L

### 6.3.4 Study Plan

### 6.3.4.1 Summary of Study Design

This study is designed to evaluate the safety and antitumor activity of enfortumab vedotin as monotherapy for the treatment of cisplatin-ineligible patients with MIBC in the neoadjuvant and perioperative settings.

Treatment with enfortumab vedotin monotherapy is evaluated in patients with MIBC. This study enrolls approximately 50 patients with cT2-T4aN0M0 or cT1T4aN1M0 MIBC. Patients are treated with neoadjuvant enfortumab vedotin (1.25 mg/kg) on Days 1 and 8 of every 3-week cycle for a total of 3 cycles prior to RC+PLND, followed by adjuvant enfortumab vedotin (1.25 mg/kg) on Days 1 and 8 of every 3-week cycle for a total of 6 cycles starting 8 weeks post-RC.

Safety is monitored by the SMC on an ongoing basis. Step-down dose levels (up to 2 levels) for enfortumab vedotin below the established expansion (1a/mUC cohort) dose of 1.25 mg/kg is allowed for any cohort if recommended by the SMC and approved by the study sponsor. Enfortumab vedotin step-down dose levels for muscle invasive bladder cancer cohorts are illustrated in Table 35, below:

TABLE 35

| Dose Level | Enfortumab Vedotin Dose (mg/kg) |
|---|---|
| 1 | 1.25 |
| −1 | 1 |
| −2 | 0.75 |

All patients should have a TURBT within 90 days prior to the first treatment dose, and tissue from the diagnostic TURBT must be confirmed to be available prior to enrollment and be deemed sufficient for pathology review and biomarker analysis. All patients undergo baseline post-TURBT radiographic screening assessment using CT with IV contrast of the chest and a CT urogram of the abdomen and pelvis for clinical staging. CT urogram or MRI urogram are acceptable imaging methods to satisfy the abdominal and pelvic scanning requirements for MIBC patients specified in the protocol.

Following neoadjuvant treatment, restaging is done via the same radiologic modalities ≤4 weeks prior to RC+PLND to exclude disease progression, which would preclude curative surgery. Patients who remain radiographically free of distant metastases proceed to RC+PLND at week 12 (84 days±7 days) from the start of study treatment. Patients may be allowed to undergo RC+PLND outside of this time frame if the delay is due to an AE. Patients with delays other than for an AE may be considered with medical monitor consultation. Surgery will consist of RC+PLND with curative intent in accordance with the AUA/ASCO/ASTRO/SUO guidelines (Chang 2017).

Any patient refusing RC+PLND no longer receives any study intervention; patients who refuse RC+PLND who have achieved a cCR, have been downstaged to NMIBC, or have incomplete evaluations enter study follow up, and are followed observationally with disease assessments according to the protocol-defined schedule until disease progression/recurrence, patient death, study closure, or withdrawal of consent, whichever occurs first.

pCR is the primary endpoint and is defined as the absence of viable tumor (pT0N0) in examined tissue from RC+PLND. pCR is assessed by central pathology review after RC+PLND with curative intent. Radiographic disease progression precluding a curative intent surgery noted prior to RC+PLND, failure to undergo RC+PLND for participants with residual muscle-invasive disease and/or any radiographic disease present, gross residual disease left behind at time of RC+PLND, local or distant recurrence post-RC as assessed radiographically and/or confirmed with biopsy (local pathology review), or death from any cause is considered events and such patients will not receive further intervention on study, but will transition into long-term follow-up.

At 6 weeks (±14 days) after RC+PLND, all patients repeat imaging (via the same radiologic modalities) as their post-RC baseline scan. At this time, patients who are noted to have new recurrent or metastatic disease are considered to have had an event and do not receive further intervention on the trial but transition to long-term follow-up. Patients who are radiographically free of disease proceed to the adjuvant phase. Adjuvant therapy should start 8 weeks (±14 days) post-RC and patients will receive adjuvant enfortumab vedotin for a total of 6 cycles.

After the post-RC baseline imaging, imaging is performed via the same radiologic modalities every 12 weeks (±7 days) until the end of 2 years from their post-RC baseline scan. After this period on study, the frequency of visits, including response assessments, is reduced to every 24 weeks (±14 days). Tumor imaging should also be performed (using RECIST Version 1.1 criteria) whenever disease progression is suspected. All imaging scans in the patients are retrospectively evaluated by BICR. Imaging will continue until disease progression/recurrence, patient death, study closure, or withdrawal of consent, whichever occurs first. For patients that do not have an EFS event, but start a new anticancer treatment anytime after RC+PNLD, imaging assessments continue according to the protocol defined schedule until disease progression/recurrence, patient death, study closure, or withdrawal of consent, whichever occurs first.

EOT visits should occur within 30-37 days after patient completes all required study intervention (study intervention includes RC+PLND) or discontinues prior to completion of all required study intervention. EOT evaluations must be performed before initiation of a new therapy for cancer, with the exception of the slit lamp examination (slit lamp examinations must be performed 4 weeks after last dose.

Imaging should be performed within ±4 weeks of treatment discontinuation or EOT. If imaging was performed within 4 weeks of treatment discontinuation, then imaging at treatment discontinuation is not necessary. Results of any additional investigative imaging (eg, urograms) should be reported in the eCRFs and submitted for central radiology assessment by BICR.

Patients complete the EQ 5D-5L PRO assessment on an electronic device (preferred medium for reporting). Assessments may be reported on paper or by clinic staff by telephone if the use of an electronic device is not feasible.

### 6.3.4.2 Discussion and Rationale for Study Design

This study provides safety and efficacy data for enfortumab vedotin monotherapy in the perioperative setting (i.e., neoadjuvant followed by adjuvant) in patients with MIBC. Enfortumab vedotin 1.25 mg/kg IV is administered on Days 1 and 8 of every 3-week cycle for 3 cycles prior to RC+PLND with curative intent. Post-RC, patients receive enfortumab vedotin 1.25 mg/kg IV on Days 1 and 8 of every 3-week cycle for 6 cycles.

An SMC monitors the safety throughout the study. The safety endpoints are appropriate for evaluating the safety of the treatment regimens. The key efficacy endpoint of this component of the study (pCR rate by central review) is a direct measure of antitumor activity. This is complemented by contrast-enhanced imaging at baseline, pre-RC as well as in the adjuvant and post-RC stages. To further assess the significance of pCR rate in this study, EFS, DFS, and OS are evaluated as secondary endpoints.

### (i) Method of Assigning Patients to Treatment Groups

This study enrolls patients sequentially.

### (ii) Blinding

This is an open-label study.

### 6.3.5 Study Population

### 6.3.5.1 Inclusion Criteria

Patients must have histologically confirmed MIBC with predominant >50% urothelial histology (previously known as transitional cell carcinoma).
Clinical stage cT2-T4aN0M0 or cT1-T4aN1M0 determined by TURBT within 90 days prior to the first dose of treatment and by CT with IV contrast of the chest and a CT urogram of the abdomen and pelvis ≤28 days of enrollment. Participants with pT1 disease are eligible only if they have N1 disease on imaging.
Mixed cell types are eligible as long as urothelial cancer is predominant: >50%
Patients with plasmacytoid and/or neuroendocrine tumors are ineligible regardless of component percentage.
Urothelial tumors not originating in the bladder (eg, upper tract tumors, urethral tumors) are ineligible.
Lymph nodes <10 mm in the short axis will be considered normal.

Patients must be cisplatin-ineligible. Patients will be considered cisplatin-ineligible if they meet at least 1 of the following criteria:
GFR<60 mL/min but ≥30 mL/min (measured by the Cockcroft-Gault formula, MDRD or 24-hour urine).
ECOG performance status of 2.
NCI CTCAE Version 4.03 Grade 2 hearing loss.
NYHA Class III heart failure.
Patients eligible for enrollment must not have received prior systemic treatment, chemoradiation, or radiation therapy for MIBC. Patients may have received prior intravesical Bacillus Calmette-Guérin (BCG) or intravesical chemotherapy for NMIBC.

Minimum age of 18 years.

An ECOG performance status of 0, 1, or 2

Anticipated life expectancy of ≥3 months as assessed by the investigator.

Tumor samples with an associated pathology report from the diagnostic TURBT done 90 days prior to the first dose of study treatment must be available prior to enrollment and determined to be sufficient for pathology review and biomarker analysis. Note: Submitted TURBT biopsy must contain the detrusor muscle for confirmation of disease stage.

Patients must be deemed eligible for RC+PLND by his/her urologist and/or oncologist and agree to undergo curative intent standard RC+PLND (including prostatectomy if applicable) as per AUA/ASTRO/ASCO/SUO guidelines (referenced in section 6.3.13).

Have adequate hematologic and organ function as defined in the following table. Specimens must be collected within 7 days prior to the start of study treatment.

TABLE 36

| System | Laboratory Value |
|---|---|
| Hematological: | |
| ANC | ≥1500/uL |
| Platelets | ≥100,000/uL |
| Hemoglobin | ≥9.0 g/dL or ≥5.6 mmol/L^{a} |
| Renal: | |
| Measured or calculated^{b} CrCl | ≥30 mL/min for participants with |
| AND/OR creatinine ≤1.5 × ULN. | creatinine levels |
| Both creatinine level and creatinine | >1.5 × institutional ULN, AND/OR |
| clearance are applicable when local | creatinine levels |
| guidelines require both assessments | ≤1.5 × upper limit of normal (ULN) |
| (GFR can also be used in place of | |
| CrCl) | |
| Hepatic: | |
| Serum Total bilirubin | ≤1.5 × ULN OR direct bilirubin ≤ |
| | ULN for participants with total |
| | bilirubin levels >1. × ULN |
| | ≤3 × ULN for patients with Gilbert's |
| | disease |
| AST (SGOT) and ALT (SGPT) | ≤3 × ULN |
| Coagulation: | |
| INR OR PT | ≤1.5 × ULN unless participant |
| aPTT OR PTT | is receiving anticoagulant therapy |
| | as long as PT or aPTT is within |
| | therapeutic range of intended use |
| | of anticoagulants |

Note:
Table 36 includes eligibility-defining laboratory value requirements for treatment; laboratory value requirements should be adapted according to local regulations and guidelines for the administration of specific chemotherapies.
ALT (SGPT) = alanine aminotransferase (serum glutamic pyruvic transaminase);
ANC = absolute neutrophil count;
aPTT = activated partial thromboplastin time;
AST (SGOT) = aspartate aminotransferase (serum glutamic oxaloacetic transaminase);
CrCl = creatinine clearance;
GFR = glomerular filtration rate
^{a}Criteria must be met without erythropoietin dependency and without packed red blood cell (pRBC) transfusion within last 2 weeks.
^{b}Creatinine clearance (CrCl) should be calculated per institutional standards using the Cockcroft-Gault method, Modification of Diet in Renal Disease equations (MDRD), or by 24 hour urine collection.

A female subject of childbearing potential is anyone born female who has experienced menarche and who has not undergone surgical sterilization (eg, hysterectomy, bilateral salpingectomy, bilateral oophorectomy) or has not completed menopause. Menopause is defined clinically as 12 months of amenorrhea in a person over age 45 in the absence of other biological, physiological, or pharmacological causes. Female subjects of childbearing potential must meet the following conditions:
Agree not to try to become pregnant during the study and for at least 6 months after the final dose of study drug.
Must have a negative urine or serum pregnancy test (minimum sensitivity of 25 mIU/mL or equivalent units of beta human chorionic gonadotropin [β-hCG]) within 3 days prior to Day 1. Female subjects with false positive results and documented verification of negative pregnancy status are eligible for participation.
If heterosexually active, must consistently use highly effective methods of birth control, with a failure rate of less than 1% (as described in section 6.3.14) starting at screening, throughout the study period, and for at least 6 months after the final dose of study drug.
Female subjects must agree not to breastfeed or donate ova starting at screening and throughout the study period, and for at least 6 months after the final dose of study drug.

A male subject who can father children is anyone born male who has testes and who has not undergone surgical sterilization (eg, vasectomy followed by a clinical test proving that the procedure was effective). Male subjects who can father children, must meet the following conditions:
Must not donate sperm starting at screening and throughout the study period, and for at least 6 months after the final dose of study drug. Male subjects are informed about the negative risk to reproductive function and fertility from the study treatment. Prior to treatment male subjects should be advised to seek information on fertility preservation and sperm cryoconservation.
Must consistently use highly effective methods of birth control, with a failure rate of less than 1% (as described in Appendix M) starting at screening and continue throughout study period and for at least 6 months after the final dose of study drug.
Male subjects with a pregnant or breastfeeding partner(s) must consistently use one of 2 contraception options for preventing secondary exposure to seminal fluid (as described in Appendix M) for the duration of the pregnancy or time partner is breastfeeding throughout the study period and for at least 6 months after the final dose of study drug.

The patient must provide written informed consent.

### 6.3.5.2 Exclusion Criteria

Received prior systemic treatment, chemoradiation, and/or radiation therapy for MIBC.

Received any prior treatment with a CPI. A CPI is defined as a PD-1 inhibitor, PD-L1 inhibitor, or PD-L2 inhibitor (including, but not limited to, atezolizumab, pembrolizumab, nivolumab, durvalumab, or avelumab).

Received any prior treatment with an agent directed to another stimulatory or co inhibitory T-cell receptor (including but not limited to CD137 agonists, CTLA 4 inhibitors, or OX-40 agonists).

Evidence of ≥N2 nodal disease on imaging.

Evidence of metastatic disease on imaging.

Has undergone partial cystectomy of the bladder to remove any NMIBC or MIBC.

Ongoing sensory or motor neuropathy Grade 2 or higher.

Patients with conditions requiring high doses of steroids (>10 mg/day of prednisone or equivalent) or other immunosuppressive medications are excluded. Inhaled or topical steroids are permitted in the absence of active autoimmune disease.

Prior treatment with enfortumab vedotin or other MMAE-based ADCs for urothelial cancer.

Subjects with a history of another invasive malignancy within 3 years before the first dose of study drug, or any evidence of residual disease from a previously diagnosed malignancy. Subjects with nonmelanoma skin cancer or carcinoma in situ of any type (if complete resection was performed) are allowed.

A history of prostate cancer (T2NXMX or lower with Gleason score ≤7) treated with definitive intent (surgically or with radiation therapy) at least 1 year prior to study entry is acceptable, provided that the subject is considered prostate cancer-free and the following criteria are met:
Participants who have undergone radical prostatectomy must have undetectable PSA for >1 year and at screening.
Participants who have had radiation must have a PSA doubling time >1 year (based on at least 3 values determined >1 month apart) and a total PSA value that does not meet Phoenix criteria for biochemical recurrence (i.e., <2.0 ng/mL above nadir).

Participants with untreated low-risk prostate cancer (Gleason score ≤6) on active surveillance with PSA doubling time >1 year (based on at least 3 values determined >1 month apart) are also eligible.

Currently receiving systemic antimicrobial treatment for active infection (viral, bacterial, or fungal) at the time of first dose of enfortumab vedotin. Routine antimicrobial prophylaxis is permitted.

Patients with a positive hepatitis B surface antigen and/or antihepatitis B core antibody; patients with a negative PCR assay are permitted with either universal prophylaxis or the use of a pre-emptive approach. The approach will be selected in accordance with regional or national guidelines for patients who receive anticancer therapies.

Active hepatitis C infection or known HIV infection. Patients who have been curatively treated for hepatitis C infection are permitted if they have documented sustained virologic response of 12 weeks. No HIV testing is required unless mandated by local health authority.

Patients with active tuberculosis.

Documented history of a cerebral vascular event (stroke or transient ischemic attack), unstable angina, myocardial infarction, or cardiac symptoms (including congestive heart failure) consistent with NYHA Class IV (see Appendix E) within 6 months prior to the first dose of enfortumab vedotin.

Patients with active keratitis or corneal ulcerations. Patients with superficial punctate keratitis are allowed if the disorder is being adequately treated in the opinion of the investigator.

Has an active autoimmune disease that has required systemic treatment in past 2 years (ie, use of disease modifying agents, corticosteroids, or immunosuppressive drugs). Replacement therapy (eg, thyroxine, insulin, or physiologic corticosteroid replacement therapy for adrenal or pituitary insufficiency) is not considered a form of systemic treatment and is allowed.

History of idiopathic pulmonary fibrosis; organizing pneumonia, drug-induced pneumonitis, idiopathic pneumonitis, or evidence of active pneumonitis on screening chest CT scan.

Prior allogeneic stem cell or solid organ transplant.

Administration of a live, attenuated vaccine within 30 days prior to first dose of study drug. Examples of live vaccines include, but are not limited to, the following: measles, mumps, rubella, varicella/zoster (chicken pox), yellow fever, rabies, BCG, and typhoid vaccine. Seasonal influenza vaccines for injection are generally killed virus vaccines and are allowed; however, intranasal influenza vaccines (eg, FluMist®) are live attenuated vaccines and are not allowed.

Other underlying medical condition that, in the opinion of the investigator, would impair the ability of the patient to receive or tolerate the planned treatment and follow-up; any known psychiatric or substance abuse disorders that would interfere with cooperating with the requirements of the study.

Patients with uncontrolled diabetes. Uncontrolled diabetes is defined as HbA1c≥8% or HbA1c 7% to <8% with associated diabetes symptoms (polyuria or polydipsia) that are not otherwise explained.

### 6.3.6 Treatments

### 6.3.6.1 Treatments Administered

Patients in this study receive a dose of 1.25 mg/kg enfortumab vedotin administered as an IV infusion on Days 1 and 8 of every 3-week cycle during 3 cycles prior to RC+PLND. Surgery consists of RC+bilateral PLND with curative intent in accordance with the AUA/ASCO/ASTRO/SUO guidelines (Chang 2017). Patients also receive enfortumab vedotin on Days 1 and 8 of every 3-week cycle for a total of 6 cycles after RC+PLND.

Step down dose levels (up to 2 levels) for enfortumab vedotin below the established expansion cohort dose of 1.25 mg/kg will be allowed for any cohort if recommended by the SMC and approved by the study sponsor.

### 6.3.6.2 Investigational Study Drug

### (i) Description

Enfortumab vedotin is generated by conjugation of a chemical intermediate that contains both the MMAE and linker subunits to cysteine residues of the antibody. The resulting ADC contains an average of 3.8 drug molecules per antibody. The enfortumab vedotin drug product is a sterile, preservative free, white to off-white lyophilized powder to be reconstituted for IV administration. Enfortumab vedotin is supplied in 30 mg single-dose vials.

### (ii) Dose and Administration

Enfortumab vedotin is administered as an IV infusion at 1.25 mg/kg over approximately 30 minutes on Days 1 and 8 of every 3-week cycle for 3 cycles prior to RC+PLND. In addition, patients receive enfortumab vedotin on Days 1 and 8 of every 3-week cycle for a total of 6 cycles post-RC.

In the absence of IRRs, the infusion rate for all patients should be calculated in order to achieve an approximate 30-minute infusion period. Enfortumab vedotin must not be administered as an IV push or bolus. Enfortumab vedotin should not be mixed with other medications. At least 1 week (7 days) must elapse between doses of enfortumab vedotin.

Enfortumab vedotin doses are calculated on the basis of a patient's actual body weight at baseline. Doses should be recalculated when a patient's body weight changes by ≥10% of baseline or the previous cycle, or when dose adjustment criteria are met. Actual weight are used except for patients weighing >100 kg; in such cases, the dose are calculated based on a weight of 100 kg. The maximum dose permitted in this study is 125 mg.

The patient should be observed during enfortumab vedotin administration and for at least 60 minutes following the infusion during the first 3 cycles. All supportive measures consistent with optimal patient care should be given throughout the study according to institutional standards.

The infusion site should be monitored closely for redness, swelling, pain, and infection during and at any time after administration. Patients should be advised to report redness or discomfort promptly at the time of administration or after infusion. Institutional guidelines will be followed for the administration of chemotherapy agents and precautions taken to prevent extravasation per institutional standards and as described in “Chemotherapy and Biotherapy Guidelines and Recommendations for Practice” (Polovich 2014) and “Management of Chemotherapy Extravasation: ESMO-EONS Clinical Practice Guidelines” (Perez Fidalgo 2012). In case of enfortumab vedotin extravasation, the combination drug(s) should be held until consultation and further discussion with the medical monitor/sponsor.

iii) Dose Modifications for Enfortumab Vedotin

Intrapatient dose reduction by 1 or 2 dose levels (see Table 35) is allowed depending on the type and severity of toxicity. Patients requiring a dose reduction may be re-escalated by 1 dose level (ie, patients reduced to 0.75 mg/kg may only be re-escalated to 1 mg/kg) provided the toxicity does not require study drug discontinuation and has returned to baseline or ≤Grade 1. If the toxicity recurs, re escalation will not be permitted. Patients with ≥Grade 2 corneal AEs will not be permitted to dose re-escalate.

Dose modification recommendations for enfortumab vedotin associated toxicity are presented in Table 37 and Table 38.

Intrapatient dose reduction or interruption for other enfortumab vedotin-associated toxicity is permitted at the discretion of the medical monitor and site investigator. On a per-patient basis, dose reductions for toxicity, including DLT, may be allowed. The DLT-evaluation period is the first treatment cycle. Patients who experience a DLT in the DLT-evaluation period should not receive further treatment unless clinical benefit is demonstrated with adequately managed toxicity, and there is approval from the medical monitor. The subsequent dose level will be defined by the medical monitor in discussion with the site investigator; the type and severity of the AE observed will be taken into consideration to inform the decision.

Dose interruptions for patients without prior dose reductions and who are responding to treatment may be dose interrupted beyond 3 weeks with approval of the medical monitor, if the patient's toxicity does not otherwise require permanent discontinuation. Patients may not receive other investigational drugs, radiotherapy (except palliative radiotherapy of symptomatic and nonprogressing nontarget bone lesions), or systemic antineoplastic therapy during dose delays. If toxicities warranting a dose delay occur after Day 1 dosing and are not resolved prior to Day 8 dosing (up to Day 10), Day 8 enfortumab vedotin administration must be skipped rather than delayed. If a patient is dose-reduced due to toxicity that subsequently resolves, the patient may resume treatment at the original dose at the discretion of the medical monitor and site investigator. If there is a dose interruption, the schedule for response assessments will not be adjusted and should still be calculated from Cycle 1 Day 1.

Dose interruptions (enfortumab vedotin, pembrolizumab, and/or chemotherapy) may be allowed for situations other than treatment-related AEs such as medical/surgical events or logistical reasons not related to study therapy. Participants should be placed back on study therapy within 3 weeks of the scheduled interruption, unless otherwise discussed with the sponsor. The reason for interruption should be documented in the patient's study record.

TABLE 37
Recommended dose modifications for enfortumab
vedotin-associated hematologic toxicitiy

| Grade 1 | Grade 2 | Grade 3 | Grade 4 |
|---|---|---|---|
| Continue at same | Continue at same | Withhold dose until | Withhold dose until |
| dose level. | dose level. | toxicity is ≤ Grade 1 | toxicity is ≤ Grade 1 |
| | For Grade 2 | or has returned to | or has returned to |
| | thrombocytopenia, | baseline, then | baseline, then reduce |
| | withhold dose until | resume treatment at | dose by 1 dose level |
| | toxicity is ≤ Grade 1 | the same dose level | and resume |
| | or has returned to | or consider dose | treatment, or |
| | baseline, then resume | reduction by 1 dose | discontinue at the |
| | treatment at the same | level. | discretion of the |
| | dose level. | Transfusions or | investigator. |
| | | growth factors may | Transfusions or |
| | | be used as indicated | growth factors may |
| | | per institutional | be used as indicated |
| | | guidelines. | per institutional |
| | | | guidelines. |
| | | | For anemia, treatment |
| | | | discontinuation |
| | | | should be strongly |
| | | | considered. |

Note:
hematologic toxicity refers to anemia, thrombocytopenia, neutropenia, and febrile neutropenia.

TABLE 38
Recommended dose modifications for enfortumab
vedotin-associated nonhematologic toxicity

| Grade 1 | Grade 2 | Grade 3 | Grade 4 |
|---|---|---|---|
| Continue at same | Continue at same | Withhold dose until | For Grade 4 AEs, |
| dose level. | dose level, except in | toxicity is ≤ Grade 1 or | discontinue |
| For Grade 1 rash or | the event of Grade 2 | has returned to | treatment.^{a} For |
| skin reactions, may | neuropathy or | baseline, then resume | confirmed SJS or |
| continue at same | corneal AEs. | treatment at the same | TEN, or Grade 4 |
| dose level. | For worsening rash | dose level or consider | rash, permanently |
| If ocular symptoms | or skin reactions, | dose reduction by 1 | discontinue treatment. |
| and/or changes in | consider withholding | dose level^{.a} | Grade 4 vomiting |
| vision are | enfortumab vedotin | For Grade 3 rash or | and/or diarrhea that |
| identified, the | until toxicity is ≤ | skin reactions withhold | improves to ≤ |
| patient should be | Grade 1 or has | enfortumab vedotin | Grade 2 within 72 |
| evaluated by a | returned to baseline, | until toxicity is ≤ | hours with supportive |
| qualified | then resume | Grade 1 or has returned | management does not |
| optometrist or | treatment at the | to baseline, then | require discontinuation. |
| ophthalmologist. | same dose level or | resume treatment at the | For Grade 4 |
| | consider dose | same dose level or | hyperglycemia or |
| | reduction by 1 | consider dose reduction | blood glucose >500 |
| | level. | by 1 level. | mg/dL, withhold |
| | For Grade 2 | For suspected Stevens | enfortumab vedotin |
| | neuropathy or | Johnson Syndrome | treatment and |
| | corneal AEs, | (SJS) or suspected | undertake a full |
| | withhold dose until | toxic epidermal | evaluation of the |
| | toxicity is ≤ Grade | necrolysis (TEN) | hyperglycemia to |
| | 1 or has returned to | withhold enfortumab | determine the |
| | baseline, then | vedotin. Consider | underlying diagnosis. |
| | resume treatment at | referral of the subject | Once the blood glucose |
| | the same dose level. | to a dermatologist/ | is ≤250 mg/dl and |
| | For the second | specialist for diagnosis | patient is clinically |
| | occurrence of Grade | and specialized care. | and metabolically |
| | 2 neuropathy or | Subjects who have | stable, dosing may |
| | corneal AEs, | confirmed SJS or | be resumed with |
| | withhold dose until | recurrent Grade 3 rash | close monitoring |
| | toxicity is ≤ Grade | events should have | after consultation |
| | 1, then reduce the | therapy permanently | with medical monitor. |
| | dose by 1 dose level | discontinued. | |
| | and resume treatment. | For Grade 3 neuropathy | |
| | If ocular symptoms | or corneal AEs, | |
| | and/or changes in | discontinue treatment. | |
| | vision are identified, | For Grade 3 | |
| | the patient should be | hyperglycemia or | |
| | evaluated by a | blood glucose >250 | |
| | qualified optometrist | mg/dL, withhold | |
| | or ophthalmologist. | enfortumab vedotin | |
| | | treatment. Resume | |
| | | treatment once | |
| | | hyperglycemia/blood | |
| | | glucose is ≤250 mg/dL | |
| | | and patient is clinically | |
| | | and metabolically stable. | |
| | | If ocular symptoms | |
| | | and/or changes in | |
| | | vision are identified, | |
| | | the patient should be | |
| | | evaluated by a | |
| | | qualified optometrist or | |
| | | ophthalmologist. | |

^{a}Grade 3/4 electrolyte imbalances/laboratory abnormalities, that are not associated with clinical sequelae or are corrected with supplementation/appropriate management within 72 hours of their onset do not require discontinuation (eg, Grade 4 hyperuricemia). Grade 4 amylase/lipase elevation that is not associated with clinical sequalae does not require discontinuation; withhold dose until toxicity is ≤ Grade 3 or has returned to baseline, then resume treatment at the same dose level. Grade 3 amylase/lipase elevation that is not associated with clinical sequelae may continue treatment at the same dose level. Hold therapy for ≥ Grade 3 amylase/lipase with clinical sequelae until evaluation is complete.

See Section 6.3.6.4 (i) for recommended management of infusion reactions. See Section 6.3.6.4 (ii) for recommended management of hyperglycemia. See Section 6.3.6.4 (iii) for recommended management of rash.

Patients who experience unacceptable toxicity that is attributable only to enfortumab vedotin should be discontinued from study drug.

### (a) Treatment Discontinuation Recommendations Related to Liver Safety

In the absence of an explanation for increased liver function tests (LFTs), such as viral hepatitis, pre-existing or acute liver disease, or exposure to other agents associated with liver injury, the patient may be discontinued from the study treatment. The investigator may determine that it is not in the patient's best interest to continue study treatment.

Discontinutation of treatment should be considered if:
ALT or aspartate aminotransferase (AST)>8×upper limit of normal (ULN)
ALT or AST>5×ULN for more than 2 weeks
ALT or AST>3×ULN and total bilirubin >2×ULN or international normalized ratio (INR)>1.5 (if INR testing is applicable/evaluated)
ALT or AST>3×ULN with the appearance of symptoms suggestive of liver injury (eg, right upper quadrant pain or tenderness) and/or eosinophilia (>5%)

These treatment discontinuation recommendations are based on the FDA Guidance for Industry (Drug-Induced Liver Injury: Premarketing Clinical Evaluation, July 2009). The recommendations are a basic guide to the investigator based on accumulated clinical experience with drugs in development, and are not specific to clinical experience with enfortumab vedotin.

See section 6.3.11 for recommended liver safety monitoring and assessment criteria in patients with Grade 2 or greater elevations in ALT, AST, or bilirubin.

### 6.3.6.3 Concomitant Therapy

All concomitant medications and blood products administered are recorded from Day 1 (predose) through the safety reporting period (EOT visit or 30 days after the last study treatment, whichever is later). Any concomitant medication given for a study protocol-related AE should be recorded from the time of informed consent.

### (i) Required Concomitant Therapy

There are no required concomitant therapies.

### (ii) Allowed Concomitant Therapy

The use of anti-emetics is permitted. The use of insulin is permitted as part of SOC. Premedications for IRRs per Section 6.3.6.4 are permitted; however, prophylactic premedication prior to study treatment on Cycle 1 Day 1 for prevention of IRRs may not be administered.

Therapy to manage enfortumab vedotin-associated toxicity as recommended in Section 6.3.6.2(iii) is permitted, including hematopoietic growth factors and transfusions.

Patients who are receiving strong CYP3A4 inhibitors or P-gp inhibitors concomitantly with enfortumab vedotin should be monitored for adverse reactions.

Routine prophylaxis with vaccines is permitted; however, patients may not be treated with a live, attenuated vaccine during the study.

Steroids may be used as clinically indicated.

(iii) Prohibited Concomitant Therapy

Medications or vaccinations specifically prohibited in the exclusion criteria are not allowed during the ongoing trial. If there is a clinical indication for any medication or vaccination specifically prohibited during the trial, discontinuation from trial therapy or vaccination may be required.

Listed below are specific restrictions for concomitant therapy or vaccination during the course of the study:
Antineoplastic systemic chemotherapy or biological therapy
Immunotherapy not specified in this protocol
Chemotherapy not specified in this protocol
Investigational agents other than pembrolizumab or enfortumab vedotin
Radiation therapy
Live vaccines within 30 days prior to the first dose of study treatment and while participating in the study. Examples of live vaccines include, but are not limited to, the following: measles, mumps, rubella, varicella/zoster, yellow fever, rabies, BCG, and typhoid vaccine. Seasonal influenza vaccines for injection are generally killed virus vaccines and are allowed; however, intranasal influenza vaccines (eg, FluMist) are live attenuated vaccines and are not allowed.
Pioglitazone

Participants who, in the assessment of the investigator, require the use of any of the aforementioned treatments for clinical management should be removed from the study.

### 6.3.6.4 Management of Adverse Reactions

### (i) Management of Adverse Reactions

An infusion-related reaction (IRR) may occur during the infusion of study treatment. The infusion should be administered at a site properly equipped and staffed to manage anaphylaxis should it occur. All supportive measures consistent with optimal patient care should be given throughout the study according to institutional standards. Supportive measures may include administering medications for IRRs.

Premedications for IRRs are permitted as described below; however, prophylactic premedication prior to study treatment on Cycle 1 Day 1 for prevention of IRRs may not be administered. Patients who have experienced an IRR may be premedicated for subsequent infusions. Premedication may include pain medication (eg, acetaminophen or equivalent), an antihistamine (e.g., diphenhydramine hydrochloride), and a corticosteroid administered approximately 30 to 60 minutes prior to each enfortumab vedotin infusion or according to institutional standards. Should a patient experience IRRs in the setting of premedication, continued treatment must be discussed with the medical monitor prior to the next planned dose.

If anaphylaxis occurs, study treatment administration should be immediately and permanently discontinued.

If a patient experiences an IRR after receiving study treatments and a single cause of the IRR cannot be determined, appropriate sets of guidelines for IRRs must be followed.

### (ii) Management of Hyperglycemia

Investigators should monitor blood glucose levels and are advised to perform additional assessments if any symptoms of hyperglycemia are observed, including a thorough evaluation for infection. In addition, if steroids are used to treat any other condition, blood glucose levels may require additional monitoring. If elevated blood glucose levels are observed, patients should be treated according to local standard of care (SOC) and referral to endocrinology may be considered.

Patients, especially those with a history of or ongoing diabetes mellitus or hyperglycemia, should be advised to immediately notify their physician if their glucose level becomes difficult to control or if they experience symptoms suggestive of hyperglycemia such as frequent urination, increased thirst, blurred vision, fatigue, and headache.

Patients who enter the study with an elevated HbA1c (≥6.5%) at baseline should be referred to an appropriate provider during Cycle 1 for glucose management. Blood glucose should be checked prior to each dosing and Dose should be withheld for blood glucose >250 mg/dL. Dosing may continue once the patient's blood glucose has improved to ≤250 mg/dL and patient is clinically and metabolically stable. The use of insulin is permitted as part of SOC. Blood glucose >500 mg/dL considered related to enfortumab vedotin requires drug interruption and a full evaluation of the hyperglycemia to determine the underlying diagnosis. Once hyperglycemia/elevated blood glucose has improved to ≤250 mg/dL, dosing may resume with close monitoring after consultation with medical monitor. If a patient experiences new onset of diabetes mellitus, evaluate with a metabolic panel, urine ketones, HbA1c, C-peptide, to assess new onset of type 1 diabetes in the setting of combination with CPI.

(iii) Management of Rash

Enfortumab vedotin is a Nectin-4 directed antibody drug conjugate. Nectin-4 is a cell adhesion molecule that is highly expressed in urothelial carcinoma. Low to moderate levels of Nectin-4 are also expressed on normal tissues, including skin keratinocytes, sweat glands and hair follicles; thus, skin reactions are anticipated events. As such, skin reactions are AEs of interest in all clinical studies with enfortumab vedotin.

Reports of severe cutaneous adverse reactions in 15 patients receiving enfortumab vedotin, some of whom had fatal outcomes. These reactions occurred predominantly during the first cycle of treatment. AEs reported in these cases included Stevens-Johnson Syndrome (SJS) (5 cases), blister (3 cases), dermatitis bullous (3 cases), symmetrical drug-related intertriginous and flexural exanthema (SDRIFE; 2 cases), and 1 case each of dermatitis exfoliative, exfoliative rash, epidermal necrosis, oropharyngeal blistering, stomatitis, and toxic epidermal necrolysis (TEN).

In enfortumab vedotin monotherapy studies of urothelial carcinoma, SAEs of severe cutaneous adverse reactions were reported in 11 of 749 subjects (1.5%) and included dermatitis bullous (0.4%), drug eruption (0.4%), blister (0.1%), conjunctivitis (0.1%), SJS (0.1%), stomatitis (0.1%), and toxic skin eruption (0.1%).

Subjects should be advised to contact the Investigator immediately if they have signs and symptoms of skin reactions, oral mucosal and ocular abnormalities including mucositis or conjunctivitis. Starting in the first cycle and throughout treatment, closely monitor subjects for skin reactions. For mild to moderate skin reactions, consider appropriate treatment, such as topical corticosteroids and antihistamines as clinically indicated. For worsening Grade 2 rash or skin reactions, consider withholding enfortumab vedotin. For severe (Grade 3) rash or skin reactions or suspected SJS or TEN, withhold enfortumab vedotin and consider referral for specialized care. Permanently discontinue enfortumab vedotin in patients with confirmed SJS or TEN, or Grade 4 or recurrent Grade 3 skin reactions.

### 6.3.6.5 Treatment Compliance

Study drug administration will be performed by study site staff and documented in source documents and the case report form (CRF).

### 6.3.7 Study Assessments

### 6.3.7.1 Screening Baseline Assessments

Only patients who meet all inclusion and exclusion criteria specified in Section 6.3.5 are enrolled in this study. Enrollment status and date are recorded in CRF.

Patient medical history includes a thorough review of significant past medical history, current conditions, any treatment for prior malignancies and response to prior treatment, and any concomitant medications.

Acquisition of a fresh tumor specimen from TURBT (tissue must be collected within 90 days of planned Cycle 1 Day 1), a complete eye examination, brain scan (if clinically indicated) (MRI with gadolinium contrast preferred), bone scan (if clinically indicated), CT with IV contrast of the chest and a CT urogram of the abdomen and pelvis for baseline tumor imaging, serology for hepatitis B surface antigen and antihepatitis B core antibody, serology for antihepatitis C antibody, HbA1c, urinalysis with reflexive microscopic analysis, and thyroid function tests are required for all patients at screening.

INR/PT/PTT, a pregnancy test (either urine or serum, for females of childbearing potential), physical examination (including weight), collection of height, vital signs, CBC with differential, serum chemistry panel, CrCl, ECOG performance status assessment, and ECG are required at baseline. An ECHO is required at baseline for subjects with NYHA Class III heart failure or a history of coronary heart disease, arrhythmia or other significant heart disease.

### 6.3.7.2 Response Efficacy Assessments

Initial staging is determined primarily from pathologic findings from the diagnostic TURBT and reviewed by local laboratory, supplemented with staging imaging from radiographic studies as per RECIST Version 1.1. CT with IV contrast of the chest and a CT urogram of the abdomen and pelvis will be used ≤28 days before enrollment for initial clinical staging. Patients must receive the same imaging modality throughout the study for response assessments.

Pre-RC scans should be completed ≤4 weeks prior to RC+PLND, to exclude disease progression, which would preclude curative surgery (RC+PLND must occur 12 weeks from the start of study treatment). Pre-RC scans should be reviewed prior to RC+PLND. The post-RC baseline scan should be performed at 6 weeks (±14 days) after RC+PLND. After the post-RC baseline imaging, patients should undergo imaging every 12 weeks (±7 days) until the end of 2 years from their post-RC baseline scan. After this period on study, the frequency of visits (including response assessments) will be reduced to every 24 weeks (+14 days). Imaging will continue until disease progression/recurrence, patient death, withdrawal of consent, or study closure, whichever occurs first.

For patients that do not have an EFS event, but start a new anticancer treatment after RC+PNLD, imaging assessments will continue at post-treatment follow-up visits until disease progression/recurrence, patient death, withdrawal of consent, or study closure, whichever occurs first. Tumor imaging should also be performed (using RECIST Version 1.1 criteria) whenever disease progression is suspected.

Tumor tissue samples collected from the patient's diagnostic TURBT within 90 days prior to the first dose of study treatment is sent for central pathology review. Tissue samples from RC+PLND is also sent for central pathology review of pathologic response assessment. Tumor response is assessed after RC+PLND with curative intent by pathology review analysis. Pathologic staging information includes TNM classification, histology, lymph node counts, and surgical margins. pCR as the primary endpoint evaluated by central pathology review.

Patients are also followed for disease recurrence using serial imaging. Disease recurrence is determined by radiologically confirmed disease progression per RECIST Version 1.1 (investigator assessment). Optional tumor biopsies at the time of disease progression, if available, may also be used to document disease progression.

Delayed planned RC+PLND with curative intent (defined as >12 weeks after the last dose of study treatment) due to treatment-related AEs is documented.

See Section 6.3.8.2 for definitions of study endpoints.

### 6.3.7.3 Pharmacokinetic and ATA Assessments

Blood samples for PK and ATA are collected throughout the study per the sample collection schedule in Table 39. Validated or qualified assays are used to measure the concentrations of enfortumab vedotin ADC, Tab, and MMAE in serum or plasma. PK samples are collected and archived for possible analysis of concomitant drug levels or other enfortumab vedotin-related species, such as circulating metabolites of MMAE. A validated assay is used to determine the levels of ATA for enfortumab vedotin in plasma. If enfortumab vedotin infusions are discontinued, the corresponding research samples no longer needs to be collected. If at some point prospective PK blood sample collection is no longer required, sites are notified.

### 6.3.7.4 Biomarker Studies

Samples for exploratory biomarker analyses are collected at protocol-specified time points (see Table 39). Biomarker assessments are not used for patient selection.

Methods of analysis may include: immunohistochemistry (IHC), Next Generation Sequencing (NGS) of DNA and RNA, T cell receptor beta chain sequencing, PCR, flow cytometry, and immunoassays.

All patients should provide tissue for biomarker analysis from an archival tissue sample or newly obtained core or excisional biopsy of a tumor lesion. A formalin-fixed paraffin embedded tumor tissue block is requested (please refer to the central laboratory manual for tumor tissue requirements). Core needle and excisional biopsies from tumor tissue (non-bone sites) are preferred. A fresh baseline core needle biopsy of a tumor lesion may be submitted, if feasible. In the event a biopsy is clinically required as part of SOC, tissue should be made available for biomarker assessment.

Exploratory, predictive, and prognostic biomarkers associated with response, resistance, or safety observations is monitored before and during treatment with enfortumab vedotin. Biomarker assessments in tumor tissue may include, but may not be limited to, measurements of enfortumab vedotin and its metabolites, as well as characterization of the tumor microenvironment (TME) and drug effects. Assays may include, but may not be limited to, IHC and next generation sequencing of RNA and DNA.

### (i) Biomarkers in Blood

The primary effects of enfortumab vedotin on tumor cells may lead to changes in the activation state of local, tumor-associated, and peripheral immune cells. Biomarker assessments in blood and urine samples may include, but may not be limited to, circulating tumor DNA, proteomic methodologies such as enzyme-linked immunosorbent assay (ELISA), immunoassays as a marker of tumor response or therapy resistance, and markers of immune function, including abundance of immune cell subsets and cytokines. These may provide insight into treatment-related changes associated with enfortumab vedotin or enfortumab vedotin in combination with pembrolizumab.

TABLE 39
Pharmacokinetic, immunogenicity, and biomarker sample collection time points

| | | | | | Pharmacokinetics and | | | |
|---|---|---|---|---|---|---|---|---|
| | | | | Relative | Immunogenicity | | Blood Biomarkers | |
| | Study Day | Time | Window | Time | EV PK | ATA EV | Serum | Plasma-1 |
| Screening | Days −28 | N/A | N/A | N/A | | | | |
| | to 1 | | | | | | | |
| Cycle N1 | Day 1 | Predose | Within | Start of EV | X | X | X | X |
| | | | 24 h | infusion | | | | |
| | | End of EV IV | Within | End of EV | X | | | |
| | | infusion | 15 min | infusion | | | | |
| | Day 8 | Predose | Within | Start of EV | X | | X | X |
| | | | 4 h | infusion | | | | |
| | | End of EV IV | Within | End of EV | X | | | |
| | | infusion | 15 min | infusion | | | | |
| Cycle N2 | Day 1 | Predose | Within | Start of EV | X | X | X | X |
| | | | 24 h | infusion | | | | |
| Cycle N3 | Day 1 | Predose | Within | Start of | | X | X | X |
| | | | 24 h | infusion | | | | |
| Radical Cystectomy^{C} | | | | | | X | X^{F} | X^{F} |
| Cycle A1 | Day 1 | Predose | Within | Start of EV | X | X | X | X |
| | | | 24 h | infusion | | | | |
| | | End of EV IV | Within | End of EV | X | | | |
| | | infusion | 15 min | infusion | | | | |
| | Day 8 | Predose | Within | Start of EV | X | | X | X |
| | | | 4 h | infusion | | | | |
| | | End of EV IV | Within | End of EV | X | | | |
| | | infusion | 15 min | infusion | | | | |
| Cycle A2 | Day 1 | Predose | Within | Start of | X | | X | X |
| | | | 24 h | infusion | | | | |
| Cycle A3 | Day 1 | Predose | Within | Start of | | X | X | X |
| | | | 24 h | infusion | | | | |
| Cycle A6 | Day 1 | Predose | Within | Start of EV | X | X | X | X |
| | | | 24 h | infusion | | | | |
| | | End of EV IV | Within | End of EV | X | | | |
| | | infusion | 15 min | infusion | | | | |
| | Day 8 | Predose | Within | Start of EV | X | | X | X |
| | | | 4 h | infusion | | | | |
| | | End of EV IV | Within | End of EV | X | | | |
| | | infusion | 15 min | infusion | | | | |
| Follow-up | Every 12 | N/A | ±7 days | N/A | | | X | X |
| | weeks (±7 d) | | | | | | | |
| | until end of 2 | | | | | | | |
| | years, then | | | | | | | |
| | every 24 | | | | | | | |
| | weeks (±14 d) | | | | | | | |
| | | | | | | | | Tissue |
| | | | | | | | Urine | Bio- |
| | | | | | | | Bio- | markers |
| | | | | Relative | Blood Biomarkers | | markers | Tumor |
| | Study Day | Time | Window | Time | Plasma-2^{E} | Plasma 3 | Urine | Tissue^{A} |
| Screening | Days −28 | N/A | N/A | N/A | | | | X^{B} |
| | to 1 | | | | | | | |
| Cycle N1 | Day 1 | Predose | Within | Start of EV | X | X | X | |
| | | | 24 h | infusion | | | | |
| | | End of EV IV | Within | End of EV | | | | |
| | | infusion | 15 min | infusion | | | | |
| | Day 8 | Predose | Within | Start of EV | X | X | X | |
| | | | 4 h | infusion | | | | |
| | | End of EV IV | Within | End of EV | | | | |
| | | infusion | 15 min | infusion | | | | |
| Cycle N2 | Day 1 | Predose | Within | Start of EV | X | X | X | |
| | | | 24 h | infusion | | | | |
| Cycle N3 | Day 1 | Predose | Within | Start of | X | X | X | |
| | | | 24 h | infusion | | | | |
| Radical Cystectomy^{C} | | | | | X^{F} | X^{F} | X^{F} | X^{D} |
| Cycle A1 | Day 1 | Predose | Within | Start of EV | X | X | X | |
| | | | 24 h | infusion | | | | |
| | | End of EV IV | Within | End of EV | | | | |
| | | infusion | 15 min | infusion | | | | |
| | Day 8 | Predose | Within | Start of EV | X | X | X | |
| | | | 4 h | infusion | | | | |
| | | End of EV IV | Within | End of EV | | | | |
| | | infusion | 15 min | infusion | | | | |
| Cycle A2 | Day 1 | Predose | Within | Start of | X | X | X | |
| | | | 24 h | infusion | | | | |
| Cycle A3 | Day 1 | Predose | Within | Start of | X | X | X | |
| | | | 24 h | infusion | | | | |
| Cycle A6 | Day 1 | Predose | Within | Start of EV | X | X | X | |
| | | | 24 h | infusion | | | | |
| | | End of EV IV | Within | End of EV | | | | |
| | | infusion | 15 min | infusion | | | | |
| | Day 8 | Predose | Within | Start of EV | X | X | X | |
| | | | 4 h | infusion | | | | |
| | | End of EV IV | Within | End of EV | | | | |
| | | infusion | 15 min | infusion | | | | |
| Follow-up | Every 12 | N/A | ±7 days | N/A | X | X | X | X^{G} |
| | weeks (±7 d) | | | | | | | |
| | until end of 2 | | | | | | | |
| | years, then | | | | | | | |
| | every 24 | | | | | | | |
| | weeks (±14 d) | | | | | | | |

ATA = antitherapeutic antibody; EV = Enfortumab vedotin; N/A = not applicable; PK = pharmacokinetics.
Cycle N1, etc = Neoadjuvant Cycle 1, etc; Cycle A1, etc = Adjuvant Cycle 1, etc. If at some point prospective PK blood sample collection is no longer required, sites will be notified.
Note:
For subjects who permanently discontinue enfortumab vedotin, no PK samples will be collected.
^{A}If nonprotocol mandated tumor tissue is collected as part of standard care, tissue may be submitted for further biomarker analysis.
^{B}Obtain tissue samples from TURBT (tissue must have been collected via TURBT within 90 days of planned Cycle 1 Day 1)
^{C}Radical cystectomy + bilateral PLND with curative intent will be done at week 12 (±7 days) from start of study treatment. All samples except for tumor tissue should be collected within 7 days prior to RC. Tumor tissue should be obtained on the day of RC.
^{D}See Section 6.3.7.4 and the lab manual for tissue collection requirements for RC.
^{E}Cell Pellet will be generated from the Plasma-2 collection tube. Further site processing instructions are documented in the Lab Manual.
^{F}Blood and urine samples to be taken within 7 days prior to RC.
^{G}Submit a biopsy in the event that non-protocol-mandated tissue is collected at the follow-up or EOT visit.

### (ii) Biomarkers in Tumor Tissue

To understand the relationship between the biological characteristics of tumors before treatment and patient outcomes, tissue from the TURBT (tumor biopsies) is examined. Biopsies are assessed for specific pharmacodynamic, predictive, and prognostic biomarkers in the tumor. If tissue is available from a standard-of-care biopsy collected after enrollment (until disease progression after treatment with enfortumab vedotin), it may also be examined.

Tumor tissue from diagnostic TURBT specimens and RC+PLND is required (fine needle aspiration is not adequate) to identify novel biomarkers. If additional post-treatment biopsies are done as part of SOC, the samples may also be used to further identify biomarkers of response and mechanism of action and resistance to treatment.

Biomarker assessments in tumor tissue may include, but are not limited to, central assessment of Nectin-4 expression by IHC and next generation sequencing, tumor subtyping, tumor microenvironment analyses, and profiling of somatic mutations or alterations in genes or RNA commonly altered in cancer.

### 6.3.7.5 Biospecimen Repository

For patients in the US who provide additional consent, remaining de-identified unused blood, urine and/or tissue is retained by the sponsors and used for future research, including but not limited to the evaluation of targets for novel therapeutic agents, the biology of ADC sensitivity and resistance mechanisms, and the identification of biomarkers of ADCs. Blood, urine, and tissue samples donated for future research is retained for a period of up to 25 years. If additional consent is not provided, any remaining biological samples are destroyed following study completion.

### 6.3.7.6 Patient Reported Outcomes (PRO)

PRO assessments are completed using EQ 5D-5L (see Section 6.3.7.6 ii for details). Assessments are completed within 2 days prior to dosing on neoadjuvant Cycles 1-3 Days 1 and 8. After completion of the neoadjuvant cycles, patients complete ePROs once at RC+PLND (if applicable) and at EOT. Following RC+PLND (if applicable) and EOT, patients complete PRO assessments once every 12 weeks for 2 years (for the Follow-Up period) and once every 24 weeks thereafter (for the Long-Term Follow-Up period). On dosing visit days, assessments should be completed prior to study drug administration.

Assessments are completed by the patient at home on an electronic device (preferred medium for reporting) that is given to each patient at the first clinic visit. The device has preset reminders for the patient to fill out their assessments at prespecified timepoints. Sites are required to monitor the patients' compliance with the ePRO devices through an online portal. Assessments may be reported on paper or by clinical staff by telephone (using approved scripts that will be made available to sites) only if the use of an electronic device is not feasible. When using paper or interview methods, patients are provided with instructions for filling in the assessments at clinic visits, prior to dosing, and at prespecified timepoints. The investigator or site designee should review the assessment data for correct completion when collected. The investigator or site designee, and site personnel are trained on how to provide support to each patient on the use of the PRO device (or paper assessment only if the use of the electronic device is not feasible).

### (i) EORTC Core Quality of Life Questionnaire, QLQ-C30

The EORTC Core Quality of Life (QLQ-C-30) assessment was developed to measure aspects of QoL pertinent to patients with a broad range of cancers who are participating in clinical trials (Aaronson 1993); (Sneeuw 1998). The current version of the core instrument (QLQ-C30, Version 3) is depicted in FIG. 3 a 30-item assessment consisting of the following:
5 functional domains (physical, role, cognitive, emotional, and social);
3 symptoms scales (fatigue, pain, nausea and vomiting);
Single items for symptoms (shortness of breath, loss of appetite, sleep disturbance, constipation, diarrhea) and financial impact of the disease, and;
2 global items (health, overall QoL)

Each domain is scored from 0 to 100. For the global health status/QoL and functional domain scores, higher scores represent better QoL and functioning, respectively. For symptom scales, higher scores represent greater symptomatology.

### (ii) EuroQol-5 Dimensions (EQ-5D-5L)

The EQ-5D is a standardized instrument developed by the EuroQol Group for use as a generic, preference-based measure of health outcomes. It is applicable to a wide range of health conditions and treatments and provides a simple descriptive profile and a single index value for health status. The EQ-5D is a 5-item self-reported measure of functioning and well being, which assesses 5 dimensions of health, including mobility, self-care, usual activities, pain/discomfort, and anxiety/depression (see FIG. 4). Each dimension comprises 3 levels (no problems, some/moderate problems, extreme problems). A unique EQ-5D health state is defined by combining 1 level from each of the 5 dimensions. Responses to the 5 items are then converted to a weighted health state index (utility score) based on values derived from general population samples (Herdman 2011). The health utility score is between 0 and 1, where 0 is death and 1 is perfect health. In addition to the utility score, this assessment also records the respondent's self-rated health status on a vertical graduated (0 to 100) visual analogue scale.

### 6.3.7.7 Safety Assessments

The assessment of safety during the course of this study consists of the surveillance and recording of AEs including SAEs (serious adverse event), recording of concomitant medication, and measurements of protocol-specified physical examination findings, cardiac monitoring, and laboratory tests.

Safety is monitored over the course of the study by the SMC as described in Section 6.3.4.1.

### (i) Adverse Events

### (a) About Adverse Events

### Adverse Event

According to the International Council for Harmonisation (ICH) E2A guideline Definitions and Standards for Expedited Reporting, and 21 Code of Federal Regulations (CFR) 312.32, Investigational New Drug (IND) Safety Reporting, an AE is any untoward medical occurrence in a patient or clinical investigational subject administered a medicinal product and which does not necessarily have a causal relationship with this treatment.

The following information should be considered when determining whether or not to record a test result, medical condition, or other incident on the AEs and Pre-existing Conditions CRF:
From the time of informed consent through the day prior to study Day 1, only study protocol related AEs should be recorded. A protocol-related AE is defined as an untoward medical event occurring as a result of a protocol mandated procedure.
All medical conditions present or ongoing predose on study Day 1 should be recorded.
All AEs (regardless of relationship to study drug) should be recorded from study Day 1 (predose) through the end of the safety reporting period (see Section 6.3.7.7 (i)(c)). Complications that occur in association with any procedure (eg, biopsy) should be recorded as AEs whether or not the procedure was protocol mandated.
Changes in medical conditions and AEs, including changes in severity, frequency, or character, during the safety reporting period should be recorded.
In general, an abnormal laboratory value should not be recorded as an AE unless it is associated with clinical signs or symptoms, requires an intervention, results in a SAE, or results in study termination or interruption/discontinuation of study treatment. When recording an AE resulting from a laboratory abnormality, the resulting medical condition rather than the abnormality itself should be recorded (eg, record “anemia” rather than “low hemoglobin”).

### Serious Adverse Events

An AE should be classified as an SAE if it meets one of the following criteria:
Fatal: AE resulted in death.
Life threatening: The AEs placed the patient at immediate risk of death. This classification does not apply to an AE that hypothetically might cause death if it were more severe.
Hospitalization: The AE resulted in hospitalization or prolonged an existing inpatient hospitalization. Hospitalizations for elective medical or surgical procedures or treatments planned before the signing of informed consent in the study or routine check-ups are not SAEs by this criterion. Admission to a palliative unit or hospice care facility is not considered to be a hospitalization. Pre-planned hospitalizations for therapeutic, diagnostic, or surgical procedures of the underlying cancer or study target disease that did not worsen during the clinical trial need not be captured as SAEs.
Disabling/incapacitating: An AE that resulted in a persistent or significant incapacity or substantial disruption of the patient's ability to conduct normal life functions.
Congenital Anomaly or birth defect: An adverse outcome in a child or fetus of a patient exposed to the molecule or study treatment regimen before conception or during pregnancy.
Medically significant: The AE did not meet any of the above criteria, but could have jeopardized the patient and might have required medical or surgical intervention to prevent one of the outcomes listed above or involves suspected transmission via a medicinal product of an infectious agent.

### Adverse Event Severity

AE severity should be graded using the NCI CTCAE Version 4.03. These criteria are provided in the operations binder.

AE severity and seriousness are assessed independently. ‘Severity’ characterizes the intensity of an AE. ‘Serious’ is a regulatory definition and serves as a guide to the sponsor for defining regulatory reporting obligations (see definition for SAEs, above).

### Relationship of the Adverse Event to Study Treatment

The relationship of each AE to enfortumab vedotin and/or pembrolizumab and/or carboplatin/cisplatin/gemcitabine should be evaluated by the investigator using the following criteria:
Related: There is evidence to suggest a causal relationship between the drug and the AE, such as a single occurrence of an event that is uncommon and known to be strongly associated with drug exposure (e.g., angioedema, hepatic injury, Stevens-Johnson Syndrome) or one or more occurrences of an event that is not commonly associated with drug exposure, but is otherwise uncommon in the population exposed to the drug (eg, tendon rupture)
Unrelated: Another cause of the AE is more plausible (e.g., due to underlying disease or occurs commonly in the study population), or a temporal sequence cannot be established with the onset of the AE and administration of the study treatment, or a causal relationship is considered biologically implausible.

### (b) Procedures for Eliciting and Recording Adverse Events

Investigator and study personnel report all AEs and SAEs whether elicited during patient questioning, discovered during physical examination, laboratory testing and/or other means by recording them on the CRF and/or SAE form, as appropriate.

### Eliciting Adverse Events

An open-ended or non-directed method of questioning should be used at each study visit to elicit the reporting of AEs.

### Recording Adverse Events

The following information should be recorded on the AEs and Pre-existing Conditions CRF:
Description including onset and resolution dates
Whether it met SAE criteria
Severity
Relationship to study treatment or other causality
Outcome
   Diagnosis Vs. Signs or Symptoms

In general, the use of a unifying diagnosis is preferred to the listing out of individual symptoms. Grouping of symptoms into a diagnosis should only be done if each component sign and/or symptom is a medically confirmed component of a diagnosis as evidenced by standard medical textbooks. If any aspect of a sign or symptom does not fit into a classic pattern of the diagnosis, report the individual symptom as a separate AE.

Important exceptions for this study are adverse reactions associated with the infusion of study drug. For IRRs, record the NCI CTCAE term of ‘infusion related reaction’ with an overall level of severity (per NCI CTCAE). In addition, record each sign or symptom of the reaction as an individual AE. If multiple signs or symptoms occur with a given infusion related event, each sign or symptom should be recorded separately with its level of severity.

### Recording Serious Adverse Events

For SAEs, record the event(s) on both the CRF and an SAE form.

The following should be considered when recording SAEs:
Death is an outcome of an event. The event that resulted in the death should be recorded and reported on both an SAE form and CRF.
For hospitalizations, surgical, or diagnostic procedures, the illness leading to the surgical or diagnostic procedure should be recorded as the SAE, not the procedure itself. The procedure should be captured in the narrative as part of the action taken in response to the illness.

### Progression of the Underlying Cancer

Since progression of the underlying malignancy is being assessed as an efficacy variable, it should not be reported as an AE or SAE. Radiographic signs of disease progression (eg, ‘tumor progression’ or ‘metastases’) should not be reported as AEs or SAEs (these data are captured in the efficacy assessment). The clinical symptoms and signs of disease progression (eg, ‘fatigue’, ‘dyspnea’) may be reported as AEs or SAEs if the symptom cannot be determined as being exclusively due to progression of the underlying malignancy or does not fit the expected pattern of progression for the disease under study; do not report disease progression as the AE term. In addition, complications from progression of the underlying malignancy should be reported as AEs or SAEs.

Death is an outcome of an event. The event that resulted in the death should be recorded and reported on both an SAE form and CRF.

### Pregnancy

Notification to Drug Safety: Complete a Pregnancy Report Form for all pregnancies that occur from the time of first study drug dose until 6 months after the last dose of study drug(s) including any pregnancies that occur in the partner of a male study patient. Only report pregnancies that occur in a male patient's partner if the estimated date of conception is after the male patient's first study drug dose. Email or fax to the Drug Safety Department within 48 hours of becoming aware of a pregnancy (see e-mail or fax number specified on the SAE report form). All pregnancies are monitored for the full duration; all perinatal and neonatal outcomes should be reported. Infants should be followed for a minimum of 8 weeks.

Collection of data on the CRF. All pregnancies (as described above) that occur within 30 days of the last dose of study drug(s) are also recorded on the AEs and Pre Existing Conditions CRF.

Abortion, whether accidental, therapeutic, or spontaneous, should be reported as an SAE. Congenital anomalies or birth defects, as defined by the ‘serious’ criterion above (see definitions Section 6.3.7.7(i)(a)) should be reported as SAEs.

### Corneal Adverse Events

Corneal ulcer or keratitis AEs & Grade 2 should be graded within their respective NCI CTCAE categories. Grade 1 corneal ulcer or keratitis AEs should be graded per “Eye disorders—Other, specify” criteria. Other corneal AEs should be recorded and graded per “Eye disorders—Other, specify” criteria.

### Diabetes and Hyperglycemia

Grading for diabetes should be based on the NCI CTCAE v4.03 event term of glucose intolerance. Grading for hyperglycemia should be based on the NCI CTCAE v4.03 event term of hyperglycemia.

### Adverse Events of Possible Hepatic Origin

If an AE is accompanied by increases in liver function test (LFT) values (eg, AST, ALT, bilirubin, etc.) or is suspected to be due to hepatic dysfunction, see section 6.3.11 for detailed information on recommended monitoring and assessment of liver abnormalities. See Section 6.3.6.2(iii)(a) for treatment discontinuation recommendations related to hepatic safety.

Patients with AEs of hepatic origin accompanied by LFT abnormalities should be carefully monitored.

### (c) Reporting Periods for Adverse Events and Serious Adverse Events

AEs and SAEs for neoadjuvant and adjuvant should be reported from study Day 1 through EOT or 30 days post-study intervention, whichever is later. SAEs that occur after the safety reporting period that are assessed as related to enfortumab vedotin or RC+PLND are to be reported.

### (d) Serious Adverse Events Require Immediate Reporting

Within 24 hours of observing or learning of an SAE, investigators are to report the event to the sponsor, regardless of the relationship of the event to the study treatment regimen.

For initial SAE reports, available case details are to be recorded on an SAE form. At a minimum, the following should be included:
Patient number
Date of event onset
Description of the event
Investigator's causality assessment
Study treatment, if known

The completed SAE form and SAE Fax Cover Sheet are to be emailed or faxed to the sponsor's Drug Safety Department within 24 hours.

Relevant follow-up information is to be submitted to the sponsor as soon as it becomes available.

### (ii) Clinical Laboratory Tests

Samples are drawn for central and local labs.

Local laboratory testing includes institutional standard tests for study eligibility, evaluating safety, and making clinical decisions. All local laboratory results must be reviewed prior to study drug administration in order to determine whether to proceed with dosing or whether dose modification is required.

The following laboratory assessments are performed by the local lab to evaluate safety at scheduled time points (see Table 41) during the course of the study:
The serum chemistry panel is to include the following tests: albumin, alkaline phosphatase, ALT, AST, bicarbonate, blood urea nitrogen, calcium, creatinine, chloride, glucose, lactate dehydrogenase, phosphorus, potassium, sodium, total bilirubin, amylase, lipase, uric acid, and GFR. Note: verify blood glucose is <250 mg/dL prior to dosing (see Section 6.3.6.4). Patients with diabetes must be tested in the clinic and blood glucose must be <250 mg/dL prior to dosing. Use of insulin is permitted as part of the SOC.
The CBC with differential is to include the following tests: white blood cell count with five part differential (neutrophils, lymphocytes, monocytes, eosinophils, and basophils), platelet count, hemoglobin, and hematocrit.
CrCl at baseline as determined per institutional standards.
Thyroid function tests, including: Triiodothyronine or free triiodothyronine, free thyroxine, and thyroid-stimulating hormone
Standard urinalysis (with reflexive microscopy).
INR/PT/PTT
Serology for hepatitis B surface antigen and antihepatitis B core antibody.
Serology for antihepatitis C antibody. If positive, follow-up with polymerase chain reaction/viral load testing.
A serum or urine beta human chorionic gonadotropin pregnancy test for females of childbearing potential.
HbA1c. If HbA1c is elevated (6.5%), refer patient to appropriate provider during Cycle 1 for glucose management.
   (iii) Physical Examination Including Weight

Physical examinations should include assessments of the following body parts/systems: skin, abdomen, extremities, head, heart, lungs, neck, and neurological. Height is only collected at the Baseline visit. Weight is collected at specified time points (see Table 41) and additionally per institutional standards, if applicable, but does not need to be collected at visits following EOT.

### (iv) ECOG Performance Status

ECOG (Eastern Cooperative Oncology Group) performance status (Table 43) will be evaluated at protocol-specified time points (see Table 41).

### (v) Cardiac Monitoring

ECGs are conducted at baseline and at the EOT visit. Additional ECGs should be conducted if clinically indicated. Routine 12-lead ECGs are performed after the patient has been in a supine position for at least 5 minutes. The ECG assessments should be performed prior to obtaining the PK and biomarker samples, if possible. For subjects with NYHA Class III heart failure (see Section 6.3.10) or a history of coronary heart disease, arrhythmia or other significant heart disease, a transthoracic ECHO is required at baseline. If clinically indicated, future testing should use the same modality.

### (vi) Complete Eye Examination

Patients have a complete eye examination at screening performed by a qualified optometrist or ophthalmologist, including but not limited to: visual acuity, slit lamp, tonometry examination, and dilated fundus examination. Subsequent eye examinations are to be conducted as clinically indicated. EOT slit lamp examinations are required for patients who experience corneal AEs during the study. EOT slit lamp examinations (only required for patients who experience corneal AEs during the study) must be performed at least 4 weeks from last dose.

### 6.3.7.8 Post-treatment Assessments

### (i) Follow-up Assessments

The assessments listed below are done every 12 weeks (±7 days) until the end of 2 years from the post-RC baseline scan and subsequently every 24 weeks (±14 days) until disease progression/recurrence, patient death, withdrawal of consent, or study closure, whichever occurs first.
Physical exam (including weight)
ECOG performance status
CT with IV contrast of the chest and a urogram of the abdomen and pelvis. If contrast media are contraindicated, please refer to the current Image Acquisition Guidelines (see section 6.3.12) for preferred scanning and contrast options. Subsequent scans are to be performed via the same radiologic modalities.
QoL PRO assessment (EQ-5D-5L)
Brain scan (if clinically indicated)
Bone scan (if clinically indicated)
   (ii) Long-term follow-up Assessments

During long term follow up, patients are contacted every 24 weeks (±14 days) for survival status, QoL PRO assessment (EQ-5D-5L) (for Cohort L), and collection of subsequent anticancer therapy information until death, study closure, or withdrawal of consent, whichever occurs first.

### 6.3.7.9 Appropriateness of Measurements

The safety measures that are used in this trial are considered standard procedures for evaluating the potential adverse effects of study medications.

The determination of antitumor activity is based on confirmed objective response assessments as defined by RECIST Version 1.1 (Eisenhauer 2009) (see Table 42) and treatment decisions by the investigator will be based on RECIST Version 1.1. RECIST criteria are considered standard in oncological practice for this type of neoplasm, and the intervals of evaluation in this protocol are appropriate for disease management.

Immunogenicity is commonly assessed for biologics; therefore, standard tests are performed to detect the possible presence of specific antibodies to enfortumab vedotin.

Pharmacokinetic assessments are also common in clinical studies to help characterize dose exposure response relationships.

Exploratory biomarker measurements in peripheral blood samples enable correlation with PK assessments and are common in clinical studies. Assessments conducted on pre-treatment tumor tissue are similarly common. Both peripheral blood and tumor biomarker samples are assessed using commonly employed, standard tests.

RC+PLND with curative intent usually occurs 8 to 12 weeks after MIBC diagnosis, offering a unique opportunity to test new perioperative therapeutic approaches.

Pathologic downstaging (≤pT1) to non-muscle-invasive disease in response to NAC is a well-recognized biomarker of improved OS and would be considered a suitable endpoint for neoadjuvant trials (Sonpavde 2009; Chism 2013).

### 6.3.8 Data Analysis Methods

### 6.3.8.1 Determination of Sample Size

Approximately 50 patients may be enrolled in this study.

The sample size is not based on power calculations for formal hypothesis testing, but is selected based on the precision of the estimate for pCRR as characterized by the 95% CIs.

For a sample size of 50 patients, and assuming a pCRR (pathological complete response rate) of 30% or 40%, the 2-sided 95% CIs are summarized below in Table 40.

TABLE 40

| pCRR | 95% Exact CI (n = 20) | 95% Exact CI (n = 50) |
|---|---|---|
| 30% | (12%, 54%) | (18%, 45%) |
| 40% | (19%, 64%) | (26%, 55%) |

### 6.3.8.2 Study Endpoint Definitions

(i) Pathological Complete Response Rate (pCRR)

The pCRR is defined as the proportion of patients having pCR. pCR is defined as the absence of viable tumor (pT0N0) in examined tissue from RC+PLND.

(ii) Pathological Downstaging Rate (pDSR)

pDSR is defined as the proportion of patients having pDS. pDS is defined as patients with <pT2 (includes pT0, pTis, pTa, pT1) and N0 in examined tissue from RC+PLND.

(iii) Disease Free Survival (DFS)

DFS is defined as the time from post-RC baseline scan to the first occurrence of either:
Local or distant recurrence as assessed by CT or 1\1RI and/or biopsy; or
Death due to any cause.

### (iv) Event Free Survival (EFS)

EFS is defined as the time from the start of study treatment to the first occurrence of any of the following events:
Radiographic disease progression precluding a curative intent surgery prior to RC+PLND
Failure to undergo RC+PLND for participants with residual muscle-invasive disease and/or any radiographic disease present
Gross residual disease left behind at time of RC+PLND (surgeon unable to complete curative intent surgery due to unresectable tumor or newly discovered metastatic disease)
Local or distant recurrence post-RC as assessed by CT or MRI and/or biopsy. If biopsy is not feasible due to participant safety, CT/MRI alone will be sufficient
Death from any cause

NOTE: A non-urothelial second primary malignancy is not considered an event.

EFS data will be censored as described below, for the primary analysis of EFS:
Patients who do not refuse surgery and do not have any EFS event are censored at the date of the last disease assessment;
Patients with no disease, with NMIBC or incomplete evaluation who refuse surgery are censored at the last disease assessment prior to refusal of surgery;
Patients with residual MIBC or progressive disease who refuse surgery are counted as events. Patients who refuse surgery but without a post-screening scan will be censored at study day 1.

### (v) Overall Survival

OS is defined as the time from start of study treatment to date of death due to any cause. In the absence of confirmation of death, OS is censored at the last date the patient is known to be alive.

### 6.3.8.3 Statistical and Analytical Plans

The statistical and analytical plans presented below summarize the more complete plans to be detailed in the statistical analytical plan (SAP). A change to the data analysis methods described in the protocol requires a protocol amendment only if it alters a principal feature of the protocol. The SAP is finalized prior to database lock. Any changes to the methods described in the final SAP are described and justified in the clinical study report.

### (i) General Considerations

Disease response is summarized based on the pathological tumor response at the time of RC+PLND by central pathology reviews. Tumor response based on imaging is also summarized per RECIST Version 1.1 criteria, as assessed by investigator and BICR.

In general, descriptive statistics are presented that include the number of observations, mean, standard deviation, median, minimum and maximum for continuous variables, and the number and percentages (of non-missing) per category for categorical variables.

Unless otherwise specified, CI is calculated at two-sided 95% level.

The two-sided 95% exact CI using Clopper-Pearson methodology (Clopper 1934) is calculated for the response rates where applicable (eg, ORR, DCR, pCRR, and pDSR).

For time-to-event endpoints, the median survival time is estimated using the Kaplan Meier method; the associated 95% CI is calculated based on the complementary log-log transformation (Collett 1994).

### Data Transformations and Derivations

Time variables based on 2 dates, e.g., Start Date and End Date, are calculated as (End Date−Start Date+1) (in days) unless otherwise specified in the planned analysis section.

Unless otherwise specified in the analysis plan, baseline values used in all analyses are the most recent non-missing measurement prior to the first dose of study drug.

### Analysis Sets

The safety analysis set includes all patients who receive any amount of study drug (either enfortumab vedotin or combination agent). The safety analysis set is used for all safety analyses.

The full analysis set (FAS) includes all patients who are enrolled in the study and received any amount of study drug (enfortumab vedotin or combination agent). The FAS is used for the analysis of efficacy endpoints. Note that the analysis population for pCR includes all patients in the FAS, with the exception of those with no disease who refuse surgery. The analysis population for pDS includes all patients in the FAS who undergo surgery and have a tissue sample examined from RC+PLND. Patient demographics and baseline disease characteristics are summarized based on the FAS.

The efficacy-evaluable analysis set includes all patients in the FAS who had at least 2 post-baseline response assessments or discontinued from treatment for any reason. The efficacy-evaluable analysis set is used for additional analyses of efficacy endpoints.

The PK analysis set includes all patients who received enfortumab vedotin and from whom at least 1 blood sample collected and assayed for enfortumab vedotin, MMAE or Tab concentration. Corresponding records of the time of dosing and sample collection must also be available for all enfortumab vedotin, MMAE, and TAb concentration. The PK analysis set is used for analyses of PK parameters.

Additional analysis sets of patients may be defined in the SAP.

### Examination of Subgroups

As exploratory analyses, subgroup analyses may be conducted for selected endpoints.

### Timing of Analyses

The primary analyses are conducted after all treated patients have completed their assigned study interventions and have had adequate follow-up on the study.

Additional cutoff dates may be defined and corresponding database locks may occur to allow for more precise estimates of time-to-event endpoints.

### (ii) Patient Disposition

An accounting of study patients by disposition is tabulated and the number of patients in each analysis set is summarized, by cohorts and overall. Patients who discontinue study treatment and patients who withdraw from the study is summarized with reason for discontinuation or withdrawal for all enrolled patients.

(iii) Patient Characteristics

Demographics and other baseline characteristics are summarized using FAS, by cohorts and overall. Details are provided in the SAP.

### (iv) Efficacy Analyses

The primary analysis of efficacy endpoints are analyzed using the FAS as defined in Section 6.3.8.3. Supplemental analyses of efficacy endpoints may be presented using the efficacy-evaluable analysis sets.

Analyses of pCRR, pDSR, DFS, EFS, and OS are performed.

The key efficacy endpoint of this component of the study is pCRR by central pathology review. The observed pCRR and the 95% CIs are provided using Clopper-Pearson methodology, by cohorts.

pDSR by central pathology review is a secondary endpoint and is analyzed similarly as mentioned above.

Other secondary endpoints, such as DFS by the investigator, DFS by BICR, EFS by the investigator, EFS by BICR, and OS, are time-to-event endpoints, and they are analyzed using Kaplan-Meier methodology and Kaplan-Meier plots are provided, by cohorts. Details on the censoring algorithm are provided in the SAP.

### (v) Pharmacokinetic and ATA Analyses

Enfortumab vedotin ADC, MMAE, and Tab concentrations are summarized with descriptive statistics at each PK sampling time point. PK parameters, including, but not limited to AUC, Cmax, and Tmax, are estimated by noncompartmental analyses and summarized by descriptive statistics. The relationship between PK and pharmacodynamics endpoints, safety, or efficacy may be explored.

The incidence of ATA are summarized by visit and cohort using the safety analysis set.

### (vi) Patient Reported Outcome Analyses

Completion and compliance rates for each assessment are summarized. PRO analyses are performed on the FAS population. If any additional analysis is planned, it is detailed in a supplemental SAP.

(vii) Biomarker Analyses

Relationships of biomarker parameters (eg, pre-treatment values, absolute and relative changes from pre-treatment) to efficacy, safety, and PK parameters are explored. Relationships and associated data that are determined to be of interest are summarized. Details of these analyses are described separately.

(viii) Safety Analyses

The safety analysis set is used to summarize all safety endpoints.

### (a) Extent of Exposure

Duration of treatment, number of cycles, total dose and dose intensity is summarized, by expansion cohorts. Dose modifications are also summarized in similar manner. Details are provided in the SAP.

### (b) Adverse Events

An overview of AEs provides a tabulation, by cohort and overall, of the incidence of all AEs, TEAEs, treatment-related AEs, Grade 3 and higher AEs, SAEs, treatment-related SAEs, deaths, and AEs leading to study treatment discontinuation. AEs are defined as treatment emergent if they are newly occurring or worsen following study treatment.

AEs are listed and summarized by Medical Dictionary for Regulatory Activities (MedDRA), preferred term, severity, and relationship to study drug. In the event of multiple occurrences of the same AE with the same preferred term in 1 patient, the AE is counted once as the occurrence. The incidence of AEs is tabulated by preferred term and cohorts. AEs leading to premature discontinuation of study drug is summarized and listed in the same manner.

### (c) Deaths and Serious Adverse Events

SAEs are listed and summarized in the same manner as all AEs. Events with a fatal outcome are listed.

### (d) Clinical Laboratory Results

Laboratory values (e.g., chemistry, hematology, and urinalysis with reflexive microscopic analysis) may be presented graphically by visit. Summary statistics may be tabulated as appropriate by scheduled visit. Laboratory values are listed with grade per NCI CTCAE Version 4.03 and flagged when values are outside the normal reference range.

### (e) Other Safety Analyses

### Vital Signs

Summary statistics and change from baseline and/or predose to postdose may be tabulated where appropriate.

### ECOG Status

ECOG status is summarized for each visit. Shifts from baseline to the best and worst post-baseline score may be tabulated, by cohort and overall.

### ECG

ECG status (normal, abnormal clinically significant, or abnormal not clinically significant) may be summarized for each scheduled and unscheduled ECG, and shifts from baseline may be tabulated, by cohort and overall.

### (ix) Interim Analyses

No formal interim analysis is planned.

TABLE 41
Schedule of Events

| | | | | | | | | | | Post-RC |
|---|---|---|---|---|---|---|---|---|---|---|
| | | | | | Neoadjuvant Period | | | | | Baseline |
| | | Screening/Baseline | | Enrollment | Every 3-Week Cycle (3 cycles) | | | | RC | Scan^{S} |
| | | Day | | | | | | | | |
| | | | | Within 1 | | | | Pre-RC ≤4 | | |
| | | D −28 | D −7 | to 2 d of | | | | wk prior | Week | 6 weeks |
| | | to 1 | to 1 | First Dose | D 1 | D 8 | D 15 | to RC | 12 | after RC |
| | | Visit Window | | | | | | | | |
| | | | | | ±2 d^{C} | +1 d | ±1 d | | ±7 d | ±14 d |
| Screening/ | Inclusion/Exclusion, | X | | Submit | | | | | | |
| Baseline | medical history | | | confirmation | | | | | | |
| Assessments | Informed consent | X | | prior to | | | | | | |
| | Initiate collection of | X | | treatment | | | | | | |
| | tumor specimen | | | | | | | | | |
| | from TURBT^{D,E} | | | | | | | | | |
| | Complete eye exam | X | | | | | | | | |
| | Brain scan if | X | | | | | | X | | X |
| | clinically indicated^{F} | | | | | | | | | |
| | Bone scan if | X | | | | | | X | | X |
| | clinically indicated^{F} | | | | | | | | | |
| | INR/PT/PTT | | X | | | | | | | |
| | Serology for | X | | | | | | | | |
| | hepatitis B surface | | | | | | | | | |
| | antigen and | | | | | | | | | |
| | antihepatitis B core | | | | | | | | | |
| | antibody | | | | | | | | | |
| | Serology for | X | | | | | | | | |
| | antihepatitis C | | | | | | | | | |
| | antibody | | | | | | | | | |
| | HbA1c^{G} | | | | | | | | | |
| | Urinalysis with | X | | | | | | | | |
| | reflexive | | | | | | | | | |
| | microscopic | | | | | | | | | |
| | analysis | | | | | | | | | |
| | Pregnancy test | | X | | X^{J} | | | | | |
| | (urine or serum; | | | | | | | | | |
| | only for females of | | | | | | | | | |
| | childbearing | | | | | | | | | |
| | potential)^{H} | | | | | | | | | |
| Safety | Physical exam | | X | | X^{I,J} | | | | | |
| Assessments | (including weight) | | | | | | | | | |
| | Height | | X | | | | | | | |
| | Vital signs | | X | | X^{I,J} | X^{J} | | | | |
| | CBC with | | X | | X^{I,J} | X^{J} | X | | | |
| | differential^{L,M,W} | | | | | | | | | |
| | Serum chemistry | | X | | X^{I,J,N} | X^{J,N} | X | | | |
| | CrCl | | X | | | | | | | |
| | Thyroid function | X | | | X^{I,J} | | | | | |
| | tests | | | | | | | | | |
| | ECOG performance | | X | | X^{I,J} | | | | | |
| | status | | | | | | | | | |
| | ECG^{O} | | X | | | | | | | |
| | Transthoracic | | X | | | | | | | |
| | ECHO^{X} | | | | | | | | | |
| | Slit lamp | | | | | | | | | |
| | examination | | | | | | | | | |
| | Concomitant | Related to study procedures^{Q} | | | AEs and SAEs for neoadjuvant and adjuvant Cohort | | | | | |
| | medications | | | | L should be reported from study Day 1 through EOT | | | | | |
| | Adverse event | | | | or 30 days post-study intervention, whichever is | | | | | |
| | collection | | | | later. SAEs that occur after the safety reporting | | | | | |
| | | | | | period that are assessed as related to enfortumab | | | | | |
| | | | | | vedotin or RC + PLND are to be reported. | | | | | |
| Treatment^{W} | Enfortumab vedotin | | | | X | X | | | | |
| | administration^{R} | | | | | | | | | |
| PK/Immunogenicity/ | Blood, urine, tissue | See PK, ATA, and biomarker tables for sample collection details^{A} | | | | | | | | |
| Biomarker | sample collection | | | | | | | | | |
| Response | Imaging^{S} | X^{S} | | | | | | X^{S} | | X^{S} |
| Assessment | Pathologic | | | | | | | | X | |
| | assessment at time | | | | | | | | | |
| | of RC | | | | | | | | | |
| | Survival status^{T} | | | | | | | | | |
| | Urine Cytology^{Y} | | | | | | | | | |
| Patient Reported | Quality of Life | | | | X^{J,Z} | X^{J,Z} | | | X^{Z} | |
| Outcome | Assessment^{Z} | | | | | | | | | |
| Assessments | | | | | | | | | | |

| | | Adjuvant Period Every 3-Week | | | | | Long- |
|---|---|---|---|---|---|---|---|
| | | Cycle (6 cycles) | | | | | Term |
| | | starting 8 weeks after | | | | | Follow- |
| | | RC ±14 days | | | EOT^{B,Z} | Follow-Up^{U,Z} | Up^{V,Z} |
| | | Day | | | | | |
| | | | | | | Every 12 weeks until | Every |
| | | | | | | end of 2 years, then | 24 |
| | | D 1 | D 8 | D 15 | | every 24 weeks | weeks |
| | | Visit Window | | | | | |
| | | | | | | ±7 d (until end | |
| | | | | | | of 2 years), | |
| | | ±2 d^{C} | +1 d | ±1 d | | then ±14 d | ±14 d |
| Screening/ | Inclusion/Exclusion, | | | | | | |
| Baseline | medical history | | | | | | |
| Assessments | Informed consent | | | | | | |
| | Initiate collection of | | | | | | |
| | tumor specimen | | | | | | |
| | from TURBT^{D,E} | | | | | | |
| | Complete eye exam | | | | | | |
| | Brain scan if | | | | | X | |
| | clinically indicated^{F} | | | | | | |
| | Bone scan if | | | | | X | |
| | clinically indicated^{F} | | | | | | |
| | INR/PT/PTT | | | | | | |
| | Serology for | | | | | | |
| | hepatitis B surface | | | | | | |
| | antigen and | | | | | | |
| | antihepatitis B core | | | | | | |
| | antibody | | | | | | |
| | Serology for | | | | | | |
| | antihepatitis C | | | | | | |
| | antibody | | | | | | |
| | HbA1c^{G} | | | | | | |
| | Urinalysis with | | | | | X | |
| | reflexive | | | | | | |
| | microscopic | | | | | | |
| | analysis | | | | | | |
| | Pregnancy test | X^{J} | | | X | | |
| | (urine or serum; | | | | | | |
| | only for females of | | | | | | |
| | childbearing | | | | | | |
| | potential)^{H} | | | | | | |
| Safety | Physical exam | X^{J} | | | X | X^{K} | |
| Assessments | (including weight) | | | | | | |
| | Height | | | | | | |
| | Vital signs | X^{J} | X^{J} | | X | | |
| | CBC with | X^{J} | X^{J} | X | X | | |
| | differential^{L,M,W} | | | | | | |
| | Serum chemistry | X^{J,N} | X^{J,N} | X | X | | |
| | CrCl | | | | | | |
| | Thyroid function | X^{J} | | | X | | |
| | tests | | | | | | |
| | ECOG performance | X^{J} | | | X | X | |
| | status | | | | | | |
| | ECG^{O} | | | | X | | |
| | Transthoracic | | | | | | |
| | ECHO^{X} | | | | | | |
| | Slit lamp | | | | X^{P} | | |
| | examination | | | | | | |
| | Concomitant | AEs and SAEs for neoadjuvant and adjuvant Cohort | | | | | |
| | medications | L should be reported from study Day 1 through EOT | | | | | |
| | Adverse event | or 30 days post-study intervention, whichever is | | | | | |
| | collection | later. SAEs that occur after the safety reporting | | | | | |
| | | period that are assessed as related to enfortumab | | | | | |
| | | vedotin or RC + PLND are to be reported. | | | | | |
| Treatment^{W} | Enfortumab vedotin | X | X | | | | |
| | administration^{R} | | | | | | |
| PK/Immunogenicity/ | Blood, urine, tissue | See PK, ATA, and biomarker tables for sample collection details^{A} | | | | | |
| Biomarker | sample collection | | | | | | |
| Response | Imaging^{S} | | | | X^{AA} | X^{S} | |
| Assessment | Pathologic | | | | | | |
| | assessment at time | | | | | | |
| | of RC | | | | | | |
| | Survival status^{T} | | | | | | X^{T} |
| | Urine Cytology^{Y} | | | | X | X | |
| Patient Reported | Quality of Life | | | | X^{Z} | X^{Z} | X^{Z} |
| Outcome | Assessment^{Z} | | | | | | |
| Assessments | | | | | | | |

^{A}See Table 20 for PK, ATA, and biomarker sampling.
^{B}EOT visits should occur within 30-37 days after patient completes all required study intervention (study intervention includes RC + PLND) or discontinues prior to completion of all required study intervention. EOT evaluations must be performed before initiation of a new therapy for cancer, with the exception of the slit lamp examination (slit lamp examinations must be performed ≥4 weeks after last dose). If EOT evaluations are completed before 30 days after last study intervention, the patient will be contacted by telephone 30 to 37 days following the last date of study intervention to complete safety follow up and assess for AEs.
^{C}Window applies to Neoadjuvant Cycles 2 and 3 and Adjuvant Cycles 1 to 6.
^{D}Within 90 days before Cycle 1 Day 1. This tissue will also be sent for PD-L1 determination by immunohistochemistry (IHC).
^{E}Invasive tumor tissue obtained at the time of the patient's original diagnosis for biomarker studies. Samples must be confirmed as available for participation in the study. Tumor block is preferred. If tumor block is not available, slides are acceptable.
^{F}Repeated as clinically indicated throughout the study.
^{G}If HbA1c is elevated (≥6.5%), refer patient to appropriate provider during Cycle 1 for glucose management.
^{H}Either serum or urine pregnancy test for females of childbearing potential. Baseline assessment Day −3 to Day 1. Either serum or urine pregnancy test. Not required for Cycle 1 if baseline assessment performed within 7 days. Repeat every month (±1 week) for 6 months after EOT. The pregnancy test may be performed within 1 day prior to administration of study drug.
^{I}Not required for Cycle 1 if baseline assessments performed within 1 day.
^{J}Procedures should be conducted pre-dose.
^{K}Follow-up physical exam will not include weight measurement.
^{L}Abnormal lab values that are clinically significant will be confirmed within 1 day during Neoadjuvant Cycle 1, as clinically indicated.
^{M}Safety labs may be collected up to 1 day prior to dosing.
^{N}Verify blood glucose is <250 mg/dL prior to dosing. Patients with diabetes must be tested in the clinic and blood glucose must be <250 mg/dL prior to dosing. Use of insulin is permitted as part of the standard of care.
^{O}Standard Baseline/EOT ECG assessments will be performed for all patients.
^{P}Only required for patients who experience corneal AEs during the study; must be performed ≥4 weeks after last dose.
^{Q}From time of informed consent.
^{R}At least 1 week must elapse between doses of enfortumab vedotin.
^{S}Baseline radiographic screening assessment will be done using CT with IV contrast of the chest and a CT urogram of the abdomen and pelvis. If contrast media are contraindicated, please refer to the current Image Acquisition Guidelines (Section 6.3.12) for preferred scanning and contrast options. Pre-RC scans should be completed ≤ 4 weeks prior to RC + PLND (RC + PLND must occur at week 12 from start of study treatment). Pre-RC scans should be reviewed prior to RC + PLND. The post-RC baseline scan should be performed at 6 weeks (±14 days) after RC + PLND. After the post-RC baseline imaging, patients will undergo imaging every 12 weeks (± 7 days) until the end of 2 years from their post-RC baseline scan. After this period on study, the frequency of visits, including response assessments, will be reduced to every 24 weeks (±14 days). Imaging will continue until disease progression/recurrence, patient death, study closure, or withdrawal of consent, whichever occurs first.
^{T}Contact patient for survival status, disease status, and collection of subsequent anticancer treatment information until death, study closure, or patient withdraws consent, whichever occurs first.
^{U}Follow-up will be done every 12 weeks (±7 days) until the end of 2 years from post-RC baseline scan. The imaging will continue until disease progression/recurrence, patient death, study closure, or withdrawal of consent, whichever occurs first. After this period on study, the frequency of visits including response assessments will be reduced to every 24 weeks (±14 days).
^{V}Patients will be contacted every 24 weeks (±14 days) after RC + PLND for survival status and collection of subsequent anticancer therapy information until death, study closure, or withdrawal of consent, whichever occurs first.
^{W}Review the results of all local laboratory tests prior to study drug administration in order to determine whether to proceed with dosing or whether dose modification is required. If there are AEs preventing dosing on Day 1 of any Cycle (Neoadjuvant Cycles 2 and 3 and Adjuvant Cycles 1 to 6), dosing should be delayed and not eliminated. If there are AEs that do not resolve within 2 days of planned dose for Day 8 of any cycle then that dose should be eliminated. If Day 8 dose is eliminated, research samples should still be drawn (for the applicable cycles). If enfortumab vedotin has been permanently discontinued, the patient should still have safety labs completed for Day 8 and 15 visits.
^{X}A transthoracic echocardiogram is required for subjects with NYHA Class III heart failure or a history of coronary heart disease, arrhythmia or other significant heart disease.
^{Y}Only required for patients who do not undergo RC and PLND surgery (see Section 7.2.2.2).
^{Z}Patient reported outcome (PRO) assessments will be completed using EQ 5D 5L. Assessments will be completed within 2 days prior to dosing on neoadjuvant Cycles 1-3 Days 1 and 8. After completion of the neoadjuvant cycles, patients will complete ePROs once at RC + PLND (if applicable) and at EOT. Following RC + PLND (if applicable) and EOT, patients will complete PRO assessments once every 12 weeks for 2 years (for the Follow-Up period) and once every 24 weeks thereafter (for the Long-Term Follow-Up period). If a visit occurs out of the assessment window, assessments should still be completed.
^{AA}For patients who are discontinuing study intervention, EOT Imaging should be done within 4 weeks of the last study intervention. Not required if imaging was performed within 4 weeks of discontinuing study intervention. If EOT imaging is completed, subsequent scans should be based on the protocol-specified imaging schedule.

TABLE 42
RECIST Criteria Summary (Version 1.1)
Response Evaluation Criteria in Solid Tumors

| Term | Definition |
|---|---|
| Complete response (CR) | Disappearance of all target lesions. Any pathological lymph nodes |
| | must have reduction in short axis to <10 mm. |
| Partial response (PR) | A ≥30% decrease in the sum of diameters of target lesions, taking |
| | as reference the baseline sum diameters. |
| Progressive disease (PD) | At least a 20% increase in the sum of diameters of target lesions, |
| | taking as reference the smallest sum of diameters on study (this |
| | includes the baseline sum if that is the smallest on study). In |
| | addition to the relative increase of 20%, the sum must also |
| | demonstrate an absolute increase of at least 5 mm. The appearance |
| | of one or more new lesions is also considered progression. |
| Stable disease (SD) | Neither sufficient shrinkage to qualify for PR nor sufficient |
| | increase to qualify for PD, taking as reference the smallest sum of |
| | diameters while on study. |
| Measurable lesion(s) | Must be accurately measured in at least 1 dimension (longest |
| | diameter in the plane of measurement is to be recorded) with a |
| | minimum size of 10 mm by CT/MRI scan (CT slice thickness no |
| | greater than 5 mm) (maximum of 5 lesions, no more than 2 per |
| | site, at least 10 mm in long axis [or 15 mm in short axis for nodal |
| | lesions]). |
| | A lymph node must be ≥15 mm in short axis when assessed by |
| | CT/MRI scan to be considered measurable. |

From RECIST Version 1.1 (Eisenhauer 2009)

A response (CR or PR) is considered confirmed if the following disease assessment (at least 4-5 weeks after the initial response) still shows response (CR or PR). In cases where the initial response is followed by SD, it is considered as confirmed if the SD is later followed by PR or CR. For example, if a patient had PR in Week 9, SD in Week 14, and PR in Week 18, this PR is considered as confirmed.

### Baseline Lesion Identification

When more than one measurable lesion is present at baseline all lesions up to a maximum of five lesions total (and a maximum of two lesions per organ) representative of all involved organs should be identified as target lesions and recorded and measured at baseline. Target lesions should be selected on the basis of their size (lesions with the longest diameter), be representative of all involved but in addition should be those that lend themselves to reproducible repeated measurements (bone lesions should not be selected as a target lesion). It may be the case that, on occasion, the largest lesion does not lend itself to reproducible measurement in which circumstance the next largest lesion which can be measured reproducibly should be selected. Lesions in the urinary bladder that are detected on CT/MRI scans may be selected as target lesions, provided the radiologist considers the lesion(s) lend to reproducible measurements at post-baseline timepoints and lesions have been measured with adequate bladder distension. The same imaging modality should be used throughout the study treatment.

Pathological lymph nodes which are defined as measurable and may be identified as target lesions must meet the criterion of a short axis of ≥15 mm by CT or MRI scan. All other pathological nodes (those with short axis ≥10 mm but <15 mm) should be considered nontarget lesions. Lymph nodes that have a short axis <10 mm are considered non-pathological and should not be recorded or followed. All other lesions (or sites of disease) including pathological lymph nodes should be identified as nontarget lesions and should also be recorded at baseline.

TABLE 43
Performance Status Scales Conversion

| Karnofsky | | Lansky | | ECOG | |
|---|---|---|---|---|---|
| Percent | Description | Percent | Description | Score | Description |
| 100 | Normal, no complaints, | 100 | Fully active, normal. | 0 | Normal activity. Fully |
| | no evidence of disease. | | | | active, able to carry on |
| 90 | Able to carry on normal | 90 | Minor restrictions in | | all pre-disease |
| | activity; minor signs or | | physically strenuous | | performance without |
| | symptoms of disease. | | activity. | | restriction. |
| 80 | Normal activity with | 80 | Active, but | 1 | Symptoms, but |
| | effort; some signs or | | tires more | | ambulatory. Restricted |
| | symptoms of disease. | | quickly. | | in physically strenuous |
| 70 | Cares for self, unable to | 70 | Both greater restriction | | activity, but ambulatory |
| | carry on normal activity | | of, and less time spent | | and able to carry out |
| | or to do active work. | | in, play activity. | | work of a light or |
| | | | | | sedentary nature (eg, |
| | | | | | light housework, office |
| | | | | | work). |
| 60 | Requires occasional | 60 | Up and around, but | 2 | In bed <50% of the |
| | assistance, but is able to | | minimal active play; | | time. Ambulatory and |
| | care for most of his/her | | keeps busy with quieter | | capable of all self-care, |
| | needs. | | activities. | | but unable to carry out |
| 50 | Requires considerable | 50 | Gets dressed, but lies | | any work activities. Up |
| | assistance and frequent | | around much of the | | and about more than |
| | medical care. | | day; no active play; | | 50% of waking hours. |
| | | | able to participate in all | | |
| | | | quiet active play and | | |
| | | | activities. | | |
| 40 | Disabled, requires | 40 | Mostly in bed, | 3 | In bed >50% of the |
| | special care and | | participates in quiet | | time. Capable of only |
| | assistance. | | activities. | | limited self-care, |
| 30 | Severely disabled, | 30 | In bed, needs assistance | | confined to bed or chair |
| | hospitalization | | even for quiet play. | | more than 50% of |
| | indicated. Death not | | | | waking hours. |
| | imminent. | | | | |
| 20 | Very sick, | 20 | Often sleeping, play | 4 | 100% bedridden. |
| | hospitalization | | entirely limited to | | Completely disabled. |
| | indicated. Death not | | very passive | | Cannot carry on any |
| | imminent. | | activities. | | self-care. Totally |
| 10 | Moribund, fatal | 10 | No play, does not get | | confined to bed or chair. |
| | processes progressing | | out of bed. | | |
| | rapidly. | | | | |
| 0 | Dead. | 0 | Dead. | 5 | Dead. |

### 6.3.10 New York Health Association Classification

A Functional and Therapeutic Classification for Prescription of Physical Activity for Cardiac Patients.
Class I: Patients with no limitation of activities; they suffer no symptoms from ordinary activities.
Class II: Patients with slight, mild limitation of activity; they are comfortable with rest or with mild exertion.
Class III: Patients with marked limitation of activity; they are comfortable only at rest.
Class IV: Patients who should be at complete rest, confined to bed or chair; any physical activity brings on discomfort and symptoms occur at rest.

On-line source: www.heart.org/HEARTORG/Conditions/HeartFailure/AboutHeartFailure/Classes-of-Heart-Failure_UCM_306328_Article.jsp

### 6.3.11 Liver Safety Monitoring and Assessment

The following recommendations are from the FDA Guidance for Industry titled “Drug-Induced Liver Injury: Premarketing Clinical Evaluation” issued July 2009.

Any patient with an increase of serum aminotransferases to >3×upper limit of normal (ULN) or bilirubin >2×ULN should undergo detailed testing for liver enzymes (including at least alanine aminotransferase [ALT], aspartate aminotransferase [AST], alkaline phosphatase [ALP], and total bilirubin). To confirm the abnormality, testing should be repeated within 72 hours of notification of the test results.

### Definition of Liver Abnormalities

Confirmed liver abnormalities will be characterized as Moderate and Severe:

Moderate:
ALT or AST>3×ULN OR Total Bilirubin >2×ULN

Severe:
ALT or AST>3×ULN AND Total Bilirubin >2×ULN (*See Hy's Law Definition)
ALT or AST>8×ULN
ALT or AST>5×ULN for more than 2 weeks
ALT or AST>3×ULN and INR>1.5 (if INR testing is applicable/evaluated)
ALT or AST>3×ULN with the appearance of symptoms suggestive of liver injury (eg, right upper quadrant pain or tenderness) and/or eosinophilia (>5%)

The investigator may determine that abnormal liver function results, other than as described above, may qualify as moderate or severe abnormalities and require additional monitoring and follow-up.

*Hy's Law: Drug-induced jaundice caused by hepatocellular injury, without a significant obstructive component, has a high rate of bad outcomes, from 10 to 50% mortality (or transplant). The 2 “requirements” for Hy's Law are: 1) Evidence that a drug can cause hepatocellular-type injury, generally shown by an increase in transaminase elevations higher 3×ULN (“2×ULN elevations are too common in treated and untreated patients to be discriminating”). 2) Cases of increased bilirubin (at least 2×ULN) with concurrent transaminase elevations at least 3×ULN and no evidence of intra- or extra-hepatic bilirubin obstruction (elevated ALP) or Gilbert's syndrome (Temple et al., Pharmacoepidemiol Drug Saf (2006); 15(4): 241-3.).

### Follow-Up Procedures

Confirmed moderate and severe abnormalities in hepatic functions should be thoroughly characterized by obtaining appropriate expert consultations, detailed pertinent history, physical examination and laboratory tests. Patients with confirmed abnormal liver function testing should be followed as described below.

Confirmed moderately abnormal LFTs should be repeated 2 to 3 times weekly then weekly or less if abnormalities stabilize or the study drug has been discontinued and the patient is asymptomatic.

Severe hepatic liver function abnormalities as defined above, in the absence of another etiology, may be considered an important medical event and may be reported as an SAE. The sponsor should be contacted and informed of all patients for whom severe hepatic liver function abnormalities possibly attributable to study drug are observed.

To further assess abnormal hepatic laboratory finding, it is recommended that the investigator:
Obtain a more detailed history of symptoms and prior or concurrent diseases. Illnesses and conditions such as hypotensive events, and decompensated cardiac disease that may lead to secondary liver abnormalities should be noted. Nonalcoholic steatohepatitis is seen in obese hyperlipoproteinemic and/or diabetic patients, and may be associated with fluctuating aminotransferase levels.
Obtain a history of concomitant drug use (including nonprescription medication, complementary and alternative medications), alcohol use, recreational drug use and special diets.
Obtain a history of exposure to environmental chemical agents.
Based on the patient's history, other testing may be appropriate including: Acute viral hepatitis (A, B, C, D, E or other infectious agents), Ultrasound or other imaging to assess biliary tract disease, or Other laboratory tests including international normalized ratio (INR), direct bilirubin.
Consider gastroenterology or hepatology consultations.

Conduct additional testing as determined by the investigator to further evaluate possible etiology.

### 6.3.12 Image Acquisition Guidelines

### Screening Scans in Decreasing Order of Preference of the Following:

1. CT Chest and CT urogram of abdomen and pelvis with IV contrast
2. CT Chest without IV contrast and MRI urogram of abdomen and pelvis with IV gadolinium contrast (if iodinated contrast is medically contraindicated)

### Post-Cystectomy Scans in Decreasing Order of Preference of the Following:

1. CT Chest-Abdomen-Pelvis with oral and IV contrast
2. CT Chest without IV contrast and MRI Abdomen-Pelvis with IV gadolinium contrast (if iodinated contrast is medically contraindicated)
3. CT chest without IV contrast and MRI Abdomen-Pelvis without IV gadolinium contrast (if iodinated contrast and gadolinium contrast medically contraindicated)
4. MRI Chest-Abdomen and Pelvis with IV gadolinium contrast
5. Chest-Abdomen-Pelvis CT without IV contrast (oral contrast is recommended) (if patient has contraindication to iodinated contrast and MRI scan)

### Brain Scan (if Clinically Indicated) in Decreasing Order of Preference of the Following:

1. Brain MRI with IV gadolinium
2. Brain CT with IV contrast (if gadolinium and/or MRI is medically contraindicated)
3. Brain MRI without IV gadolinium (if gadolinium is medically contraindicated)
4. Brain CT without IV contrast (if gadolinium is medically contraindicated)

Imaging modality, anatomical coverage, and acquisition parameters should remain consistent across all imaging visits for each patient.

### 6.3.13 Surgery

### Surgical Guidelines:

Surgical approach for RC+PLND (eg, open, laparoscopic with robot-assistance) and urinary diversion (eg, ileal conduit, neobladder) is per discretion of the treating urologist and the participant.

RC+PLND are done in accordance with the AUA/ASCO/ASTRO/SUO guidelines*, as follows:

“When performing a standard radical cystectomy, clinicians should remove the bladder, prostate, and seminal vesicles in males and should remove the bladder, uterus, fallopian tubes, ovaries, and anterior vaginal wall in females. When performing bilateral pelvic lymphadenectomy, clinicians should remove, at a minimum, the external and internal iliac and obturator lymph nodes (standard lymphadenectomy).” In the case of neobladder reconstruction, sparing of female sex organs may be allowed based on standard of care and the treating urologist's judgment. Any deviation from this guidance should be documented in the medical record and eCRFs.

All postoperative complications are reported and graded, if applicable, in the same way as other AEs (CTCAE version 4.0) and are identified by the investigator and/or surgeon as post-surgical complications.

*https://www.auanet.org/guidelines/bladder-cancer-non-metastatic-muscle-invasive-(2017)

### 6.3.14 Guidance on Contraception

For the purposes of this guidance, complete abstinence, if consistent with the patient's preferred lifestyle, is an acceptable form of contraception. Complete abstinence is defined as abstinence starting from the time of informed consent and continuing throughout the study and until the end of systemic exposure (at least 6 months after the final dose of study drug).

### Acceptable Methods for Highly Effective Birth Control (Preventing Conception)

Patients who are of childbearing potentiala or whose partners are of childbearing potentiala and who are sexually active in a way that could lead to pregnancy may choose any TWO of the following methods (please see acceptable combinations below):
Hormonal methods of contraception (excluding progestin-only pills; method must be associated with inhibition of ovulation), unless contraindicated
Intrauterine device with failure rate <1%
Tubal ligation
Vasectomy (at least 90 days from the date of surgery with a semen analysis documenting azoospermia)
Barrier method (male or female condom with or without spermicide, cervical cap with or without spermicide, diaphragm with or without spermicide)b
A person of childbearing potential is defined as anyone born female who has experienced menarche and who has not undergone surgical sterilization (eg, hysterectomy, bilateral salpingectomy, bilateral oophorectomy) or has not completed menopause. Menopause is defined clinically as 12 months of amenorrhea in a person born female over age 45 in the absence of other biological, physiological, or pharmacological causes.
A barrier method should only be used with a highly effective birth control method that is not a barrier method. Barrier methods alone, including a double-barrier method, are not considered highly effective contraceptive measures (see unacceptable methods of contraception).

Acceptable combinations of contraceptive methods include: hormonal method and vasectomy, hormonal method and barrier method, intrauterine device and vasectomy, intrauterine device and barrier method, tubal ligation and vasectomy, and tubal ligation and barrier method.

Acceptable methods for preventing secondary exposure to seminal fluid are as follows: option 1—male condom (with or without spermicide) and cervical cap and option 2—male condom (with or without spermicide) and diaphragm. Subjects born male and who are sexually active with a pregnant or breastfeeding person must use the contraceptives in Options 1 or 2.

Unacceptable methods of contraception include periodic abstinence, spermicide only, no method, progestin-only pills, withdrawal, concomitant use of female and male condoms, rhythm, and barrier methods alone (including double-barrier methods).

1. A method of treating muscle invasive urothelial cancer (MIUC) or muscle invasive bladder cancer (MIBC) in a human subject, comprising (a) administering to the subject an effective amount of an antibody drug conjugate (ADC);
wherein the ADC comprises an antibody or antigen binding fragment thereof that binds to 191P4D12 (Nectin-4) conjugated to one or more units of monomethyl auristatin E (MMAE);
wherein the subject is ineligible to receive cisplatin treatment (cisplatin ineligible); and
wherein the pathological complete response rate (pCRR) is at least 30%.

2. A method of treating muscle invasive urothelial cancer (MIUC) or muscle invasive bladder cancer (MIBC) in a human subject, comprising (a) administering to the subject an effective amount of an antibody drug conjugate (ADC);
wherein the ADC comprises an antibody or antigen binding fragment thereof that binds to 191P4D12 (Nectin-4) conjugated to one or more units of monomethyl auristatin E (MMAE);
wherein the subject is ineligible to receive cisplatin treatment (cisplatin ineligible); and
wherein the pathological downstaging rate (pDSR) is at least 50%.

3. The method of claim 1 or claim 2, wherein the method comprises administering to the subject 3 cycles of the ADC.

4. The method of any one of claims 1 to 3, wherein the treatment of the cancer further comprises radical cystectomy and pelvic lymph node dissection (RC+PLND).

5. The method of claim 4, wherein the human subject receives the RC+PLND about four (4) to about twelve (12) weeks after the ADC is administered to the human subject.

6. The method of claim 1 or 2, further comprising (b) performing radical cystectomy and pelvic lymph node dissection (RC+PLND) on the subject.

7. The method of claim 6, wherein (b) is performed about four (4) to about twelve (12) weeks after (a).

8. A method of treating muscle invasive urothelial cancer (MIUC) or muscle invasive bladder cancer (MIBC) in a human subject, comprising: administering to the subject multiple cycles of an effective amount of an antibody drug conjugate (ADC), wherein
(a) the ADC comprises an antibody or antigen binding fragment thereof that binds to 191P4D12 (Nectin-4) conjugated to one or more units of monomethyl auristatin E (MMAE);
(b) the subject is ineligible to receive cisplatin treatment (cisplatin ineligible); and
(c) the number of cycles of ADC treatment administered to the cisplatin ineligible subject is equal to or less than the number of cycles of standard-of-care (SOC) therapy used to treat cisplatin eligible subjects with MIUC or MIBC.

9. The method of claim 8, wherein the SOC therapy for a cisplatin eligible subject comprises:
i. cisplatin;
ii. methotrexate, vinblastine, doxorubicin, and cisplatin (MVAC);
iii. gemcitabine plus cisplatin;
iv. administering a programmed cell death 1 (PD-1) inhibitor; or
v. administering a programmed cell death-ligand 1 (PD-L1) inhibitor.

10. The method of claim 8 or 9, wherein the number of cycles of ADC treatment administered to the cisplatin ineligible subject is 3 or 4.

11. A method of administering neoadjuvant or perioperative therapy to treat muscle invasive urothelial cancer (MIUC) or muscle invasive bladder cancer (MIBC) in a human subject, comprising: administering to the subject multiple cycles of an effective amount of an antibody drug conjugate (ADC), wherein:
(a) the ADC comprises an antibody or antigen binding fragment thereof that binds to 191P4D12 (Nectin-4) conjugated to one or more units of monomethyl auristatin E (MMAE);
(b) the subject is ineligible to receive cisplatin treatment (cisplatin ineligible); and
(c) the subject remains eligible for radical cystectomy and pelvic lymph node dissection (RC+PLND) surgery following the multiple cycles of treatment with the ADC.

12. The method of claim 11, wherein the ADC is administered as a neoadjuvant therapy (i) prior to the surgery or (ii) prior to and following the surgery.

13. A method of treating muscle invasive urothelial cancer (MIUC) or muscle invasive bladder cancer (MIBC) in a human subject, comprising: administering to the subject multiple cycles of an effective amount of an antibody drug conjugate (ADC), wherein:
(a) the ADC comprises an antibody or antigen binding fragment thereof that binds to 191P4D12 (Nectin-4) conjugated to one or more units of monomethyl auristatin E (MMAE);
(b) the subject is ineligible to receive cisplatin treatment (cisplatin ineligible); and
(c) the efficacy of the treatment of the subject with the effective amount of the ADC is similar to the efficacy of treatment observed with cisplatin eligible patients treated with standard-of-care (SOC) therapy used to treat cisplatin eligible subjects with MIUC or MIBC.

14. The method of claim 13, wherein the efficacy of the treatment is at least as efficacious as the efficacy of the treatment observed with SOC for cisplatin eligible subjects.

15. The method of claim 13 or 14, wherein the measure of efficacy of treatment is one or more of: pathological complete response rate (pCRR), pathological downstaging rates (pDSR), disease free survival (DFS), event-free survival (EFS), overall survival (OS), progression free survival (PFS), and duration of response (DoR).

16. The method of claim 15, wherein the pCRR for the cisplatin ineligible subject is at least 30%.

17. The method of claim 15, wherein pDSR for the cisplatin ineligible subject is at least 50%.

18. The method of any one of claims 14 to 17, wherein the SOC therapy for the cisplatin eligible subject comprises:
(i) cisplatin;
(ii) methotrexate, vinblastine, doxorubicin, and ciasplatin (MVAC);
(iii) gemcitabine plus cisplatin;
(iv) a programmed cell death 1 (PD-1) inhibitor; or
(v) a programmed cell death-ligand 1 (PD-L1) inhibitor.

19. The method of claim 18, wherein (i) the PD-1 inhibitor is nivolumab or pembrolizumab; or (ii) the PD-L1 inhibitor is selected from the group consisting of atezolizumab, avelumab, and durvalumab.

20. The method of any one of claims 1 to 19, wherein the antibody or antigen binding fragment thereof comprises a heavy chain variable region comprising complementarity determining regions (CDRs) comprising the amino acid sequences of the CDRs of the heavy chain variable region set forth in SEQ ID NO:22 and a light chain variable region comprising CDRs comprising the amino acid sequences of the CDRs of the light chain variable region set forth in SEQ ID NO:23.

21. The method of any one of claims 1 to 20, wherein:
(i) the antibody or antigen binding fragment thereof comprises CDR-H1 comprising the amino acid sequence of SEQ ID NO:9, CDR-H2 comprising the amino acid sequence of SEQ ID NO:10, CDR-H3 comprising the amino acid sequence of SEQ ID NO:11; CDR-L1 comprising the amino acid sequence of SEQ ID NO:12, CDR-L2 comprising the amino acid sequence of SEQ ID NO:13, and CDR-L3 comprising the amino acid sequence of SEQ ID NO:14;
(ii) the antibody or antigen binding fragment thereof comprises CDR-H1 comprising the amino acid sequence of SEQ ID NO:16, CDR-H2 comprising the amino acid sequence of SEQ ID NO:17, CDR-H3 comprising the amino acid sequence of SEQ ID NO:18; CDR-L1 comprising the amino acid sequence of SEQ ID NO:19, CDR-L2 comprising the amino acid sequence of SEQ ID NO:20, and CDR-L3 comprising the amino acid sequence of SEQ ID NO:21;
(iii) the antibody or antigen binding fragment thereof comprises CDR-H1 consisting of the amino acid sequence of SEQ ID NO:9, CDR-H2 consisting of the amino acid sequence of SEQ ID NO:10, CDR-H3 consisting of the amino acid sequence of SEQ ID NO:11; CDR-L1 consisting of the amino acid sequence of SEQ ID NO:12, CDR-L2 consisting of the amino acid sequence of SEQ ID NO:13, and CDR-L3 consisting of the amino acid sequence of SEQ ID NO:14, or
(iv) the antibody or antigen binding fragment thereof comprises CDR-H1 consisting of the amino acid sequence of SEQ ID NO:16, CDR-H2 consisting of the amino acid sequence of SEQ ID NO:17, CDR-H3 consisting of the amino acid sequence of SEQ ID NO:18; CDR-L1 consisting of the amino acid sequence of SEQ ID NO:19, CDR-L2 consisting of the amino acid sequence of SEQ ID NO:20, and CDR-L3 consisting of the amino acid sequence of SEQ ID NO:21.

22. The method of any one of claims 1 to 21, wherein the antibody or antigen binding fragment thereof comprises a heavy chain variable region comprising the amino acid sequence of SEQ ID NO:22 and a light chain variable region comprising the amino acid sequence of SEQ ID NO:23.

23. The method of any one of claims 1 to 22, wherein the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8.

24. The method of any one of claims 1 to 22, wherein the antigen binding fragment is an Fab, F(ab′)2, Fv, or scFv.

25. The method of any one of claims 1 to 23, wherein the antibody is a fully human antibody.

26. The method of any one of claims 1 to 23 and 25, wherein the antibody is an IgG1 and the light chain is a kappa light chain.

27. The method of any one of claims 1 to 26, wherein the antibody or antigen binding fragment thereof is recombinantly produced.

28. The method of any one of claims 1 to 27, wherein the antibody or antigen binding fragment is conjugated to each unit of MMAE via a linker; optionally wherein the linker is an enzyme-cleavable linker, and wherein the linker forms a bond with a sulfur atom of the antibody or antigen binding fragment thereof; and optionally wherein the linker has a formula of: -Aₐ-W_{w}-Y_{y}-; wherein -A- is a stretcher unit, a is 0 or 1; -W- is an amino acid unit, w is an integer ranging from 0 to 12; and -Y- is a spacer unit, y is 0, 1, or 2.

29. The method of claim 28, wherein the stretcher unit has the structure of Formula (1) below; the amino acid unit is valine-citrulline; and the spacer unit is a PAB group comprising the structure of Formula (2) below: 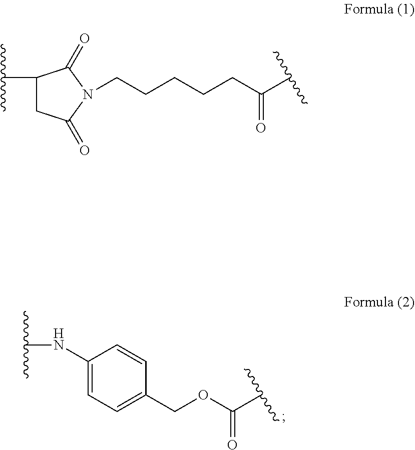 [SMILES: *C(=O)CCCCCN1C(=O)CC(*)C1=O ; *Nc1ccc(COC(*)=O)cc1]
wherein optionally the stretcher unit forms a bond with a sulfur atom of the antibody or antigen binding fragment thereof; and wherein the spacer unit is linked to MMAE via a carbamate group.

30. The method of any one of claims 1 to 29, wherein the ADC comprises:
(i) from 1 to 20 units of MMAE per antibody or antigen binding fragment thereof;
(ii) from 1 to 10 units of MMAE per antibody or antigen binding fragment thereof;
(iii) from 2 to 8 units of MMAE per antibody or antigen binding fragment thereof; or
(iv) from 3 to 5 units of MMAE per antibody or antigen binding fragment thereof.

31. The method of any one of claims 1 to 30, wherein the ADC has the following structure: 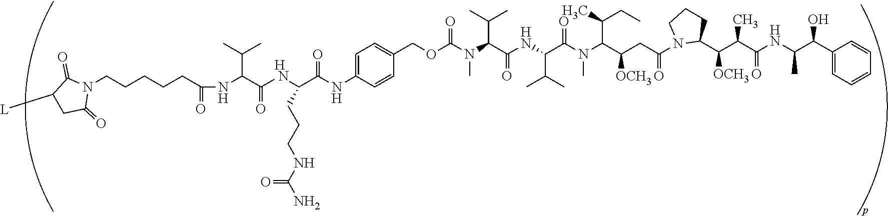
wherein L- represents the antibody or antigen binding fragment thereof and p is from 1 to 10.

32. The method of claim 31, wherein:
(i) p is from 2 to 8;
(ii) p is from 3 to 5;
(iii) p is from 3 to 4; or
(iv) p is about 4.

33. The method of claim 31 or 32, wherein the average p value of the effective amount of the antibody drug conjugate is about 3.8.

34. The method of any one of claims 1 to 33, wherein the ADC is formulated in a pharmaceutical composition comprising L-histidine, polysorbate-20 (TWEEN-20), and trehalose dehydrate.

35. The method of any one of claims 1 to 34, wherein the ADC is formulated in (i) a pharmaceutical composition comprising about 20 mM L-histidine, about 0.02% (w/v) TWEEN-20, about 5.5% (w/v) trehalose dihydrate, and hydrochloride, and wherein the pH of the pharmaceutical composition is about 6.0 at 25° C.; or (ii) a pharmaceutical composition comprising about 9 mM histidine, about 11 mM histidine hydrochloride monohydrate, about 0.02% (w/v) TWEEN-20, and about 5.5% (w/v) trehalose dihydrate, and wherein the pH of the pharmaceutical composition is about 6.0 at 25° C.

36. The method of any one of claims 1 to 35, wherein the ADC has the following structure: 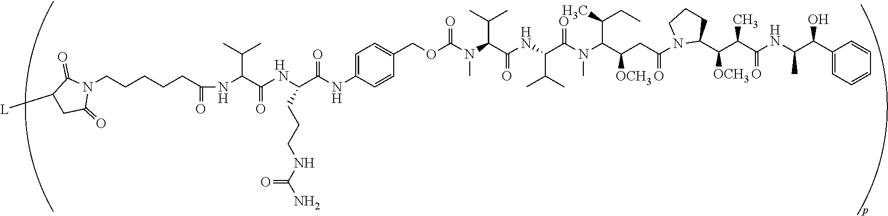
wherein L- represents the antibody or antigen binding fragment thereof and p is from about 3 to about 4, the antibody comprises a heavy chain comprising the amino acid sequence ranging from the 20th amino acid (glutamic acid) to the 466th amino acid (lysine) of SEQ ID NO:7 and a light chain comprising the amino acid sequence ranging from the 23rd amino acid (aspartic acid) to the 236th amino acid (cysteine) of SEQ ID NO:8, wherein the ADC is administered at a dose of about 1.25 mg/kg of the subject's body weight, and wherein the dose is administered by an IV infusion on Days 1 and 8 of every three-week cycle.

37. The method of any one of claims 1 to 36, wherein the subject has cT2-T4aN0M0 stage MIBC.

38. The method of any one of claims 1 to 37, wherein the subject is considered cisplatin ineligible if one or more of the following criteria are satisfied: (a) GFR<60 mL/min but ≥30 mL/min, (wherein the GFR is measured by the Cockcroft-Gault formula, Modification of Diet in Renal Disease equations (MDRD), or 24-hour urine collection); (b) ECOG performance status of 2; (c) NCI CTCAE Version 4.03 Grade ≥2 hearing loss; and (d) NYHA Class III heart failure.

39. The method of any one of claims 1 to 38, wherein the subject has not received prior systemic treatment, chemoradiation, and/or radiation therapy for MIBC.

40. The method of any one of claims 1 to 39, wherein the subject has not received an immune checkpoint inhibitor (CPI); wherein optionally the CPI is a programmed cell death 1 (PD-1) inhibitor or a programmed cell death-ligand 1 (PD-L1) inhibitor.

41. The method of any one of claims 1 to 40, wherein the subject has not received a CD137 agonist, a CTLA-4 inhibitor, or an OX-40 agonist.

42. The method of any one of claims 1 to 41, wherein the effective amount of the ADC is about 1 to about 10 mg/kg, about 1 to about 5 mg/kg, about 1 to about 2.5 mg/kg, about 1 to about 1.25 mg/kg, about 0.25 mg/kg, about 0.5 mg/kg, about 0.75 mg/kg, about 1.0 mg/kg, about 1.25 mg/kg, about 1.5 mg/kg, about 1.75 mg/kg, about 2.0 mg/kg, about 2.25 mg/kg, or about 2.5 mg/kg of the subject's body weight.

43. The method of any one of claims 1 to 41, wherein the effective amount of the ADC is about 1 mg/kg of the subject's body weight.

44. The method of any one of claims 1 to 41, wherein the effective amount of the ADC is about 1.25 mg/kg of the subject's body weight.

45. The method of claim 1 or claim 2, wherein in (a) the effective amount of the ADC is administered to the subject on days 1 and 8 of every 3 week cycle for a total of 3 cycles.

46. The method of claim 45, wherein the effective amount of the ADC is about 1.25 mg/kg of the subject's body weight.

47. The method of claim 4 or claim 5, wherein in (a) the effective amount of the ADC is administered to the subject on days 1 and 8 of every 3 week cycle for a total of 3 cycles.

48. The method of any one of claims 4, 5, or 47, wherein the method further comprises (b) about 8 weeks after the subject receives the RC+PLND, administering to the subject on days 1 and 8 of every 3 week cycle the effective amount of the ADC for a total of 6 cycles.

49. The method of claim 47 or claim 48, wherein the effective amount of the ADC is about 1.25 mg/kg of the subject's body weight.

50. The method of claim 6 or claim 7, wherein in (a) the effective amount of the ADC is administered to the subject on days 1 and 8 of every 3 week cycle for a total of 3 cycles.

51. The method of any one of claims 6, 7, or 50, further comprising (c) about 8 weeks after (b), administering to the subject on days 1 and 8 of every 3 week cycle the effective amount of the ADC for a total of 3 cycles.

52. The method of claim 50 or claim 51, wherein the effective amount of the ADC is about 1.25 mg/kg of the subject's body weight.

53. The method of any one of claims 8 to 44, wherein the effective amount of the ADC is administered to the human subject on days 1 and 8 of a 3 week cycle for a total of 3 cycles.

54. The method of any one of claims 1 to 53, wherein the ADC is administered by intravenous (IV) injection or infusion.

55. The method of any one of claims 1 to 54, wherein the cancer is muscle invasive urothelial cancer (MIUC).

56. The method of any one of claims 1 to 54, wherein the cancer is muscle invasive bladder cancer (MIBC).

57. The method of any one of claims 1 to 56, wherein the ADC is enfortumab vedotin (EV).

58. The method of any one of claims 1 to 57, wherein the overall survival of the subject is extended by at least 2, at least 4, at least 6, at least 8, at least 10, or at least 12 months.

59. The method of any one of claims 1 to 58, wherein the ADC is administered as a monotherapy.
